SEQUENCE LISTING (743 sequences)

SEQ ID NO: 1 - protein, 122 aa, synthetic construct
  REGION 1..122: note = artificial antibody-related sequence
QVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSS

SEQ ID NO: 2 - protein, 122 aa, synthetic construct
  REGION 1..122: note = artificial antibody-related sequence
QVQLQESGPGLVKPSETLSLTCKVSGFSFNSDYWIYWIRQPPGKGLEWIGSIYGGSSGNTQYASWAQGRVTISVDSSKNQFSLKLSSVTAADSAVYYCARGYVDYGGATDLWGQGTLVKVSS

SEQ ID NO: 3 - protein, 114 aa, synthetic construct
  REGION 1..114: note = artificial antibody-related sequence
DIQMTQSPSSLSASVGDRVTITCQASQSIGTYLAWYQQKPGKAPKLLIYRAFILASGVPSRFSGSGSGTDFTLTISSLQPEDFATYYCQSNFYSDSTTIGPNAFGTGTKVTVLG

SEQ ID NO: 4 - protein, 114 aa, synthetic construct
  REGION 1..114: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLG

SEQ ID NO: 5 - protein, 256 aa, synthetic construct
  REGION 1..256: note = artificial antibody-related sequence
DIQMTQSPSSLSASVGDRVTITCQASQSIGTYLAWYQQKPGKAPKLLIYRAFILASGVPSRFSGSGSGTDFTLTISSLQPEDFATYYCQSNFYSDSTTIGPNAFGTGTKVTVLGGGGGSGGGGSGGGGSGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSS

SEQ ID NO: 6 - protein, 256 aa, synthetic construct
  REGION 1..256: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSGGGGSGGGGSGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSS

SEQ ID NO: 7 - protein, 256 aa, synthetic construct
  REGION 1..256: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSGGGGSGGGGSGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADSAVYYCARGYVDYGGATDLWGQGTLVKVSS

SEQ ID NO: 8 - protein, 122 aa, synthetic construct
  REGION 1..122: note = artificial antibody-related sequence
QVQLVQSGAEVKKPGASVKVSCKASGFSFNSDYWIYWVRQAPGQGLEWMGSIYGGSSGNTQYASWAQGRVTMTRDTSISTAYMELSSLRSEDTAVYYCARGYVDYGGATDLWGQGTLVTVSS

SEQ ID NO: 9 - protein, 122 aa, synthetic construct
  REGION 1..122: note = artificial antibody-related sequence
QVQLVQSGAEVKKPGASVKVSCKASGFSFNSDYWIYWVRQAPGQGLEWMGSIYGGSSGNTQYASWAQGRVTMTRDTSISTAYMELSSLRSEDSAVYYCARGYVDYGGATDLWGQGTLVKVSS

SEQ ID NO: 10 - protein, 114 aa, synthetic construct; identical to SEQ ID NO: 3
  REGION 1..114: note = artificial antibody-related sequence

SEQ ID NO: 11 - protein, 114 aa, synthetic construct; identical to SEQ ID NO: 4
  REGION 1..114: note = artificial antibody-related sequence

SEQ ID NO: 12 - protein, 256 aa, synthetic construct
  REGION 1..256: note = artificial antibody-related sequence
DIQMTQSPSSLSASVGDRVTITCQASQSIGTYLAWYQQKPGKAPKLLIYRAFILASGVPSRFSGSGSGTDFTLTISSLQPEDFATYYCQSNFYSDSTTIGPNAFGTGTKVTVLGGGGGSGGGGSGGGGSGGGGSQVQLVQSGAEVKKPGASVKVSCKASGFSFNSDYWIYWVRQAPGQGLEWMGSIYGGSSGNTQYASWAQGRVTMTRDTSISTAYMELSSLRSEDTAVYYCARGYVDYGGATDLWGQGTLVTVSS

SEQ ID NO: 13 - protein, 256 aa, synthetic construct
  REGION 1..256: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSGGGGSGGGGSGGGGSQVQLVQSGAEVKKPGASVKVSCKASGFSFNSDYWIYWVRQAPGQGLEWMGSIYGGSSGNTQYASWAQGRVTMTRDTSISTAYMELSSLRSEDTAVYYCARGYVDYGGATDLWGQGTLVTVSS

SEQ ID NO: 14 - protein, 256 aa, synthetic construct
  REGION 1..256: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSGGGGSGGGGSGGGGSQVQLVQSGAEVKKPGASVKVSCKASGFSFNSDYWIYWVRQAPGQGLEWMGSIYGGSSGNTQYASWAQGRVTMTRDTSISTAYMELSSLRSEDSAVYYCARGYVDYGGATDLWGQGTLVKVSS

SEQ ID NO: 15 - protein, 122 aa, synthetic construct
  REGION 1..122: note = artificial antibody-related sequence
EVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 16 - protein, 122 aa, synthetic construct
  REGION 1..122: note = artificial antibody-related sequence
EVQLVESGGGLVQPGGSLRLSCAASGFSFNSDYWISWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 17 - protein, 122 aa, synthetic construct
  REGION 1..122: note = artificial antibody-related sequence
EVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSS

SEQ ID NO: 18 - protein, 122 aa, synthetic construct
  REGION 1..122: note = artificial antibody-related sequence
EVQLVESGGGLVQPGGSLRLSCAASGFSFNSDYWISWVRQAPGKGLEWIASIYGGSSGNTQYASWAQGRFTISRDNSKNTVYLQMNSLRAEDSAVYFCARGYVDYGGATDLWGQGTLVKVSS

SEQ ID NO: 19 - protein, 114 aa, synthetic construct
  REGION 1..114: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLG

SEQ ID NO: 20 - protein, 114 aa, synthetic construct
  REGION 1..114: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLG

SEQ ID NO: 21 - protein, 114 aa, synthetic construct
  REGION 1..114: note = artificial antibody-related sequence
DIQMTQSPSSLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLG

SEQ ID NO: 22 - protein, 114 aa, synthetic construct
  REGION 1..114: note = artificial antibody-related sequence
DIQMTQSPSSLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLG

SEQ ID NO: 23 - protein, 256 aa, synthetic construct
  REGION 1..256: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 24 - protein, 256 aa, synthetic construct
  REGION 1..256: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 25 - protein, 256 aa, synthetic construct
  REGION 1..256: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSS

SEQ ID NO: 26 - protein, 256 aa, synthetic construct
  REGION 1..256: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWISWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 27 - protein, 256 aa, synthetic construct
  REGION 1..256: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWISWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 28 - protein, 256 aa, synthetic construct
  REGION 1..256: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWISWVRQAPGKGLEWIASIYGGSSGNTQYASWAQGRFTISRDNSKNTVYLQMNSLRAEDSAVYFCARGYVDYGGATDLWGQGTLVKVSS

SEQ ID NO: 29 - protein, 256 aa, synthetic construct
  REGION 1..256: note = artificial antibody-related sequence
DIQMTQSPSSLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 30 - protein, 256 aa, synthetic construct
  REGION 1..256: note = artificial antibody-related sequence
DIQMTQSPSSLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSS

SEQ ID NO: 31 - protein, 121 aa, synthetic construct
  REGION 1..121: note = artificial antibody-related sequence
QQLVESGGGLVQPGGSLRLSCAASGFSFNSDYWISWVRQAPGKGLEWIASIYGGSSGNTQYASWAQGRFTISKDNSKNTVYLQMNSLRAEDSAVYFCARGYVDYGGATDLWGQGTLVKVSS

SEQ ID NO: 32 - protein, 114 aa, synthetic construct; identical to SEQ ID NO: 21
  REGION 1..114: note = artificial antibody-related sequence

SEQ ID NO: 33 - protein, 114 aa, synthetic construct; identical to SEQ ID NO: 22
  REGION 1..114: note = artificial antibody-related sequence

SEQ ID NO: 34 - protein, 255 aa, synthetic construct
  REGION 1..255: note = artificial antibody-related sequence
DIQMTQSPSSLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSGGGGSGGGGSGGGGSQQLVESGGGLVQPGGSLRLSCAASGFSFNSDYWISWVRQAPGKGLEWIASIYGGSSGNTQYASWAQGRFTISKDNSKNTVYLQMNSLRAEDSAVYFCARGYVDYGGATDLWGQGTLVKVSS

SEQ ID NO: 35 - protein, 255 aa, synthetic construct
  REGION 1..255: note = artificial antibody-related sequence
DIQMTQSPSSLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSQQLVESGGGLVQPGGSLRLSCAASGFSFNSDYWISWVRQAPGKGLEWIASIYGGSSGNTQYASWAQGRFTISKDNSKNTVYLQMNSLRAEDSAVYFCARGYVDYGGATDLWGQGTLVKVSS

SEQ ID NO: 36 - protein, 121 aa, synthetic construct
  REGION 1..121: note = artificial antibody-related sequence
QSLVESGGGLVQPGGSLRLSCAASGFSFNSDYWISWVRQAPGKGLEWIASIYGGSSGNTQYASWAQGRFTISKDNSKNTVYLQMNSLRAEDTAVYFCARGYVDYGGATDLWGQGTLVTVSS

SEQ ID NO: 37 - protein, 121 aa, synthetic construct
  REGION 1..121: note = artificial antibody-related sequence
QSLVESGGGLVQPGGSLRLSCAASGFSFNSDYWISWVRQAPGKGLEWIASIYGGSSGNTQYASWAQGRFTISKDNSKNTVYLQMNSLRAEDSAVYFCARGYVDYGGATDLWGQGTLVKVSS

SEQ ID NO: 38 - protein, 114 aa, synthetic construct; identical to SEQ ID NO: 21
  REGION 1..114: note = artificial antibody-related sequence

SEQ ID NO: 39 - protein, 114 aa, synthetic construct; identical to SEQ ID NO: 22
  REGION 1..114: note = artificial antibody-related sequence

SEQ ID NO: 40 - protein, 255 aa, synthetic construct
  REGION 1..255: note = artificial antibody-related sequence
DIQMTQSPSSLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSGGGGSGGGGSGGGGSQSLVESGGGLVQPGGSLRLSCAASGFSFNSDYWISWVRQAPGKGLEWIASIYGGSSGNTQYASWAQGRFTISKDNSKNTVYLQMNSLRAEDTAVYFCARGYVDYGGATDLWGQGTLVTVSS

SEQ ID NO: 41 - protein, 255 aa, synthetic construct
  REGION 1..255: note = artificial antibody-related sequence
DIQMTQSPSSLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSQSLVESGGGLVQPGGSLRLSCAASGFSFNSDYWISWVRQAPGKGLEWIASIYGGSSGNTQYASWAQGRFTISKDNSKNTVYLQMNSLRAEDTAVYFCARGYVDYGGATDLWGQGTLVTVSS

SEQ ID NO: 42 - protein, 255 aa, synthetic construct
  REGION 1..255: note = artificial antibody-related sequence
DIQMTQSPSSLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSQSLVESGGGLVQPGGSLRLSCAASGFSFNSDYWISWVRQAPGKGLEWIASIYGGSSGNTQYASWAQGRFTISKDNSKNTVYLQMNSLRAEDSAVYFCARGYVDYGGATDLWGQGTLVKVSS

SEQ ID NO: 43 - protein, 122 aa, synthetic construct
  REGION 1..122: note = artificial antibody-related sequence
EVQLVESGGGLVQPGGSLRLSCAASGFSFNSDYWIYWVRQAPGKGLEWIACIYGGSSGNTQYASWAQGRFTISRDNSKNTVYLQMNSLRAEDTAVYFCARGYVDYGGATDLWGQGTLVTVSS

SEQ ID NO: 44 - protein, 122 aa, synthetic construct
  REGION 1..122: note = artificial antibody-related sequence
EVQLVESGGGLVQPGGSLRLSCAASGFSFNSDYWIYWVRQAPGKGLEWIACIYGGSSGNTQYASWAQGRFTISRDNSKNTVYLQMNSLRAEDSAVYFCARGYVDYGGATDLWGQGTLVKVSS

SEQ ID NO: 45 - protein, 114 aa, synthetic construct; identical to SEQ ID NO: 19
  REGION 1..114: note = artificial antibody-related sequence

SEQ ID NO: 46 - protein, 114 aa, synthetic construct; identical to SEQ ID NO: 20
  REGION 1..114: note = artificial antibody-related sequence

SEQ ID NO: 47 - protein, 255 aa, synthetic construct
  REGION 1..255: note = artificial antibody-related sequence
IQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIACIYGGSSGNTQYASWAQGRFTISRDNSKNTVYLQMNSLRAEDTAVYFCARGYVDYGGATDLWGQGTLVTVSS

SEQ ID NO: 48 - protein, 256 aa, synthetic construct
  REGION 1..256: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIACIYGGSSGNTQYASWAQGRFTISRDNSKNTVYLQMNSLRAEDTAVYFCARGYVDYGGATDLWGQGTLVTVSS

SEQ ID NO: 49 - protein, 256 aa, synthetic construct
  REGION 1..256: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIACIYGGSSGNTQYASWAQGRFTISRDNSKNTVYLQMNSLRAEDSAVYFCARGYVDYGGATDLWGQGTLVKVSS

SEQ ID NO: 50 - protein, 121 aa, synthetic construct
  REGION 1..121: note = artificial antibody-related sequence
QVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSS

SEQ ID NO: 51 - protein, 121 aa, synthetic construct
  REGION 1..121: note = artificial antibody-related sequence
QVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSS

SEQ ID NO: 52 - protein, 111 aa, synthetic construct
  REGION 1..111: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLG

SEQ ID NO: 53 - protein, 111 aa, synthetic construct
  REGION 1..111: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLG

SEQ ID NO: 54 - protein, 252 aa, synthetic construct
  REGION 1..252: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSS

SEQ ID NO: 55 - protein, 252 aa, synthetic construct
  REGION 1..252: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSS

SEQ ID NO: 56 - protein, 252 aa, synthetic construct
  REGION 1..252: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSS

SEQ ID NO: 57 - protein, 121 aa, synthetic construct
  REGION 1..121: note = artificial antibody-related sequence
QSQLQESGPGLVKPSETLSLTCKASGFSFSSGYDMCWVRQPPGKGLEWIACVVAGSVDITYYASWAKGRVTISKDSSKNQVSLKLSSVTAADTAVYFCARKDAYSDAFNLWGQGTLVTVSS

SEQ ID NO: 58 - protein, 121 aa, synthetic construct
  REGION 1..121: note = artificial antibody-related sequence
QSQLQESGPGLVKPSETLSLTCKASGFSFSSGYDMCWVRQPPGKGLEWIACVVAGSVDITYYASWAKGRVTISKDSSKNQVSLKLSSVTAADSAVYFCARKDAYSDAFNLWGQGTLVKVSS

SEQ ID NO: 59 - protein, 111 aa, synthetic construct
  REGION 1..111: note = artificial antibody-related sequence
DFQLTQSPSSLSASVGDRVTITCQASQSINDYLAWYQQKPGKSPKLLIYKASTLASGVPSRFSGSGSGTDFTLTISSLQPEDFATYYCQQGYIITDIDNVFGTGTKVTVLG

SEQ ID NO: 60 - protein, 111 aa, synthetic construct
  REGION 1..111: note = artificial antibody-related sequence
DFQLTQSPSSLSASVGDRVTITCQASQSINDYLAWYQQKPGKSPKLLIYKGGTLASGVPSRFSGSGSGTDFTLTISSLQPEDFATYYCQQGYIITDIDNVFGTGTKVTVLG

SEQ ID NO: 61 - protein, 252 aa, synthetic construct
  REGION 1..252: note = artificial antibody-related sequence
DFQLTQSPSSLSASVGDRVTITCQASQSINDYLAWYQQKPGKSPKLLIYKASTLASGVPSRFSGSGSGTDFTLTISSLQPEDFATYYCQQGYIITDIDNVFGTGTKVTVLGGGGGSGGGGSGGGGSGGGGSQSQLQESGPGLVKPSETLSLTCKASGFSFSSGYDMCWVRQPPGKGLEWIACVVAGSVDITYYASWAKGRVTISKDSSKNQVSLKLSSVTAADTAVYFCARKDAYSDAFNLWGQGTLVTVSS

SEQ ID NO: 62 - protein, 252 aa, synthetic construct
  REGION 1..252: note = artificial antibody-related sequence
DFQLTQSPSSLSASVGDRVTITCQASQSINDYLAWYQQKPGKSPKLLIYKGGTLASGVPSRFSGSGSGTDFTLTISSLQPEDFATYYCQQGYIITDIDNVFGTGTKVTVLGGGGGSGGGGSGGGGSGGGGSQSQLQESGPGLVKPSETLSLTCKASGFSFSSGYDMCWVRQPPGKGLEWIACVVAGSVDITYYASWAKGRVTISKDSSKNQVSLKLSSVTAADTAVYFCARKDAYSDAFNLWGQGTLVTVSS

SEQ ID NO: 63 - protein, 252 aa, synthetic construct
  REGION 1..252: note = artificial antibody-related sequence
DFQLTQSPSSLSASVGDRVTITCQASQSINDYLAWYQQKPGKSPKLLIYKGGTLASGVPSRFSGSGSGTDFTLTISSLQPEDFATYYCQQGYIITDIDNVFGTGTKVTVLGGGGGSGGGGSGGGGSGGGGSQSQLQESGPGLVKPSETLSLTCKASGFSFSSGYDMCWVRQPPGKGLEWIACVVAGSVDITYYASWAKGRVTISKDSSKNQVSLKLSSVTAADSAVYFCARKDAYSDAFNLWGQGTLVKVSS

SEQ ID NO: 64 - protein, 121 aa, synthetic construct
  REGION 1..121: note = artificial antibody-related sequence
QVQLQESGPGLVKPSETLSLTCKASGFSFSSGYDMCWVRQPPGKGLEWIACVVAGSVDITYYASWAKGRVTISKDSSKNQVSLKLSSVTAADTAVYYCARKDAYSDAFNLWGQGTLVTVSS

SEQ ID NO: 65 - protein, 121 aa, synthetic construct
  REGION 1..121: note = artificial antibody-related sequence
QVQLQESGPGLVKPSETLSLTCKASGFSFSSGYDMCWVRQPPGKGLEWIACVVAGSVDITYYADSVKGRVTISKDSSKNQVSLKLSSVTAADTAVYYCARKDAYSDAFNLWGQGTLVTVSS

SEQ ID NO: 66 - protein, 121 aa, synthetic construct
  REGION 1..121: note = artificial antibody-related sequence
QVQLQESGPGLVKPSETLSLTCKASGFSFSSGYDMCWVRQPPGKGLEWIACVVAGSVDITYYASWAKGRVTISKDSSKNQVSLKLSSVTAADSAVYYCARKDAYSDAFNLWGQGTLVKVSS

SEQ ID NO: 67 - protein, 111 aa, synthetic construct; identical to SEQ ID NO: 52
  REGION 1..111: note = artificial antibody-related sequence

SEQ ID NO: 68 - protein, 111 aa, synthetic construct; identical to SEQ ID NO: 53
  REGION 1..111: note = artificial antibody-related sequence

SEQ ID NO: 69 - protein, 111 aa, synthetic construct
  REGION 1..111: note = artificial antibody-related sequence
DIQMTQSPSSLSASVGDRVTITCQASQSINDYLAWYQQKPGKAPKLLIYKGGTLASGVPSRFSGRGSGTDFTLTISSLQPEDFATYYCQQGYIITDIDNVFGTGTKVTVLG

SEQ ID NO: 70 - protein, 252 aa, synthetic construct
  REGION 1..252: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSGGGGSGGGGSGGGGSQVQLQESGPGLVKPSETLSLTCKASGFSFSSGYDMCWVRQPPGKGLEWIACVVAGSVDITYYASWAKGRVTISKDSSKNQVSLKLSSVTAADTAVYYCARKDAYSDAFNLWGQGTLVTVSS

SEQ ID NO: 71 - protein, 252 aa, synthetic construct
  REGION 1..252: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSGGGGSGGGGSGGGGSQVQLQESGPGLVKPSETLSLTCKASGFSFSSGYDMCWVRQPPGKGLEWIACVVAGSVDITYYASWAKGRVTISKDSSKNQVSLKLSSVTAADTAVYYCARKDAYSDAFNLWGQGTLVTVSS

SEQ ID NO: 72 - protein, 252 aa, synthetic construct
  REGION 1..252: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSGGGGSGGGGSGGGGSQVQLQESGPGLVKPSETLSLTCKASGFSFSSGYDMCWVRQPPGKGLEWIACVVAGSVDITYYASWAKGRVTISKDSSKNQVSLKLSSVTAADSAVYYCARKDAYSDAFNLWGQGTLVKVSS

SEQ ID NO: 73 - protein, 252 aa, synthetic construct
  REGION 1..252: note = artificial antibody-related sequence
DIQMTQSPSSLSASVGDRVTITCQASQSINDYLAWYQQKPGKAPKLLIYKGGTLASGVPSRFSGRGSGTDFTLTISSLQPEDFATYYCQQGYIITDIDNVFGTGTKVTVLGGGGGSGGGGSGGGGSGGGGSQVQLQESGPGLVKPSETLSLTCKASGFSFSSGYDMCWVRQPPGKGLEWIACVVAGSVDITYYADSVKGRVTISKDSSKNQVSLKLSSVTAADTAVYYCARKDAYSDAFNLWGQGTLVTVSS

SEQ ID NO: 74 - protein, 122 aa, synthetic construct
  REGION 1..122: note = artificial antibody-related sequence
EVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSS

SEQ ID NO: 75 - protein, 122 aa, synthetic construct
  REGION 1..122: note = artificial antibody-related sequence
EVQLVESGGGLVQPGGSLRLSCAASGFSFSANYYPCWVRQAPGKCLEWIGCIYGGSSDITYDANWTKGRFTISRDNSKNTVYLQMNSLRAEDTAVYYCARSAWYSGWGGDLWGQGTLVTVSS

SEQ ID NO: 76 - protein, 122 aa, synthetic construct
  REGION 1..122: note = artificial antibody-related sequence
EVQLVESGGGLVQPGGSLRLSCAASGFSFSANYYPCWVRQAPGKGLEWIGCIYGGSSDITYDAQWTKGRFTISRDNSKNTVYLQMNSLRAEDTAVYYCARSAWYSGWGGDLWGQGTLVTVSS

SEQ ID NO: 77 - protein, 122 aa, synthetic construct
  REGION 1..122: note = artificial antibody-related sequence
EVQLVESGGGLVQPGGSLRLSCAASGFSFSANYYPCWVRQAPGKCLEWIGCIYGGSSDITYDAQWTKGRFTISRDNSKNTVYLQMNSLRAEDTAVYYCARSAWYSGWGGDLWGQGTLVTVSS

SEQ ID NO: 78 - protein, 110 aa, synthetic construct
  REGION 1..110: note = artificial antibody-related sequence
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLG

SEQ ID NO: 79 - protein, 110 aa, synthetic construct
  REGION 1..110: note = artificial antibody-related sequence
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGCGTKVTVLG

SEQ ID NO: 80 - protein, 110 aa, synthetic construct
  REGION 1..110: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLG

SEQ ID NO: 81 - protein, 110 aa, synthetic construct
  REGION 1..110: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGCGTKVTVLG

SEQ ID NO: 82 - protein, 252 aa, synthetic construct
  REGION 1..252: note = artificial antibody-related sequence
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSGGGGSGGGGSGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSS

SEQ ID NO: 83 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSGGGGSGGGGSGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSS

SEQ ID NO: 84 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSGGGGSGGGGSGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDSAVYYCARSAWYSGWGGDLWGQGTLVKVSS

SEQ ID NO: 85 - protein, 252 aa, synthetic construct
  REGION 1..252: note = artificial antibody-related sequence
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSGGGGSGGGGSGGGGSEVQLVESGGGLVQPGGSLRLSCAASGFSFSANYYPCWVRQAPGKTLEWIGCIYGGSSDITYDAQWTKGRFTISRDNSKNTVYLQMNSLRAEDTAVYYCARSAWYSGWGGDLWGQGTLVTVSS

SEQ ID NO: 86 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSGGGGSGGGGSGGGGSEVQLVESGGGLVQPGGSLRLSCAASGFSFSANYYPCWVRQAPGKGLEWIGCIYGGSSDITYDAQWTKGRFTISRDNSKNTVYLQMNSLRAEDTAVYYCARSAWYSGWGGDLWGQGTLVTVSS

SEQ ID NO: 87 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSGGGGSGGGGSGGGGSEVQLVESGGGLVQPGGSLRLSCAASGFSFSANYYPCWVRQAPGKGLEWIGCIYGGSSDITYDAQWTKGRFTISRDNSKNTVYLQMNSLRAEDSAVYYCARSAWYSGWGGDLWGQGTLVKVSS

SEQ ID NO: 88 - protein, 252 aa, synthetic construct
  REGION 1..252: note = artificial antibody-related sequence
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGCGTKVTVLGGGGGSGGGGSGGGGSGGGGSEVQLVESGGGLVQPGGSLRLSCAASGFSFSANYYPCWVRQAPGKCLEWIGCIYGGSSDITYDANWTKGRFTISRDNSKNTVYLQMNSLRAEDTAVYYCARSAWYSGWGGDLWGQGTLVTVSS

SEQ ID NO: 89 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGCGTKVTVLGGGGGSGGGGSGGGGSGGGGSEVQLVESGGGLVQPGGSLRLSCAASGFSFSANYYPCWVRQAPGKCLEWIGCIYGGSSDITYDANWTKGRFTISRDNSKNTVYLQMNSLRAEDTAVYYCARSAWYSGWGGDLWGQGTLVTVSS

SEQ ID NO: 90 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGCGTKVTVLGGGGGSGGGGSGGGGSGGGGSEVQLVESGGGLVQPGGSLRLSCAASGFSFSANYYPCWVRQAPGKCLEWIGCIYGGSSDITYDANWTKGRFTISRDNSKNTVYLQMNSLRAEDSAVYYCARSAWYSGWGGDLWGQGTLVKVSS

SEQ ID NO: 91 - protein, 252 aa, synthetic construct
  REGION 1..252: note = artificial antibody-related sequence
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGCGTKVTVLGGGGGSGGGGSGGGGSGGGGSEVQLVESGGGLVQPGGSLRLSCAASGFSFSANYYPCWVRQAPGKCLEWIGCIYGGSSDITYDAQWTKGRFTISRDNSKNTVYLQMNSLRAEDTAVYYCARSAWYSGWGGDLWGQGTLVTVSS

SEQ ID NO: 92 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGCGTKVTVLGGGGGSGGGGSGGGGSGGGGSEVQLVESGGGLVQPGGSLRLSCAASGFSFSANYYPCWVRQAPGKCLEWIGCIYGGSSDITYDAQWTKGRFTISRDNSKNTVYLQMNSLRAEDTAVYYCARSAWYSGWGGDLWGQGTLVTVSS

SEQ ID NO: 93 - protein, 252 aa, synthetic construct
  REGION 1..252: note = artificial antibody-related sequence
DIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGCGTKVTVLGGGGGSGGGGSGGGGSGGGGSEVQLVESGGGLVQPGGSLRLSCAASGFSFSANYYPCWVRQAPGKCLEWIGCIYGGSSDITYDAQWTKGRFTISRDNSKNTVYLQMNSLRAEDSAVYYCARSAWYSGWGGDLWGQGTLVKVSS

SEQ ID NO: 94 - protein, 122 aa, synthetic construct
  REGION 1..122: note = artificial antibody-related sequence
ESQLVESGGGLVQPGGSLRLSCAASGFSFSANYYPCWVRQAPGKGLEWIGCIYGGSSDITYDANWTKGRFTISRDNSKNTVYLQMNSLRAEDTAVYFCARSAWYSGWGGDLWGPGTLVTVSS

SEQ ID NO: 95 - protein, 122 aa, synthetic construct
  REGION 1..122: note = artificial antibody-related sequence
ESQLVESGGGLVQPGGSLRLSCAASGFSFSANYYPCWVRQAPGKGLEWIGCIYGGSSDITYDANWTKGRFTISRDNSKNTVYLQMNSLRAEDSAVYFCARSAWYSGWGGDLWGPGTLVKVSS

SEQ ID NO: 96 - protein, 110 aa, synthetic construct
  REGION 1..110: note = artificial antibody-related sequence
DFQLTQSPSSLSASVGDRVTITCQASQSISNRLAWYQQKPGKPPKLLIYSASTLASGVPSRFSGSGSGTDFTLTISSLQPEDFATYYCQSTYYGNDGNAFGTGTKVTVLG

SEQ ID NO: 97 - protein, 110 aa, synthetic construct
  REGION 1..110: note = artificial antibody-related sequence
DFQLTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGTGTKVTVLG

SEQ ID NO: 98 - protein, 252 aa, synthetic construct
  REGION 1..252: note = artificial antibody-related sequence
DFQLTQSPSSLSASVGDRVTITCQASQSISNRLAWYQQKPGKPPKLLIYSASTLASGVPSRFSGSGSGTDFTLTISSLQPEDFATYYCQSTYYGNDGNAFGTGTKVTVLGGGGGSGGGGSGGGGSGGGGSESQLVESGGGLVQPGGSLRLSCAASGFSFSANYYPCWVRQAPGKGLEWIGCIYGGSSDITYDANWTKGRFTISRDNSKNTVYLQMNSLRAEDTAVYFCARSAWYSGWGGDLWGPGTLVTVSS

SEQ ID NO: 99 - protein, 252 aa, synthetic construct
  REGION 1..252: note = artificial antibody-related sequence
DFQLTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGTGTKVTVLGGGGGSGGGGSGGGGSGGGGSESQLVESGGGLVQPGGSLRLSCAASGFSFSANYYPCWVRQAPGKGLEWIGCIYGGSSDITYDANWTKGRFTISRDNSKNTVYLQMNSLRAEDTAVYFCARSAWYSGWGGDLWGPGTLVTVSS

SEQ ID NO: 100 - protein, 252 aa, synthetic construct
  REGION 1..252: note = artificial antibody-related sequence
DFQLTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGTGTKVTVLGGGGGSGGGGSGGGGSGGGGSESQLVESGGGLVQPGGSLRLSCAASGFSFSANYYPCWVRQAPGKGLEWIGCIYGGSSDITYDANWTKGRFTISRDNSKNTVYLQMNSLRAEDSAVYFCARSAWYSGWGGDLWGPGTLVKVSS

SEQ ID NO: 101 - protein, 121 aa, synthetic construct
  REGION 1..121: note = artificial antibody-related sequence
QSLVESGGGLVQPGGSLRLSCAASGFSFSANYYPCWVRQAPGKGLEWIACIYGGSSDITYDANWTKGRFTISKDNSKNTVYLQMNSLRAEDSAVYFCARSAWYSGWGGDLWGQGTLVKVSS

SEQ ID NO: 102 - protein, 121 aa, synthetic construct
  REGION 1..121: note = artificial antibody-related sequence
QSLVESGGGLVQPGGSLRLSCAASGFSFSANYYPCWVRQAPGKCLEWIACIYGGSSDITYDANWTKGRFTISKDNSKNTVYLQMNSLRAEDSAVYFCARSAWYSGWGGDLWGQGTLVKVSS

SEQ ID NO: 103 - protein, 110 aa, synthetic construct
  REGION 1..110: note = artificial antibody-related sequence
DIQMTQSPSSLSASVGDRVTITCQASQSISNRLAWYQQKPGKPPKLLIYSASTLASGVPSRFSGSGSGTDFTLTISSLQPEDFATYYCQSTYYGNDGNAFGTGTKVTVLG

SEQ ID NO: 104 - protein, 110 aa, synthetic construct
  REGION 1..110: note = artificial antibody-related sequence
DIQMTQSPSSLSASVGDRVTITCQASQSISNRLAWYQQKPGKPPKLLIYSASTLASGVPSRFSGSGSGTDFTLTISSLQPEDFATYYCQSTYYGNDGNAFGCGTKVTVLG

SEQ ID NO: 105 - protein, 110 aa, synthetic construct
  REGION 1..110: note = artificial antibody-related sequence
DIQMTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGTGTKVTVLG

SEQ ID NO: 106 - protein, 110 aa, synthetic construct
  REGION 1..110: note = artificial antibody-related sequence
DIQMTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGCGTKVTVLG

SEQ ID NO: 107 - protein, 251 aa, synthetic construct
  REGION 1..251: note = artificial antibody-related sequence
DIQMTQSPSSLSASVGDRVTITCQASQSISNRLAWYQQKPGKPPKLLIYSASTLASGVPSRFSGSGSGTDFTLTISSLQPEDFATYYCQSTYYGNDGNAFGTGTKVTVLGGGGGSGGGGSGGGGSGGGGSQSLVESGGGLVQPGGSLRLSCAASGFSFSANYYPCWVRQAPGKGLEWIACIYGGSSDITYDANWTKGRFTISKDNSKNTVYLQMNSLRAEDSAVYFCARSAWYSGWGGDLWGQGTLVKVSS

SEQ ID NO: 108 - protein, 251 aa, synthetic construct
  REGION 1..251: note = artificial antibody-related sequence
DIQMTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGTGTKVTVLGGGGGSGGGGSGGGGSGGGGSQSLVESGGGLVQPGGSLRLSCAASGFSFSANYYPCWVRQAPGKGLEWIACIYGGSSDITYDANWTKGRFTISKDNSKNTVYLQMNSLRAEDSAVYFCARSAWYSGWGGDLWGQGTLVKVSS

SEQ ID NO: 109 - protein, 250 aa, synthetic construct
  REGION 1..250: note = artificial antibody-related sequence
IQMTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGCGTKVTVLGGGGGSGGGGSGGGGSGGGGSQSLVESGGGLVQPGGSLRLSCAASGFSFSANYYPCWVRQAPGKCLEWIACIYGGSSDITYDANWTKGRFTISKDNSKNTVYLQMNSLRAEDSAVYFCARSAWYSGWGGDLWGQGTLVKVSS

SEQ ID NO: 110 - protein, 124 aa, synthetic construct
  REGION 1..124: note = artificial antibody-related sequence
QVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSS

SEQ ID NO: 111 - protein, 124 aa, synthetic construct
  REGION 1..124: note = artificial antibody-related sequence
QVQLQESGPGLVKPSETLSLTCKVSGFSFSNSYWICWIRQPPGKGLEWIGCTFVGSSDSTYYANWAKGRVTISVDSSKNQFSLKLSSVTAADSAVYYCARHPSDAVYGYANNLWGQGTLVKVSS

SEQ ID NO: 112 - protein, 108 aa, synthetic construct
  REGION 1..108: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGTGTKVTVLG

SEQ ID NO: 113 - protein, 108 aa, synthetic construct
  REGION 1..108: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLG

SEQ ID NO: 114 - protein, 252 aa, synthetic construct
  REGION 1..252: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSS

SEQ ID NO: 115 - protein, 252 aa, synthetic construct
  REGION 1..252: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSS

SEQ ID NO: 116 - protein, 252 aa, synthetic construct
  REGION 1..252: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADSAVYYCARHPSDAVYGYANNLWGQGTLVKVSS

SEQ ID NO: 117 - protein, 124 aa, synthetic construct
  REGION 1..124: note = artificial antibody-related sequence
QVQLQESGPGLVKPSETLSLTCKVSGFSFSNSYWICWVRQPPGKGLEWIGCTFVGSSDSTYYANWAKGRVTISVDSSKNQVSLKLSSVTAADTAVYFCARHPSDAVYGYANNLWGQGTLVTVSS

SEQ ID NO: 118 - protein, 124 aa, synthetic construct
  REGION 1..124: note = artificial antibody-related sequence
QVQLQESGPGLVKPSETLSLTCKVSGFSFSNSYWICWVRQPPGKGLEWIGCTFVGSSDSTYYANWAKGRVTISVDSSKNQVSLKLSSVTAADSAVYFCARHPSDAVYGYANNLWGQGTLVKVSS

SEQ ID NO: 119 - protein, 108 aa, synthetic construct
  REGION 1..108: note = artificial antibody-related sequence
DIQMTQSPSSLSASVGDRVTITCQASQSINNVLAWYQQKPGKPPKLLIYRASTLASGVPSRFSGSGSGTDFTLTISSLQPEDFATYYCQSSYGNYGDFGTGTKVTVLG

SEQ ID NO: 120 - protein, 108 aa, synthetic construct
  REGION 1..108: note = artificial antibody-related sequence
DIQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGTGTKVTVLG

SEQ ID NO: 121 - protein, 252 aa, synthetic construct
  REGION 1..252: note = artificial antibody-related sequence
DIQMTQSPSSLSASVGDRVTITCQASQSINNVLAWYQQKPGKPPKLLIYRASTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKVSGFSFSNSYWICWVRQPPGKGLEWIGCTFVGSSDSTYYANWAKGRVTISVDSSKNQVSLKLSSVTAADTAVYFCARHPSDAVYGYANNLWGQGTLVTVSS

SEQ ID NO: 122 - protein, 252 aa, synthetic construct
  REGION 1..252: note = artificial antibody-related sequence
DIQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKVSGFSFSNSYWICWVRQPPGKGLEWIGCTFVGSSDSTYYANWAKGRVTISVDSSKNQVSLKLSSVTAADTAVYFCARHPSDAVYGYANNLWGQGTLVTVSS

SEQ ID NO: 123 - protein, 252 aa, synthetic construct
  REGION 1..252: note = artificial antibody-related sequence
DIQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKVSGFSFSNSYWICWVRQPPGKGLEWIGCTFVGSSDSTYYANWAKGRVTISVDSSKNQVSLKLSSVTAADSAVYFCARHPSDAVYGYANNLWGQGTLVKVSS

SEQ ID NO: 124 - protein, 124 aa, synthetic construct
  REGION 1..124: note = artificial antibody-related sequence
QVQLQESGPGLVKPSETLSLTCKASGFSFSNSYWICWVRQPPGKGLEWIGCTFVGSSDSTYYANWAKGRVTISKDSSKNQVSLKLSSVTAADTAVYFCARHPSDAVYGYANNLWGQGTLVTVSS

SEQ ID NO: 125 - protein, 124 aa, synthetic construct
  REGION 1..124: note = artificial antibody-related sequence
QVQLQESGPGLVKPSETLSLTCKASGFSFSNSYWICWVRQPPGKGLEWIGCTFVGSSDSTYYANWAKGRVTISKDSSKNQVSLKLSSVTAADSAVYFCARHPSDAVYGYANNLWGQGTLVKVSS

SEQ ID NO: 126 - protein, 108 aa, synthetic construct
  REGION 1..108: note = artificial antibody-related sequence
DLQMTQSPSSLSASVGDRVTITCQASQSINNVLAWYQQKPGKPPKLLIYRASTLASGVPSRFSGSGSGTDFTLTISSLQPEDFATYYCQSSYGNYGDFGTGTKVTVLG

SEQ ID NO: 127 - protein, 108 aa, synthetic construct
  REGION 1..108: note = artificial antibody-related sequence
DLQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGTGTKVTVLG

SEQ ID NO: 128 - protein, 252 aa, synthetic construct
  REGION 1..252: note = artificial antibody-related sequence
DLQMTQSPSSLSASVGDRVTITCQASQSINNVLAWYQQKPGKPPKLLIYRASTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKASGFSFSNSYWICWVRQPPGKGLEWIGCTFVGSSDSTYYANWAKGRVTISKDSSKNQVSLKLSSVTAADTAVYFCARHPSDAVYGYANNLWGQGTLVTVSS

SEQ ID NO: 129 - protein, 252 aa, synthetic construct
  REGION 1..252: note = artificial antibody-related sequence
DLQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKASGFSFSNSYWICWVRQPPGKGLEWIGCTFVGSSDSTYYANWAKGRVTISKDSSKNQVSLKLSSVTAADTAVYFCARHPSDAVYGYANNLWGQGTLVTVSS

SEQ ID NO: 130 - protein, 252 aa, synthetic construct
  REGION 1..252: note = artificial antibody-related sequence
DLQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGTGTKVTVLGGGGGSGGGGSGGGGSGGGGSQVQLQESGPGLVKPSETLSLTCKASGFSFSNSYWICWVRQPPGKGLEWIGCTFVGSSDSTYYANWAKGRVTISKDSSKNQVSLKLSSVTAADSAVYFCARHPSDAVYGYANNLWGQGTLVKVSS

SEQ ID NO: 131 - protein, 124 aa, synthetic construct
  REGION 1..124: note = artificial antibody-related sequence
EVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSS

SEQ ID NO: 132 - protein, 108 aa, synthetic construct
  REGION 1..108: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLG

SEQ ID NO: 133 - protein, 108 aa, synthetic construct
  REGION 1..108: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLG

SEQ ID NO: 134 - protein, 252 aa, synthetic construct
  REGION 1..252: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSGGGGSGGGGSGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSS

SEQ ID NO: 135 - protein, 252 aa, synthetic construct
  REGION 1..252: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSGGGGSGGGGSGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSS

SEQ ID NO: 136 - protein, 124 aa, synthetic construct
  REGION 1..124: note = artificial antibody-related sequence
QEQLVESGGGLVQPGGSLTLSCAASGFSFSNSYWICWVRQAPGKGLEWIGCTFVGSSDSTYYANWAKGRFTISKDNSKNTVYLQMNNLRAEDSAVYFCARHPSDAVYGYANNLWGQGTLVKVSS

SEQ ID NO: 137 - protein, 124 aa, synthetic construct
  REGION 1..124: note = artificial antibody-related sequence
QEQLVESGGGLVQPGGSLTLSCAASGFSFSNSYWICWVRQAPGKCLEWIGCTFVGSSDSTYYANWAKGRFTISKDNSKNTVYLQMNNLRAEDSAVYFCARHPSDAVYGYANNLWGQGTLVKVSS

SEQ ID NO: 138 - protein, 108 aa, synthetic construct; identical to SEQ ID NO: 119
  REGION 1..108: note = artificial antibody-related sequence

SEQ ID NO: 139 - protein, 108 aa, synthetic construct
  REGION 1..108: note = artificial antibody-related sequence
DIQMTQSPSSLSASVGDRVTITCQASQSINNVLAWYQQKPGKPPKLLIYRASTLASGVPSRFSGSGSGTDFTLTISSLQPEDFATYYCQSSYGNYGDFGCGTKVTVLG

SEQ ID NO: 140 - protein, 108 aa, synthetic construct; identical to SEQ ID NO: 120
  REGION 1..108: note = artificial antibody-related sequence

SEQ ID NO: 141 - protein, 108 aa, synthetic construct
  REGION 1..108: note = artificial antibody-related sequence
DIQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGCGTKVTVLG

SEQ ID NO: 142 - protein, 252 aa, synthetic construct
  REGION 1..252: note = artificial antibody-related sequence
DIQMTQSPSSLSASVGDRVTITCQASQSINNVLAWYQQKPGKPPKLLIYRASTLASGVPSRFSGSGSGTDFTLTISSLQPEDFATYYCQSSYGNYGDFGTGTKVTVLGGGGGSGGGGSGGGGSGGGGSQEQLVESGGGLVQPGGSLTLSCAASGFSFSNSYWICWVRQAPGKGLEWIGCTFVGSSDSTYYANWAKGRFTISKDNSKNTVYLQMNNLRAEDSAVYFCARHPSDAVYGYANNLWGQGTLVKVSS

SEQ ID NO: 143 - protein, 252 aa, synthetic construct
  REGION 1..252: note = artificial antibody-related sequence
DIQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGTGTKVTVLGGGGGSGGGGSGGGGSGGGGSQEQLVESGGGLVQPGGSLTLSCAASGFSFSNSYWICWVRQAPGKGLEWIGCTFVGSSDSTYYANWAKGRFTISKDNSKNTVYLQMNNLRAEDSAVYFCARHPSDAVYGYANNLWGQGTLVKVSS

SEQ ID NO: 144 - protein, 252 aa, synthetic construct
  REGION 1..252: note = artificial antibody-related sequence
DIQMTQSPSSLSASVGDRVTITCQASQSINNVLAWYQQKPGKPPKLLIYRGGTLASGVPSRFSGSGSGTDFTLTISSLQPEDFATYYCQSSYGNYGDFGCGTKVTVLGGGGGSGGGGSGGGGSGGGGSQEQLVESGGGLVQPGGSLTLSCAASGFSFSNSYWICWVRQAPGKCLEWIGCTFVGSSDSTYYANWAKGRFTISKDNSKNTVYLQMNNLRAEDSAVYFCARHPSDAVYGYANNLWGQGTLVKVSS

SEQ ID NO: 145 - protein, 125 aa, synthetic construct
  REGION 1..125: note = artificial antibody-related sequence
QVQLQESGPGLVKPSETLSLTCKVSGFSFNNDYDMCWIRQPPGKGLEWIGCIDTGDGSTYYASWAKGRVTISVDSSKNQFSLKLSSVTAADTAVYYCAREAASSSGYGMGYFDLWGQGTLVTVSS

SEQ ID NO: 146 - protein, 125 aa, synthetic construct
  REGION 1..125: note = artificial antibody-related sequence
QVQLQESGPGLVKPSETLSLTCKVSGFSFNNDYDMCWIRQPPGKGLEWIGCIDTGDGSTYYASWAKGRVTISVDSSKNQFSLKLSSVTAADSAVYYCAREAASSSGYGMGYFDLWGQGTLVKVSS

SEQ ID NO: 147 - protein, 113 aa, synthetic construct
  REGION 1..113: note = artificial antibody-related sequence
DIQMTQSPSSLSASVGDRVTITCQSSQSVYDNNWLAWYQQKPGKAPKLLIYRASNLASGVPSRFSGSGSGTDFTLTISSLQPEDFATYYCQGTYLSSNWYWAFGTGTKVTVLG

SEQ ID NO: 148 - protein, 113 aa, synthetic construct
  REGION 1..113: note = artificial antibody-related sequence
DIQMTQSPSSLSASVGDRVTITCQSSQSVYDNNWLAWYQQKPGKAPKLLIYRGGNLASGVPSRFSGSGSGTDFTLTISSLQPEDFATYYCQGTYLSSNWYWAFGTGTKVTVLG

SEQ ID NO: 149 - protein, 258 aa, synthetic construct
  REGION 1..258: note = artificial antibody-related sequence
DIQMTQSPSSLSASVGDRVTITCQSSQSVYDNNWLAWYQQKPGKAPKLLIYRASNLASGVPSRFSGSGSGTDFTLTISSLQPEDFATYYCQGTYLSSNWYWAFGTGTKVTVLGGGGGSGGGGSGGGGSGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADTAVYYCAREAASSSGYGMGYFDLWGQGTLVTVSS

SEQ ID NO: 150 - protein, 258 aa, synthetic construct
  REGION 1..258: note = artificial antibody-related sequence
DIQMTQSPSSLSASVGDRVTITCQSSQSVYDNNWLAWYQQKPGKAPKLLIYRGGNLASGVPSRFSGSGSGTDFTLTISSLQPEDFATYYCQGTYLSSNWYWAFGTGTKVTVLGGGGGSGGGGSGGGGSGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADTAVYYCAREAASSSGYGMGYFDLWGQGTLVTVSS

SEQ ID NO: 151 - protein, 258 aa, synthetic construct
  REGION 1..258: note = artificial antibody-related sequence
DIQMTQSPSSLSASVGDRVTITCQSSQSVYDNNWLAWYQQKPGKAPKLLIYRGGNLASGVPSRFSGSGSGTDFTLTISSLQPEDFATYYCQGTYLSSNWYWAFGTGTKVTVLGGGGGSGGGGSGGGGSGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADSAVYYCAREAASSSGYGMGYFDLWGQGTLVKVSS

SEQ ID NO: 152 - protein, 119 aa, synthetic construct
  REGION 1..119: note = artificial antibody-related sequence
EVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSS

SEQ ID NO: 153 - protein, 119 aa, synthetic construct
  REGION 1..119: note = artificial antibody-related sequence
EVQLVESGGGLVQPGGSLRLSCAASGFSLSSYDMSWVRQAPGKGLAWIGASYASGPTYYASWAKGRFTISRDNSKNTVYLQMNSLRAEDSATYFCARGGWTGTSHSNIWGQGTLVKVSS

SEQ ID NO: 154 - protein, 114 aa, synthetic construct
  REGION 1..114: note = artificial antibody-related sequence
DIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 155 - protein, 114 aa, synthetic construct
  REGION 1..114: note = artificial antibody-related sequence
DIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 156 - protein, 253 aa, synthetic construct
  REGION 1..253: note = artificial antibody-related sequence
DIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSGGGGSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSS

SEQ ID NO: 157 - protein, 253 aa, synthetic construct
  REGION 1..253: note = artificial antibody-related sequence
DIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLGGGGGSGGGGSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSS

SEQ ID NO: 158 - protein, 253 aa, synthetic construct
  REGION 1..253: note = artificial antibody-related sequence
DIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLGGGGGSGGGGSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDSATYFCARGGWTGTSHSNIWGQGTLVKVSS

SEQ ID NO: 159 - protein, 118 aa, synthetic construct
  REGION 1..118: note = artificial antibody-related sequence
QSVVESGGGLVQPGGSLRLSCAVSGFSLSSYDMSWVRQAPGKGLEWIGASYASGPTYYASWAKGRFTISRDNSKNTVYLQMNSLRAEDTAVYFCARGGWTGTSHSNIWGQGTLVTVSS

SEQ ID NO: 160 - protein, 118 aa, synthetic construct
  REGION 1..118: note = artificial antibody-related sequence
QSVVESGGGLVQPGGSLRLSCAVSGFSLSSYDMSWVRQAPGKGLEWIGASYASGPTYYASWAKGRFTISRDNSKNTVYLQMNSLRAEDSAVYFCARGGWTGTSHSNIWGQGTLVKVSS

SEQ ID NO: 161 - protein, 118 aa, synthetic construct
  REGION 1..118: note = artificial antibody-related sequence
QSVVESGGGLVQPGGSLRLSCAVSGFSLSSYDMSWVRQAPGKCLEWIGASYASGPTYYASWAKGRFTISRDNSKNTVYLQMNSLRAEDSAVYFCARGGWTGTSHSNIWGQGTLVKVSS

SEQ ID NO: 162 - protein, 114 aa, synthetic construct
  REGION 1..114: note = artificial antibody-related sequence
DIQMTQSPSSLSASVGDRVTITCQSSQSVFSNNYLAWFQQKPGKSPKRLIYSASTLASGVSSRFSGRGSGTDFTLTISSLQPEDFATYYCLGSYACSSADCYVFGTGTKVTVLG

SEQ ID NO: 163 - protein, 114 aa, synthetic construct
  REGION 1..114: note = artificial antibody-related sequence
DIQMTQSPSSLSASVGDRVTITCQSSQSVFSNNYLAWFQQKPGKSPKRLIYSGGTLASGVSSRFSGRGSGTDFTLTISSLQPEDFATYYCLGSYACSSADCYVFGTGTKVTVLG

SEQ ID NO: 164 - protein, 114 aa, synthetic construct
  REGION 1..114: note = artificial antibody-related sequence
DIQMTQSPSSLSASVGDRVTITCQSSQSVFSNNYLAWFQQKPGKSPKRLIYSGGTLASGVSSRFSGRGSGTDFTLTISSLQPEDFATYYCLGSYACSSADCYVFGCGTKVTVLG

SEQ ID NO: 165 - protein, 252 aa, synthetic construct
  REGION 1..252: note = artificial antibody-related sequence
DIQMTQSPSSLSASVGDRVTITCQSSQSVFSNNYLAWFQQKPGKSPKRLIYSASTLASGVSSRFSGRGSGTDFTLTISSLQPEDFATYYCLGSYACSSADCYVFGTGTKVTVLGGGGGSGGGGSGGGGSGGGGSQSVVESGGGLVQPGGSLRLSCAVSGFSLSSYDMSWVRQAPGKGLEWIGASYASGPTYYASWAKGRFTISRDNSKNTVYLQMNSLRAEDTAVYFCARGGWTGTSHSNIWGQGTLVTVSS

SEQ ID NO: 166 - protein, 252 aa, synthetic construct
  REGION 1..252: note = artificial antibody-related sequence
DIQMTQSPSSLSASVGDRVTITCQSSQSVFSNNYLAWFQQKPGKSPKRLIYSGGTLASGVSSRFSGRGSGTDFTLTISSLQPEDFATYYCLGSYACSSADCYVFGTGTKVTVLGGGGGSGGGGSGGGGSGGGGSQSVVESGGGLVQPGGSLRLSCAVSGFSLSSYDMSWVRQAPGKGLEWIGASYASGPTYYASWAKGRFTISRDNSKNTVYLQMNSLRAEDTAVYFCARGGWTGTSHSNIWGQGTLVTVSS

SEQ ID NO: 167 - protein, 252 aa, synthetic construct
  REGION 1..252: note = artificial antibody-related sequence
DIQMTQSPSSLSASVGDRVTITCQSSQSVFSNNYLAWFQQKPGKSPKRLIYSASTLASGVSSRFSGRGSGTDFTLTISSLQPEDFATYYCLGSYACSSADCYVFGTGTKVTVLGGGGGSGGGGSGGGGSGGGGSQSVVESGGGLVQPGGSLRLSCAVSGFSLSSYDMSWVRQAPGKGLEWIGASYASGPTYYASWAKGRFTISRDNSKNTVYLQMNSLRAEDSAVYFCARGGWTGTSHSNIWGQGTLVKVSS

SEQ ID NO: 168 - protein, 252 aa, synthetic construct
  REGION 1..252: note = artificial antibody-related sequence
DIQMTQSPSSLSASVGDRVTITCQSSQSVFSNNYLAWFQQKPGKSPKRLIYSGGTLASGVSSRFSGRGSGTDFTLTISSLQPEDFATYYCLGSYACSSADCYVFGTGTKVTVLGGGGGSGGGGSGGGGSGGGGSQSVVESGGGLVQPGGSLRLSCAVSGFSLSSYDMSWVRQAPGKGLEWIGASYASGPTYYASWAKGRFTISRDNSKNTVYLQMNSLRAEDSAVYFCARGGWTGTSHSNIWGQGTLVKVSS

SEQ ID NO: 169 - protein, 252 aa, synthetic construct
  REGION 1..252: note = artificial antibody-related sequence
DIQMTQSPSSLSASVGDRVTITCQSSQSVFSNNYLAWFQQKPGKSPKRLIYSGGTLASGVSSRFSGRGSGTDFTLTISSLQPEDFATYYCLGSYACSSADCYVFGCGTKVTVLGGGGGSGGGGSGGGGSGGGGSQSVVESGGGLVQPGGSLRLSCAVSGFSLSSYDMSWVRQAPGKCLEWIGASYASGPTYYASWAKGRFTISRDNSKNTVYLQMNSLRAEDSAVYFCARGGWTGTSHSNIWGQGTLVKVSS

SEQ ID NO: 170 - protein, 120 aa, synthetic construct
  REGION 1..120: note = artificial antibody-related sequence
EVQLVESGGGLVQPGGSLRLSCAASGFSLSSYAMNWVRQAPGKGLEWIGHINAGDIAYYATWAKGRFTISRDNSKNTVYLQMNSLRAEDTAVYFCARGAGGFSTGPFKLWGQGTLVTVSS

SEQ ID NO: 171 - protein, 120 aa, synthetic construct
  REGION 1..120: note = artificial antibody-related sequence
EVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 172 - protein, 120 aa, synthetic construct
  REGION 1..120: note = artificial antibody-related sequence
EVQLVESGGGLVQPGGSLRLSCAASGFSLSSYAMNWVRQAPGKGLEWIGHINAGDIAYYATWAKGRFTISRDNSKNTVYLQMNSLRAEDSAVYFCARGAGGFSTGPFKLWGQGTLVKVSS

SEQ ID NO: 173 - protein, 120 aa, synthetic construct
  REGION 1..120: note = artificial antibody-related sequence
EVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSS

SEQ ID NO: 174 - protein, 109 aa, synthetic construct
  REGION 1..109: note = artificial antibody-related sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGGGTKLTVLG

SEQ ID NO: 175 - protein, 109 aa, synthetic construct
  REGION 1..109: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGGGTKLTVLG

SEQ ID NO: 176 - protein, 109 aa, synthetic construct
  REGION 1..109: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLG

SEQ ID NO: 177 - protein, 249 aa, synthetic construct
  REGION 1..249: note = artificial antibody-related sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGGGTKLTVLGGGGGSGGGGSGGGGSGGGGSEVQLVESGGGLVQPGGSLRLSCAASGFSLSSYAMNWVRQAPGKGLEWIGHINAGDIAYYATWAKGRFTISRDNSKNTVYLQMNSLRAEDTAVYFCARGAGGFSTGPFKLWGQGTLVTVSS

SEQ ID NO: 178 - protein, 249 aa, synthetic construct
  REGION 1..249: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGGGTKLTVLGGGGGSGGGGSGGGGSGGGGSEVQLVESGGGLVQPGGSLRLSCAASGFSLSSYAMNWVRQAPGKGLEWIGHINAGDIAYYATWAKGRFTISRDNSKNTVYLQMNSLRAEDTAVYFCARGAGGFSTGPFKLWGQGTLVTVSS

SEQ ID NO: 179 - protein, 249 aa, synthetic construct
  REGION 1..249: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGGGTKLTVLGGGGGSGGGGSGGGGSGGGGSEVQLVESGGGLVQPGGSLRLSCAASGFSLSSYAMNWVRQAPGKGLEWIGHINAGDIAYYATWAKGRFTISRDNSKNTVYLQMNSLRAEDSAVYFCARGAGGFSTGPFKLWGQGTLVKVSS

SEQ ID NO: 180 - protein, 249 aa, synthetic construct
  REGION 1..249: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGGGSGGGGSGGGGSGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 181 - protein, 249 aa, synthetic construct
  REGION 1..249: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGGGSGGGGSGGGGSGGGGSEVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSS

SEQ ID NO: 182 - protein, 119 aa, synthetic construct
  REGION 1..119: note = artificial antibody-related sequence
QQLVESGGGLVQPGGSLRLSCAASGFSLSSYAMNWVRQAPGKCLEWIGHINAGDIAYYATWAKGRFTISKDNSKNTVYLQMNNLRAEDSAVYFCARGAGGFSTGPFKLWGQGTLVKVSS

SEQ ID NO: 183 - protein, 119 aa, synthetic construct
  REGION 1..119: note = artificial antibody-related sequence
QSVVESGGGLVQPGGSLRLSCAASGFSLSSYAMNWVRQAPGKCLEWIGHINAGDIAYYATWAKGRFTISKDNSKNTVYLQMNSLRAEDSAVYFCARGAGGFSTGPFKLWGQGTLVKVSS

SEQ ID NO: 184 - protein, 109 aa, synthetic construct
  REGION 1..109: note = artificial antibody-related sequence
AFQLTQSPSSLSASVGDRVTITCQASESINSRLAWYQQKPGKPPKLLIYDASDLTSGVPSRFSGRGSGTDFTLTISSLQPEDFATYYCQGYGGSSTTTFGCGTKVTVLG

SEQ ID NO: 185 - protein, 109 aa, synthetic construct
  REGION 1..109: note = artificial antibody-related sequence
AFQLTQSPSSLSASVGDRVTITCQASESINSRLAWYQQKPGKPPKLLIYDGGDLTSGVPSRFSGRGSGTDFTLTISSLQPEDFATYYCQGYGGSSTTTFGCGTKVTVLG

SEQ ID NO: 186 - protein, 248 aa, synthetic construct
  REGION 1..248: note = artificial antibody-related sequence
AFQLTQSPSSLSASVGDRVTITCQASESINSRLAWYQQKPGKPPKLLIYDASDLTSGVPSRFSGRGSGTDFTLTISSLQPEDFATYYCQGYGGSSTTTFGCGTKVTVLGGGGGSGGGGSGGGGSGGGGSQQLVESGGGLVQPGGSLRLSCAASGFSLSSYAMNWVRQAPGKCLEWIGHINAGDIAYYATWAKGRFTISKDNSKNTVYLQMNNLRAEDSAVYFCARGAGGFSTGPFKLWGQGTLVKVSS

SEQ ID NO: 187 - protein, 248 aa, synthetic construct
  REGION 1..248: note = artificial antibody-related sequence
AFQLTQSPSSLSASVGDRVTITCQASESINSRLAWYQQKPGKPPKLLIYDGGDLTSGVPSRFSGRGSGTDFTLTISSLQPEDFATYYCQGYGGSSTTTFGCGTKVTVLGGGGGSGGGGSGGGGSGGGGSQQLVESGGGLVQPGGSLRLSCAASGFSLSSYAMNWVRQAPGKCLEWIGHINAGDIAYYATWAKGRFTISKDNSKNTVYLQMNNLRAEDSAVYFCARGAGGFSTGPFKLWGQGTLVKVSS

SEQ ID NO: 188 - protein, 248 aa, synthetic construct
  REGION 1..248: note = artificial antibody-related sequence
AFQLTQSPSSLSASVGDRVTITCQASESINSRLAWYQQKPGKPPKLLIYDASDLTSGVPSRFSGRGSGTDFTLTISSLQPEDFATYYCQGYGGSSTTTFGCGTKVTVLGGGGGSGGGGSGGGGSGGGGSQSVVESGGGLVQPGGSLRLSCAASGFSLSSYAMNWVRQAPGKCLEWIGHINAGDIAYYATWAKGRFTISKDNSKNTVYLQMNSLRAEDSAVYFCARGAGGFSTGPFKLWGQGTLVKVSS

SEQ ID NO: 189 - protein, 248 aa, synthetic construct
  REGION 1..248: note = artificial antibody-related sequence
AFQLTQSPSSLSASVGDRVTITCQASESINSRLAWYQQKPGKPPKLLIYDGGDLTSGVPSRFSGRGSGTDFTLTISSLQPEDFATYYCQGYGGSSTTTFGCGTKVTVLGGGGGSGGGGSGGGGSGGGGSQSVVESGGGLVQPGGSLRLSCAASGFSLSSYAMNWVRQAPGKCLEWIGHINAGDIAYYATWAKGRFTISKDNSKNTVYLQMNSLRAEDSAVYFCARGAGGFSTGPFKLWGQGTLVKVSS

SEQ ID NO: 190 - protein, 121 aa, synthetic construct
  REGION 1..121: note = artificial antibody-related sequence
EVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 191 - protein, 121 aa, synthetic construct
  REGION 1..121: note = artificial antibody-related sequence
EVQLVESGGGLVQPGGSLRLSCAASGFSLSSNAMGWVRQAPGKCLEYIGIISVGGFTYYASWAKGRFTISRDNSKNTVYLQMNSLRAEDTATYFCARDRHGGDSSGAFYLWGQGTLVTVSS

SEQ ID NO: 192 - protein, 121 aa, synthetic construct
  REGION 1..121: note = artificial antibody-related sequence
EVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 193 - protein, 121 aa, synthetic construct
  REGION 1..121: note = artificial antibody-related sequence
EVQLVESGGGLVQPGGSLRLSCAASGFSLSSNAMGWVRQAPGKCLEYIGIISVGGFTYYASWAKGRFTISRDNSKNTVYLQMNSLRAEDSATYFCARDRHGGDSSGAFYLWGQGTLVKVSS

SEQ ID NO: 194 - protein, 112 aa, synthetic construct
  REGION 1..112: note = artificial antibody-related sequence
DIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLG

SEQ ID NO: 195 - protein, 112 aa, synthetic construct
  REGION 1..112: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLG

SEQ ID NO: 196 - protein, 112 aa, synthetic construct
  REGION 1..112: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGCGTKLTVLG

SEQ ID NO: 197 - protein, 253 aa, synthetic construct
  REGION 1..253: note = artificial antibody-related sequence
DIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 198 - protein, 253 aa, synthetic construct
  REGION 1..253: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 199 - protein, 253 aa, synthetic construct
  REGION 1..253: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 200 - protein, 253 aa, synthetic construct
  REGION 1..253: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGCGTKLTVLGGGGGSGGGGSGGGGSGGGGSEVQLVESGGGLVQPGGSLRLSCAASGFSLSSNAMGWVRQAPGKCLEYIGIISVGGFTYYASWAKGRFTISRDNSKNTVYLQMNSLRAEDTATYFCARDRHGGDSSGAFYLWGQGTLVTVSS

SEQ ID NO: 201 - protein, 253 aa, synthetic construct
  REGION 1..253: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGCGTKLTVLGGGGGSGGGGSGGGGSGGGGSEVQLVESGGGLVQPGGSLRLSCAASGFSLSSNAMGWVRQAPGKCLEYIGIISVGGFTYYASWAKGRFTISRDNSKNTVYLQMNSLRAEDSATYFCARDRHGGDSSGAFYLWGQGTLVKVSS

SEQ ID NO: 202 - protein, 121 aa, synthetic construct
  REGION 1..121: note = artificial antibody-related sequence
EVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 203 - protein, 121 aa, synthetic construct
  REGION 1..121: note = artificial antibody-related sequence
EVQLVESGGGLVQPGGSLRLSCAASGFSFSSSYWICWVRQAPGKCLEWVGCVFTGDGTTYYASWAKGRFTISRDNSKNTVYLQMNSLRAEDTATYFCARPVSVYYYGMDLWGQGTLVTVSS

SEQ ID NO: 204 - protein, 121 aa, synthetic construct
  REGION 1..121: note = artificial antibody-related sequence
EVQLVESGGGLVQPGGSLRLSCAASGFSFSSSYWICWVRQAPGKGLEWVGCVFTGDGTTYYASWAKGRFTISRDNSKNTVYLQMNSLRAEDSATYFCARPVSVYYYGMDLWGQGTLVKVSS

SEQ ID NO: 205 - protein, 121 aa, synthetic construct
  REGION 1..121: note = artificial antibody-related sequence
EVQLVESGGGLVQPGGSLRLSCAASGFSFSSSYWICWVRQAPGKCLEWVGCVFTGDGTTYYASWAKGRFTISRDNSKNTVYLQMNSLRAEDSATYFCARPVSVYYYGMDLWGQGTLVKVSS

SEQ ID NO: 206 - protein, 110 aa, synthetic construct
  REGION 1..110: note = artificial antibody-related sequence
DVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGGGTKLTVLG

SEQ ID NO: 207 - protein, 110 aa, synthetic construct
  REGION 1..110: note = artificial antibody-related sequence
DVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLG

SEQ ID NO: 208 - protein, 110 aa, synthetic construct
  REGION 1..110: note = artificial antibody-related sequence
DVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGCGTKLTVLG

SEQ ID NO: 209 - protein, 251 aa, synthetic construct
  REGION 1..251: note = artificial antibody-related sequence
DVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 210 - protein, 251 aa, synthetic construct
  REGION 1..251: note = artificial antibody-related sequence
DVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 211 - protein, 251 aa, synthetic construct
  REGION 1..251: note = artificial antibody-related sequence
DVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDSATYFCARPVSVYYYGMDLWGQGTLVKVSS

SEQ ID NO: 212 - protein, 251 aa, synthetic construct
  REGION 1..251: note = artificial antibody-related sequence
DVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGCGTKLTVLGGGGGSGGGGSGGGGSGGGGSEVQLVESGGGLVQPGGSLRLSCAASGFSFSSSYWICWVRQAPGKCLEWVGCVFTGDGTTYYASWAKGRFTISRDNSKNTVYLQMNSLRAEDTATYFCARPVSVYYYGMDLWGQGTLVTVSS

SEQ ID NO: 213 - protein, 251 aa, synthetic construct
  REGION 1..251: note = artificial antibody-related sequence
DVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGCGTKLTVLGGGGGSGGGGSGGGGSGGGGSEVQLVESGGGLVQPGGSLRLSCAASGFSFSSSYWICWVRQAPGKCLEWVGCVFTGDGTTYYASWAKGRFTISRDNSKNTVYLQMNSLRAEDSATYFCARPVSVYYYGMDLWGQGTLVKVSS

SEQ ID NO: 214 - protein, 122 aa, synthetic construct
  REGION 1..122: note = artificial antibody-related sequence
EVQLVESGGGLVQPGGSLRLSCAASGFSFSTTYYMCWVRQAPGKGLEWIGCTNTASSVRTYYATWAKGRFTISRDNSKNTVYLQMNSLRAEDTAVYYCARDMGFADYALNLWGQGTLVTVSS

SEQ ID NO: 215 - protein, 122 aa, synthetic construct
  REGION 1..122: note = artificial antibody-related sequence
EVQLVESGGGLVQPGGSLRLSCAASGFSFSTTYYMCWVRQAPGKCLEWIGCTNTASSVRTYYATWAKGRFTISRDNSKNTVYLQMNSLRAEDTAVYYCARDMGFADYALNLWGQGTLVTVSS

SEQ ID NO: 216 - protein, 122 aa, synthetic construct
  REGION 1..122: note = artificial antibody-related sequence
EVQLVESGGGLVQPGGSLRLSCAASGFSFSTTYYMCWVRQAPGKGLEWIGCTNTASSVRTYYATWAKGRFTISRDNSKNTVYLQMNSLRAEDSAVYYCARDMGFADYALNLWGQGTLVKVSS

SEQ ID NO: 217 - protein, 122 aa, synthetic construct
  REGION 1..122: note = artificial antibody-related sequence
EVQLVESGGGLVQPGGSLRLSCAASGFSFSTTYYMCWVRQAPGKCLEWIGCTNTASSVRTYYATWAKGRFTISRDNSKNTVYLQMNSLRAEDSAVYYCARDMGFADYALNLWGQGTLVKVSS

SEQ ID NO: 218 - protein, 112 aa, synthetic construct
  REGION 1..112: note = artificial antibody-related sequence
DIQMTQSPSSLSASVGDRVTITCQASESIYSSLAWYQQKPGKAPKLLIYLASTLASGVPSRFSGSGSGTDFTLTISSLQPEDFATYYCQSTDYTTSTHRNSFGTGTKVTVLG

SEQ ID NO: 219 - protein, 112 aa, synthetic construct
  REGION 1..112: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGTGTKVTVLG

SEQ ID NO: 220 - protein, 112 aa, synthetic construct
  REGION 1..112: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGCGTKVTVLG

SEQ ID NO: 221 - protein, 254 aa, synthetic construct
  REGION 1..254: note = artificial antibody-related sequence
DIQMTQSPSSLSASVGDRVTITCQASESIYSSLAWYQQKPGKAPKLLIYLASTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDTAVYYCARDMGFADYALNLWGQGTLVTVSS

SEQ ID NO: 222 - protein, 254 aa, synthetic construct
  REGION 1..254: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDTAVYYCARDMGFADYALNLWGQGTLVTVSS

SEQ ID NO: 223 - protein, 254 aa, synthetic construct
  REGION 1..254: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDSAVYYCARDMGFADYALNLWGQGTLVKVSS

SEQ ID NO: 224 - protein, 254 aa, synthetic construct
  REGION 1..254: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGCGTKVTVLGGGGGSGGGGSGGGGSGGGGSEVQLVESGGGLVQPGGSLRLSCAASGFSFSTTYYMCWVRQAPGKCLEWIGCTNTASSVRTYYATWAKGRFTISRDNSKNTVYLQMNSLRAEDTAVYYCARDMGFADYALNLWGQGTLVTVSS

SEQ ID NO: 225 - protein, 254 aa, synthetic construct
  REGION 1..254: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGCGTKVTVLGGGGGSGGGGSGGGGSGGGGSEVQLVESGGGLVQPGGSLRLSCAASGFSFSTTYYMCWVRQAPGKCLEWIGCTNTASSVRTYYATWAKGRFTISRDNSKNTVYLQMNSLRAEDSAVYYCARDMGFADYALNLWGQGTLVKVSS

SEQ ID NO: 226 - protein, 122 aa, synthetic construct
  REGION 1..122: note = artificial antibody-related sequence
QSQLVESGGGLVQPGGSLRLSCAVSGFSLSSYAMGWVRQAPGKGLEYIGYISKIGTTYYASWAKGRFTISKDNSKNTVYLQMNSLRAEDTAVYFCARGSSSGGYLDDGFDPWGQGTLVTVSS

SEQ ID NO: 227 - protein, 122 aa, synthetic construct
  REGION 1..122: note = artificial antibody-related sequence
QSQLVESGGGLVQPGGSLRLSCAVSGFSLSSYAMGWVRQAPGKGLEYIGYISKIGTTYYASWAKGRFTISKDNSKNTVYLQMNSLRAEDSAVYFCARGSSSGGYLDDGFDPWGQGTLVKVSS

SEQ ID NO: 228 - protein, 111 aa, synthetic construct
  REGION 1..111: note = artificial antibody-related sequence
ALQMTQSPSSLSASVGDRVTITCQASQSISNYLAWYQQKPGKPPKFLIYDASDLASGVSSRFSGSGSGTDFTLTISSLQPEDFATYYCQQVYDSNNVENVFGTGTKVTVLG

SEQ ID NO: 229 - protein, 111 aa, synthetic construct
  REGION 1..111: note = artificial antibody-related sequence
ALQMTQSPSSLSASVGDRVTITCQASQSISNYLAWYQQKPGKPPKFLIYDGGDLASGVSSRFSGSGSGTDFTLTISSLQPEDFATYYCQQVYDSNNVENVFGTGTKVTVLG

SEQ ID NO: 230 - protein, 253 aa, synthetic construct
  REGION 1..253: note = artificial antibody-related sequence
ALQMTQSPSSLSASVGDRVTITCQASQSISNYLAWYQQKPGKPPKFLIYDASDLASGVSSRFSGSGSGTDFTLTISSLQPEDFATYYCQQVYDSNNVENVFGTGTKVTVLGGGGGSGGGGSGGGGSGGGGSQSQLVESGGGLVQPGGSLRLSCAVSGFSLSSYAMGWVRQAPGKGLEYIGYISKIGTTYYASWAKGRFTISKDNSKNTVYLQMNSLRAEDTAVYFCARGSSSGGYLDDGFDPWGQGTLVTVSS

SEQ ID NO: 231 - protein, 253 aa, synthetic construct
  REGION 1..253: note = artificial antibody-related sequence
ALQMTQSPSSLSASVGDRVTITCQASQSISNYLAWYQQKPGKPPKFLIYDGGDLASGVSSRFSGSGSGTDFTLTISSLQPEDFATYYCQQVYDSNNVENVFGTGTKVTVLGGGGGSGGGGSGGGGSGGGGSQSQLVESGGGLVQPGGSLRLSCAVSGFSLSSYAMGWVRQAPGKGLEYIGYISKIGTTYYASWAKGRFTISKDNSKNTVYLQMNSLRAEDTAVYFCARGSSSGGYLDDGFDPWGQGTLVTVSS

SEQ ID NO: 232 - protein, 253 aa, synthetic construct
  REGION 1..253: note = artificial antibody-related sequence
ALQMTQSPSSLSASVGDRVTITCQASQSISNYLAWYQQKPGKPPKFLIYDGGDLASGVSSRFSGSGSGTDFTLTISSLQPEDFATYYCQQVYDSNNVENVFGTGTKVTVLGGGGGSGGGGSGGGGSGGGGSQSQLVESGGGLVQPGGSLRLSCAVSGFSLSSYAMGWVRQAPGKGLEYIGYISKIGTTYYASWAKGRFTISKDNSKNTVYLQMNSLRAEDSAVYFCARGSSSGGYLDDGFDPWGQGTLVKVSS

SEQ ID NO: 233 - protein, 122 aa, synthetic construct; identical to SEQ ID NO: 226
  REGION 1..122: note = artificial antibody-related sequence

SEQ ID NO: 234 - protein, 122 aa, synthetic construct
  REGION 1..122: note = artificial antibody-related sequence
QSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSS

SEQ ID NO: 235 - protein, 122 aa, synthetic construct; identical to SEQ ID NO: 227
  REGION 1..122: note = artificial antibody-related sequence

SEQ ID NO: 236 - protein, 122 aa, synthetic construct
  REGION 1..122: note = artificial antibody-related sequence
QSQLVESGGGLVQPGGSLRLSCAVSGFSLSSYAMGWVRQAPGKCLEYIGYISKIGTTYYASWAKGRFTISKDNSKNTVYLQMNSLRAEDSAVYFCARGSSSGGYLDDGFDPWGQGTLVKVSS

SEQ ID NO: 237 - protein, 111 aa, synthetic construct
  REGION 1..111: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGTGTKVTVLG

SEQ ID NO: 238 - protein, 111 aa, synthetic construct
  REGION 1..111: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLG

SEQ ID NO: 239 - protein, 111 aa, synthetic construct
  REGION 1..111: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGTGTKVTVLG

SEQ ID NO: 240 - protein, 111 aa, synthetic construct
  REGION 1..111: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLG

SEQ ID NO: 241 - protein, 253 aa, synthetic construct
  REGION 1..253: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGTGTKVTVLGGGGGSGGGGSGGGGSGGGGSQSQLVESGGGLVQPGGSLRLSCAVSGFSLSSYAMGWVRQAPGKGLEYIGYISKIGTTYYASWAKGRFTISKDNSKNTVYLQMNSLRAEDTAVYFCARGSSSGGYLDDGFDPWGQGTLVTVSS

SEQ ID NO: 242 - protein, 253 aa, synthetic construct
  REGION 1..253: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGTGTKVTVLGGGGGSGGGGSGGGGSGGGGSQSQLVESGGGLVQPGGSLRLSCAVSGFSLSSYAMGWVRQAPGKGLEYIGYISKIGTTYYASWAKGRFTISKDNSKNTVYLQMNSLRAEDTAVYFCARGSSSGGYLDDGFDPWGQGTLVTVSS

SEQ ID NO: 243 - protein, 253 aa, synthetic construct
  REGION 1..253: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGTGTKVTVLGGGGGSGGGGSGGGGSGGGGSQSQLVESGGGLVQPGGSLRLSCAVSGFSLSSYAMGWVRQAPGKGLEYIGYISKIGTTYYASWAKGRFTISKDNSKNTVYLQMNSLRAEDSAVYFCARGSSSGGYLDDGFDPWGQGTLVKVSS

SEQ ID NO: 244 - protein, 253 aa, synthetic construct
  REGION 1..253: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSS

SEQ ID NO: 245 - protein, 253 aa, synthetic construct
  REGION 1..253: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSS

SEQ ID NO: 246 - protein, 253 aa, synthetic construct
  REGION 1..253: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDSAVYFCARGSSSGGYLDDGFDPWGQGTLVKVSS

SEQ ID NO: 247 - protein, 121 aa, synthetic construct
  REGION 1..121: note = artificial antibody-related sequence
QVQLVESGGGLVQPGGSLRLSCAASGFSLSSYAMGWVRQAPGKGLEWIGYISTINNTYYASWAKGRFTISRDNSKNTVYLQMNSLRAEDTAVYYCAREIRSGWVDYGFSIWGQGTLVTVSS

SEQ ID NO: 248 - protein, 121 aa, synthetic construct
  REGION 1..121: note = artificial antibody-related sequence
QVQLVESGGGLVQPGGSLRLSCAASGFSLSSYAMGWVRQAPGKGLEWIGYISTIANTYYASWAKGRFTISRDNSKNTVYLQMNSLRAEDTAVYYCAREIRSGWVDYGFSIWGQGTLVTVSS

SEQ ID NO: 249 - protein, 121 aa, synthetic construct
  REGION 1..121: note = artificial antibody-related sequence
QVQLVESGGGLVQPGGSLRLSCAASGFSLSSYAMGWVRQAPGKGLEWIGYISTINNTYYASWAKGRFTISRDNSKNTVYLQMNSLRAEDSAVYYCAREIRSGWVDYGFSIWGQGTLVKVSS

SEQ ID NO: 250 - protein, 121 aa, synthetic construct
  REGION 1..121: note = artificial antibody-related sequence
QVQLVESGGGLVQPGGSLRLSCAASGFSLSSYAMGWVRQAPGKGLEWIGYISTIANTYYASWAKGRFTISRDNSKNTVYLQMNSLRAEDSAVYYCAREIRSGWVDYGFSIWGQGTLVKVSS

SEQ ID NO: 251 - protein, 111 aa, synthetic construct
  REGION 1..111: note = artificial antibody-related sequence
DIQMTQSPSSLSASVGDRVTITCQASQNIYSNLAWYQQKPGKAPKLLIYDASDLASGVPSRFSGSGSGTDFTLTISSLQPEDFATYYCQQVRSSSDIDNPFGTGTKVTVLG

SEQ ID NO: 252 - protein, 111 aa, synthetic construct
  REGION 1..111: note = artificial antibody-related sequence
DIQMTQSPSSLSASVGDRVTITCQASQNIYSNLAWYQQKPGKAPKLLIYDGGDLASGVPSRFSGSGSGTDFTLTISSLQPEDFATYYCQQVRSSSDIDNPFGTGTKVTVLG

SEQ ID NO: 253 - protein, 252 aa, synthetic construct
  REGION 1..252: note = artificial antibody-related sequence
DIQMTQSPSSLSASVGDRVTITCQASQNIYSNLAWYQQKPGKAPKLLIYDASDLASGVPSRFSGSGSGTDFTLTISSLQPEDFATYYCQQVRSSSDIDNPFGTGTKVTVLGGGGGSGGGGSGGGGSGGGGSQVQLVESGGGLVQPGGSLRLSCAASGFSLSSYAMGWVRQAPGKGLEWIGYISTINNTYYASWAKGRFTISRDNSKNTVYLQMNSLRAEDTAVYYCAREIRSGWVDYGFSIWGQGTLVTVSS

SEQ ID NO: 254 - protein, 252 aa, synthetic construct
  REGION 1..252: note = artificial antibody-related sequence
DIQMTQSPSSLSASVGDRVTITCQASQNIYSNLAWYQQKPGKAPKLLIYDGGDLASGVPSRFSGSGSGTDFTLTISSLQPEDFATYYCQQVRSSSDIDNPFGTGTKVTVLGGGGGSGGGGSGGGGSGGGGSQVQLVESGGGLVQPGGSLRLSCAASGFSLSSYAMGWVRQAPGKGLEWIGYISTINNTYYASWAKGRFTISRDNSKNTVYLQMNSLRAEDTAVYYCAREIRSGWVDYGFSIWGQGTLVTVSS

SEQ ID NO: 255 - protein, 252 aa, synthetic construct
  REGION 1..252: note = artificial antibody-related sequence
DIQMTQSPSSLSASVGDRVTITCQASQNIYSNLAWYQQKPGKAPKLLIYDGGDLASGVPSRFSGSGSGTDFTLTISSLQPEDFATYYCQQVRSSSDIDNPFGTGTKVTVLGGGGGSGGGGSGGGGSGGGGSQVQLVESGGGLVQPGGSLRLSCAASGFSLSSYAMGWVRQAPGKGLEWIGYISTIANTYYASWAKGRFTISRDNSKNTVYLQMNSLRAEDTAVYYCAREIRSGWVDYGFSIWGQGTLVTVSS

SEQ ID NO: 256 - protein, 252 aa, synthetic construct
  REGION 1..252: note = artificial antibody-related sequence
DIQMTQSPSSLSASVGDRVTITCQASQNIYSNLAWYQQKPGKAPKLLIYDGGDLASGVPSRFSGSGSGTDFTLTISSLQPEDFATYYCQQVRSSSDIDNPFGTGTKVTVLGGGGGSGGGGSGGGGSGGGGSQVQLVESGGGLVQPGGSLRLSCAASGFSLSSYAMGWVRQAPGKGLEWIGYISTIANTYYASWAKGRFTISRDNSKNTVYLQMNSLRAEDSAVYYCAREIRSGWVDYGFSIWGQGTLVKVSS

SEQ ID NO: 257 - protein, 123 aa, synthetic construct
  REGION 1..123: note = artificial antibody-related sequence
EVQLVESGGGLVQPGGSLRLSCAASGISVSNDYYMCWVRQAPGKGLEWIGCISTYIGNTHYASWAKGRFTISRDNSKNTVYLQMNSLRAEDTAVYYCAKNAGYPGYRYAIDLWGQGTLVTVSS

SEQ ID NO: 258 - protein, 123 aa, synthetic construct
  REGION 1..123: note = artificial antibody-related sequence
EVQLVESGGGLVQPGGSLRLSCAASGISVSNDYYMCWVRQAPGKGLEWIGCISTYIGNTHYASWAKGRFTISRDNSKNTVYLQMNSLRAEDSAVYYCAKNAGYPGYRYAIDLWGQGTLVKVSS

SEQ ID NO: 259 - protein, 109 aa, synthetic construct
  REGION 1..109: note = artificial antibody-related sequence
DIQMTQSPSSLSASVGDRVTITCQASESIGNYLAWYQQKPGKAPKLLIYSASTLASGVPSRFSGSGSGTDFTLTISSLQPEDFATYYCQSTDYGDSYIFGTGTKVTVLG

SEQ ID NO: 260 - protein, 109 aa, synthetic construct
  REGION 1..109: note = artificial antibody-related sequence
DIQMTQSPSSLSASVGDRVTITCQASESIGNYLAWYQQKPGKAPKLLIYSGGTLASGVPSRFSGSGSGTDFTLTISSLQPEDFATYYCQSTDYGDSYIFGTGTKVTVLG

SEQ ID NO: 261 - protein, 252 aa, synthetic construct
  REGION 1..252: note = artificial antibody-related sequence
DIQMTQSPSSLSASVGDRVTITCQASESIGNYLAWYQQKPGKAPKLLIYSASTLASGVPSRFSGSGSGTDFTLTISSLQPEDFATYYCQSTDYGDSYIFGTGTKVTVLGGGGGSGGGGSGGGGSGGGGSEVQLVESGGGLVQPGGSLRLSCAASGISVSNDYYMCWVRQAPGKGLEWIGCISTYIGNTHYASWAKGRFTISRDNSKNTVYLQMNSLRAEDTAVYYCAKNAGYPGYRYAIDLWGQGTLVTVSS

SEQ ID NO: 262 - protein, 252 aa, synthetic construct
  REGION 1..252: note = artificial antibody-related sequence
DIQMTQSPSSLSASVGDRVTITCQASESIGNYLAWYQQKPGKAPKLLIYSGGTLASGVPSRFSGSGSGTDFTLTISSLQPEDFATYYCQSTDYGDSYIFGTGTKVTVLGGGGGSGGGGSGGGGSGGGGSEVQLVESGGGLVQPGGSLRLSCAASGISVSNDYYMCWVRQAPGKGLEWIGCISTYIGNTHYASWAKGRFTISRDNSKNTVYLQMNSLRAEDTAVYYCAKNAGYPGYRYAIDLWGQGTLVTVSS

SEQ ID NO: 263 - protein, 252 aa, synthetic construct
  REGION 1..252: note = artificial antibody-related sequence
DIQMTQSPSSLSASVGDRVTITCQASESIGNYLAWYQQKPGKAPKLLIYSGGTLASGVPSRFSGSGSGTDFTLTISSLQPEDFATYYCQSTDYGDSYIFGTGTKVTVLGGGGGSGGGGSGGGGSGGGGSEVQLVESGGGLVQPGGSLRLSCAASGISVSNDYYMCWVRQAPGKGLEWIGCISTYIGNTHYASWAKGRFTISRDNSKNTVYLQMNSLRAEDSAVYYCAKNAGYPGYRYAIDLWGQGTLVKVSS

SEQ ID NO: 264 - protein, 118 aa, synthetic construct
  REGION 1..118: note = artificial antibody-related sequence
QSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSS

SEQ ID NO: 265 - protein, 118 aa, synthetic construct
  REGION 1..118: note = artificial antibody-related sequence
QSQLVESGGGLVQPGGSLRLSCAVSGLSLSRNAMSWVRQAPGKCLEWIGIILTSGSTYYASWAKGRFTISKDNSKNTVYLQMNSLRAEDTAVYFCVRGIASSSLKSFWGQGTLVTVSS

SEQ ID NO: 266 - protein, 118 aa, synthetic construct
  REGION 1..118: note = artificial antibody-related sequence
QSQLVESGGGLVQPGGSLRLSCAVSGLSLSRNAMSWVRQAPGKGLEWIGIILTSGSTYYASWAKGRFTISKDNSKNTVYLQMNSLRAEDSAVYFCVRGIASSSLKSFWGQGTLVKVSS

SEQ ID NO: 267 - protein, 118 aa, synthetic construct
  REGION 1..118: note = artificial antibody-related sequence
QSQLVESGGGLVQPGGSLRLSCAVSGLSLSRNAMSWVRQAPGKCLEWIGIILTSGSTYYASWAKGRFTISKDNSKNTVYLQMNSLRAEDSAVYFCVRGIASSSLKSFWGQGTLVKVSS

SEQ ID NO: 268 - protein, 111 aa, synthetic construct
  REGION 1..111: note = artificial antibody-related sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLG

SEQ ID NO: 269 - protein, 111 aa, synthetic construct
  REGION 1..111: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGTGTKVTVLG

SEQ ID NO: 270 - protein, 111 aa, synthetic construct
  REGION 1..111: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGCGTKVTVLG

SEQ ID NO: 271 - protein, 249 aa, synthetic construct
  REGION 1..249: note = artificial antibody-related sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSS

SEQ ID NO: 272 - protein, 249 aa, synthetic construct
  REGION 1..249: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDTAVYFCVRGIASSSLKSFWGQGTLVTVSS

SEQ ID NO: 273 - protein, 249 aa, synthetic construct
  REGION 1..249: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGLSLSRNAMSWVRQAPGKGLEWIGIILTSGSTYYASWAKGRFTISKDNSKNTVYLQMNSLRAEDSAVYFCVRGIASSSLKSFWGQGTLVKVSS

SEQ ID NO: 274 - protein, 249 aa, synthetic construct
  REGION 1..249: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGLSLSRNAMSWVRQAPGKCLEWIGIILTSGSTYYASWAKGRFTISKDNSKNTVYLQMNSLRAEDSAVYFCVRGIASSSLKSFWGQGTLVKVSS

SEQ ID NO: 275 - protein, 116 aa, synthetic construct
  REGION 1..116: note = artificial antibody-related sequence
QSQLVESGGGLVQPGGSLRLSCAVSGIDLSRNAMSWVRQAPGKGLEWIGIILTSGSTYYASWAKGRFTISKTSTTLDLQMNSLRAEDTAVYFCVRGIASSSLKSFWGQGTLVTVSS

SEQ ID NO: 276 - protein, 116 aa, synthetic construct
  REGION 1..116: note = artificial antibody-related sequence
QSQLVESGGGLVQPGGSLRLSCAVSGIDLSRNAMSWVRQAPGKCLEWIGIILTSGSTYYASWAKGRFTISKTSTTLDLQMNSLRAEDTAVYFCVRGIASSSLKSFWGQGTLVTVSS

SEQ ID NO: 277 - protein, 116 aa, synthetic construct
  REGION 1..116: note = artificial antibody-related sequence
QSQLVESGGGLVQPGGSLRLSCAVSGIDLSRNAMSWVRQAPGKGLEWIGIILTSGSTYYASWAKGRFTISKTSTTLDLQMNSLRAEDSAVYFCVRGIASSSLKSFWGQGTLVKVSS

SEQ ID NO: 278 - protein, 116 aa, synthetic construct
  REGION 1..116: note = artificial antibody-related sequence
QSQLVESGGGLVQPGGSLRLSCAVSGIDLSRNAMSWVRQAPGKCLEWIGIILTSGSTYYASWAKGRFTISKTSTTLDLQMNSLRAEDSAVYFCVRGIASSSLKSFWGQGTLVKVSS

SEQ ID NO: 279 - protein, 111 aa, synthetic construct; identical to SEQ ID NO: 268
  REGION 1..111: note = artificial antibody-related sequence

SEQ ID NO: 280 - protein, 111 aa, synthetic construct; identical to SEQ ID NO: 269
  REGION 1..111: note = artificial antibody-related sequence

SEQ ID NO: 281 - protein, 111 aa, synthetic construct; identical to SEQ ID NO: 270
  REGION 1..111: note = artificial antibody-related sequence

SEQ ID NO: 282 - protein, 247 aa, synthetic construct
  REGION 1..247: note = artificial antibody-related sequence
AQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGIDLSRNAMSWVRQAPGKGLEWIGIILTSGSTYYASWAKGRFTISKTSTTLDLQMNSLRAEDTAVYFCVRGIASSSLKSFWGQGTLVTVSS

SEQ ID NO: 283 - protein, 247 aa, synthetic construct
  REGION 1..247: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGIDLSRNAMSWVRQAPGKGLEWIGIILTSGSTYYASWAKGRFTISKTSTTLDLQMNSLRAEDTAVYFCVRGIASSSLKSFWGQGTLVTVSS

SEQ ID NO: 284 - protein, 247 aa, synthetic construct
  REGION 1..247: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGTGTKVTVLGGGGGSGGGGSGGGGSGGGGSQSQLVESGGGLVQPGGSLRLSCAVSGIDLSRNAMSWVRQAPGKGLEWIGIILTSGSTYYASWAKGRFTISKTSTTLDLQMNSLRAEDSAVYFCVRGIASSSLKSFWGQGTLVKVSS

SEQ ID NO: 285 - protein, 247 aa, synthetic construct
  REGION 1..247: note = artificial antibody-related sequence
AQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGIDLSRNAMSWVRQAPGKGLEWIGIILTSGSTYYASWAKGRFTISKTSTTLDLQMNSLRAEDSAVYFCVRGIASSSLKSFWGQGTLVKVSS

SEQ ID NO: 286 - protein, 124 aa, synthetic construct
  REGION 1..124: note = artificial antibody-related sequence
QSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSS

SEQ ID NO: 287 - protein, 124 aa, synthetic construct
  REGION 1..124: note = artificial antibody-related sequence
QSQVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGQGTLVKVSS

SEQ ID NO: 288 - protein, 124 aa, synthetic construct
  REGION 1..124: note = artificial antibody-related sequence
QSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGQGTLVTVSS

SEQ ID NO: 289 - protein, 124 aa, synthetic construct
  REGION 1..124: note = artificial antibody-related sequence
QSQVVESGGGLVQPGGSLRLSCAVSGFDLSSYAVSWVRQAPGKCLEWIGIIYPRANTYYASWAKGRFTISKDNSKNTVYLQMNSLRAEDSAVYFCARDRYDSGAYLYTTYFNLWGQGTLVKVSS

SEQ ID NO: 290 - protein, 123 aa, synthetic construct
  REGION 1..123: note = artificial antibody-related sequence
QSVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGQGTLVKVSS

SEQ ID NO: 291 - protein, 123 aa, synthetic construct
  REGION 1..123: note = artificial antibody-related sequence
QSVVESGGGLVQPGGSLRLSCAVSGFDLSSYAVSWVRQAPGKCLEWIGIIYPRANTYYASWAKGRFTISKDNSKNTVYLQMNSLRAEDSAVYFCARDRYDSGAYLYTTYFNLWGQGTLVKVSS

SEQ ID NO: 292 - protein, 112 aa, synthetic construct
  REGION 1..112: note = artificial antibody-related sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLG

SEQ ID NO: 293 - protein, 112 aa, synthetic construct
  REGION 1..112: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLG

SEQ ID NO: 294 - protein, 112 aa, synthetic construct
  REGION 1..112: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGCGTKVTVLG

SEQ ID NO: 295 - protein, 255 aa, synthetic construct
  REGION 1..255: note = artificial antibody-related sequence
VQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSS

SEQ ID NO: 296 - protein, 256 aa, synthetic construct
  REGION 1..256: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSS

SEQ ID NO: 297 - protein, 256 aa, synthetic construct
  REGION 1..256: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGQGTLVKVSS

SEQ ID NO: 298 - protein, 256 aa, synthetic construct
  REGION 1..256: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDSAVYFCARDRYDSGAYLYTTYFNLWGQGTLVKVSS

SEQ ID NO: 299 - protein, 255 aa, synthetic construct
  REGION 1..255: note = artificial antibody-related sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGQGTLVKVSS

SEQ ID NO: 300 - protein, 255 aa, synthetic construct
  REGION 1..255: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGQGTLVKVSS

SEQ ID NO: 301 - protein, 255 aa, synthetic construct
  REGION 1..255: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGCGTKVTVLGGGGGSGGGGSGGGGSGGGGSQSVVESGGGLVQPGGSLRLSCAVSGFDLSSYAVSWVRQAPGKCLEWIGIIYPRANTYYASWAKGRFTISKDNSKNTVYLQMNSLRAEDSAVYFCARDRYDSGAYLYTTYFNLWGQGTLVKVSS

SEQ ID NO: 302 - protein, 120 aa, synthetic construct [sequence omitted]

SEQ ID NO: 303 - protein, 108 aa, synthetic construct
  REGION 1..108: note = artificial antibody-related sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGGGTKLTVLG

SEQ ID NO: 304 - protein, 108 aa, synthetic construct
  REGION 1..108: note = artificial antibody-related sequence
DIQMTQSPSSLSASVGDRVTITCRASQDVNTAVAWYQQKPGKAPKLLIYSGGFLYSGVPSRFSGSRSGTDFTLTISSLQPEDFATYYCQQHYTTPPTFGGGTKLTVLG

SEQ ID NO: 305 - protein, 248 aa, synthetic construct
  REGION 1..248: note = artificial antibody-related sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGGGTKLTVLGGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 306 - protein, 248 aa, synthetic construct
  REGION 1..248: note = artificial antibody-related sequence
DIQMTQSPSSLSASVGDRVTITCRASQDVNTAVAWYQQKPGKAPKLLIYSGGFLYSGVPSRFSGSRSGTDFTLTISSLQPEDFATYYCQQHYTTPPTFGGGTKLTVLGGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 307 - protein, 119 aa, synthetic construct [sequence omitted]

SEQ ID NO: 308 - protein, 108 aa, synthetic construct
  REGION 1..108: note = artificial antibody-related sequence
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGTGTKVTVLG

SEQ ID NO: 309 - protein, 108 aa, synthetic construct
  REGION 1..108: note = artificial antibody-related sequence
DIQMTQSPSSLSASVGDRVTITCKASQDVSIGVAWYQQKPGKAPKLLIYSGGYRYTGVPSRFSGSGSGTDFTLTISSLQPEDFATYYCQQYYIYPYTFGTGTKVTVLG

SEQ ID NO: 310 - protein, 247 aa, synthetic construct
  REGION 1..247: note = artificial antibody-related sequence
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGTGTKVTVLGGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 311 - protein, 247 aa, synthetic construct
  REGION 1..247: note = artificial antibody-related sequence
DIQMTQSPSSLSASVGDRVTITCKASQDVSIGVAWYQQKPGKAPKLLIYSGGYRYTGVPSRFSGSGSGTDFTLTISSLQPEDFATYYCQQYYIYPYTFGTGTKVTVLGGGGGSGGGGSGGGGSGGGGSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 312 - protein, 121 aa, synthetic construct
  REGION 1..121: note = artificial antibody-related sequence
EVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 313 - protein, 121 aa, synthetic construct
  REGION 1..121: note = artificial antibody-related sequence
EVQLVESGGGLVQPGGSLRLSCAASGIDFNSNYYMCWVRQAPGKGLEWIGCIYVGSHVNTYYANWAKGRFTISRDNSKNTVYLQMNSLRAEDSAVYYCATSGSSVLYFKFWGQGTLVKVSS

SEQ ID NO: 314 - protein, 113 aa, synthetic construct
  REGION 1..113: note = artificial antibody-related sequence
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGQGTKLTVLG

SEQ ID NO: 315 - protein, 113 aa, synthetic construct
  REGION 1..113: note = artificial antibody-related sequence
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGTGTKVTVLG

SEQ ID NO: 316 - protein, 113 aa, synthetic construct
  REGION 1..113: note = artificial antibody-related sequence
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGGGTKLTVLG

SEQ ID NO: 317 - protein, 113 aa, synthetic construct
  REGION 1..113: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGQGTKLTVLG

SEQ ID NO: 318 - protein, 113 aa, synthetic construct
  REGION 1..113: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGTGTKVTVLG

SEQ ID NO: 319 - protein, 113 aa, synthetic construct
  REGION 1..113: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGGGTKLTVLG

SEQ ID NO: 320 - protein, 254 aa, synthetic construct
  REGION 1..254: note = artificial antibody-related sequence
DIQMTQSPSSLSASVGDRVTITCQASENIYSFLAWYQQKPGKAPKLLIYSASKLAAGVPSRFSGSGSGTDFTLTISSLQPEDFATYYCQQTNRYSNPDIYNVFGGGTKLTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 321 - protein, 254 aa, synthetic construct
  REGION 1..254: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGQGTKLTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 322 - protein, 254 aa, synthetic construct
  REGION 1..254: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGTGTKVTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 323 - protein, 254 aa, synthetic construct
  REGION 1..254: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGGGTKLTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDTAVYYCATSGSSVLYFKFWGQGTLVTVSS

SEQ ID NO: 324 - protein, 254 aa, synthetic construct
  REGION 1..254: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGQGTKLTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDSAVYYCATSGSSVLYFKFWGQGTLVKVSS

SEQ ID NO: 325 - protein, 254 aa, synthetic construct
  REGION 1..254: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGTGTKVTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDSAVYYCATSGSSVLYFKFWGQGTLVKVSS

SEQ ID NO: 326 - protein, 254 aa, synthetic construct
  REGION 1..254: note = artificial antibody-related sequence
DIQMTQSPSSLSASVGDRVTITCQASENIYSFLAWYQQKPGKAPKLLIYSGGKLAAGVPSRFSGSGSGTDFTLTISSLQPEDFATYYCQQTNRYSNPDIYNVFGGGTKLTVLGGGGGSGGGGSGGGGSGGGGSEVQLVESGGGLVQPGGSLRLSCAASGIDFNSNYYMCWVRQAPGKGLEWIGCIYVGSHVNTYYANWAKGRFTISRDNSKNTVYLQMNSLRAEDSAVYYCATSGSSVLYFKFWGQGTLVKVSS

SEQ ID NO: 327 - protein, 122 aa, synthetic construct
  REGION 1..122: note = artificial antibody-related sequence
EVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSS

SEQ ID NO: 328 - protein, 122 aa, synthetic construct
  REGION 1..122: note = artificial antibody-related sequence
EVQLVESGGGLVQPGGSLRLSCAASGFSFSSDYWMCWVRQAPGKGLEWIGCIYAGDVDDTDYASWARGRFTISRDNSKNTVYLQMNSLRAEDSAVYYCARVDGFDITYFDLWGQGTLVKVSS

SEQ ID NO: 329 - protein, 122 aa, synthetic construct
  REGION 1..122: note = artificial antibody-related sequence
EVQLVESGGGLVKPEGSLRLSCAASGFSFSSDYWMCWVRQAPGKGLEWIACIYAGDVDDTDYASWARGRFTISRDNSKNTVYLQMNSLRAEDTAVYYCARVDGFDITYFDLWGQGTLVTVSS

SEQ ID NO: 330 - protein, 122 aa, synthetic construct
  REGION 1..122: note = artificial antibody-related sequence
EVQLVESGGGLVKPEGSLRLSCAASGFSFSSDYWMCWVRQAPGKGLEWIACIYAGDVDDTDYASWARGRFTISRDNSKNTVYLQMNSLRAEDSAVYYCARVDGFDITYFDLWGQGTLVKVSS

SEQ ID NO: 331 - protein, 111 aa, synthetic construct
  REGION 1..111: note = artificial antibody-related sequence
DIQMTQSPSSLSASVGDRVTITCQASQTINYWLAWYQQKPGKAPKLLIYRASTLASGVPSRFSGSGSGTDFTLTISSLQPEDFATYYCQNYYYSTSRSYGFGGGTKLTVLG

SEQ ID NO: 332 - protein, 111 aa, synthetic construct
  REGION 1..111: note = artificial antibody-related sequence
DIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLG

SEQ ID NO: 333 - protein, 253 aa, synthetic construct
  REGION 1..253: note = artificial antibody-related sequence
DIQMTQSPSSLSASVGDRVTITCQASQTINYWLAWYQQKPGKAPKLLIYRASTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSS

SEQ ID NO: 334 - protein, 253 aa, synthetic construct
  REGION 1..253: note = artificial antibody-related sequence
DIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSS

SEQ ID NO: 335 - protein, 253 aa, synthetic construct
  REGION 1..253: note = artificial antibody-related sequence
DIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDSAVYYCARVDGFDITYFDLWGQGTLVKVSS

SEQ ID NO: 336 - protein, 253 aa, synthetic construct
  REGION 1..253: note = artificial antibody-related sequence
DIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVKPEGSLRLSCAASGFSFSSDYWMCWVRQAPGKGLEWIACIYAGDVDDTDYASWARGRFTISRDNSKNTVYLQMNSLRAEDTAVYYCARVDGFDITYFDLWGQGTLVTVSS

SEQ ID NO: 337 - protein, 253 aa, synthetic construct
  REGION 1..253: note = artificial antibody-related sequence
DIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVKPEGSLRLSCAASGFSFSSDYWMCWVRQAPGKGLEWIACIYAGDVDDTDYASWARGRFTISRDNSKNTVYLQMNSLRAEDSAVYYCARVDGFDITYFDLWGQGTLVKVSS

SEQ ID NO: 338 - protein, 126 aa, synthetic construct
  REGION 1..126: note = artificial antibody-related sequence
EVQLVESGGGLVQPGGSLRLSCAASGFSFSGFYYMCWVRQAPGKGLEWIGCIDTGDGTTYYASWAKGRFTISRDNSKNTVYLQMNSLRAEDTAVYYCARDAAYGGYAYPAHGMDLWGQGTLVTVSS

SEQ ID NO: 339 - protein, 126 aa, synthetic construct
  REGION 1..126: note = artificial antibody-related sequence
EVQLVESGGGLVQPGGSLRLSCAASGFSFSGFYYMCWVRQAPGKGLEWIGCIDTGDGTTYYASWAKGRFTISRDNSKNTVYLQMNSLRAEDSAVYYCARDAAYGGYAYPAHGMDLWGQGTLVKVSS

SEQ ID NO: 340 - protein, 126 aa, synthetic construct
  REGION 1..126: note = artificial antibody-related sequence
EVQLVESGGGLVQPGGSLTLSCAASGFSFSGFYYMCWVRQAPGKGLEWIGCIDTGDGTTYYASWAKGRFTISRDNSKNTVYLQMNSLRAEDTAVYYCARDAAYGGYAYPAHGMDLWGPGTLVTVSS

SEQ ID NO: 341 - protein, 126 aa, synthetic construct
  REGION 1..126: note = artificial antibody-related sequence
EVQLVESGGGLVQPGGSLTLSCAASGFSFSGFYYMCWVRQAPGKGLEWIGCIDTGDGTTYYASWAKGRFTISRDNSKNTVYLQMNSLRAEDSAVYYCARDAAYGGYAYPAHGMDLWGPGTLVKVSS

SEQ ID NO: 342 - protein, 113 aa, synthetic construct
  REGION 1..113: note = artificial antibody-related sequence
DIQMTQSPSSLSASVGDRVTITCQASESIYRYLSWYQQKPGKAPKLLIYLASTLTSGVPSRFSGSGSGTDFTLTISSLQPEDFATYYCQSNFGTASTTYYNTFGGGTKLTVLG

SEQ ID NO: 343 - protein, 113 aa, synthetic construct
  REGION 1..113: note = artificial antibody-related sequence
DIQMTQSPSSLSASVGDRVTITCQASESIYRYLSWYQQKPGKAPKLLIYLGGTLTSGVPSRFSGSGSGTDFTLTISSLQPEDFATYYCQSNFGTASTTYYNTFGGGTKLTVLG

SEQ ID NO: 344 - protein, 259 aa, synthetic construct
  REGION 1..259: note = artificial antibody-related sequence
DIQMTQSPSSLSASVGDRVTITCQASESIYRYLSWYQQKPGKAPKLLIYLASTLTSGVPSRFSGSGSGTDFTLTISSLQPEDFATYYCQSNFGTASTTYYNTFGGGTKLTVLGGGGGSGGGGSGGGGSGGGGSEVQLVESGGGLVQPGGSLRLSCAASGFSFSGFYYMCWVRQAPGKGLEWIGCIDTGDGTTYYASWAKGRFTISRDNSKNTVYLQMNSLRAEDTAVYYCARDAAYGGYAYPAHGMDLWGQGTLVTVSS

SEQ ID NO: 345 - protein, 259 aa, synthetic construct
  REGION 1..259: note = artificial antibody-related sequence
DIQMTQSPSSLSASVGDRVTITCQASESIYRYLSWYQQKPGKAPKLLIYLGGTLTSGVPSRFSGSGSGTDFTLTISSLQPEDFATYYCQSNFGTASTTYYNTFGGGTKLTVLGGGGGSGGGGSGGGGSGGGGSEVQLVESGGGLVQPGGSLRLSCAASGFSFSGFYYMCWVRQAPGKGLEWIGCIDTGDGTTYYASWAKGRFTISRDNSKNTVYLQMNSLRAEDTAVYYCARDAAYGGYAYPAHGMDLWGQGTLVTVSS

SEQ ID NO: 346 - protein, 259 aa, synthetic construct
  REGION 1..259: note = artificial antibody-related sequence
DIQMTQSPSSLSASVGDRVTITCQASESIYRYLSWYQQKPGKAPKLLIYLGGTLTSGVPSRFSGSGSGTDFTLTISSLQPEDFATYYCQSNFGTASTTYYNTFGGGTKLTVLGGGGGSGGGGSGGGGSGGGGSEVQLVESGGGLVQPGGSLRLSCAASGFSFSGFYYMCWVRQAPGKGLEWIGCIDTGDGTTYYASWAKGRFTISRDNSKNTVYLQMNSLRAEDSAVYYCARDAAYGGYAYPAHGMDLWGQGTLVKVSS

SEQ ID NO: 347 - protein, 259 aa, synthetic construct
  REGION 1..259: note = artificial antibody-related sequence
DIQMTQSPSSLSASVGDRVTITCQASESIYRYLSWYQQKPGKAPKLLIYLGGTLTSGVPSRFSGSGSGTDFTLTISSLQPEDFATYYCQSNFGTASTTYYNTFGGGTKLTVLGGGGGSGGGGSGGGGSGGGGSEVQLVESGGGLVQPGGSLTLSCAASGFSFSGFYYMCWVRQAPGKGLEWIGCIDTGDGTTYYASWAKGRFTISRDNSKNTVYLQMNSLRAEDTAVYYCARDAAYGGYAYPAHGMDLWGPGTLVTVSS

SEQ ID NO: 348 - protein, 259 aa, synthetic construct
  REGION 1..259: note = artificial antibody-related sequence
DIQMTQSPSSLSASVGDRVTITCQASESIYRYLSWYQQKPGKAPKLLIYLGGTLTSGVPSRFSGSGSGTDFTLTISSLQPEDFATYYCQSNFGTASTTYYNTFGGGTKLTVLGGGGGSGGGGSGGGGSGGGGSEVQLVESGGGLVQPGGSLTLSCAASGFSFSGFYYMCWVRQAPGKGLEWIGCIDTGDGTTYYASWAKGRFTISRDNSKNTVYLQMNSLRAEDSAVYYCARDAAYGGYAYPAHGMDLWGPGTLVKVSS

SEQ ID NO: 349 - protein, 121 aa, synthetic construct; identical to SEQ ID NO: 50
  REGION 1..121: note = artificial antibody-related sequence

SEQ ID NO: 350 - protein, 121 aa, synthetic construct; identical to SEQ ID NO: 51
  REGION 1..121: note = artificial antibody-related sequence

SEQ ID NO: 351 - protein, 111 aa, synthetic construct
  REGION 1..111: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQAAYIITDIDNVFGTGTKVTVLG

SEQ ID NO: 352 - protein, 111 aa, synthetic construct
  REGION 1..111: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQAAYIITDIDNVFGTGTKVTVLG

SEQ ID NO: 353 - protein, 252 aa, synthetic construct
  REGION 1..252: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQAAYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSS

SEQ ID NO: 354 - protein, 252 aa, synthetic construct
  REGION 1..252: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQAAYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSS

SEQ ID NO: 355 - protein, 252 aa, synthetic construct
  REGION 1..252: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQAAYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSS

SEQ ID NO: 356 - protein, 125 aa, synthetic construct
  REGION 1..125: note = artificial antibody-related sequence
EVQLVESGGGLVQPGGSLRLSCAASGIDFSTYGISWVRQAPGKGLEWIGYIYPDYGITDYASWVNGRFTISRDNSKNTVYLQMNSLRAEDTAVYYCARSGSYYSRGWGAHYFNLWGQGTLVTVSS

SEQ ID NO: 357 - protein, 125 aa, synthetic construct
  REGION 1..125: note = artificial antibody-related sequence
EVQLVESGGGLVQPGGSLRLSCAASGIDFSTYGISWVRQAPGKGLEWIGYIYPDYGITDYASWVNGRFTISRDNSKNTVYLQMNSLRAEDSAVYYCARSGSYYSRGWGAHYFNLWGQGTLVKVSS

SEQ ID NO: 358 - protein, 112 aa, synthetic construct
  REGION 1..112: note = artificial antibody-related sequence
DIQMTQSPSSLSASVGDRVTITCQASESISSWLAWYQQKPGKAPKLLIYKASTLASGVPSRFSGSGSGTDFTLTISSLQPEDFATYYCQGYYLDSSVDDNVFGGGTKLTVLG

SEQ ID NO: 359 - protein, 112 aa, synthetic construct
  REGION 1..112: note = artificial antibody-related sequence
DIQMTQSPSSLSASVGDRVTITCQASESISSWLAWYQQKPGKAPKLLIYKGGTLASGVPSRFSGSGSGTDFTLTISSLQPEDFATYYCQGYYLDSSVDDNVFGGGTKLTVLG

SEQ ID NO: 360 - protein, 257 aa, synthetic construct
  REGION 1..257: note = artificial antibody-related sequence
DIQMTQSPSSLSASVGDRVTITCQASESISSWLAWYQQKPGKAPKLLIYKASTLASGVPSRFSGSGSGTDFTLTISSLQPEDFATYYCQGYYLDSSVDDNVFGGGTKLTVLGGGGGSGGGGSGGGGSGGGGSEVQLVESGGGLVQPGGSLRLSCAASGIDFSTYGISWVRQAPGKGLEWIGYIYPDYGITDYASWVNGRFTISRDNSKNTVYLQMNSLRAEDTAVYYCARSGSYYSRGWGAHYFNLWGQGTLVTVSS

SEQ ID NO: 361 - protein, 257 aa, synthetic construct
  REGION 1..257: note = artificial antibody-related sequence
DIQMTQSPSSLSASVGDRVTITCQASESISSWLAWYQQKPGKAPKLLIYKGGTLASGVPSRFSGSGSGTDFTLTISSLQPEDFATYYCQGYYLDSSVDDNVFGGGTKLTVLGGGGGSGGGGSGGGGSGGGGSEVQLVESGGGLVQPGGSLRLSCAASGIDFSTYGISWVRQAPGKGLEWIGYIYPDYGITDYASWVNGRFTISRDNSKNTVYLQMNSLRAEDTAVYYCARSGSYYSRGWGAHYFNLWGQGTLVTVSS

SEQ ID NO: 362 - protein, 257 aa, synthetic construct
  REGION 1..257: note = artificial antibody-related sequence
DIQMTQSPSSLSASVGDRVTITCQASESISSWLAWYQQKPGKAPKLLIYKGGTLASGVPSRFSGSGSGTDFTLTISSLQPEDFATYYCQGYYLDSSVDDNVFGGGTKLTVLGGGGGSGGGGSGGGGSGGGGSEVQLVESGGGLVQPGGSLRLSCAASGIDFSTYGISWVRQAPGKGLEWIGYIYPDYGITDYASWVNGRFTISRDNSKNTVYLQMNSLRAEDSAVYYCARSGSYYSRGWGAHYFNLWGQGTLVKVSS

SEQ ID NO: 363 - protein, 125 aa, synthetic construct
  REGION 1..125: note = artificial antibody-related sequence
EVQLVESGGGLVQPGGSLRLSCKASGIDFSTYGISWVRQAPGKGLEWIAYIYPDYGITDYASWVNGRFTISLDNSKNTVYLQMNSLRAEDTAVYYCARSGSYYSRGWGAHYFNLWGQGTLVTVSS

SEQ ID NO: 364 - protein, 125 aa, synthetic construct
  REGION 1..125: note = artificial antibody-related sequence
EVQLVESGGGLVQPGGSLRLSCKASGIDFSTYGISWVRQAPGKGLEWIAYIYPDYGITDYASWVNGRFTISLDNSKNTVYLQMNSLRAEDSAVYYCARSGSYYSRGWGAHYFNLWGQGTLVKVSS

SEQ ID NO: 365 - protein, 112 aa, synthetic construct
  REGION 1..112: note = artificial antibody-related sequence
DIQMTQSPSSLSASVGDRVTINCQASESISSWLAWYQQKPGKAPKLLIYKASTLASGVPSRFSGSGSGTEFTLTISGLQPADFATYYCQGYYLDSSVDDNVFGGGTKLTVLG

SEQ ID NO: 366 - protein, 112 aa, synthetic construct
  REGION 1..112: note = artificial antibody-related sequence
DIQMTQSPSSLSASVGDRVTINCQASESISSWLAWYQQKPGKAPKLLIYKGGTLASGVPSRFSGSGSGTEFTLTISGLQPADFATYYCQGYYLDSSVDDNVFGGGTKLTVLG

SEQ ID NO: 367 - protein, 257 aa, synthetic construct
  REGION 1..257: note = artificial antibody-related sequence
DIQMTQSPSSLSASVGDRVTINCQASESISSWLAWYQQKPGKAPKLLIYKASTLASGVPSRFSGSGSGTEFTLTISGLQPADFATYYCQGYYLDSSVDDNVFGGGTKLTVLGGGGGSGGGGSGGGGSGGGGSEVQLVESGGGLVQPGGSLRLSCKASGIDFSTYGISWVRQAPGKGLEWIAYIYPDYGITDYASWVNGRFTISLDNSKNTVYLQMNSLRAEDTAVYYCARSGSYYSRGWGAHYFNLWGQGTLVTVSS

SEQ ID NO: 368 - protein, 257 aa, synthetic construct
  REGION 1..257: note = artificial antibody-related sequence
DIQMTQSPSSLSASVGDRVTINCQASESISSWLAWYQQKPGKAPKLLIYKGGTLASGVPSRFSGSGSGTEFTLTISGLQPADFATYYCQGYYLDSSVDDNVFGGGTKLTVLGGGGGSGGGGSGGGGSGGGGSEVQLVESGGGLVQPGGSLRLSCKASGIDFSTYGISWVRQAPGKGLEWIAYIYPDYGITDYASWVNGRFTISLDNSKNTVYLQMNSLRAEDTAVYYCARSGSYYSRGWGAHYFNLWGQGTLVTVSS

SEQ ID NO: 369 - protein, 257 aa, synthetic construct
  REGION 1..257: note = artificial antibody-related sequence
DIQMTQSPSSLSASVGDRVTINCQASESISSWLAWYQQKPGKAPKLLIYKGGTLASGVPSRFSGSGSGTEFTLTISGLQPADFATYYCQGYYLDSSVDDNVFGGGTKLTVLGGGGGSGGGGSGGGGSGGGGSEVQLVESGGGLVQPGGSLRLSCKASGIDFSTYGISWVRQAPGKGLEWIAYIYPDYGITDYASWVNGRFTISLDNSKNTVYLQMNSLRAEDSAVYYCARSGSYYSRGWGAHYFNLWGQGTLVKVSS

SEQ ID NO: 370 - protein, 125 aa, synthetic construct
  REGION 1..125: note = artificial antibody-related sequence
EVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSS

SEQ ID NO: 371 - protein, 125 aa, synthetic construct
  REGION 1..125: note = artificial antibody-related sequence
EVQLVESGGGLVQPGGSLRLSCAASGIDFSTYGISWVRQAPGKGLEWIAYIYPDYGITDYASWVNGRFTISLDNAQNTVYLQMNSLRAEDSAVYYCARSGSYYSRGWGAHYFNLWGQGTLVKVSS

SEQ ID NO: 372 - protein, 112 aa, synthetic construct
  REGION 1..112: note = artificial antibody-related sequence
DIQMTQSPSSLSASVGDRVTINCQASESISSWLAWYQQKPGKRPKLLIYKASTLASGVPSRFSGSGSGTDYTLTISSLQPEDFATYYCQGYYLDSSVDDNVFGGGTKLTVLG

SEQ ID NO: 373 - protein, 112 aa, synthetic construct
  REGION 1..112: note = artificial antibody-related sequence
DIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLG

SEQ ID NO: 374 - protein, 257 aa, synthetic construct
  REGION 1..257: note = artificial antibody-related sequence
DIQMTQSPSSLSASVGDRVTINCQASESISSWLAWYQQKPGKRPKLLIYKASTLASGVPSRFSGSGSGTDYTLTISSLQPEDFATYYCQGYYLDSSVDDNVFGGGTKLTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSS

SEQ ID NO: 375 - protein, 257 aa, synthetic construct
  REGION 1..257: note = artificial antibody-related sequence
DIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSS

SEQ ID NO: 376 - protein, 257 aa, synthetic construct
  REGION 1..257: note = artificial antibody-related sequence
DIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDSAVYYCARSGSYYSRGWGAHYFNLWGQGTLVKVSS

SEQ ID NO: 377 - protein, 125 aa, synthetic construct; identical to SEQ ID NO: 370
  REGION 1..125: note = artificial antibody-related sequence

SEQ ID NO: 378 - protein, 125 aa, synthetic construct; identical to SEQ ID NO: 371
  REGION 1..125: note = artificial antibody-related sequence

SEQ ID NO: 379 - protein, 112 aa, synthetic construct
  REGION 1..112: note = artificial antibody-related sequence
DIQMTQSPSSLSASVGDRVTINCQASESISSWLAWYQQKPGKRPKLLIYKASTLASGVPSRFSGSGSGTDYTLTISGLQPEDFATYYCQGYYLDSSVDDNVFGTGTKVTVLG

SEQ ID NO: 380 - protein, 112 aa, synthetic construct
  REGION 1..112: note = artificial antibody-related sequence
DIQMTQSPSSLSASVGDRVTINCQASESISSWLAWYQQKPGKRPKLLIYKGGTLASGVPSRFSGSGSGTDYTLTISGLQPEDFATYYCQGYYLDSSVDDNVFGTGTKVTVLG

SEQ ID NO: 381 - protein, 257 aa, synthetic construct
  REGION 1..257: note = artificial antibody-related sequence
DIQMTQSPSSLSASVGDRVTINCQASESISSWLAWYQQKPGKRPKLLIYKASTLASGVPSRFSGSGSGTDYTLTISGLQPEDFATYYCQGYYLDSSVDDNVFGTGTKVTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSS

SEQ ID NO: 382 - protein, 257 aa, synthetic construct
  REGION 1..257: note = artificial antibody-related sequence
DIQMTQSPSSLSASVGDRVTINCQASESISSWLAWYQQKPGKRPKLLIYKGGTLASGVPSRFSGSGSGTDYTLTISGLQPEDFATYYCQGYYLDSSVDDNVFGTGTKVTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSS

SEQ ID NO: 383 - protein, 257 aa, synthetic construct
  REGION 1..257: note = artificial antibody-related sequence
DIQMTQSPSSLSASVGDRVTINCQASESISSWLAWYQQKPGKRPKLLIYKGGTLASGVPSRFSGSGSGTDYTLTISGLQPEDFATYYCQGYYLDSSVDDNVFGTGTKVTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDSAVYYCARSGSYYSRGWGAHYFNLWGQGTLVKVSS

SEQ ID NO: 384 - protein, 125 aa, synthetic construct; identical to SEQ ID NO: 370
  REGION 1..125: note = artificial antibody-related sequence

SEQ ID NO: 385 - protein, 125 aa, synthetic construct; identical to SEQ ID NO: 371
  REGION 1..125: note = artificial antibody-related sequence

SEQ ID NO: 386 - protein, 112 aa, synthetic construct
  REGION 1..112: note = artificial antibody-related sequence
DIQMTQSPSSLSASVGDRVTINCQASESISSWLAWYQQKPGQRPKLLIYKASTLASGVPSRFSGSGSGTDYTLTISSLQPEDFATYYCQGYYLDSSVDDNVFGTGTKVTVLG

SEQ ID NO: 387 - protein, 112 aa, synthetic construct
  REGION 1..112: note = artificial antibody-related sequence
DIQMTQSPSSLSASVGDRVTINCQASESISSWLAWYQQKPGQRPKLLIYKGGTLASGVPSRFSGSGSGTDYTLTISSLQPEDFATYYCQGYYLDSSVDDNVFGTGTKVTVLG

SEQ ID NO: 388 - protein, 257 aa, synthetic construct
  REGION 1..257: note = artificial antibody-related sequence
DIQMTQSPSSLSASVGDRVTINCQASESISSWLAWYQQKPGQRPKLLIYKASTLASGVPSRFSGSGSGTDYTLTISSLQPEDFATYYCQGYYLDSSVDDNVFGTGTKVTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSS

SEQ ID NO: 389 - protein, 257 aa, synthetic construct
  REGION 1..257: note = artificial antibody-related sequence
DIQMTQSPSSLSASVGDRVTINCQASESISSWLAWYQQKPGQRPKLLIYKGGTLASGVPSRFSGSGSGTDYTLTISSLQPEDFATYYCQGYYLDSSVDDNVFGTGTKVTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSS

SEQ ID NO: 390 - protein, 257 aa, synthetic construct
  REGION 1..257: note = artificial antibody-related sequence
DIQMTQSPSSLSASVGDRVTINCQASESISSWLAWYQQKPGQRPKLLIYKGGTLASGVPSRFSGSGSGTDYTLTISSLQPEDFATYYCQGYYLDSSVDDNVFGTGTKVTVLGGGGGSGGGGSGGGGSGGGGSEVQLVESGGGLVQPGGSLRLSCAASGIDFSTYGISWVRQAPGKGLEWIAYIYPDYGITDYASWVNGRFTISLDNAQNTVYLQMNSLRAEDSAVYYCARSGSYYSRGWGAHYFNLWGQGTLVKVSS

SEQ ID NO: 391 - protein, 125 aa, synthetic construct
  REGION 1..125: note = artificial antibody-related sequence
EVQLVESGGGLVQPGGSLRLSCAASGIDFSRYGISWVRQAPGKGLEWIGYIYPDYGTTDYANWVNGRFTISRDNSKNTVYLQMNSLRAEDTAVYYCARSGSYYSSGWGAEYFNLWGQGTLVTVSS

SEQ ID NO: 392 - protein, 125 aa, synthetic construct
  REGION 1..125: note = artificial antibody-related sequence
EVQLVESGGGLVQPGGSLRLSCAASGIDFSRYGISWVRQAPGKGLEWIGYIYPDYGTTDYANWVNGRFTISRDNSKNTVYLQMNSLRAEDSAVYYCARSGSYYSSGWGAEYFNLWGQGTLVKVSS

SEQ ID NO: 393 - protein, 112 aa, synthetic construct
  REGION 1..112: note = artificial antibody-related sequence
DIQMTQSPSSLSASVGDRVTITCQASQSISNWLAWYQQKPGKAPKLLIYKASTLASGVPSRFSGSGSGTDFTLTISSLQPEDFATYYCQGYYYSSSGDDNAFGGGTKLTVLG

SEQ ID NO: 394 - protein, 112 aa, synthetic construct
  REGION 1..112: note = artificial antibody-related sequence
DIQMTQSPSSLSASVGDRVTITCQASQSISNWLAWYQQKPGKAPKLLIYKGGTLASGVPSRFSGSGSGTDFTLTISSLQPEDFATYYCQGYYYSSSGDDNAFGGGTKLTVLG

SEQ ID NO: 395 - protein, 257 aa, synthetic construct
  REGION 1..257: note = artificial antibody-related sequence
DIQMTQSPSSLSASVGDRVTITCQASQSISNWLAWYQQKPGKAPKLLIYKASTLASGVPSRFSGSGSGTDFTLTISSLQPEDFATYYCQGYYYSSSGDDNAFGGGTKLTVLGGGGGSGGGGSGGGGSGGGGSEVQLVESGGGLVQPGGSLRLSCAASGIDFSRYGISWVRQAPGKGLEWIGYIYPDYGTTDYANWVNGRFTISRDNSKNTVYLQMNSLRAEDTAVYYCARSGSYYSSGWGAEYFNLWGQGTLVTVSS

SEQ ID NO: 396 - protein, 257 aa, synthetic construct
  REGION 1..257: note = artificial antibody-related sequence
DIQMTQSPSSLSASVGDRVTITCQASQSISNWLAWYQQKPGKAPKLLIYKGGTLASGVPSRFSGSGSGTDFTLTISSLQPEDFATYYCQGYYYSSSGDDNAFGGGTKLTVLGGGGGSGGGGSGGGGSGGGGSEVQLVESGGGLVQPGGSLRLSCAASGIDFSRYGISWVRQAPGKGLEWIGYIYPDYGTTDYANWVNGRFTISRDNSKNTVYLQMNSLRAEDTAVYYCARSGSYYSSGWGAEYFNLWGQGTLVTVSS

SEQ ID NO: 397 - protein, 257 aa, synthetic construct
  REGION 1..257: note = artificial antibody-related sequence
DIQMTQSPSSLSASVGDRVTITCQASQSISNWLAWYQQKPGKAPKLLIYKGGTLASGVPSRFSGSGSGTDFTLTISSLQPEDFATYYCQGYYYSSSGDDNAFGGGTKLTVLGGGGGSGGGGSGGGGSGGGGSEVQLVESGGGLVQPGGSLRLSCAASGIDFSRYGISWVRQAPGKGLEWIGYIYPDYGTTDYANWVNGRFTISRDNSKNTVYLQMNSLRAEDSAVYYCARSGSYYSSGWGAEYFNLWGQGTLVKVSS

SEQ ID NO: 398 - protein, 125 aa, synthetic construct
  REGION 1..125: note = artificial antibody-related sequence
EVQLVESGGGLVQPGGSLRLSCKASGIDFSRYGISWVRQAPGKGLEWIAYIYPDYGTTDYANWVNGRFTISLDNSKNTVYLQMNSLRAEDTAVYYCARSGSYYSSGWGAEYFNLWGQGTLVTVSS

SEQ ID NO: 399 - protein, 125 aa, synthetic construct
  REGION 1..125: note = artificial antibody-related sequence
EVQLVESGGGLVQPGGSLRLSCKASGIDFSRYGISWVRQAPGKGLEWIAYIYPDYGTTDYANWVNGRFTISLDNSKNTVYLQMNSLRAEDSAVYYCARSGSYYSSGWGAEYFNLWGQGTLVKVSS

SEQ ID NO: 400 - protein, 112 aa, synthetic construct
  REGION 1..112: note = artificial antibody-related sequence
DIQMTQSPSSLSASVGDRVTINCQASQSISNWLAWYQQKPGKAPKLLIYKASTLASGVPSRFSGSGSGTEFTLTISGLQPADFATYYCQGYYYSSSGDDNAFGGGTKLTVLG

SEQ ID NO: 401 - protein, 112 aa, synthetic construct
  REGION 1..112: note = artificial antibody-related sequence
DIQMTQSPSSLSASVGDRVTINCQASQSISNWLAWYQQKPGKAPKLLIYKGGTLASGVPSRFSGSGSGTEFTLTISGLQPADFATYYCQGYYYSSSGDDNAFGGGTKLTVLG

SEQ ID NO: 402 - protein, 257 aa, synthetic construct
  REGION 1..257: note = artificial antibody-related sequence
DIQMTQSPSSLSASVGDRVTINCQASQSISNWLAWYQQKPGKAPKLLIYKASTLASGVPSRFSGSGSGTEFTLTISGLQPADFATYYCQGYYYSSSGDDNAFGGGTKLTVLGGGGGSGGGGSGGGGSGGGGSEVQLVESGGGLVQPGGSLRLSCKASGIDFSRYGISWVRQAPGKGLEWIAYIYPDYGTTDYANWVNGRFTISLDNSKNTVYLQMNSLRAEDTAVYYCARSGSYYSSGWGAEYFNLWGQGTLVTVSS

SEQ ID NO: 403 - protein, 257 aa, synthetic construct
  REGION 1..257: note = artificial antibody-related sequence
DIQMTQSPSSLSASVGDRVTINCQASQSISNWLAWYQQKPGKAPKLLIYKGGTLASGVPSRFSGSGSGTEFTLTISGLQPADFATYYCQGYYYSSSGDDNAFGGGTKLTVLGGGGGSGGGGSGGGGSGGGGSEVQLVESGGGLVQPGGSLRLSCKASGIDFSRYGISWVRQAPGKGLEWIAYIYPDYGTTDYANWVNGRFTISLDNSKNTVYLQMNSLRAEDTAVYYCARSGSYYSSGWGAEYFNLWGQGTLVTVSS

SEQ ID NO: 404 - protein, 257 aa, synthetic construct
  REGION 1..257: note = artificial antibody-related sequence
DIQMTQSPSSLSASVGDRVTINCQASQSISNWLAWYQQKPGKAPKLLIYKGGTLASGVPSRFSGSGSGTEFTLTISGLQPADFATYYCQGYYYSSSGDDNAFGGGTKLTVLGGGGGSGGGGSGGGGSGGGGSEVQLVESGGGLVQPGGSLRLSCKASGIDFSRYGISWVRQAPGKGLEWIAYIYPDYGTTDYANWVNGRFTISLDNSKNTVYLQMNSLRAEDSAVYYCARSGSYYSSGWGAEYFNLWGQGTLVKVSS

SEQ ID NO: 405 - protein, 125 aa, synthetic construct
  REGION 1..125: note = artificial antibody-related sequence
EVQLVESGGGLVQPGGSLRLSCAASGIDFSRYGISWVRQAPGKGLEWIAYIYPDYGTTDYANWVNGRFTISLDNAQNTVYLQMNSLRAEDTAVYYCARSGSYYSSGWGAEYFNLWGQGTLVTVSS

SEQ ID NO: 406 - protein, 125 aa, synthetic construct
  REGION 1..125: note = artificial antibody-related sequence
EVQLVESGGGLVQPGGSLRLSCAASGIDFSRYGISWVRQAPGKGLEWIAYIYPDYGTTDYANWVNGRFTISLDNAQNTVYLQMNSLRAEDSAVYYCARSGSYYSSGWGAEYFNLWGQGTLVKVSS

SEQ ID NO: 407 - protein, 112 aa, synthetic construct
  REGION 1..112: note = artificial antibody-related sequence
DIQMTQSPSSLSASVGDRVTINCQASQSISNWLAWYQQKPGKRPKLLIYKASTLASGVPSRFSGSGSGTDYTLTISSLQPEDFATYYCQGYYYSSSGDDNAFGGGTKLTVLG

SEQ ID NO: 408 - protein, 112 aa, synthetic construct
  REGION 1..112: note = artificial antibody-related sequence
DIQMTQSPSSLSASVGDRVTINCQASQSISNWLAWYQQKPGKRPKLLIYKGGTLASGVPSRFSGSGSGTDYTLTISSLQPEDFATYYCQGYYYSSSGDDNAFGGGTKLTVLG

SEQ ID NO: 409 - protein, 257 aa, synthetic construct
  REGION 1..257: note = artificial antibody-related sequence
DIQMTQSPSSLSASVGDRVTINCQASQSISNWLAWYQQKPGKRPKLLIYKASTLASGVPSRFSGSGSGTDYTLTISSLQPEDFATYYCQGYYYSSSGDDNAFGGGTKLTVLGGGGGSGGGGSGGGGSGGGGSEVQLVESGGGLVQPGGSLRLSCAASGIDFSRYGISWVRQAPGKGLEWIAYIYPDYGTTDYANWVNGRFTISLDNAQNTVYLQMNSLRAEDTAVYYCARSGSYYSSGWGAEYFNLWGQGTLVTVSS

SEQ ID NO: 410 - protein, 257 aa, synthetic construct
  REGION 1..257: note = artificial antibody-related sequence
DIQMTQSPSSLSASVGDRVTINCQASQSISNWLAWYQQKPGKRPKLLIYKGGTLASGVPSRFSGSGSGTDYTLTISSLQPEDFATYYCQGYYYSSSGDDNAFGGGTKLTVLGGGGGSGGGGSGGGGSGGGGSEVQLVESGGGLVQPGGSLRLSCAASGIDFSRYGISWVRQAPGKGLEWIAYIYPDYGTTDYANWVNGRFTISLDNAQNTVYLQMNSLRAEDTAVYYCARSGSYYSSGWGAEYFNLWGQGTLVTVSS

SEQ ID NO: 411 - protein, 257 aa, synthetic construct
  REGION 1..257: note = artificial antibody-related sequence
DIQMTQSPSSLSASVGDRVTINCQASQSISNWLAWYQQKPGKRPKLLIYKGGTLASGVPSRFSGSGSGTDYTLTISSLQPEDFATYYCQGYYYSSSGDDNAFGGGTKLTVLGGGGGSGGGGSGGGGSGGGGSEVQLVESGGGLVQPGGSLRLSCAASGIDFSRYGISWVRQAPGKGLEWIAYIYPDYGTTDYANWVNGRFTISLDNAQNTVYLQMNSLRAEDSAVYYCARSGSYYSSGWGAEYFNLWGQGTLVKVSS

SEQ ID NO: 412 - protein, 126 aa, synthetic construct
  REGION 1..126: note = artificial antibody-related sequence
EVQLVESGGGLVQPGGSLRLSCAASGIDFNKYYFMCWVRQAPGKGLEWIGCINTGSGSTYYATWAKGRFTISRDNSKNTVYLQMNSLRAEDTAVYYCARASGSSSVVYIGVYFSLWGQGTLVTVSS

SEQ ID NO: 413 - protein, 126 aa, synthetic construct
  REGION 1..126: note = artificial antibody-related sequence
EVQLVESGGGLVQPGGSLRLSCAASGIDFNKYYFMCWVRQAPGKGLEWIGCINTGSGSTYYATWAKGRFTISRDNSKNTVYLQMNSLRAEDSAVYYCARASGSSSVVYIGVYFSLWGQGTLVKVSS

SEQ ID NO: 414 - protein, 112 aa, synthetic construct
  REGION 1..112: note = artificial antibody-related sequence
DIQMTQSPSSLSASVGDRVTITCQASESIGNALAWYQQKPGKAPKLLIYSASTLASGVPSRFSGSGSGTDFTLTISSLQPEDFATYYCQSYYYSSSGSYVAFGGGTKLTVLG

SEQ ID NO: 415 - protein, 112 aa, synthetic construct
  REGION 1..112: note = artificial antibody-related sequence
DIQMTQSPSSLSASVGDRVTITCQASESIGNALAWYQQKPGKAPKLLIYSGGTLASGVPSRFSGSGSGTDFTLTISSLQPEDFATYYCQSYYYSSSGSYVAFGGGTKLTVLG

SEQ ID NO: 416 - protein, 258 aa, synthetic construct
  REGION 1..258: note = artificial antibody-related sequence
DIQMTQSPSSLSASVGDRVTITCQASESIGNALAWYQQKPGKAPKLLIYSASTLASGVPSRFSGSGSGTDFTLTISSLQPEDFATYYCQSYYYSSSGSYVAFGGGTKLTVLGGGGGSGGGGSGGGGSGGGGSEVQLVESGGGLVQPGGSLRLSCAASGIDFNKYYFMCWVRQAPGKGLEWIGCINTGSGSTYYATWAKGRFTISRDNSKNTVYLQMNSLRAEDTAVYYCARASGSSSVVYIGVYFSLWGQGTLVTVSS

SEQ ID NO: 417 - protein, 258 aa, synthetic construct
  REGION 1..258: note = artificial antibody-related sequence
DIQMTQSPSSLSASVGDRVTITCQASESIGNALAWYQQKPGKAPKLLIYSGGTLASGVPSRFSGSGSGTDFTLTISSLQPEDFATYYCQSYYYSSSGSYVAFGGGTKLTVLGGGGGSGGGGSGGGGSGGGGSEVQLVESGGGLVQPGGSLRLSCAASGIDFNKYYFMCWVRQAPGKGLEWIGCINTGSGSTYYATWAKGRFTISRDNSKNTVYLQMNSLRAEDTAVYYCARASGSSSVVYIGVYFSLWGQGTLVTVSS

SEQ ID NO: 418 - protein, 258 aa, synthetic construct
  REGION 1..258: note = artificial antibody-related sequence
DIQMTQSPSSLSASVGDRVTITCQASESIGNALAWYQQKPGKAPKLLIYSGGTLASGVPSRFSGSGSGTDFTLTISSLQPEDFATYYCQSYYYSSSGSYVAFGGGTKLTVLGGGGGSGGGGSGGGGSGGGGSEVQLVESGGGLVQPGGSLRLSCAASGIDFNKYYFMCWVRQAPGKGLEWIGCINTGSGSTYYATWAKGRFTISRDNSKNTVYLQMNSLRAEDSAVYYCARASGSSSVVYIGVYFSLWGQGTLVKVSS

SEQ ID NO: 419 - protein, 126 aa, synthetic construct
  REGION 1..126: note = artificial antibody-related sequence
EVQLVESGGGLVQPGGSLRLSCKASGIDFNKYYFMCWVRQAPGKGLEWIGCINTGSGSTYYATWAKGRFTISKDSSKNTVYLQMNSLRAEDTAVYYCARASGSSSVVYIGVYFSLWGQGTLVTVSS

SEQ ID NO: 420 - protein, 126 aa, synthetic construct
  REGION 1..126: note = artificial antibody-related sequence
EVQLVESGGGLVQPGGSLRLSCKASGIDFNKYYFMCWVRQAPGKGLEWIGCINTGSGSTYYATWAKGRFTISKDSSKNTVYLQMNSLRAEDSAVYYCARASGSSSVVYIGVYFSLWGQGTLVKVSS

SEQ ID NO: 421 - protein, 112 aa, synthetic construct
  REGION 1..112: note = artificial antibody-related sequence
DIQMTQSPSSLSASVGDRVTIKCQASESIGNALAWYQQKPGKAPKLLIYSASTLASGVPSRFSGSGSGTEFTLTISDLEPADFATYYCQSYYYSSSGSYVAFGGGTKLTVLG

SEQ ID NO: 422 - protein, 112 aa, synthetic construct
  REGION 1..112: note = artificial antibody-related sequence
DIQMTQSPSSLSASVGDRVTIKCQASESIGNALAWYQQKPGKAPKLLIYSGGTLASGVPSRFSGSGSGTEFTLTISDLEPADFATYYCQSYYYSSSGSYVAFGGGTKLTVLG

SEQ ID NO: 423 - protein, 258 aa, synthetic construct
  REGION 1..258: note = artificial antibody-related sequence
DIQMTQSPSSLSASVGDRVTIKCQASESIGNALAWYQQKPGKAPKLLIYSASTLASGVPSRFSGSGSGTEFTLTISDLEPADFATYYCQSYYYSSSGSYVAFGGGTKLTVLGGGGGSGGGGSGGGGSGGGGSEVQLVESGGGLVQPGGSLRLSCKASGIDFNKYYFMCWVRQAPGKGLEWIGCINTGSGSTYYATWAKGRFTISKDSSKNTVYLQMNSLRAEDTAVYYCARASGSSSVVYIGVYFSLWGQGTLVTVSS

SEQ ID NO: 424 - protein, 258 aa, synthetic construct
  REGION 1..258: note = artificial antibody-related sequence
DIQMTQSPSSLSASVGDRVTIKCQASESIGNALAWYQQKPGKAPKLLIYSGGTLASGVPSRFSGSGSGTEFTLTISDLEPADFATYYCQSYYYSSSGSYVAFGGGTKLTVLGGGGGSGGGGSGGGGSGGGGSEVQLVESGGGLVQPGGSLRLSCKASGIDFNKYYFMCWVRQAPGKGLEWIGCINTGSGSTYYATWAKGRFTISKDSSKNTVYLQMNSLRAEDTAVYYCARASGSSSVVYIGVYFSLWGQGTLVTVSS

SEQ ID NO: 425 - protein, 258 aa, synthetic construct
  REGION 1..258: note = artificial antibody-related sequence
DIQMTQSPSSLSASVGDRVTIKCQASESIGNALAWYQQKPGKAPKLLIYSGGTLASGVPSRFSGSGSGTEFTLTISDLEPADFATYYCQSYYYSSSGSYVAFGGGTKLTVLGGGGGSGGGGSGGGGSGGGGSEVQLVESGGGLVQPGGSLRLSCKASGIDFNKYYFMCWVRQAPGKGLEWIGCINTGSGSTYYATWAKGRFTISKDSSKNTVYLQMNSLRAEDSAVYYCARASGSSSVVYIGVYFSLWGQGTLVKVSS

SEQ ID NO: 426 - protein, 126 aa, synthetic construct
  REGION 1..126: note = artificial antibody-related sequence
EVQLVESGGGLVQPGGSLRLSCAASGIDFNKYYFMCWVRQAPGKCLEWIGCINTGSGSTYYATWAKGRFTISRDNSKNTVYLQMNSLRAEDTAVYYCARASGSSSVVYIGVYFSLWGQGTLVTVSS

SEQ ID NO: 427 - protein, 126 aa, synthetic construct
  REGION 1..126: note = artificial antibody-related sequence
EVQLVESGGGLVQPGGSLRLSCAASGIDFNKYYFMCWVRQAPGKCLEWIGCINTGSGSTYYATWAKGRFTISRDNSKNTVYLQMNSLRAEDSAVYYCARASGSSSVVYIGVYFSLWGQGTLVKVSS

SEQ ID NO: 428 - protein, 112 aa, synthetic construct
  REGION 1..112: note = artificial antibody-related sequence
DIQMTQSPSSLSASVGDRVTITCQASESIGNALAWYQQKPGKAPKLLIYSASTLASGVPSRFSGSGSGTDFTLTISSLQPEDFATYYCQSYYYSSSGSYVAFGCGTKLTVLG

SEQ ID NO: 429 - protein, 112 aa, synthetic construct
  REGION 1..112: note = artificial antibody-related sequence
DIQMTQSPSSLSASVGDRVTITCQASESIGNALAWYQQKPGKAPKLLIYSGGTLASGVPSRFSGSGSGTDFTLTISSLQPEDFATYYCQSYYYSSSGSYVAFGCGTKLTVLG

SEQ ID NO: 430 - protein, 258 aa, synthetic construct
  REGION 1..258: note = artificial antibody-related sequence
DIQMTQSPSSLSASVGDRVTITCQASESIGNALAWYQQKPGKAPKLLIYSASTLASGVPSRFSGSGSGTDFTLTISSLQPEDFATYYCQSYYYSSSGSYVAFGCGTKLTVLGGGGGSGGGGSGGGGSGGGGSEVQLVESGGGLVQPGGSLRLSCAASGIDFNKYYFMCWVRQAPGKCLEWIGCINTGSGSTYYATWAKGRFTISRDNSKNTVYLQMNSLRAEDTAVYYCARASGSSSVVYIGVYFSLWGQGTLVTVSS

SEQ ID NO: 431 - protein, 258 aa, synthetic construct
  REGION 1..258: note = artificial antibody-related sequence
DIQMTQSPSSLSASVGDRVTITCQASESIGNALAWYQQKPGKAPKLLIYSGGTLASGVPSRFSGSGSGTDFTLTISSLQPEDFATYYCQSYYYSSSGSYVAFGCGTKLTVLGGGGGSGGGGSGGGGSGGGGSEVQLVESGGGLVQPGGSLRLSCAASGIDFNKYYFMCWVRQAPGKCLEWIGCINTGSGSTYYATWAKGRFTISRDNSKNTVYLQMNSLRAEDTAVYYCARASGSSSVVYIGVYFSLWGQGTLVTVSS

SEQ ID NO: 432 - protein, 258 aa, synthetic construct
  REGION 1..258: note = artificial antibody-related sequence
DIQMTQSPSSLSASVGDRVTITCQASESIGNALAWYQQKPGKAPKLLIYSGGTLASGVPSRFSGSGSGTDFTLTISSLQPEDFATYYCQSYYYSSSGSYVAFGCGTKLTVLGGGGGSGGGGSGGGGSGGGGSEVQLVESGGGLVQPGGSLRLSCAASGIDFNKYYFMCWVRQAPGKCLEWIGCINTGSGSTYYATWAKGRFTISRDNSKNTVYLQMNSLRAEDSAVYYCARASGSSSVVYIGVYFSLWGQGTLVKVSS

SEQ ID NO: 433 - protein, 126 aa, synthetic construct
  REGION 1..126: note = artificial antibody-related sequence
EVQLVESGGGLVQPGGSLRLSCAASGIDFNKYYFICWVRQAPGKGLEWIGCINTGTGSTYFASWAKGRFTISRDNSKNTVYLQMNSLRAEDTAVYYCARASGSSSVYYIGVYFHLWGQGTLVTVSS

SEQ ID NO: 434 - protein, 126 aa, synthetic construct
  REGION 1..126: note = artificial antibody-related sequence
EVQLVESGGGLVQPGGSLRLSCAASGIDFNKYYFICWVRQAPGKGLEWIGCINTGTGSTYFASWAKGRFTISRDNSKNTVYLQMNSLRAEDSAVYYCARASGSSSVYYIGVYFHLWGQGTLVKVSS

SEQ ID NO: 435 - protein, 112 aa, synthetic construct
  REGION 1..112: note = artificial antibody-related sequence
DIQMTQSPSSLSASVGDRVTITCQASQNIYSDLAWYQQKPGKAPKLLIYGASTLASGVPSRFSGSGSGTDFTLTISSLQPEDFATYYCQSYYYSSSGSDVAFGGGTKLTVLG

SEQ ID NO: 436 - protein, 112 aa, synthetic construct
  REGION 1..112: note = artificial antibody-related sequence
DIQMTQSPSSLSASVGDRVTITCQASQNIYSDLAWYQQKPGKAPKLLIYGGGTLASGVPSRFSGSGSGTDFTLTISSLQPEDFATYYCQSYYYSSSGSDVAFGGGTKLTVLG

SEQ ID NO: 437 - protein, 258 aa, synthetic construct
  REGION 1..258: note = artificial antibody-related sequence
DIQMTQSPSSLSASVGDRVTITCQASQNIYSDLAWYQQKPGKAPKLLIYGASTLASGVPSRFSGSGSGTDFTLTISSLQPEDFATYYCQSYYYSSSGSDVAFGGGTKLTVLGGGGGSGGGGSGGGGSGGGGSEVQLVESGGGLVQPGGSLRLSCAASGIDFNKYYFICWVRQAPGKGLEWIGCINTGTGSTYFASWAKGRFTISRDNSKNTVYLQMNSLRAEDTAVYYCARASGSSSVYYIGVYFHLWGQGTLVTVSS

SEQ ID NO: 438 - protein, 258 aa, synthetic construct
  REGION 1..258: note = artificial antibody-related sequence
DIQMTQSPSSLSASVGDRVTITCQASQNIYSDLAWYQQKPGKAPKLLIYGGGTLASGVPSRFSGSGSGTDFTLTISSLQPEDFATYYCQSYYYSSSGSDVAFGGGTKLTVLGGGGGSGGGGSGGGGSGGGGSEVQLVESGGGLVQPGGSLRLSCAASGIDFNKYYFICWVRQAPGKGLEWIGCINTGTGSTYFASWAKGRFTISRDNSKNTVYLQMNSLRAEDTAVYYCARASGSSSVYYIGVYFHLWGQGTLVTVSS

SEQ ID NO: 439 - protein, 258 aa, synthetic construct
  REGION 1..258: note = artificial antibody-related sequence
DIQMTQSPSSLSASVGDRVTITCQASQNIYSDLAWYQQKPGKAPKLLIYGGGTLASGVPSRFSGSGSGTDFTLTISSLQPEDFATYYCQSYYYSSSGSDVAFGGGTKLTVLGGGGGSGGGGSGGGGSGGGGSEVQLVESGGGLVQPGGSLRLSCAASGIDFNKYYFICWVRQAPGKGLEWIGCINTGTGSTYFASWAKGRFTISRDNSKNTVYLQMNSLRAEDSAVYYCARASGSSSVYYIGVYFHLWGQGTLVKVSS

SEQ ID NO: 440 - protein, 126 aa, synthetic construct
  REGION 1..126: note = artificial antibody-related sequence
EVQLVESGGGLVQPGGSLRLSCKASGIDFNKYYFICWVRQAPGKGLEWIGCINTGTGSTYFASWAKGRFTISKDSSKNTVYLQMNSLRAEDTAVYYCARASGSSSVYYIGVYFHLWGQGTLVTVSS

SEQ ID NO: 441 - protein, 126 aa, synthetic construct
  REGION 1..126: note = artificial antibody-related sequence
EVQLVESGGGLVQPGGSLRLSCKASGIDFNKYYFICWVRQAPGKGLEWIGCINTGTGSTYFASWAKGRFTISKDSSKNTVYLQMNSLRAEDSAVYYCARASGSSSVYYIGVYFHLWGQGTLVKVSS

SEQ ID NO: 442 - protein, 112 aa, synthetic construct
  REGION 1..112: note = artificial antibody-related sequence
DIQMTQSPSSLSASVGDRVTIKCQASQNIYSDLAWYQQKPGKAPKLLIYGASTLASGVPSRFSGSGSGTEFTLTISDLEPADFATYYCQSYYYSSSGSDVAFGGGTKLTVLG

SEQ ID NO: 443 - protein, 112 aa, synthetic construct
  REGION 1..112: note = artificial antibody-related sequence
DIQMTQSPSSLSASVGDRVTIKCQASQNIYSDLAWYQQKPGKAPKLLIYGGGTLASGVPSRFSGSGSGTEFTLTISDLEPADFATYYCQSYYYSSSGSDVAFGGGTKLTVLG

SEQ ID NO: 444 - protein, 258 aa, synthetic construct
  REGION 1..258: note = artificial antibody-related sequence
DIQMTQSPSSLSASVGDRVTIKCQASQNIYSDLAWYQQKPGKAPKLLIYGASTLASGVPSRFSGSGSGTEFTLTISDLEPADFATYYCQSYYYSSSGSDVAFGGGTKLTVLGGGGGSGGGGSGGGGSGGGGSEVQLVESGGGLVQPGGSLRLSCKASGIDFNKYYFICWVRQAPGKGLEWIGCINTGTGSTYFASWAKGRFTISKDSSKNTVYLQMNSLRAEDTAVYYCARASGSSSVYYIGVYFHLWGQGTLVTVSS

SEQ ID NO: 445 - protein, 258 aa, synthetic construct
  REGION 1..258: note = artificial antibody-related sequence
DIQMTQSPSSLSASVGDRVTIKCQASQNIYSDLAWYQQKPGKAPKLLIYGGGTLASGVPSRFSGSGSGTEFTLTISDLEPADFATYYCQSYYYSSSGSDVAFGGGTKLTVLGGGGGSGGGGSGGGGSGGGGSEVQLVESGGGLVQPGGSLRLSCKASGIDFNKYYFICWVRQAPGKGLEWIGCINTGTGSTYFASWAKGRFTISKDSSKNTVYLQMNSLRAEDTAVYYCARASGSSSVYYIGVYFHLWGQGTLVTVSS

SEQ ID NO: 446 - protein, 258 aa, synthetic construct
  REGION 1..258: note = artificial antibody-related sequence
DIQMTQSPSSLSASVGDRVTIKCQASQNIYSDLAWYQQKPGKAPKLLIYGGGTLASGVPSRFSGSGSGTEFTLTISDLEPADFATYYCQSYYYSSSGSDVAFGGGTKLTVLGGGGGSGGGGSGGGGSGGGGSEVQLVESGGGLVQPGGSLRLSCKASGIDFNKYYFICWVRQAPGKGLEWIGCINTGTGSTYFASWAKGRFTISKDSSKNTVYLQMNSLRAEDSAVYYCARASGSSSVYYIGVYFHLWGQGTLVKVSS

SEQ ID NO: 447 - protein, 126 aa, synthetic construct
  REGION 1..126: note = artificial antibody-related sequence
EVQLVESGGGLVQPGGSLRLSCAASGIDFNKYYFICWVRQAPGKCLEWIGCINTGTGSTYFASWAKGRFTISRDNSKNTVYLQMNSLRAEDTAVYYCARASGSSSVYYIGVYFHLWGQGTLVTVSS

SEQ ID NO: 448 - protein, 126 aa, synthetic construct
  REGION 1..126: note = artificial antibody-related sequence
EVQLVESGGGLVQPGGSLRLSCAASGIDFNKYYFICWVRQAPGKCLEWIGCINTGTGSTYFASWAKGRFTISRDNSKNTVYLQMNSLRAEDSAVYYCARASGSSSVYYIGVYFHLWGQGTLVKVSS

SEQ ID NO: 449 - protein, 112 aa, synthetic construct
  REGION 1..112: note = artificial antibody-related sequence
DIQMTQSPSSLSASVGDRVTITCQASQNIYSDLAWYQQKPGKAPKLLIYGASTLASGVPSRFSGSGSGTDFTLTISSLQPEDFATYYCQSYYYSSSGSDVAFGCGTKLTVLG

SEQ ID NO: 450 - protein, 112 aa, synthetic construct
  REGION 1..112: note = artificial antibody-related sequence
DIQMTQSPSSLSASVGDRVTITCQASQNIYSDLAWYQQKPGKAPKLLIYGGGTLASGVPSRFSGSGSGTDFTLTISSLQPEDFATYYCQSYYYSSSGSDVAFGCGTKLTVLG

SEQ ID NO: 451 - protein, 258 aa, synthetic construct
  REGION 1..258: note = artificial antibody-related sequence
DIQMTQSPSSLSASVGDRVTITCQASQNIYSDLAWYQQKPGKAPKLLIYGASTLASGVPSRFSGSGSGTDFTLTISSLQPEDFATYYCQSYYYSSSGSDVAFGCGTKLTVLGGGGGSGGGGSGGGGSGGGGSEVQLVESGGGLVQPGGSLRLSCAASGIDFNKYYFICWVRQAPGKCLEWIGCINTGTGSTYFASWAKGRFTISRDNSKNTVYLQMNSLRAEDTAVYYCARASGSSSVYYIGVYFHLWGQGTLVTVSS

SEQ ID NO: 452 - protein, 258 aa, synthetic construct
  REGION 1..258: note = artificial antibody-related sequence
DIQMTQSPSSLSASVGDRVTITCQASQNIYSDLAWYQQKPGKAPKLLIYGGGTLASGVPSRFSGSGSGTDFTLTISSLQPEDFATYYCQSYYYSSSGSDVAFGCGTKLTVLGGGGGSGGGGSGGGGSGGGGSEVQLVESGGGLVQPGGSLRLSCAASGIDFNKYYFICWVRQAPGKCLEWIGCINTGTGSTYFASWAKGRFTISRDNSKNTVYLQMNSLRAEDTAVYYCARASGSSSVYYIGVYFHLWGQGTLVTVSS

SEQ ID NO: 453 - protein, 258 aa, synthetic construct
  REGION 1..258: note = artificial antibody-related sequence
DIQMTQSPSSLSASVGDRVTITCQASQNIYSDLAWYQQKPGKAPKLLIYGGGTLASGVPSRFSGSGSGTDFTLTISSLQPEDFATYYCQSYYYSSSGSDVAFGCGTKLTVLGGGGGSGGGGSGGGGSGGGGSEVQLVESGGGLVQPGGSLRLSCAASGIDFNKYYFICWVRQAPGKCLEWIGCINTGTGSTYFASWAKGRFTISRDNSKNTVYLQMNSLRAEDSAVYYCARASGSSSVYYIGVYFHLWGQGTLVKVSS

SEQ ID NO: 454 - protein, 119 aa, synthetic construct
  REGION 1..119: note = artificial antibody-related sequence
EVQLVESGGGLVQPGGSLRLSCAASGIDLSSYAMGWVRQAPGKGLEWIGTISVGGSTYYANWAEGRFTISRDNSKNTVYLQMNSLRAEDTAVYYCAGGDYINFFYFNIWGQGTLVTVSS

SEQ ID NO: 455 - protein, 119 aa, synthetic construct
  REGION 1..119: note = artificial antibody-related sequence
EVQLVESGGGLVQPGGSLRLSCAASGIDLSSYAMGWVRQAPGKGLEWIGTISVGGSTYYANWAEGRFTISRDNSKNTVYLQMNSLRAEDSAVYYCAGGDYINFFYFNIWGQGTLVKVSS

SEQ ID NO: 456 - protein, 112 aa, synthetic construct
  REGION 1..112: note = artificial antibody-related sequence
DIQMTQSPSSLSASVGDRVTITCQSSQSVYSSNLAWYQQKPGKAPKLLIYSASKLASGVPSRFSGSGSGTDFTLTISSLQPEDEATYYCQGTYYTSNWLNAFGGGTKLTVLG

SEQ ID NO: 457 - protein, 112 aa, synthetic construct
  REGION 1..112: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGGGTKLTVLG

SEQ ID NO: 458 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSASKLASGVPSRFSGSGSGTDFTLTISSLQPEDEATYYCQGTYYTSNWLNAFGGGTKLTVLGGGGGSGGGGSGGGGSGGGGSEVQLVESGGGLVQPGGSLRLSCAASGIDLSSYAMGWVRQAPGKGLEWIGTISVGGSTYYANWAEGRFTISRDNSKNTVYLQMNSLRAEDTAVYYCAGGDYINFFYFNIWGQGTLVTVSS

SEQ ID NO: 459 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGGGTKLTVLGGGGGSGGGGSGGGGSGGGGSEVQLVESGGGLVQPGGSLRLSCAASGIDLSSYAMGWVRQAPGKGLEWIGTISVGGSTYYANWAEGRFTISRDNSKNTVYLQMNSLRAEDTAVYYCAGGDYINFFYFNIWGQGTLVTVSS

SEQ ID NO: 460 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGGGTKLTVLGGGGGSGGGGSGGGGSGGGGSEVQLVESGGGLVQPGGSLRLSCAASGIDLSSYAMGWVRQAPGKGLEWIGTISVGGSTYYANWAEGRFTISRDNSKNTVYLQMNSLRAEDSAVYYCAGGDYINFFYFNIWGQGTLVKVSS

SEQ ID NO: 461 - protein, 119 aa, synthetic construct
  REGION 1..119: note = artificial antibody-related sequence
EVQLVESGGGLVQPGGSLRLSCTASGIDLSSYAMGWVRQAPGKGLEWIGTISVGGSTYYANWAEGRFTISKDTSKNTVYLQMNSLRAEDTAVYYCAGGDYINFFYFNIWGQGTLVTVSS

SEQ ID NO: 462 - protein, 119 aa, synthetic construct
  REGION 1..119: note = artificial antibody-related sequence
EVQLVESGGGLVQPGGSLRLSCTASGIDLSSYAMGWVRQAPGKGLEWIGTISVGGSTYYANWAEGRFTISKDTSKNTVYLQMNSLRAEDSAVYYCAGGDYINFFYFNIWGQGTLVKVSS

SEQ ID NO: 463 - protein, 112 aa, synthetic construct
  REGION 1..112: note = artificial antibody-related sequence
DIQMTQSPSSLSASVGDRVTINCQSSQSVYSSNLAWYQQKPGKAPKLLIYSASKLASGVPSRFSGSGSGTQFTLTISGLQPDDFATYYCQGTYYTSNWLNAFGGGTKLTVLG

SEQ ID NO: 464 - protein, 112 aa, synthetic construct
  REGION 1..112: note = artificial antibody-related sequence
DIQMTQSPSSLSASVGDRVTINCQSSQSVYSSNLAWYQQKPGKAPKLLIYSGGKLASGVPSRFSGSGSGTQFTLTISGLQPDDFATYYCQGTYYTSNWLNAFGGGTKLTVLG

SEQ ID NO: 465 - protein, 251 aa, synthetic construct
  REGION 1..251: note = artificial antibody-related sequence
DIQMTQSPSSLSASVGDRVTINCQSSQSVYSSNLAWYQQKPGKAPKLLIYSASKLASGVPSRFSGSGSGTQFTLTISGLQPDDFATYYCQGTYYTSNWLNAFGGGTKLTVLGGGGGSGGGGSGGGGSGGGGSEVQLVESGGGLVQPGGSLRLSCTASGIDLSSYAMGWVRQAPGKGLEWIGTISVGGSTYYANWAEGRFTISKDTSKNTVYLQMNSLRAEDTAVYYCAGGDYINFFYFNIWGQGTLVTVSS

SEQ ID NO: 466 - protein, 251 aa, synthetic construct
  REGION 1..251: note = artificial antibody-related sequence
DIQMTQSPSSLSASVGDRVTINCQSSQSVYSSNLAWYQQKPGKAPKLLIYSGGKLASGVPSRFSGSGSGTQFTLTISGLQPDDFATYYCQGTYYTSNWLNAFGGGTKLTVLGGGGGSGGGGSGGGGSGGGGSEVQLVESGGGLVQPGGSLRLSCTASGIDLSSYAMGWVRQAPGKGLEWIGTISVGGSTYYANWAEGRFTISKDTSKNTVYLQMNSLRAEDTAVYYCAGGDYINFFYFNIWGQGTLVTVSS

SEQ ID NO: 467 - protein, 251 aa, synthetic construct
  REGION 1..251: note = artificial antibody-related sequence
DIQMTQSPSSLSASVGDRVTINCQSSQSVYSSNLAWYQQKPGKAPKLLIYSGGKLASGVPSRFSGSGSGTQFTLTISGLQPDDFATYYCQGTYYTSNWLNAFGGGTKLTVLGGGGGSGGGGSGGGGSGGGGSEVQLVESGGGLVQPGGSLRLSCTASGIDLSSYAMGWVRQAPGKGLEWIGTISVGGSTYYANWAEGRFTISKDTSKNTVYLQMNSLRAEDSAVYYCAGGDYINFFYFNIWGQGTLVKVSS

SEQ ID NO: 468 - protein, 119 aa, synthetic construct
  REGION 1..119: note = artificial antibody-related sequence
EVQLVESGGGLVQPGGSLRLSCAASGIDLSSYAMGWVRQAPGKCLEWIGTISVGGSTYYANWAEGRFTISRDNSKNTVYLQMNSLRAEDTAVYYCAGGDYINFFYFNIWGQGTLVTVSS

SEQ ID NO: 469 - protein, 119 aa, synthetic construct
  REGION 1..119: note = artificial antibody-related sequence
EVQLVESGGGLVQPGGSLRLSCAASGIDLSSYAMGWVRQAPGKCLEWIGTISVGGSTYYANWAEGRFTISRDNSKNTVYLQMNSLRAEDSAVYYCAGGDYINFFYFNIWGQGTLVKVSS

SEQ ID NO: 470 - protein, 112 aa, synthetic construct
  REGION 1..112: note = artificial antibody-related sequence
DIQMTQSPSSLSASVGDRVTITCQSSQSVYSSNLAWYQQKPGKAPKLLIYSASKLASGVPSRFSGSGSGTDFTLTISSLQPEDEATYYCQGTYYTSNWLNAFGCGTKLTVLG

SEQ ID NO: 471 - protein, 112 aa, synthetic construct
  REGION 1..112: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGCGTKLTVLG

SEQ ID NO: 472 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSASKLASGVPSRFSGSGSGTDFTLTISSLQPEDEATYYCQGTYYTSNWLNAFGCGTKLTVLGGGGGSGGGGSGGGGSGGGGSEVQLVESGGGLVQPGGSLRLSCAASGIDLSSYAMGWVRQAPGKCLEWIGTISVGGSTYYANWAEGRFTISRDNSKNTVYLQMNSLRAEDTAVYYCAGGDYINFFYFNIWGQGTLVTVSS

SEQ ID NO: 473 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGCGTKLTVLGGGGGSGGGGSGGGGSGGGGSEVQLVESGGGLVQPGGSLRLSCAASGIDLSSYAMGWVRQAPGKCLEWIGTISVGGSTYYANWAEGRFTISRDNSKNTVYLQMNSLRAEDTAVYYCAGGDYINFFYFNIWGQGTLVTVSS

SEQ ID NO: 474 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGCGTKLTVLGGGGGSGGGGSGGGGSGGGGSEVQLVESGGGLVQPGGSLRLSCAASGIDLSSYAMGWVRQAPGKCLEWIGTISVGGSTYYANWAEGRFTISRDNSKNTVYLQMNSLRAEDSAVYYCAGGDYINFFYFNIWGQGTLVKVSS

SEQ ID NO: 475 - protein, 119 aa, synthetic construct
  REGION 1..119: note = artificial antibody-related sequence
EVQLVESGGGLVQPGGSLRLSCAASGIDLSSYAMGWVRQAPGKGLEWIGTISIGGNTYYANWAEGRFTISRDNSKNTVYLQMNSLRAEDTAVYYCAGGDYINFFYFNIWGQGTLVTVSS

SEQ ID NO: 476 - protein, 119 aa, synthetic construct
  REGION 1..119: note = artificial antibody-related sequence
EVQLVESGGGLVQPGGSLRLSCAASGIDLSSYAMGWVRQAPGKGLEWIGTISIGGNTYYANWAEGRFTISRDNSKNTVYLQMNSLRAEDSAVYYCAGGDYINFFYFNIWGQGTLVKVSS

SEQ ID NO: 477 - protein, 112 aa, synthetic construct; identical to SEQ ID NO: 456
  REGION 1..112: note = artificial antibody-related sequence

SEQ ID NO: 478 - protein, 112 aa, synthetic construct; identical to SEQ ID NO: 457
  REGION 1..112: note = artificial antibody-related sequence

SEQ ID NO: 479 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSASKLASGVPSRFSGSGSGTDFTLTISSLQPEDEATYYCQGTYYTSNWLNAFGGGTKLTVLGGGGGSGGGGSGGGGSGGGGSEVQLVESGGGLVQPGGSLRLSCAASGIDLSSYAMGWVRQAPGKGLEWIGTISIGGNTYYANWAEGRFTISRDNSKNTVYLQMNSLRAEDTAVYYCAGGDYINFFYFNIWGQGTLVTVSS

SEQ ID NO: 480 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGGGTKLTVLGGGGGSGGGGSGGGGSGGGGSEVQLVESGGGLVQPGGSLRLSCAASGIDLSSYAMGWVRQAPGKGLEWIGTISIGGNTYYANWAEGRFTISRDNSKNTVYLQMNSLRAEDTAVYYCAGGDYINFFYFNIWGQGTLVTVSS

SEQ ID NO: 481 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGGGTKLTVLGGGGGSGGGGSGGGGSGGGGSEVQLVESGGGLVQPGGSLRLSCAASGIDLSSYAMGWVRQAPGKGLEWIGTISIGGNTYYANWAEGRFTISRDNSKNTVYLQMNSLRAEDSAVYYCAGGDYINFFYFNIWGQGTLVKVSS

SEQ ID NO: 482 - protein, 119 aa, synthetic construct
  REGION 1..119: note = artificial antibody-related sequence
EVQLVESGGGLVQPGGSLRLSCTASGIDLSSYAMGWVRQAPGKGLEWIGTISIGGNTYYANWAEGRFTISKDTSKNTVYLQMNSLRAEDTAVYYCAGGDYINFFYFNIWGQGTLVTVSS

SEQ ID NO: 483 - protein, 119 aa, synthetic construct
  REGION 1..119: note = artificial antibody-related sequence
EVQLVESGGGLVQPGGSLRLSCTASGIDLSSYAMGWVRQAPGKGLEWIGTISIGGNTYYANWAEGRFTISKDTSKNTVYLQMNSLRAEDSAVYYCAGGDYINFFYFNIWGQGTLVKVSS

SEQ ID NO: 484 - protein, 112 aa, synthetic construct; identical to SEQ ID NO: 463
  REGION 1..112: note = artificial antibody-related sequence

SEQ ID NO: 485 - protein, 112 aa, synthetic construct; identical to SEQ ID NO: 464
  REGION 1..112: note = artificial antibody-related sequence

SEQ ID NO: 486 - protein, 251 aa, synthetic construct
  REGION 1..251: note = artificial antibody-related sequence
DIQMTQSPSSLSASVGDRVTINCQSSQSVYSSNLAWYQQKPGKAPKLLIYSASKLASGVPSRFSGSGSGTQFTLTISGLQPDDFATYYCQGTYYTSNWLNAFGGGTKLTVLGGGGGSGGGGSGGGGSGGGGSEVQLVESGGGLVQPGGSLRLSCTASGIDLSSYAMGWVRQAPGKGLEWIGTISIGGNTYYANWAEGRFTISKDTSKNTVYLQMNSLRAEDTAVYYCAGGDYINFFYFNIWGQGTLVTVSS

SEQ ID NO: 487 - protein, 251 aa, synthetic construct
  REGION 1..251: note = artificial antibody-related sequence
DIQMTQSPSSLSASVGDRVTINCQSSQSVYSSNLAWYQQKPGKAPKLLIYSGGKLASGVPSRFSGSGSGTQFTLTISGLQPDDFATYYCQGTYYTSNWLNAFGGGTKLTVLGGGGGSGGGGSGGGGSGGGGSEVQLVESGGGLVQPGGSLRLSCTASGIDLSSYAMGWVRQAPGKGLEWIGTISIGGNTYYANWAEGRFTISKDTSKNTVYLQMNSLRAEDTAVYYCAGGDYINFFYFNIWGQGTLVTVSS

SEQ ID NO: 488 - protein, 251 aa, synthetic construct
  REGION 1..251: note = artificial antibody-related sequence
DIQMTQSPSSLSASVGDRVTINCQSSQSVYSSNLAWYQQKPGKAPKLLIYSGGKLASGVPSRFSGSGSGTQFTLTISGLQPDDFATYYCQGTYYTSNWLNAFGGGTKLTVLGGGGGSGGGGSGGGGSGGGGSEVQLVESGGGLVQPGGSLRLSCTASGIDLSSYAMGWVRQAPGKGLEWIGTISIGGNTYYANWAEGRFTISKDTSKNTVYLQMNSLRAEDSAVYYCAGGDYINFFYFNIWGQGTLVKVSS

SEQ ID NO: 489 - protein, 119 aa, synthetic construct
  REGION 1..119: note = artificial antibody-related sequence
EVQLVESGGGLVQPGGSLRLSCAASGIDLSSYAMGWVRQAPGKCLEWIGTISIGGNTYYANWAEGRFTISRDNSKNTVYLQMNSLRAEDTAVYYCAGGDYINFFYFNIWGQGTLVTVSS

SEQ ID NO: 490 - protein, 119 aa, synthetic construct
  REGION 1..119: note = artificial antibody-related sequence
EVQLVESGGGLVQPGGSLRLSCAASGIDLSSYAMGWVRQAPGKCLEWIGTISIGGNTYYANWAEGRFTISRDNSKNTVYLQMNSLRAEDSAVYYCAGGDYINFFYFNIWGQGTLVKVSS

SEQ ID NO: 491 - protein, 112 aa, synthetic construct; identical to SEQ ID NO: 470
  REGION 1..112: note = artificial antibody-related sequence

SEQ ID NO: 492 - protein, 112 aa, synthetic construct; identical to SEQ ID NO: 471
  REGION 1..112: note = artificial antibody-related sequence

SEQ ID NO: 493 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSASKLASGVPSRFSGSGSGTDFTLTISSLQPEDEATYYCQGTYYTSNWLNAFGCGTKLTVLGGGGGSGGGGSGGGGSGGGGSEVQLVESGGGLVQPGGSLRLSCAASGIDLSSYAMGWVRQAPGKCLEWIGTISIGGNTYYANWAEGRFTISRDNSKNTVYLQMNSLRAEDTAVYYCAGGDYINFFYFNIWGQGTLVTVSS

SEQ ID NO: 494 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGCGTKLTVLGGGGGSGGGGSGGGGSGGGGSEVQLVESGGGLVQPGGSLRLSCAASGIDLSSYAMGWVRQAPGKCLEWIGTISIGGNTYYANWAEGRFTISRDNSKNTVYLQMNSLRAEDTAVYYCAGGDYINFFYFNIWGQGTLVTVSS

SEQ ID NO: 495 - protein, 251 aa, synthetic construct
  REGION 1..251: note = artificial antibody-related sequence
DIQMTQSPSSLSASVGDRVTITCQSSQSVYSSNLAWYQQKPGKAPKLLIYSGGKLASGVPSRFSGSGSGTDFTLTISSLQPEDEATYYCQGTYYTSNWLNAFGCGTKLTVLGGGGGSGGGGSGGGGSGGGGSEVQLVESGGGLVQPGGSLRLSCAASGIDLSSYAMGWVRQAPGKCLEWIGTISIGGNTYYANWAEGRFTISRDNSKNTVYLQMNSLRAEDSAVYYCAGGDYINFFYFNIWGQGTLVKVSS

SEQ ID NO: 496 - protein, 122 aa, synthetic construct; identical to SEQ ID NO: 74
  REGION 1..122: note = artificial antibody-related sequence

SEQ ID NO: 497 - protein, 122 aa, synthetic construct; identical to SEQ ID NO: 75
  REGION 1..122: note = artificial antibody-related sequence

SEQ ID NO: 498 - protein, 122 aa, synthetic construct
  REGION 1..122: note = artificial antibody-related sequence
EVQLVESGGGLVQPGGSLRLSCAASGFSFSANYYPCWVRQAPGKGLEWIACIYGGSSDITYDANWTKGRFTISRDNSKNTVYLQMNSLRAEDTAVYYCARSAWYSGWGGDLWGQGTLVTVSS

SEQ ID NO: 499 - protein, 122 aa, synthetic construct
  REGION 1..122: note = artificial antibody-related sequence
EVQLVESGGGLVQPGGSLRLSCAASGFSFSANYYPCWVRQAPGKCLEWIACIYGGSSDITYDANWTKGRFTISRDNSKNTVYLQMNSLRAEDTAVYYCARSAWYSGWGGDLWGQGTLVTVSS

SEQ ID NO: 500 - protein, 122 aa, synthetic construct
  REGION 1..122: note = artificial antibody-related sequence
EVQLVESGGGLVQPGGSLRLSCAASGFSFSANYYPCWVRQAPGKGLEWIGCIYGGSSDITYDANWTKGRFTISRDNSKNTVYLQMNSLRAEDTAVYFCARSAWYSGWGGDLWGQGTLVTVSS

SEQ ID NO: 501 - protein, 122 aa, synthetic construct
  REGION 1..122: note = artificial antibody-related sequence
EVQLVESGGGLVQPGGSLRLSCAASGFSFSANYYPCWVRQAPGKCLEWIGCIYGGSSDITYDANWTKGRFTISRDNSKNTVYLQMNSLRAEDTAVYFCARSAWYSGWGGDLWGQGTLVTVSS

SEQ ID NO: 502 - protein, 122 aa, synthetic construct
  REGION 1..122: note = artificial antibody-related sequence
EVQLVESGGGLVQPGGSLRLSCAASGFSFSANYYPCWVRQAPGKGLEWIGCIYGGSSDITYDANWTKGRFTISRDNSKNTLYLQMNSLRAEDTAVYYCARSAWYSGWGGDLWGQGTLVTVSS

SEQ ID NO: 503 - protein, 122 aa, synthetic construct
  REGION 1..122: note = artificial antibody-related sequence
EVQLVESGGGLVQPGGSLRLSCAASGFSFSANYYPCWVRQAPGKCLEWIGCIYGGSSDITYDANWTKGRFTISRDNSKNTLYLQMNSLRAEDTAVYYCARSAWYSGWGGDLWGQGTLVTVSS

SEQ ID NO: 504 - protein, 122 aa, synthetic construct
  REGION 1..122: note = artificial antibody-related sequence
EVQLVESGGGLVQPGGSLRLSCAASGFSFSANYYPCWVRQAPGKGLEWIACIYGGSSDITYDANWTKGRFTISRDNSKNTLYLQMNSLRAEDTAVYYCARSAWYSGWGGDLWGQGTLVTVSS

SEQ ID NO: 505 - protein, 122 aa, synthetic construct
  REGION 1..122: note = artificial antibody-related sequence
EVQLVESGGGLVQPGGSLRLSCAASGFSFSANYYPCWVRQAPGKCLEWIACIYGGSSDITYDANWTKGRFTISRDNSKNTLYLQMNSLRAEDTAVYYCARSAWYSGWGGDLWGQGTLVTVSS

SEQ ID NO: 506 - protein, 122 aa, synthetic construct
  REGION 1..122: note = artificial antibody-related sequence
EVQLVESGGGLVQPGGSLRLSCAASGFSFSANYYPCWVRQAPGKGLEWIACIYGGSSDITYDANWTKGRFTISRDNSKNTVYLQMNSLRAEDTAVYFCARSAWYSGWGGDLWGQGTLVTVSS

SEQ ID NO: 507 - protein, 122 aa, synthetic construct
  REGION 1..122: note = artificial antibody-related sequence
EVQLVESGGGLVQPGGSLRLSCAASGFSFSANYYPCWVRQAPGKCLEWIACIYGGSSDITYDANWTKGRFTISRDNSKNTVYLQMNSLRAEDTAVYFCARSAWYSGWGGDLWGQGTLVTVSS

SEQ ID NO: 508 - protein, 122 aa, synthetic construct
  REGION 1..122: note = artificial antibody-related sequence
EVQLVESGGGLVQPGGSLRLSCAASGFSFSANYYPCWVRQAPGKGLEWIACIYGGSSDITYDANWTKGRFTISRDNSKNTLYLQMNSLRAEDTAVYFCARSAWYSGWGGDLWGQGTLVTVSS

SEQ ID NO: 509 - protein, 122 aa, synthetic construct
  REGION 1..122: note = artificial antibody-related sequence
EVQLVESGGGLVQPGGSLRLSCAASGFSFSANYYPCWVRQAPGKCLEWIACIYGGSSDITYDANWTKGRFTISRDNSKNTLYLQMNSLRAEDTAVYFCARSAWYSGWGGDLWGQGTLVTVSS

SEQ ID NO: 510 - protein, 121 aa, synthetic construct
  REGION 1..121: note = artificial antibody-related sequence
QVQLVQSGAEVKKPGSSVKVSCKASGIDFNSNYYMCWVRQAPGQGLEWMGCIYVGSHVNTYYANWAKGRVTITADESTSTAYMELSSLRSEDTAVYYCATSGSSVLYFKFWGQGTLVTVSS

SEQ ID NO: 511 - protein, 121 aa, synthetic construct
  REGION 1..121: note = artificial antibody-related sequence
QVQLVQSGAEVKKPGASVKVSCKASGIDFNSNYYMCWVRQAPGQGLEWMGCIYVGSHVNTYYANWAKGRVTMTRDTSISTAYMELSSLRSEDTAVYYCATSGSSVLYFKFWGQGTLVTVSS

SEQ ID NO: 512 - protein, 121 aa, synthetic construct
  REGION 1..121: note = artificial antibody-related sequence
QVQLQESGPGLVKPSETLSLTCTVSGIDFNSNYYMCWIRQPPGKGLEWIGCIYVGSHVNTYYANWAKGRVTISVDTSKNQFSLKLSSVTAADTAVYYCATSGSSVLYFKFWGQGTLVTVSS

SEQ ID NO: 513 - protein, 121 aa, synthetic construct
  REGION 1..121: note = artificial antibody-related sequence
EVQLVQSGAEVKKPGESLKISCKGSGIDFNSNYYMCWVRQMPGKGLEWMGCIYVGSHVNTYYANWAKGQVTISADKSISTAYLQWSSLKASDTAMYYCATSGSSVLYFKFWGQGTLVTVSS

SEQ ID NO: 514 - protein, 121 aa, synthetic construct
  REGION 1..121: note = artificial antibody-related sequence
QVQLQQSGPGLVKPSQTLSLTCAISGIDFNSNYYMCWIRQSPGRGLEWLGCIYVGSHVNTYYANWAKGRITINPDTSKNQFSLQLNSVTPEDTAVYYCATSGSSVLYFKFWGQGTLVTVSS

SEQ ID NO: 515 - protein, 108 aa, synthetic construct
  REGION 1..108: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGQGTKVEIKR

SEQ ID NO: 516 - protein, 108 aa, synthetic construct
  REGION 1..108: note = artificial antibody-related sequence
DIQMTQSPSSLSASVGDRVTITCRASQSINNVLAWYQQKPGKAPKLLIYRASTLASGVPSRFSGSGSGTDFTLTISSLQPEDFATYYCQSSYGNYGDFGQGTKVEIKR

SEQ ID NO: 517 - protein, 114 aa, synthetic construct
  REGION 1..114: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGQGTKVEIKR

SEQ ID NO: 518 - protein, 114 aa, synthetic construct
  REGION 1..114: note = artificial antibody-related sequence
DIQMTQSPASLSASVGDRVTITCRASQSIGTYLAWYQQKPGKPPKLLIYRAFILASGVPSRFSGSGSGTDFTLTISSLQPEDFATYYCQSNFYSDSTTIGPNAFGQGTKVEIKR

SEQ ID NO: 519 - protein, 108 aa, synthetic construct; identical to SEQ ID NO: 112
  REGION 1..108: note = artificial antibody-related sequence

SEQ ID NO: 520 - protein, 108 aa, synthetic construct; identical to SEQ ID NO: 132
  REGION 1..108: note = artificial antibody-related sequence

SEQ ID NO: 521 - protein, 108 aa, synthetic construct
  REGION 1..108: note = artificial antibody-related sequence
DIQMTQSPSSLSASVGDRVTITCRASQSINNVLAWYQQKPGKAPKLLIYRASTLASGVPSRFSGSGSGTDFTLTISSLQPEDFATYYCQSSYGNYGDFGTGTKVTVLG

SEQ ID NO: 522 - protein, 108 aa, synthetic construct
  REGION 1..108: note = artificial antibody-related sequence
DIQMTQSPSSLSASVGDRVTITCRASQSINNVLAWYQQKPGKAPKLLIYRASTLASGVPSRFSGSGSGTDFTLTISSLQPEDFATYYCQSSYGNYGDFGCGTKVTVLG

SEQ ID NO: 523 - protein, 114 aa, synthetic construct; identical to SEQ ID NO: 19
  REGION 1..114: note = artificial antibody-related sequence

SEQ ID NO: 524 - protein, 114 aa, synthetic construct
  REGION 1..114: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGCGTKVTVLG

SEQ ID NO: 525 - protein, 114 aa, synthetic construct
  REGION 1..114: note = artificial antibody-related sequence
DIQMTQSPASLSASVGDRVTITCRASQSIGTYLAWYQQKPGKPPKLLIYRAFILASGVPSRFSGSGSGTDFTLTISSLQPEDFATYYCQSNFYSDSTTIGPNAFGTGTKVTVLG

SEQ ID NO: 526 - protein, 114 aa, synthetic construct
  REGION 1..114: note = artificial antibody-related sequence
DIQMTQSPASLSASVGDRVTITCRASQSIGTYLAWYQQKPGKPPKLLIYRAFILASGVPSRFSGSGSGTDFTLTISSLQPEDFATYYCQSNFYSDSTTIGPNAFGCGTKVTVLG

SEQ ID NO: 527 - protein, 11 aa, synthetic construct
  REGION 1..11: note = artificial antibody-related sequence
FGTGTKVTVLG

SEQ ID NO: 528 - protein, 11 aa, synthetic construct
  REGION 1..11: note = artificial antibody-related sequence
FGGGTKLTVLG

SEQ ID NO: 529 - protein, 11 aa, synthetic construct
  REGION 1..11: note = artificial antibody-related sequence
FGGGTQLIILG

SEQ ID NO: 530 - protein, 11 aa, synthetic construct
  REGION 1..11: note = artificial antibody-related sequence
FGEGTELTVLG

SEQ ID NO: 531 - protein, 11 aa, synthetic construct
  REGION 1..11: note = artificial antibody-related sequence
FGSGTKVTVLG

SEQ ID NO: 532 - protein, 11 aa, synthetic construct
  REGION 1..11: note = artificial antibody-related sequence
FGGGTQLTVLG

SEQ ID NO: 533 - protein, 11 aa, synthetic construct
  REGION 1..11: note = artificial antibody-related sequence
FGGGTQLTALG

SEQ ID NO: 534 - protein, 11 aa, synthetic construct
  REGION 1..11: note = artificial antibody-related sequence
FGCGTKVTVLG

SEQ ID NO: 535 - protein, 11 aa, synthetic construct
  REGION 1..11: note = artificial antibody-related sequence
FGTGTKLTVLG

SEQ ID NO: 536 - skipped

SEQ ID NO: 537 - protein, 5 aa, synthetic construct
  REGION 1..5: note = artificial antibody-related sequence
  REGION 1..5: note = repeat - linker (GGGGS)n, wherein n=1, 2, 3, 4 or 5
GGGGS

SEQ ID NO: 538 - protein, 10 aa, synthetic construct
  REGION 1..10: note = artificial antibody-related sequence
GGGGSGGGGS

SEQ ID NO: 539 - protein, 5 aa, synthetic construct
  REGION 1..5: note = artificial antibody-related sequence
  REGION 1..5: note = Repeat - linker (GGGGS)n, wherein n=1, 2, 3, 4, 5, 6, 7 or 8
GGGGS

SEQ ID NO: 540 - protein, 20 aa, synthetic construct
  REGION 1..20: note = artificial antibody-related sequence
GGGGSGGGGSGGGGSGGGGS

SEQ ID NO: 541 - protein, 5 aa, synthetic construct
  REGION 1..5: note = artificial antibody-related sequence
  REGION 1..5: note = Repeat - linker (GSGGS)n, wherein n is integer of at least 1
GSGGS

SEQ ID NO: 542 - protein, 5 aa, synthetic construct
  REGION 1..5: note = artificial antibody-related sequence
  REGION 1..5: note = Repeat - linker (GGGGS)n, wherein n is integer of at least 1
GGGGS

SEQ ID NO: 543 - protein, 5 aa, synthetic construct
  REGION 1..5: note = artificial antibody-related sequence
  REGION 1..4: note = Repeat - linker (GGGS)n, wherein n is integer of at least 1
XGGGS

SEQ ID NO: 544 - protein, 759 aa, synthetic construct
  REGION 1..759: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 545 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 546 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSSGGGGSGGGGSGGGGSGGGGSIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 547 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDSAVYYCARSAWYSGWGGDLWGQGTLVKVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 548 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDAQWTKGRFTISRDNSKNTVYLQMNSLRAEDSAVYYCARSAWYSGWGGDLWGQGTLVKVSSGGGGSGGGGSGGGGSGGGGSIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 549 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDAQWTKGRFTISRDNSKNTVYLQMNSLRAEDSAVYYCARSAWYSGWGGDLWGQGTLVKVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 550 - protein, 759 aa, synthetic construct; identical to SEQ ID NO: 548
  REGION 1..759: note = artificial antibody-related sequence

SEQ ID NO: 551 - protein, 759 aa, synthetic construct
  REGION 1..759: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDAQWTKGRFTISRDNSKNTVYLQMNSLRAEDSAVYYCARSAWYSGWGGDLWGQGTLVKVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGCGTKLTVLGGGGGSGGGGSGGGGSGGGGSEVQLVESGGGLVQPGGSLRLSCAASGFSLSSNAMGWVRQAPGKCLEYIGIISVGGFTYYASWAKGRFTISRDNSKNTVYLQMNSLRAEDSATYFCARDRHGGDSSGAFYLWGQGTLVKVSS

SEQ ID NO: 552 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDAQWTKGRFTISRDNSKNTVYLQMNSLRAEDSAVYYCARSAWYSGWGGDLWGQGTLVKVSSGGGGSGGGGSGGGGSGGGGSIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGCGTKLTVLGGGGGSGGGGSGGGGSGGGGSEVQLVESGGGLVQPGGSLRLSCAASGFSLSSNAMGWVRQAPGKCLEYIGIISVGGFTYYASWAKGRFTISRDNSKNTVYLQMNSLRAEDSATYFCARDRHGGDSSGAFYLWGQGTLVKVSS

SEQ ID NO: 553 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDAQWTKGRFTISRDNSKNTVYLQMNSLRAEDSAVYYCARSAWYSGWGGDLWGQGTLVKVSSGGGGSGGGGSGGGGSGGGGSIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGCGTKLTVLGGGGGSGGGGSGGGGSGGGGSEVQLVESGGGLVQPGGSLRLSCAASGFSLSSNAMGWVRQAPGKCLEYIGIISVGGFTYYASWAKGRFTISRDNSKNTVYLQMNSLRAEDSATYFCARDRHGGDSSGAFYLWGQGTLVKVSS

SEQ ID NO: 554 - protein, 755 aa, synthetic construct
  REGION 1..755: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 555 - protein, 755 aa, synthetic construct
  REGION 1..755: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 556 - protein, 755 aa, synthetic construct
  REGION 1..755: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADSAVYYCARHPSDAVYGYANNLWGQGTLVKVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 557 - protein, 761 aa, synthetic construct
  REGION 1..761: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADTAVYYCAREAASSSGYGMGYFDLWGQGTLVTVSSGGGGSGGGGSGGGGSGGGGSIQMTQSPSSLSASVGDRVTITCQSSQSVYDNNWLAWYQQKPGKAPKLLIYRASNLASGVPSRFSGSGSGTDFTLTISSLQPEDFATYYCQGTYLSSNWYWAFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 558 - protein, 761 aa, synthetic construct
  REGION 1..761: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADTAVYYCAREAASSSGYGMGYFDLWGQGTLVTVSSGGGGSGGGGSGGGGSGGGGSIQMTQSPSSLSASVGDRVTITCQSSQSVYDNNWLAWYQQKPGKAPKLLIYRGGNLASGVPSRFSGSGSGTDFTLTISSLQPEDFATYYCQGTYLSSNWYWAFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 559 - protein, 761 aa, synthetic construct
  REGION 1..761: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADSAVYYCAREAASSSGYGMGYFDLWGQGTLVKVSSGGGGSGGGGSGGGGSGGGGSIQMTQSPSSLSASVGDRVTITCQSSQSVYDNNWLAWYQQKPGKAPKLLIYRGGNLASGVPSRFSGSGSGTDFTLTISSLQPEDFATYYCQGTYLSSNWYWAFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 560 - protein, 753 aa, synthetic construct
  REGION 1..753: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 561 - protein, 753 aa, synthetic construct
  REGION 1..753: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 562 - protein, 753 aa, synthetic construct
  REGION 1..753: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADSAVYYCARHPSDAVYGYANNLWGQGTLVKVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSSGGGGSGGGGSVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDSATYFCARPVSVYYYGMDLWGQGTLVKVSS

SEQ ID NO: 563 - protein, 759 aa, synthetic construct
  REGION 1..759: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADTAVYYCAREAASSSGYGMGYFDLWGQGTLVTVSSGGGGSGGGGSGGGGSGGGGSIQMTQSPSSLSASVGDRVTITCQSSQSVYDNNWLAWYQQKPGKAPKLLIYRASNLASGVPSRFSGSGSGTDFTLTISSLQPEDFATYYCQGTYLSSNWYWAFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 564 - protein, 759 aa, synthetic construct
  REGION 1..759: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADTAVYYCAREAASSSGYGMGYFDLWGQGTLVTVSSGGGGSGGGGSGGGGSGGGGSIQMTQSPSSLSASVGDRVTITCQSSQSVYDNNWLAWYQQKPGKAPKLLIYRGGNLASGVPSRFSGSGSGTDFTLTISSLQPEDFATYYCQGTYLSSNWYWAFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 565 - protein, 759 aa, synthetic construct
  REGION 1..759: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSQVQLQESGPGLVKPSETLSLTCKVSGFSFNNDYDMCWIRQPPGKGLEWIGCIDTGDGSTYYASWAKGRVTISVDSSKNQFSLKLSSVTAADSAVYYCAREAASSSGYGMGYFDLWGQGTLVKVSSGGGGSGGGGSGGGGSGGGGSIQMTQSPSSLSASVGDRVTITCQSSQSVYDNNWLAWYQQKPGKAPKLLIYRGGNLASGVPSRFSGSGSGTDFTLTISSLQPEDFATYYCQGTYLSSNWYWAFGTGTKVTVLGGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSSGGGGSGGGGSVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDSATYFCARPVSVYYYGMDLWGQGTLVKVSS

SEQ ID NO: 566 - protein, 759 aa, synthetic construct
  REGION 1..759: note = artificial antibody-related sequence
DIQMTQSPSSLSASVGDRVTITCQASQSIGTYLAWYQQKPGKAPKLLIYRAFILASGVPSRFSGSGSGTDFTLTISSLQPEDFATYYCQSNFYSDSTTIGPNAFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGTGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 567 - protein, 759 aa, synthetic construct
  REGION 1..759: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 568 - protein, 759 aa, synthetic construct
  REGION 1..759: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADSAVYYCARHPSDAVYGYANNLWGQGTLVKVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADSAVYYCARGYVDYGGATDLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 569 - protein, 758 aa, synthetic construct
  REGION 1..758: note = artificial antibody-related sequence
DIQMTQSPSSLSASVGDRVTITCQASQSIGTYLAWYQQKPGKAPKLLIYRAFILASGVPSRFSGSGSGTDFTLTISSLQPEDFATYYCQSNFYSDSTTIGPNAFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSDIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGTGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSSGGGGSGGGGSVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 570 - protein, 758 aa, synthetic construct
  REGION 1..758: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADTAVYYCARHPSDAVYGYANNLWGQGTLVTVSSGGGGSGGGGSGGGGSGGGGSDIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSSGGGGSGGGGSVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDTATYFCARPVSVYYYGMDLWGQGTLVTVSS

SEQ ID NO: 571 - protein, 758 aa, synthetic construct
  REGION 1..758: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSQVQLQESGPGLVKPSETLSLTCKVSGFSFSNSYWICWIRQPPGKGLEWIGCTFVGSSDSTYYANWAKGRVTISVDSSKNQFSLKLSSVTAADSAVYYCARHPSDAVYGYANNLWGQGTLVKVSSGGGGSGGGGSGGGGSGGGGSDIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGTGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADSAVYYCARGYVDYGGATDLWGQGTLVKVSSGGGGSGGGGSVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGGGTKLTVLGGGGGSGGGGSGGGGSGGGGSEVQLVESGGGLVQPGGSLRLSCAASGFSFSSSYWICWVRQAPGKGLEWVGCVFTGDGTTYYASWAKGRFTISRDNSKNTVYLQMNSLRAEDSATYFCARPVSVYYYGMDLWGQGTLVKVSS

SEQ ID NO: 572 - protein, 759 aa, synthetic construct
  REGION 1..759: note = artificial antibody-related sequence
DIQMTQSPSSLSASVGDRVTITCQASQSIGTYLAWYQQKPGKAPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 573 - protein, 759 aa, synthetic construct
  REGION 1..759: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADTAVYYCARGYVDYGGATDLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 574 - protein, 759 aa, synthetic construct
  REGION 1..759: note = artificial antibody-related sequence
DIQMTQSPSSLSASVGDRVTITCQASQSIGTYLAWYQQKPGKAPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDSAVYYCARHPSDAVYGYANNLWGQGTLVKVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQVQLQESGPGLVKPSETLSLTCKVSGFSFNSDYWIYWIRQPPGKGLEWIGSIYGGSSGNTQYASWAQGRVTISVDSSKNQFSLKLSSVTAADSAVYYCARGYVDYGGATDLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 575 - protein, 759 aa, synthetic construct
  REGION 1..759: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 576 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 577 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDSAVYYCARHPSDAVYGYANNLWGQGTLVKVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 578 - protein, 759 aa, synthetic construct
  REGION 1..759: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSGGGGSGGGGSIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 579 - protein, 759 aa, synthetic construct
  REGION 1..759: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSGGGGSGGGGSIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 580 - protein, 759 aa, synthetic construct
  REGION 1..759: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSGGGGSGGGGSIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDSAVYYCARHPSDAVYGYANNLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 581 - protein, 755 aa, synthetic construct
  REGION 1..755: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSQVQLQESGPGLVKPSETLSLTCKASGFSFSSGYDMCWVRQPPGKGLEWIACVVAGSVDITYYASWAKGRVTISKDSSKNQVSLKLSSVTAADTAVYYCARKDAYSDAFNLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 582 - protein, 755 aa, synthetic construct
  REGION 1..755: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQVQLQESGPGLVKPSETLSLTCKASGFSFSSGYDMCWVRQPPGKGLEWIACVVAGSVDITYYASWAKGRVTISKDSSKNQVSLKLSSVTAADTAVYYCARKDAYSDAFNLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 583 - protein, 755 aa, synthetic construct
  REGION 1..755: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDSAVYYCARHPSDAVYGYANNLWGQGTLVKVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQVQLQESGPGLVKPSETLSLTCKASGFSFSSGYDMCWVRQPPGKGLEWIACVVAGSVDITYYASWAKGRVTISKDSSKNQVSLKLSSVTAADSAVYYCARKDAYSDAFNLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 584 - protein, 755 aa, synthetic construct
  REGION 1..755: note = artificial antibody-related sequence
DFQLTQSPSSLSASVGDRVTITCQASQSINDYLAWYQQKPGKSPKLLIYKASTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSQSQLQESGPGLVKPSETLSLTCKASGFSFSSGYDMCWVRQPPGKGLEWIACVVAGSVDITYYASWAKGRVTISKDSSKNQVSLKLSSVTAADTAVYFCARKDAYSDAFNLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 585 - protein, 755 aa, synthetic construct
  REGION 1..755: note = artificial antibody-related sequence
DFQLTQSPSSLSASVGDRVTITCQASQSINDYLAWYQQKPGKSPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQSQLQESGPGLVKPSETLSLTCKASGFSFSSGYDMCWVRQPPGKGLEWIACVVAGSVDITYYASWAKGRVTISKDSSKNQVSLKLSSVTAADTAVYFCARKDAYSDAFNLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 586 - protein, 755 aa, synthetic construct
  REGION 1..755: note = artificial antibody-related sequence
DFQLTQSPSSLSASVGDRVTITCQASQSINDYLAWYQQKPGKSPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDSAVYYCARHPSDAVYGYANNLWGQGTLVKVSSGGGGSGGGGSGGGGSGGGGSIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQSQLQESGPGLVKPSETLSLTCKASGFSFSSGYDMCWVRQPPGKGLEWIACVVAGSVDITYYASWAKGRVTISKDSSKNQVSLKLSSVTAADSAVYFCARKDAYSDAFNLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 587 - protein, 755 aa, synthetic construct
  REGION 1..755: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSQSQLQESGPGLVKPSETLSLTCKASGFSFSSGYDMCWVRQPPGKGLEWIACVVAGSVDITYYASWAKGRVTISKDSSKNQVSLKLSSVTAADTAVYFCARKDAYSDAFNLWGQGTLVTVSSGGGGSGGGGSGGGGSGGGGSFQLTQSPSSLSASVGDRVTITCQASQSINDYLAWYQQKPGKSPKLLIYKASTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 588 - protein, 755 aa, synthetic construct
  REGION 1..755: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQSQLQESGPGLVKPSETLSLTCKASGFSFSSGYDMCWVRQPPGKGLEWIACVVAGSVDITYYASWAKGRVTISKDSSKNQVSLKLSSVTAADTAVYFCARKDAYSDAFNLWGQGTLVTVSSGGGGSGGGGSGGGGSGGGGSFQLTQSPSSLSASVGDRVTITCQASQSINDYLAWYQQKPGKSPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 589 - protein, 755 aa, synthetic construct
  REGION 1..755: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQSQLQESGPGLVKPSETLSLTCKASGFSFSSGYDMCWVRQPPGKGLEWIACVVAGSVDITYYASWAKGRVTISKDSSKNQVSLKLSSVTAADSAVYFCARKDAYSDAFNLWGQGTLVKVSSGGGGSGGGGSGGGGSGGGGSFQLTQSPSSLSASVGDRVTITCQASQSINDYLAWYQQKPGKSPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDSAVYYCARHPSDAVYGYANNLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 590 - protein, 755 aa, synthetic construct
  REGION 1..755: note = artificial antibody-related sequence
DIQMTQSPSSLSASVGDRVTITCQASQSINNVLAWYQQKPGKAPKLLIYRASTLASGVPSRFSGSGSGTDFTLTISSLQPEDFATYYCQSSYGNYGDFGCGTKVTVLGGGGGSQVQLQESGPGLVKPSETLSLTCKASGFSFSSGYDMCWVRQPPGKGLEWIACVVAGSVDITYYASWAKGRVTISKDSSKNQVSLKLSSVTAADTAVYYCARKDAYSDAFNLWGQGTLVTVSSGGGGSGGGGSGGGGSGGGGSIQMTQSPSSLSASVGDRVTITCQASQSINDYLAWYQQKPGKAPKLLIYKASTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 591 - protein, 755 aa, synthetic construct
  REGION 1..755: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQVQLQESGPGLVKPSETLSLTCKASGFSFSSGYDMCWVRQPPGKGLEWIACVVAGSVDITYYASWAKGRVTISKDSSKNQVSLKLSSVTAADTAVYYCARKDAYSDAFNLWGQGTLVTVSSGGGGSGGGGSGGGGSGGGGSIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDTAVYYCARHPSDAVYGYANNLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 592 - protein, 755 aa, synthetic construct
  REGION 1..755: note = artificial antibody-related sequence
DIQMTQSPSSLSASVGDRVTITCQASQSINNVLAWYQQKPGKAPKLLIYRGGTLASGVPSRFSGSGSGTDFTLTISSLQPEDFATYYCQSSYGNYGDFGCGTKVTVLGGGGGSQVQLQESGPGLVKPSETLSLTCKASGFSFSSGYDMCWVRQPPGKGLEWIACVVAGSVDITYYASWAKGRVTISKDSSKNQVSLKLSSVTAADSAVYYCARKDAYSDAFNLWGQGTLVKVSSGGGGSGGGGSGGGGSGGGGSIQMTQSPSSLSASVGDRVTITCQASQSINDYLAWYQQKPGKAPKLLIYKGGTLASGVPSRFSGSGSGTDFTLTISSLQPEDFATYYCQQGYIITDIDNVFGTGTKVTVLGGGGGSEVQLVESGGGLVQPGGSLRLSCAASGFSFSNSYWICWVRQAPGKCLEWIGCTFVGSSDSTYYANWAKGRFTISRDNSKNTVYLQMNSLRAEDSAVYYCARHPSDAVYGYANNLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 593 - protein, 759 aa, synthetic construct
  REGION 1..759: note = artificial antibody-related sequence
DIQMTQSPASLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSESQLVESGGGLVQPGGSLRLSCAASGFSFSANYYPCWVRQAPGKGLEWIGCIYGGSSDITYDANWTKGRFTISRDNSKNTVYLQMNSLRAEDTAVYFCARSAWYSGWGGDLWGPGTLVTVSSGGGGSGGGGSGGGGSGGGGSFQLTQSPSSLSASVGDRVTITCQASQSISNRLAWYQQKPGKPPKLLIYSASTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 594 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSESQLVESGGGLVQPGGSLRLSCAASGFSFSANYYPCWVRQAPGKGLEWIGCIYGGSSDITYDANWTKGRFTISRDNSKNTVYLQMNSLRAEDTAVYFCARSAWYSGWGGDLWGPGTLVTVSSGGGGSGGGGSGGGGSGGGGSFQLTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 595 - protein, 759 aa, synthetic construct
  REGION 1..759: note = artificial antibody-related sequence
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSESQLVESGGGLVQPGGSLRLSCAASGFSFSANYYPCWVRQAPGKGLEWIGCIYGGSSDITYDANWTKGRFTISRDNSKNTVYLQMNSLRAEDSAVYFCARSAWYSGWGGDLWGPGTLVKVSSGGGGSGGGGSGGGGSGGGGSFQLTQSPSSLSASVGDRVTITCQASQSISNRLAWYQQKPGKPPKLLIYSGGTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDSAVYFCARGYVDYGGATDLWGQGTLVKVSSGGGGSGGGGSIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 596 - protein, 484 aa, synthetic construct
  REGION 1..484: note = artificial antibody-related sequence
DIQMTQSPSSLSASVGDRVTINCQASESISSWLAWYQQKPGKRPKLLIYKASTLASGVPSRFSGSGSGTDYTLTISSLQPEDFATYYCQGYYLDSSVDDNVFGGGTKLTVLGRTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 597 - protein, 484 aa, synthetic construct
  REGION 1..484: note = artificial antibody-related sequence
DIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGRTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 598 - protein, 484 aa, synthetic construct
  REGION 1..484: note = artificial antibody-related sequence
DIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGRTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 599 - protein, 491 aa, synthetic construct
  REGION 1..491: note = artificial antibody-related sequence
EVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDIQMTQSPSSLSASVGDRVTITCQASQTINYWLAWYQQKPGKAPKLLIYRASTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSS

SEQ ID NO: 600 - protein, 491 aa, synthetic construct
  REGION 1..491: note = artificial antibody-related sequence
EVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSS

SEQ ID NO: 601 - protein, 491 aa, synthetic construct
  REGION 1..491: note = artificial antibody-related sequence
EVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDSAVYYCARVDGFDITYFDLWGQGTLVKVSS

SEQ ID NO: 602 - protein, 763 aa, synthetic construct
  REGION 1..763: note = artificial antibody-related sequence
DIQMTQSPSSLSASVGDRVTINCQASESISSWLAWYQQKPGKRPKLLIYKASTLASGVPSRFSGSGSGTDYTLTISSLQPEDFATYYCQGYYLDSSVDDNVFGGGTKLTVLGGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSSGGGGSGGGGSGGGGSGGGGSDIQMTQSPSSLSASVGDRVTITCQASQTINYWLAWYQQKPGKAPKLLIYRASTLASGVPSRFSGSGSGTDFTLTISSLQPEDFATYYCQNYYYSTSRSYGFGGGTKLTVLGGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSSGGGGSGGGGSDIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 603 - protein, 763 aa, synthetic construct
  REGION 1..763: note = artificial antibody-related sequence
DIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSSGGGGSGGGGSGGGGSGGGGSDIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSSGGGGSGGGGSDIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

SEQ ID NO: 604 - protein, 763 aa, synthetic construct
  REGION 1..763: note = artificial antibody-related sequence
DIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDSAVYYCARVDGFDITYFDLWGQGTLVKVSSGGGGSGGGGSGGGGSGGGGSDIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSEVQLVESGGGLVQPGGSLRLSCAASGIDFSTYGISWVRQAPGKGLEWIAYIYPDYGITDYASWVNGRFTISLDNAQNTVYLQMNSLRAEDSAVYYCARSGSYYSRGWGAHYFNLWGQGTLVKVSSGGGGSGGGGSDIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSS

SEQ ID NO: 605 - protein, 763 aa, synthetic construct
  REGION 1..763: note = artificial antibody-related sequence
DIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSSGGGGSGGGGSGGGGSGGGGSDIQMTQSPSSLSASVGDRVTINCQASESISSWLAWYQQKPGKRPKLLIYKASTLASGVPSRFSGSGSGTDYTLTISSLQPEDFATYYCQGYYLDSSVDDNVFGGGTKLTVLGGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSSGGGGSGGGGSDIQMTQSPSSLSASVGDRVTITCQASQTINYWLAWYQQKPGKAPKLLIYRASTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSS

SEQ ID NO: 606 - protein, 763 aa, synthetic construct
  REGION 1..763: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSEVQLVESGGGLVQPGGSLRLSCAASGIDFSTYGISWVRQAPGKGLEWIAYIYPDYGITDYASWVNGRFTISLDNAQNTVYLQMNSLRAEDTAVYYCARSGSYYSRGWGAHYFNLWGQGTLVTVSSGGGGSGGGGSGGGGSGGGGSDIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSSGGGGSGGGGSDIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDTAVYYCARVDGFDITYFDLWGQGTLVTVSS

SEQ ID NO: 607 - protein, 763 aa, synthetic construct
  REGION 1..763: note = artificial antibody-related sequence
DIQMTQSPSSLSASVGDRVTITCQSSESVYSNNQLSWYQQKPGQPPKLLIYDGGDLASGVPSRFSGSGSGTDFTLTISSLQPEDFATYYCAGGFSSSSDTAFGGGTKLTVLGGGGGSEVQLVESGGGLVQPGGSLRLSCAASGIDFSTYGISWVRQAPGKGLEWIAYIYPDYGITDYASWVNGRFTISLDNAQNTVYLQMNSLRAEDSAVYYCARSGSYYSRGWGAHYFNLWGQGTLVKVSSGGGGSGGGGSGGGGSGGGGSDIQMTQSPSSLSASVGDRVTINCQASESISSWLAWYQQKPGKRPKLLIYKGGTLASGVPSRFSGSGSGTDYTLTISSLQPEDFATYYCQGYYLDSSVDDNVFGGGTKLTVLGGGGGSEVQLVESGGGLVQPGGSLRLSCAASGFSLSSNAMGWVRQAPGKGLEYIGIISVGGFTYYASWAKGRFTISRDNSKNTVYLQMNSLRAEDSATYFCARDRHGGDSSGAFYLWGQGTLVKVSSGGGGSGGGGSDIQMTQSPSSLSASVGDRVTITCQASQTINYWLAWYQQKPGKAPKLLIYRGGTLASGVPSRFSGSGSGTDFTLTISSLQPEDFATYYCQNYYYSTSRSYGFGGGTKLTVLGGGGGSGGGGSGGGGSGGGGSEVQLVESGGGLVQPGGSLRLSCAASGFSFSSDYWMCWVRQAPGKGLEWIGCIYAGDVDDTDYASWARGRFTISRDNSKNTVYLQMNSLRAEDSAVYYCARVDGFDITYFDLWGQGTLVKVSS

SEQ ID NO: 608 - protein, 488 aa, synthetic construct
  REGION 1..488: note = artificial antibody-related sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKLTVLGGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSDVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 609 - protein, 488 aa, synthetic construct
  REGION 1..488: note = artificial antibody-related sequence
DIQMTQSPSSLSASVGDRVTITCRASQDVNTAVAWYQQKPGKAPKLLIYSGGFLYSGVPSRFSGSRSGTDFTLTISSLQPEDFATYYCQQHYTTPPTFGCGTKLTVLGGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSDVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 610 - protein, 488 aa, synthetic construct
  REGION 1..488: note = artificial antibody-related sequence
DIQMTQSPSSLSASVGDRVTITCRASQDVNTAVAWYQQKPGKAPKLLIYSGGFLYSGVPSRFSGSRSGTDFTLTISSLQPEDFATYYCQQHYTTPPTFGCGTKLTVLGGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDSAVYYCSRWGGDGFYAMDYWGQGTLVKVSSGGGGSGGGGSDVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 611 - protein, 510 aa, synthetic construct
  REGION 1..510: note = artificial antibody-related sequence
DIQMTQSPSSLSASVGDRVTITCQASQSINDYLAWYQQKPGKAPKLLIYKASTLASGVPSRFSGSGSGTDFTLTISSLQPEDFATYYCQAAYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSEVQLVESGGGLVQPGGSLRLSCAASGFSFSSSYWICWVRQAPGKCLEWVGCVFTGDGTTYYASWAKGRFTISRDNSKNTVYLQMNSLRAEDTATYFCARPVSVYYYGMDLWGQGTLVTVSS

SEQ ID NO: 612 - protein, 510 aa, synthetic construct
  REGION 1..510: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQAAYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADTAVYYCARKDAYSDAFNLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSEVQLVESGGGLVQPGGSLRLSCAASGFSFSSSYWICWVRQAPGKCLEWVGCVFTGDGTTYYASWAKGRFTISRDNSKNTVYLQMNSLRAEDTATYFCARPVSVYYYGMDLWGQGTLVTVSS

SEQ ID NO: 613 - protein, 510 aa, synthetic construct
  REGION 1..510: note = artificial antibody-related sequence
DIQMTQSPSSLSASVGDRVTITCQASQSINDYLAWYQQKPGKAPKLLIYKGGTLASGVPSRFSGSGSGTDFTLTISSLQPEDFATYYCQAAYIITDIDNVFGTGTKVTVLGGGGGSGGGGSGGGGSGGGGSQVQLQESGPGLVKPSETLSLTCKVSGFSFSSGYDMCWIRQPPGKGLEWIGCVVAGSVDITYYASWAKGRVTISVDSSKNQFSLKLSSVTAADSAVYYCARKDAYSDAFNLWGQGTLVKVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDSATYFCARGGWTGTSHSNIWGQGTLVKVSSGGGSGGGSEVQLVESGGGLVQPGGSLRLSCAASGFSFSSSYWICWVRQAPGKCLEWVGCVFTGDGTTYYASWAKGRFTISRDNSKNTVYLQMNSLRAEDSATYFCARPVSVYYYGMDLWGQGTLVKVSS

SEQ ID NO: 614 - protein, 487 aa, synthetic construct
  REGION 1..487: note = artificial antibody-related sequence
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVTVLGGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSGGGGSGGGGSDVVMTQSPSSLSASVGDRVTITCQASQIISSRSAWYQQKPGQPPKLLIYQASKLASGVPSRFSGSGSGTDFTLTISSLQPEDFATYYCQCTYIDSNFGAFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 615 - protein, 487 aa, synthetic construct
  REGION 1..487: note = artificial antibody-related sequence
DIQMTQSPSSLSASVGDRVTITCKASQDVSIGVAWYQQKPGKAPKLLIYSGGYRYTGVPSRFSGSGSGTDFTLTISSLQPEDFATYYCQQYYIYPYTFGCGTKVTVLGGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSGGGGSGGGGSDVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 616 - protein, 487 aa, synthetic construct
  REGION 1..487: note = artificial antibody-related sequence
DIQMTQSPSSLSASVGDRVTITCKASQDVSIGVAWYQQKPGKAPKLLIYSGGYRYTGVPSRFSGSGSGTDFTLTISSLQPEDFATYYCQQYYIYPYTFGCGTKVTVLGGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDSAVYYCARNLGPSFYFDYWGQGTLVKVSSGGGGSGGGGSDVVMTQSPSSLSASVGDRVTITCQASQIISSRSAWYQQKPGQPPKLLIYQGGKLASGVPSRFSGSGSGTDFTLTISSLQPEDFATYYCQCTYIDSNFGAFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 617 - protein, 510 aa, synthetic construct; identical to SEQ ID NO: 611
  REGION 1..510: note = artificial antibody-related sequence

SEQ ID NO: 618 - protein, 510 aa, synthetic construct; identical to SEQ ID NO: 612
  REGION 1..510: note = artificial antibody-related sequence

SEQ ID NO: 619 - protein, 510 aa, synthetic construct; identical to SEQ ID NO: 613
  REGION 1..510: note = artificial antibody-related sequence

SEQ ID NO: 620 - protein, 515 aa, synthetic construct
  REGION 1..515: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMCSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 621 - protein, 515 aa, synthetic construct
  REGION 1..515: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMCSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 622 - protein, 515 aa, synthetic construct
  REGION 1..515: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDSAVYFCARGSSSGGYLDDGFDPWGQGTLVKVSSGGGGSGGGGSEVQLVESGGGLVQPGGSLRLSCAASGFSLSSYDMSWVRQAPGKGLAWIGASYASGPTYYASWAKGRFTISRDNSKNTVYLQMCSLRAEDSATYFCARGGWTGTSHSNIWGQGTLVKVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSS

SEQ ID NO: 623 - protein, 499 aa, synthetic construct
  REGION 1..499: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSAFELTQSPSSLSASVGDRVTITCQASESINSRLAWYQQKPGQPPKLLIYDASDLTSGVPSRFSGSGSGTDFTLTISCLQPEDFATYYCQGYGGSSTTTFGCGTKLTVLGGGSGGSGGSGGSGDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 624 - protein, 499 aa, synthetic construct
  REGION 1..499: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSAFELTQSPSSLSASVGDRVTITCQASESINSRLAWYQQKPGQPPKLLIYDGGDLTSGVPSRFSGSGSGTDFTLTISCLQPEDFATYYCQGYGGSSTTTFGCGTKLTVLGGGSGGSGGSGGSGDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 625 - protein, 499 aa, synthetic construct
  REGION 1..499: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDSAVYFCARGSSSGGYLDDGFDPWGQGTLVKVSSGGGGSGGGGSAFELTQSPSSLSASVGDRVTITCQASESINSRLAWYQQKPGQPPKLLIYDGGDLTSGVPSRFSGSGSGTDFTLTISCLQPEDFATYYCQGYGGSSTTTFGCGTKLTVLGGGSGGSGGSGGSGDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 626 - protein, 513 aa, synthetic construct
  REGION 1..513: note = artificial antibody-related sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGTGTKLTVLGGGGGSGGGGSGGGGSGGGGSEVQLVESGGGLVQPGGSLRLSCAASGFSLSSYAMNWVRQAPGKGLEWIGHINAGDIAYYATWAKGRFTISRDNSKNTVYLQMNSLRAEDTAVYFCARGAGGFSTGPFKLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSS

SEQ ID NO: 627 - protein, 513 aa, synthetic construct
  REGION 1..513: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGTGTKLTVLGGGGGSGGGGSGGGGSGGGGSEVQLVESGGGLVQPGGSLRLSCAASGFSLSSYAMNWVRQAPGKGLEWIGHINAGDIAYYATWAKGRFTISRDNSKNTVYLQMNSLRAEDTAVYFCARGAGGFSTGPFKLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSS

SEQ ID NO: 628 - protein, 513 aa, synthetic construct
  REGION 1..513: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGTGTKLTVLGGGGGSGGGGSGGGGSGGGGSEVQLVESGGGLVQPGGSLRLSCAASGFSLSSYAMNWVRQAPGKGLEWIGHINAGDIAYYATWAKGRFTISRDNSKNTVYLQMNSLRAEDSAVYFCARGAGGFSTGPFKLWGQGTLVKVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDSATYFCARGGWTGTSHSNIWGQGTLVKVSSGGGSGGGSGGGSGQSQLVESGGGLVQPGGSLRLSCAVSGFSLSSYAMGWVRQAPGKCLEYIGYISKIGTTYYASWAKGRFTISKDNSKNTVYLQMNSLRAEDSAVYFCARGSSSGGYLDDGFDPWGQGTLVKVSS

SEQ ID NO: 629 - protein, 501 aa, synthetic construct
  REGION 1..501: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLGGGSGGSGGSGGSGDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 630 - protein, 501 aa, synthetic construct
  REGION 1..501: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGSGGSGGSGGSGDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 631 - protein, 501 aa, synthetic construct
  REGION 1..501: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDSAVYFCARGSSSGGYLDDGFDPWGQGTLVKVSSGGGGSGGGGSALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGSGGSGGSGGSGDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 632 - protein, 513 aa, synthetic construct; identical to SEQ ID NO: 626
  REGION 1..513: note = artificial antibody-related sequence

SEQ ID NO: 633 - protein, 513 aa, synthetic construct; identical to SEQ ID NO: 627
  REGION 1..513: note = artificial antibody-related sequence

SEQ ID NO: 634 - protein, 513 aa, synthetic construct; identical to SEQ ID NO: 628
  REGION 1..513: note = artificial antibody-related sequence

SEQ ID NO: 635 - protein, 494 aa, synthetic construct
  REGION 1..494: note = artificial antibody-related sequence
ALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSALQMTQSPSSLSARVGDRVTIKCQASQSISNYLAWYQQKPGKPPKFLIYDASDLASGVPSRFSGSGSGRDFTLTISSLQPEDFATYYCQQVYDSNNVENVFGCGTKVTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 636 - protein, 494 aa, synthetic construct
  REGION 1..494: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDTAVYFCARGSSSGGYLDDGFDPWGQGTLVTVSSGGGGSGGGGSALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 637 - protein, 494 aa, synthetic construct
  REGION 1..494: note = artificial antibody-related sequence
ALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGGGSGGGGSGGGGSGGGGSQSQLVESGGGLVQPGGSLRLSCAVSGFSLSSYAMGWVRQAPGKCLEYIGYISKIGTTYYASWAKGRFTISKDNSKNTVYLQMNSLRAEDSAVYFCARGSSSGGYLDDGFDPWGQGTLVKVSSGGGGSGGGGSALQMTQSPSSLSARVGDRVTIKCQASQSISNYLAWYQQKPGKPPKFLIYDGGDLASGVPSRFSGSGSGRDFTLTISSLQPEDFATYYCQQVYDSNNVENVFGCGTKVTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 638 - protein, 516 aa, synthetic construct
  REGION 1..516: note = artificial antibody-related sequence
DIQMTQSPSSLSASVGDRVTITCQASESIYSSLAWYQQKPGKAPKLLIYLASTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDTAVYYCARDMGFADYALNLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 639 - protein, 516 aa, synthetic construct
  REGION 1..516: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDTAVYYCARDMGFADYALNLWGQGTLVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTATYFCARGGWTGTSHSNIWGQGTLVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 640 - protein, 516 aa, synthetic construct
  REGION 1..516: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDSAVYYCARDMGFADYALNLWGQGTLVKVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDSATYFCARGGWTGTSHSNIWGQGTLVKVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSS

SEQ ID NO: 641 - protein, 493 aa, synthetic construct
  REGION 1..493: note = artificial antibody-related sequence
DIQMTQSPSSLSASVGDRVTITCQASESIYSSLAWYQQKPGKAPKLLIYLASTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDTAVYYCARDMGFADYALNLWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 642 - protein, 493 aa, synthetic construct
  REGION 1..493: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDTAVYYCARDMGFADYALNLWGQGTLVTVSSGGGGSGGGGSAFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 643 - protein, 493 aa, synthetic construct
  REGION 1..493: note = artificial antibody-related sequence
DIQMTQSPSSLSASVGDRVTITCQASESIYSSLAWYQQKPGKAPKLLIYLGGTLASGVPSRFSGSGSGTDFTLTISSLQPEDFATYYCQSTDYTTSTHRNSFGTGTKVTVLGGGGGSGGGGSGGGGSGGGGSEVQLVESGGGLVQPGGSLRLSCAASGFSFSTTYYMCWVRQAPGKGLEWIGCTNTASSVRTYYATWAKGRFTISRDNSKNTVYLQMNSLRAEDSAVYYCARDMGFADYALNLWGQGTLVKVSSGGGGSGGGGSAFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 644 - protein, 758 aa, synthetic construct
  REGION 1..758: note = artificial antibody-related sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSDVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSS

SEQ ID NO: 645 - protein, 758 aa, synthetic construct
  REGION 1..758: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSDVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSS

SEQ ID NO: 646 - protein, 758 aa, synthetic construct
  REGION 1..758: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDSAVYFCARGGWTGTSHSNIWGQGTLVKVSSGGGGSGGGGSGGGGSGGGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSSGGGGSGGGGSDVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGQGTLVKVSS

SEQ ID NO: 647 - protein, 751 aa, synthetic construct
  REGION 1..751: note = artificial antibody-related sequence
AFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSAQQLTQSPSSLSASVGDRVTITCQASQNVWNNNYLSWFQQKPGKPPKLLIVTASTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGLSLSRNAMSWVRQAPGKCLEWIGIILTSGSTYYASWAKGRFTISKDNSKNTVYLQMNSLRAEDTAVYFCVRGIASSSLKSFWGQGTLVTVSS

SEQ ID NO: 648 - protein, 751 aa, synthetic construct
  REGION 1..751: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGQGTLVTVSSGGGGSGGGGSGGGGSGGGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSSGGGGSGGGGSAQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGLSLSRNAMSWVRQAPGKCLEWIGIILTSGSTYYASWAKGRFTISKDNSKNTVYLQMNSLRAEDTAVYFCVRGIASSSLKSFWGQGTLVTVSS

SEQ ID NO: 649 - protein, 751 aa, synthetic construct
  REGION 1..751: note = artificial antibody-related sequence
AFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGGGSEVQLVESGGGLVQPGGSLRLSCAASGFSLSSYDMSWVRQAPGKGLAWIGASYASGPTYYASWAKGRFTISRDNSKNTVYLQMNSLRAEDSAVYFCARGGWTGTSHSNIWGQGTLVKVSSGGGGSGGGGSGGGGSGGGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLGGGGGSEVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSSGGGGSGGGGSAQQLTQSPSSLSASVGDRVTITCQASQNVWNNNYLSWFQQKPGKPPKLLIVTGGTLASGVSSRFSGSGSGTDFTLTISSLQPEDFATYYCAGGFSGEIRAFGCGTKVTVLGGGGGSGGGGSGGGGSGGGGSQSQLVESGGGLVQPGGSLRLSCAVSGLSLSRNAMSWVRQAPGKCLEWIGIILTSGSTYYASWAKGRFTISKDNSKNTVYLQMNSLRAEDSAVYFCVRGIASSSLKSFWGQGTLVKVSS

SEQ ID NO: 650 - protein, 495 aa, synthetic construct
  REGION 1..495: note = artificial antibody-related sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 651 - protein, 495 aa, synthetic construct
  REGION 1..495: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGQGTLVTVSSGGGGSGGGGSAFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 652 - protein, 495 aa, synthetic construct
  REGION 1..495: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGQGTLVKVSSGGGGSGGGGSAFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 653 - protein, 518 aa, synthetic construct
  REGION 1..518: note = artificial antibody-related sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGLGTQVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGLGTQVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 654 - protein, 518 aa, synthetic construct
  REGION 1..518: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDTAVYFCARDRYDSGAYLYTTYFNLWGLGTQVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGLGTQVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 655 - protein, 518 aa, synthetic construct
  REGION 1..518: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGTGTKVTVLGGGGGSGGGGSGGGGSGGGGSQSQVVESGGGLVQPGGSLRLSCAVSGFDLSSYAVSWVRQAPGKGLEWIGIIYPRANTYYASWAKGRFTISKDNSKNTVYLQMNSLRAEDSAVYFCARDRYDSGAYLYTTYFNLWGLGTQVKVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDSAVYFCARGGWTGTSHSNIWGLGTQVKVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSS

SEQ ID NO: 656 - protein, 495 aa, synthetic construct
  REGION 1..495: note = artificial antibody-related sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGLGTQVTVSSGGGGSGGGGSAFELTQSPSSLSASVGDRVTITCQASESINSRLAWYQQKPGQPPKLLIYDASDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSASTLASGVPSRFSGSGSGTDFTLTISSLQPEDFATYYCLGSYACSSADCYVFGTGTKVTVLG

SEQ ID NO: 657 - protein, 495 aa, synthetic construct
  REGION 1..495: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGLGTQVTVSSGGGGSGGGGSAFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 658 - protein, 495 aa, synthetic construct
  REGION 1..495: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDSAVYFCARDRYDSGAYLYTTYFNLWGLGTQVKVSSGGGGSGGGGSAFELTQSPSSLSASVGDRVTITCQASESINSRLAWYQQKPGQPPKLLIYDGGDLTSGVPSRFSGSGSGTDFTLTISSLQPEDFATYYCQGYGGSSTTTFGCGTKLTVLGGGSGGSDIQMTQSPSSLSASVGDRVTITCQSSQSVFSNNYLAWFQQKPGQSPKRLIYSGGTLASGVPSRFSGSGSGTDFTLTISSLQPEDFATYYCLGSYACSSADCYVFGTGTKVTVLG

SEQ ID NO: 659 - protein, 518 aa, synthetic construct
  REGION 1..518: note = artificial antibody-related sequence
DVQMTQSPSSLSASVGDRVTITCRASENIYSGLAWYQQKPGKPPKLLIYRASTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGLGTQVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGLGTQVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 660 - protein, 518 aa, synthetic construct
  REGION 1..518: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDTAVYFCARDRYDSGAYLYTTYFNLWGLGTQVTVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDTAVYFCARGGWTGTSHSNIWGLGTQVTVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDTAVYFCARGAGGFSTGPFKLWGQGTLVTVSS

SEQ ID NO: 661 - protein, 518 aa, synthetic construct
  REGION 1..518: note = artificial antibody-related sequence
DVQMTQSPSSLSASVGDRVTITCRASENIYSGLAWYQQKPGKPPKLLIYRGGTLASGVSSRFSGSGSGTDFTLTISSLQPEDFATYYCQGGYYSSSSTYIAFGCGTKVTVLGGGGGSGGGGSGGGGSGGGGSQSQVVESGGGLVQPGGSLRLSCAVSGFDLSSYAVSWVRQAPGKCLEWIGIIYPRANTYYASWAKGRFTISKDNSKNTVYLQMNSLRAEDSAVYFCARDRYDSGAYLYTTYFNLWGLGTQVKVSSGGGGSGGGGSEVQLVESGGGLVQPGGSLRLSCAASGFSLSSYDMSWVRQAPGKGLAWIGASYASGPTYYASWAKGRFTISRDNSKNTVYLQMNSLRAEDSAVYFCARGGWTGTSHSNIWGLGTQVKVSSGGGSGGGSGGGSGEVQLVESGGGLVQPGGSLRLSCAASGFSLSSYAMNWVRQAPGKCLEWIGHINAGDIAYYATWAKGRFTISRDNSKNTVYLQMNSLRAEDSAVYFCARGAGGFSTGPFKLWGQGTLVKVSS

SEQ ID NO: 662 - protein, 256 aa, synthetic construct
DIQMTQSPSSLSASVGDRVTITCQASQSIGTYLAWYQQKPGKPPKLLIYRAFILASGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 663 - protein, 256 aa, synthetic construct
DIQMTQSPASLSASVGDRVTITCQASQSIGTYLAWYQQKPGKPPKLLIYAASSLQSGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 664 - protein, 256 aa, synthetic construct
DIQMTQSPASLSASVGDRVTITCQASQSIGTYLAWYQQKPGKPPKLLIYAASSLQSGVPSRFSGSGSGTDFTLTISSLQPEDFATYYCQSNFYSDSTTIGPNAFGTGTKVTVLGGGGGSGGGGSGGGGSGGGGSEVQLVESGGGLVQPGGSLRLSCAASGFSFNSDYWISWVRQAPGKGLEWIASIYGGSSGNTQYASWAQGRFTISRDNSKNTVYLQMNSLRAEDTAVYFCARGYVDYGGATDLWGQGTLVTVSS

SEQ ID NO: 665 - protein, 7 aa, synthetic construct
LGGNRAA

SEQ ID NO: 666 - protein, 7 aa, synthetic construct
RGGERVS

SEQ ID NO: 667 - protein, 7 aa, synthetic construct
RGGILAS

SEQ ID NO: 668 - protein, 7 aa, synthetic construct
KGGTLAS

SEQ ID NO: 669 - protein, 7 aa, synthetic construct
RGGTLAS

SEQ ID NO: 670 - protein, 7 aa, synthetic construct
SGGTLAS

SEQ ID NO: 671 - protein, 7 aa, synthetic construct
LGGTLAS

SEQ ID NO: 672 - protein, 7 aa, synthetic construct
TGGTLAS

SEQ ID NO: 673 - protein, 7 aa, synthetic construct
GGGTLAS

SEQ ID NO: 674 - protein, 7 aa, synthetic construct
RGGNLAS

SEQ ID NO: 675 - protein, 7 aa, synthetic construct
DGGDLAS

SEQ ID NO: 676 - protein, 7 aa, synthetic construct
DGGKLAS

SEQ ID NO: 677 - protein, 7 aa, synthetic construct
QGGKLAS

SEQ ID NO: 678 - protein, 7 aa, synthetic construct
RGGKLAS

SEQ ID NO: 679 - protein, 7 aa, synthetic construct
LGGKLAS

SEQ ID NO: 680 - protein, 7 aa, synthetic construct
SGGKLAS

SEQ ID NO: 681 - protein, 7 aa, synthetic construct
GGGKLAS

SEQ ID NO: 682 - protein, 7 aa, synthetic construct
DGGRLAS

SEQ ID NO: 683 - protein, 7 aa, synthetic construct
DGGNRAT

SEQ ID NO: 684 - protein, 7 aa, synthetic construct
RGGTLES

SEQ ID NO: 685 - protein, 7 aa, synthetic construct
KGGTLES

SEQ ID NO: 686 - protein, 7 aa, synthetic construct
DGGDLTS

SEQ ID NO: 687 - protein, 7 aa, synthetic construct
SGGFLYS

SEQ ID NO: 688 - protein, 7 aa, synthetic construct
SGGYRYT

SEQ ID NO: 689 - protein, 7 aa, synthetic construct
SGGKLAA

SEQ ID NO: 690 - protein, 7 aa, synthetic construct
SGGTLVS

SEQ ID NO: 691 - protein, 7 aa, synthetic construct
RGGTLAY

SEQ ID NO: 692 - protein, 7 aa, synthetic construct
AGGTLAS

SEQ ID NO: 693 - protein, 7 aa, synthetic construct
AGGYLAS

SEQ ID NO: 694 - protein, 7 aa, synthetic construct
RGGYLES

SEQ ID NO: 695 - protein, 7 aa, synthetic construct
EGGKLAS

SEQ ID NO: 696 - protein, 7 aa, synthetic construct
RGGNLES

SEQ ID NO: 697 - protein, 7 aa, synthetic construct
RGGILES

SEQ ID NO: 698 - protein, 7 aa, synthetic construct
RGGNRES

SEQ ID NO: 699 - protein, 7 aa, synthetic construct
RGGTLDS

SEQ ID NO: 700 - protein, 7 aa, synthetic construct
LGGKLES

SEQ ID NO: 701 - protein, 7 aa, synthetic construct
LGGKMES

SEQ ID NO: 702 - protein, 7 aa, synthetic construct
TGGSLAS

SEQ ID NO: 703 - protein, 7 aa, synthetic construct
GGGTLES

SEQ ID NO: 704 - protein, 7 aa, synthetic construct
AASSLQS

SEQ ID NO: 705 - protein, 7 aa, synthetic construct
RAFILAS

SEQ ID NO: 706 - protein, 7 aa, synthetic construct
KASTLAS

SEQ ID NO: 707 - protein, 7 aa, synthetic construct
SASTLAS

SEQ ID NO: 708 - protein, 7 aa, synthetic construct
RASTLAS

SEQ ID NO: 709 - protein, 7 aa, synthetic construct
DASDLTS

SEQ ID NO: 710 - protein, 7 aa, synthetic construct
LASTLAS

SEQ ID NO: 711 - protein, 7 aa, synthetic construct
DASDLAS

SEQ ID NO: 712 - protein, 7 aa, synthetic construct
TASTLAS

SEQ ID NO: 713 - protein, 7 aa, synthetic construct
SASFLYS

SEQ ID NO: 714 - protein, 7 aa, synthetic construct
SASYRYT

SEQ ID NO: 715 - protein, 7 aa, synthetic construct
SASKLAA

SEQ ID NO: 716 - protein, 15 aa, synthetic construct
IYRAFILASGVPSRF

SEQ ID NO: 717 - protein, 9 aa, synthetic construct
FILASGVPS

SEQ ID NO: 718 - protein, 9 aa, synthetic construct
ILASGVPSR

SEQ ID NO: 719 - protein, 9 aa, synthetic construct
AFILASGVP

SEQ ID NO: 720 - protein, 9 aa, synthetic construct
YRAFILASG

SEQ ID NO: 721 - protein, 15 aa, synthetic construct
PKLLIYKASTLASGV

SEQ ID NO: 722 - protein, 15 aa, synthetic construct
PKLLIYSASTLASGV

SEQ ID NO: 723 - protein, 15 aa, synthetic construct
KLLIYRASTLASGVP

SEQ ID NO: 724 - protein, 15 aa, synthetic construct
PKRLIYSASTLASGV

SEQ ID NO: 725 - protein, 15 aa, synthetic construct
ASESIYSSLAWYQQK

SEQ ID NO: 726 - protein, 15 aa, synthetic construct
PKLLIYLASTLASGV

SEQ ID NO: 727 - protein, 15 aa, synthetic construct
PKFLIYDASDLASGV

SEQ ID NO: 728 - protein, 15 aa, synthetic construct
PKLLIVTASTLASGV

SEQ ID NO: 729 - protein, 15 aa, synthetic construct
KLLIYRASTLASGVS

SEQ ID NO: 730 - protein, 15 aa, synthetic construct
PKLLIYSASFLYSGV

SEQ ID NO: 731 - protein, 15 aa, synthetic construct
SGGFLYSGVPSRFSG

SEQ ID NO: 732 - protein, 15 aa, synthetic construct
PKLLIYSASYRYTGV

SEQ ID NO: 733 - protein, 15 aa, synthetic construct
SENIYSFLAWYQQKP

SEQ ID NO: 734 - protein, 15 aa, synthetic construct
PKLLIYSASKLAAGV

SEQ ID NO: 735 - protein, 15 aa, synthetic construct
SQTINYWLAWYQQKP

SEQ ID NO: 736 - protein, 15 aa, synthetic construct
YCQNYYYSTSRSYGF

SEQ ID NO: 737 - protein, 15 aa, synthetic construct
FATYYCQAAYIITDI

SEQ ID NO: 738 - protein, 15 aa, synthetic construct
VDITYYASWAKGRVT

SEQ ID NO: 739 - protein, 15 aa, synthetic construct
ASGFSLSSYAMNWVR

SEQ ID NO: 740 - protein, 15 aa, synthetic construct
VRTYYATWAKGRFTI

SEQ ID NO: 741 - protein, 15 aa, synthetic construct
VSGLSLSRNAMSWVR

SEQ ID NO: 742 - protein, 15 aa, synthetic construct
EWIGIILTSGSTYYA

SEQ ID NO: 743 - protein, 759 aa, synthetic construct
DIQMTQSPASLSASVGDRVTITCQASQSIGTYLAWYQQKPGKPPKLLIYRGGILASGVPSRFSGSGSGTDFTLTISSLQPEDFATYYCQSNFYSDSTTIGPNAFGTGTKVTVLGGGGGSEVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSSGGGGSGGGGSGGGGSGGGGSIQMTQSPSSLSASVGDRVTITCQASQSISNRLAWYQQKPGKAPKLLIYSGGTLASGVPSRFSGSGSGTDFTLTISSLQPEDFATYYCQSTYYGNDGNAFGTGTKVTVLGGGGGSEVQLVESGGGLVQPGGSLRLSCAASGFSFNSDYWIYWVRQAPGKGLEWIASIYGGSSGNTQYASWAQGRFTISRDNSKNTVYLQMNSLRAEDTAVYFCARGYVDYGGATDLWGQGTLVTVSSGGGGSGGGGSIQMTQSPSSLSASVGDRVTITCQSSESVYSNNQLSWYQQKPGQPPKLLIYDASDLASGVPSRFSGSGSGTDFTLTISSLQPEDFATYYCAGGFSSSSDTAFGGGTKLTVLGGGGGSGGGGSGGGGSGGGGSEVQLVESGGGLVQPGGSLRLSCAASGFSLSSNAMGWVRQAPGKGLEYIGIISVGGFTYYASWAKGRFTISRDNSKNTVYLQMNSLRAEDTATYFCARDRHGGDSSGAFYLWGQGTLVTVSS

(19) United States
(12) Patent Application Publication
(10) Pub. No.: US 2026/0327324 A1
(43) Pub. Date: Oct. 22, 2026

(54) ANTIBODY VARIABLE DOMAINS AND ANTIBODIES HAVING DECREASED IMMUNOGENICITY

(71) Applicant: Numab Therapeutics AG, Horgen (CH)

(72) Inventors: Stefan Warmuth, Au (CH); Maria Johansson, Denges (CH)

(21) Appl. No.: 19/485,524

(22) PCT Filed: May 17, 2024
(86) PCT No.: PCT/EP2024/063788

§ 371 Date: Nov. 18, 2025

(30) Foreign Application Priority Data
May 19, 2023 (EP) 23174324.6

(51) Int. Cl. C07K 16/28; C07K 16/18

(52) U.S. Cl. CPC C07K 16/2878; C07K 16/18; C07K 16/2827; C07K 2317/31; C07K 2317/622; C07K 2317/92

(57) ABSTRACT

The present invention relates to antibody variable domains, which exhibit a reduced binding to anti-drug antibodies (ADA), to antibodies comprising one or more of said antibody variable domains, and to pharmaceutical compositions comprising said antibodies. The present invention further relates to nucleic acids encoding said antibody variable domains or said antibodies, vector(s) comprising said nucleic acids, host cell(s) comprising said nucleic acids or said vector(s), and a method of producing said antibody variable domains or said multispecific antibodies. Additionally, the present invention relates to a method for generating said antibody variable domains and antibodies.

### FIELD OF THE INVENTION

The present invention relates to antibody variable domains, which exhibit a reduced binding to anti-drug antibodies (ADA), to antibodies comprising one or more of said antibody variable domains, and to pharmaceutical compositions comprising said antibodies. The present invention further relates to nucleic acids encoding said antibody variable domains or said antibodies, vector(s) comprising said nucleic acids, host cell(s) comprising said nucleic acids or said vector(s), and a method of producing said antibody variable domains or said multispecific antibodies. Additionally, the present invention relates to a method for generating said antibody variable domains and antibodies.

### BACKGROUND OF THE INVENTION

In the past forty years since the development of the first monoclonal antibodies (“mAbs”; Köhler & Milstein, Nature, 1975, Vol. 256, pp. 495-497), antibodies have become an increasingly important class of biomolecules for research, diagnostic and therapeutic purposes. Initially, antibodies were exclusively obtained by immunizing animals with the corresponding antigen of interest. While antibodies of non-human origin can be used in research and diagnostics, in therapeutic approaches the human body typically recognizes non-human antibodies as foreign and raise an immune response against the non-human antibody drug substance, rendering it less or not effective. Even if the administered antibody therapeutics have been humanized, e. g. by grafting of CDRs of non-human origin into human immunoglobulin frameworks to minimize the non-human component, they can still elicit an immune response, which compromises the efficacy and/or safety of these therapeutics.

Such an immune response typically involves the binding of anti-drug antibodies (ADAs) to the therapeutic agent. These ADAs can be antibodies, which are already present in human serum (so called pre-existing ADAs) and/or antibodies, which are formed during the course of the therapy, also referred to as treatment-emerging ADAs.

The risk of pre-existing ADA-binding and/or of treatment-emerging ADA formation can be significantly enhanced for therapeutic antibodies that comprise or are built of portions of a naturally occurring human antibody, e. g. Fab or Fv antibody fragments. It is believed that one of the main reasons for this increase in ADA-binding and ADA-formation is that in antibody fragments, typically a significant number of amino acids that are formerly shielded by the contact to other antibody portions or domains, become exposed to the solvent and are present to the immune system as potential epitopes.

Furthermore, the risk of pre-existing ADA-binding and/or of treatment-emerging ADA formation can also be enhanced for therapeutic antibodies that comprise complementarity-determining regions (CDRs) that have been obtained from non-human species, such as from mouse or rabbit, because CDRs derived from non-human antibody repertoires represent potential B-cell and/or T-cell epitopes. This risk for ADA-binding and ADA-formation applies to all types of antibodies, to antibody fragments as well as to full-length antibodies or IgG-based antibodies.

According to literature, antibody responses in patients are dependent on the presence of both B-cell epitopes and T-cell epitopes. When a B-cell receptor recognizes and binds an antigen such as an administered therapeutic antibody, the antigen is internalized into the B cell by receptor-mediated endocytosis and undergoes proteolytic processing. The resulting peptides are subsequently presented by MHC class II molecules. Upon recognition of the T cell epitope by a T helper cell, the latter stimulates the corresponding B cells to proliferate and differentiate into antibody producing plasma cells.

Several strategies have been provided in the prior art to further lower the response of the immune system of a patient to the administered antibodies.

WO 2002/066514 (A2) for example discloses immunogenically modified fusion proteins, which exhibit reduced immunogenicity relative to their non-modified parent fusion protein, when exposed to the immune system of a given species, by having a reduced number of T-cell epitopes within their amino acid sequence. The T-cell epitopes are specified therein to be peptide sequences able to bind to MCH class II molecule binding groups.

WO 2002/066514 (A2) further discloses a computational method that profiles the likelihood of peptide regions to contain T-cell epitopes and suggests modifying said peptide regions to potentially alter the MHC Class II binding characteristics of the protein containing them.

WO 2002/079232 (A2) discloses a method for reducing the immunogenicity of a fusion protein by first identifying a candidate T-cell epitope within a junction region spanning a fusion junction of a fusion protein; and then changing an amino acid within the junction region to reduce the ability of the candidate T-cell epitope to interact with a T cell receptor.

Furthermore, several computational methods for the identification of amino acid stretches within an antibody that may potentially bind to MHC molecules (so called T-cell hot spots) have been developed.

Nielsen et al., PLoS Computational Biology, 2008, 4(7), pp. 1-10, for example, disclose a computational method, referred to as NetMHCIIpan, that allows for pan-specific predictions of peptide binding to any HLA-DR molecule of known sequence. Knowledge of these critical structural elements forms the basis for strategies to avoid them.

Cassotta et al., Nature Med., 2019, 25(9), pp. 1402-1407, disclose the identification of a single immunodominant T cell epitope spanning the framework 2-CDR2 region of the light chain of the antibody therapeutic Natalizumab, using the T-cell epitope prediction algorithm NetMHCIIpan. Natalizumab is known to induce persistent neutralizing anti-drug antibodies (ADAs) in about 6% of the treated patients. It is further disclosed that two light chain CDR2-modified versions of Natalizumab, which had been engineered by structure-guided design, were not recognized by T cells, while their target-binding properties are retained.

Although the currently available methods provide useful indications, how the immunogenicity of a given antibody variable domain can be reduced, their proposed solutions for immunogenicity reduction are highly case dependent, i. e. their proposed solutions for reducing the immunogenicity of a given antibody variable domain are not described to be generally applicable to other antibody variable domains. Thus, there is still a large unmet need for antibody variable domains, that exhibit low immunogenicity, and for mutational schemes which can generally be applied in the construction of antibodies and antibody fragments. More specifically, it would be desirable to have antibody variable domains at hand, which have no predicted T-cell epitopes, or which have a significantly reduced number of predicted T-cell epitopes, and which can generally be applied in the construction of antibodies and antibody fragments. It is furthermore desirable that these antibody variable domains provide a high stability, when integrated in the final antibody format, which would allow their application in the construction of stable antibody fragments and fragment-based multispecific antibodies suitable for therapeutic development. It would further be desirable to have a generally applicable method at hand that allows the reliable design and production of said antibody variable domains.

### SUMMARY OF THE INVENTION

It is an object of the present invention to provide antibody variable domain variants, which generally exhibit reduced immunogenicity, more specifically, antibody variable domain variants that are significantly less recognized by MHC class II molecules, i. e. have no predicted T-cell epitopes or at least a significantly reduced number of predicted T-cell epitopes, when compared to their unmodified variants. It is a further important object of the present invention that the functional properties of said antibody variable domain variants, in particular their inhibition potency and/or their binding affinity to their respective antigen targets, is not or not significantly reduced, when compared to their unmodified variants. Furthermore, these antibody variable domain variants should be highly stable to allow their application in the construction of antibody fragments and multispecific antibodies suitable for pharmaceutical development.

The inventors have surprisingly found that antibody variable domains, which comprise a light chain complementarity-determining region 2 (LCDR2) that has a glycine (G) at amino acid position 67 and a glycine (G) at amino acid position 68 (AHo numbering), hereinafter also referred to as “GG variants” or “GG motif”, exhibit a significantly lower predicted binding to MHC class II proteins, when compared to their original versions that do not comprise said GG motif. This advantageous property could be observed for all tested antibody variable domains comprising said GG motif, where their non-GG variants have been predicted to bind to (human) MHC class II proteins, irrespective of their antigen target specificity. It was further found that the stability of said GG variants as well as their functional properties, in particular their inhibition potency and/or their binding affinity to their respective antigen targets, are not or not significantly reduced, when compared to their unmodified versions. These findings were surprising, since said GG motif is located in a CDR region involved in antigen binding, and the presence of two glycine residues at adjacent position bears the risk of increased flexibility that can result in stability reduction.

Besides, the universal applicability of this modification has been tested with a set of about 200 sequences of antibodies that have been in clinical trials. Most of these antibody sequences had no glycine at the light chain amino acid positions 67 and 68 (AHo numbering) and just a few of these antibody sequences had one glycine either at position 67 or 68 (AHo numbering). None of them had glycine residues at both positions. Introduction of the GG motif into these 200 antibody sequences mostly eliminated or at least reduced their predicted binding to MHC class II proteins.

To further explore the above findings, a subset of fragment based multispecific antibodies comprising at least one modified binding domain of the present invention has been tested against patient-derived serum samples, showing a clear reduction in their binding to pre-existing ADAs and/or to treatment-emerged ADAs, when compared to their unmodified versions (see Example 3). In summary, this and the above findings demonstrate the universal applicability of the antibody variable domains of the present invention for decreasing the risk of adverse immunogenic reactions in preclinical and clinical development.

In antibody sequence searches using the antibody sequence analysis tool abYsis (www.abYsis.org), only two prior art antibodies could be identified that accidentally have in their original LCDR2 sequence a glycine (G) at amino acid position 67 and a glycine (G) at amino acid position 68 (AHo numbering). These two LCDR2 sequence have been excluded by way of disclaimer from the claimed subject matter. However, the inventors of the present invention cannot exclude that further antibody sequences may exist that also have an LCDR2 with a glycine (G) at amino acid position 67 and a glycine (G) at amino acid position 68 (AHo numbering). Thus, as a precaution, the inventors preserve the right to disclaim any further LCDR2 sequences of known antibodies or antibody fragments that accidentally have a glycine (G) at amino acid position 67 and a glycine (G) at amino acid position 68 (AHo numbering).

In addition to the two antibodies mentioned above, some additional antibody sequences have been identified that comprise GG motifs in a region corresponding to LCDR2 (see SEQ ID NOs: 11, 19, 27 and 39 of WO 2017/173359). However, in that case those sequences show extended versions of LCDR2 comprising 20 amino acid residues (SEQ ID NOs: 11, 19, 27) and 26 amino acid residues (SEQ ID NO: 39 of WO 2017/173359) instead of the maximum number of amino acid residues in accordance with the LCDR2 definition used in the present application (LCDR2: positions 58 to 72 according to the AHo numbering scheme, resulting in a maximum length of 15 amino acids).

Accordingly, in a first aspect, the present invention relates to an antibody variable domain, which binds to a target antigen, comprising:
(i) a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region;
and
(ii) a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region;
wherein said LCDR2 is defined by light chain amino acid positions 58 to 72 according to the AHo numbering scheme, resulting in a maximum length of 15 amino acids, and wherein said LCDR2 has a glycine (G) at amino acid position 67 and a glycine (G) at amino acid position 68 (AHo numbering);
and wherein said light chain complementarity-determining region LCDR2, i. e. amino acid positions 58, 67, 68, 69, 70, 71, 72 (AHo numbering), does not have a sequence selected from LGGNRAA (SEQ ID NO: 665) and RGGERVS (SEQ ID NO: 666).

In a second aspect, the present invention relates to an antibody comprising one or more antibody variable domains of the present invention.

In a third aspect, the present invention relates to a nucleic acid or two nucleic acids encoding the antibody variable domain or the antibody of the present invention.

In a fourth aspect, the present invention relates to a vector or two vectors comprising the nucleic acid or the two nucleic acids of the present invention.

In a fifth aspect, the present invention relates to a host cell or host cells comprising the vector or the two vectors of the present invention.

In a sixth aspect, the present invention relates to a method for producing the antibody variable domain of the present invention or the antibody of the present invention, comprising (i) providing the nucleic acid or the two nucleic acids of the present invention, or the vector or the two vectors of the present invention, expressing said nucleic acid or said two nucleic acids, or said vector or vectors, and collecting said antibody variable domain or said antibody from the expression system, or (ii) providing a host cell or host cells of the present invention, culturing said host cell or said host cells; and collecting said antibody variable domain or said antibody from the cell culture.

In a seventh aspect, the present invention relates to a pharmaceutical composition comprising the antibody of the present invention and a pharmaceutically acceptable carrier.

In an eighth aspect, the present invention relates to the pharmaceutical composition of the present invention for use as a medicament.

In a ninth aspect, the present invention relates to a method for generating a modified antibody variable domain, wherein the method comprises the steps of
1) providing an unmodified antibody variable domain, which binds to a target antigen, comprising:
   (i) a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region;
   and
   (ii) a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region, wherein the light chain complementarity-determining region LCDR2 does not comprise a glycine (G) at both amino acid position 67 and 68 (AHo numbering);
2) introducing into the light chain complementarity-determining region LCDR2 of the unmodified antibody variable domain provided in step 1) a glycine (G) at amino acid position 67 and/or a glycine (G) at amino acid position 68 (AHo numbering), such that there is a glycine (G) at both amino acid positions 67 and 68, wherein said LCDR2 is defined by light chain amino acid positions 58 to 72 according to the AHo numbering scheme, resulting in a maximum length of 15 amino acids.

In a tenth aspect, the present invention relates to a method for generating a modified antibody from an unmodified antibody, the method comprises the step of
1) selecting one or more antibody variable domains comprised in said unmodified antibody, which do not comprise a glycine (G) at both amino acid position 67 and 68 (AHo numbering) in the LCDR2, for modification;
2) introducing into each of said one or more antibody variable domains selected in step 1) a glycine (G) at amino acid position 67 and/or a glycine (G) at amino acid position 68 (AHo numbering) in the LCDR2, such that there is a glycine (G) at both amino acid positions 67 and 68 (AHo numbering) in the LCDR2.

The aspects, advantageous features and preferred embodiments of the present invention summarized in the following items, respectively alone or in combination, further contribute to solving the object of the invention:
1. An antibody variable domain, which binds to a target antigen, comprising:
   a. a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region;
   and
   b. a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region;
   wherein said LCDR2 is defined by light chain amino acid positions 58 to 72 according to the AHo numbering scheme, resulting in a maximum length of 15 amino acids, and wherein said LCDR2 has a glycine (G) at amino acid position 67 and a glycine (G) at amino acid position 68 (AHo numbering), particularly wherein said light chain complementarity-determining region LCDR2 consists of seven amino acids; and wherein said light chain complementarity-determining region LCDR2, i. e. amino acid positions 58, 67, 68, 69, 70, 71, 72 (AHo numbering), does not have a sequence selected from LGGNRAA (SEQ ID NO: 665) and RGGERVS (SEQ ID NO: 666).
2. The antibody variable domain of item 1, wherein said variable heavy chain framework regions HFW1, HFW2, HFW3 and HFW4 and/or said variable light chain framework regions LFW1, LFW2, LFW3 and LFW4 are selected from
   a) human antibody framework regions; and
b) human antibody framework regions comprising a total of 1 to 20, particularly 1 to 15, particularly 1 to 10, amino acid positions where the amino acids are taken from a non-human antibody, in particular from a lagomorph or rodent antibody, in particular from a rabbit or mouse antibody, in particular from a rabbit antibody (back mutations), said non-human antibody representing the source of the CDR sequences comprised in the antibody variable domain.
3. The antibody variable domain of item 1 or 2, wherein said variable heavy chain framework regions HFW1, HFW2, HFW3 and HFW4 and/or said variable light chain framework regions LFW1, LFW2, LFW3 and LFW4 are selected from framework regions of an antibody that has been tested in clinical trials.
4. The antibody variable domain of any one of the items 1 to 3, wherein
   a. said variable light chain framework regions LFW1, LFW2, LFW3 and LFW4 together are selected from a human antibody Vκ framework or a human antibody VA framework, in particular a human antibody Vκ framework; or
   b. said variable light chain framework regions LFW1, LFW2 and LFW3 are selected from a human antibody Vκ framework, and said variable light chain framework region LFW4 is selected from a human antibody Vλ framework.
5. The antibody variable domain of item 4, wherein said human antibody Vκ framework is selected from the Vκ framework subtypes Vκ1, Vκ2, Vκ3 and Vκ4.
6. The antibody variable domain of item 4, wherein said human antibody Vκ framework is of the Vκ framework subtype Vκ1.
7. The antibody variable domain of item 4, wherein said human antibody Vλ framework is selected from the Vλ framework subtypes VA1, VA2 and VA3.
8. The antibody variable domain of item 4, wherein said variable light chain framework region LFW4, when selected from a human antibody Vλ framework, has a sequence selected from
   a) any one of SEQ ID NOs: 527, 528, 529, 530, 531, 532, 533, 534 and 535; or
   b) any one of SEQ ID NOs: 527, 528, 529, 530, 531, 532, 533, 534 and 535, which has one or two mutations, particularly one mutation, particularly one or two mutations at one or two positions selected from positions 141, 144, 145, 146 and 147 (AHo numbering), more particularly one mutation at position 141 (AHo numbering).
9. The antibody variable domain of item 8, wherein said variable light chain framework regions LFW1, LFW2, LFW3 and LFW4 are selected from
   a. the combination of framework regions LFW1, LFW2, LFW3 and LFW4 (i. e. the residues corresponding to the non-italicized residues of SEQ ID NO: 3 or of the VL sequences shown in Table 2) of any one of the SEQ ID NOs: 304, 309, 515, 516, 517, 518, 519, 520, 521, 522, 523, 524, 525 and 526; and
   b. the combination of framework regions LFW1, LFW2, LFW3 and LFW4 (i. e. the residues corresponding to the non-italicized residues of SEQ ID NO: 3 or of the VL sequences shown in Table 2) of any one of the SEQ ID NOs: 304, 309, 515, 516, 517, 518, 519, 520, 521, 522, 523, 524, 525 and 526 having 1, 2, 3, 4 or 5 mutations within the framework regions.
10. The antibody variable domain of any one of the preceding items, wherein said variable heavy chain framework regions HFW1, HFW2, HFW3 and HFW4 are selected from a human VH framework.
11. The antibody variable domain of any one of the preceding items, wherein said variable heavy chain framework regions HFW1, HFW2, HFW3 and HFW4 are selected from the human VH framework subtypes VH1a, VH1b, VH3, VH4, VH5 and VH6.
12. The antibody variable domain of any one of the preceding items, wherein said variable heavy chain framework regions HFW1, HFW2, HFW3 and HFW4 are selected from the human VH framework subtypes VH1a, VH1b, VH3 and VH4.
13. The antibody variable domain of any one of the preceding items, wherein said variable heavy chain framework regions HFW1, HFW2, HFW3 and HFW4 are of the human VH framework subtype VH3.
14. The antibody variable domain of item 1, wherein said variable heavy chain framework regions HFW1, HFW2, HFW3 and HFW4 are selected from
   a. the combination of framework regions HFW1, HFW2, HFW3 and HFW4 (i. e. the residues corresponding to the non-italicized residues of SEQ ID NO: 1 or of the VH sequences shown in Table 2) of any one of the SEQ ID NOs: 302, 306, 496, 497, 498, 499, 500, 501, 502, 503, 504, 505, 506, 507, 508, 509, 510, 511, 512, 513 and 514; and
   b. the combination of framework regions HFW1, HFW2, HFW3 and HFW4 (i. e. the residues corresponding to the non-italicized residues of SEQ ID NO: 1 or of the VH sequences shown in Table 2) of any one of the SEQ ID NOs: 302, 306, 496, 497, 498, 499, 500, 501, 502, 503, 504, 505, 506, 507, 508, 509, 510, 511, 512, 513 and 514 having 1, 2, 3, 4 or 5 mutations within the framework regions.
15. The antibody variable domain of item 1, wherein
   (i) said variable light chain framework regions LFW1, LFW2 and LFW3 are selected from a human antibody Vκ framework, in particular are of the Vκ framework subtype Vκ1;
   (ii) said variable light chain framework region LFW4 is selected from a human antibody Vλ framework, in particular has a sequence selected from
      a. any one of SEQ ID NOs: 527, 528, 529, 530, 531, 532, 533, 534 and 535; or b. any one of SEQ ID NOs: 527, 528, 529, 530, 531, 532, 533, 534 and 535, which
      has one or two mutations, particularly one mutation, particularly one or two mutations at one or two positions selected from positions 141, 144, 145, 146 and 147 (AHo numbering), more particularly one mutation at position 141 (AHo numbering); and
   (iii) said variable heavy chain framework regions HFW1, HFW2, HFW3 and HFW4 are of the human VH framework subtype VH3.
16. The antibody variable domain of item 1, wherein
   (i) said variable light chain framework regions LFW1, LFW2, LFW3 and LFW4 are selected from
      a. the combination of framework regions LFW1, LFW2, LFW3 and LFW4 (i. e. the residues corresponding to the non-italicized residues of SEQ ID NO: 3 or of the VL sequences shown in Table 2) of any one of the SEQ ID NOs: 304, 309, 515, 516, 517, 518, 519, 520, 521, 522, 523, 524, 525 and 526; and
      b. the combination of framework regions LFW1, LFW2, LFW3 and LFW4 (i. e. the residues corresponding to the non-italicized residues of SEQ ID NO: 3 or of the VL sequences shown in Table 2) of any one of the SEQ ID NOs: 304, 309, 515, 516, 517, 518, 519, 520, 521, 522, 523, 524, 525 and 526 having 1, 2, 3, 4 or 5 mutations within the framework regions; and
   (ii) said variable heavy chain framework regions HFW1, HFW2, HFW3 and HFW4 are selected from
      a. the combination of framework regions HFW1, HFW2, HFW3 and HFW4 (i. e. the residues corresponding to the non-italicized residues of SEQ ID NO: 1 or of the VH sequences shown in Table 2) of any one of the SEQ ID NOs: 302, 306, 496, 497, 498, 499, 500, 501, 502, 503, 504, 505, 506, 507, 508, 509, 510, 511, 512, 513 and 514; and
      b. the combination of framework regions HFW1, HFW2, HFW3 and HFW4 (i. e. the residues corresponding to the non-italicized residues of SEQ ID NO: 1 or of the VH sequences shown in Table 2) of any one of the SEQ ID NOs: 302, 306, 496, 497, 498, 499, 500, 501, 502, 503, 504, 505, 506, 507, 508, 509, 510, 511, 512, 513 and 514 having 1, 2, 3, 4 or 5 mutations within the framework regions.
17. The antibody variable domain of any one of items 1 to 16, wherein the light chain complementarity-determining region LCDR2 has a sequence selected from X₁GGX₂X₃X₄X₅(SEQ ID NO: 536), wherein X₁, X₂, X₃, X₄ and X₅ correspond to amino acid positions 58, 69, 70, 71 and 72 (AHo numbering), and wherein said X₁, X₂, X₃, X₄ and X₅ of LCDR2 are selected from:
   (i) the corresponding positions of an LCDR2 region of a mammalian antibody, in particular of an LCDR2 region of a lagomorph, rodent or human antibody, in particular of an LCDR2 region of a rabbit, mouse or human antibody, in particular of an LCDR2 region of a rabbit antibody; or
   (ii) the corresponding positions of an LCDR2 region of an antibody that has been tested in clinical trials; or
   (iii) the corresponding positions of an LCDR2 region of an antibody according to (i) or (ii), wherein one, two or three of said amino acid positions, in particular one or two of said amino acid positions, have been altered.
18. The antibody variable domain of any one of item 17, wherein the other light chain complementarity-determining regions LCDR1 and LCDR3 are selected from:
   (i) LCDR1 and LCDR3 regions of said mammalian antibody; or
   (ii) LCDR1 and LCDR3 regions of said antibody that has been tested in clinical trials; or
   (iii) LCDR1 and LCDR3 regions of an antibody according to (i) or (ii), wherein at least in one of said LCDR1 and LCDR3 regions, independently from each other, one, two or three amino acid positions, in particular one or two amino acid positions, have been altered.
19. The antibody variable domain of items 17 or 18, wherein the heavy chain complementarity-determining regions HCDR1, HCDR2 and HCDR3 are selected from
   (i) complementarity-determining regions of said mammalian antibody; or
   (ii) complementarity-determining regions of said antibody that has been tested in clinical trials; or
   (iii) complementarity-determining regions of an antibody according to (i) or (ii), wherein at least in one of said complementarity-determining regions, independently from each other, one, two or three amino acid positions, in particular one or two amino acid positions, have been altered.
20. The antibody variable domain of any one of the preceding items, wherein said light chain complementarity-determining region LCDR2 has one or more of the following features:
   (a) has a lysine (K), leucine (L), methionine (M) or arginine (R), in particular a lysine (K), methionine (M) or leucine (L), in particular a leucine (L), at amino acid position 70 (AHo numbering);
   (b) has a sequence selected from X₁GGX₂X₃X₄X₅(SEQ ID NO: 536), wherein X₁ is selected from lysine (K), arginine (R), serine (S), threonine (T), alanine (A), leucine (L), glycine (G), glutamine (Q), glutamate (E), tyrosine (Y) and aspartate (D); in particular from lysine (K), arginine (R), serine (S), tyrosine (Y), glycine (G), and aspartate (D);
      X₂ is selected from lysine (K), threonine (T), serine (S), isoleucine (I), aspartate (D), asparagine (N), phenylalanine (F) and tyrosine (Y); in particular from lysine (K), threonine (T), serine (S), aspartate (D), asparagine (N) and tyrosine (Y); X₃ is selected from lysine (K), leucine (L), methionine (M) and arginine (R); in particular from lysine (K), methionine (M) and leucine (L); in particular is leucine (L); X₄ is selected from alanine (A), glycine (G), valine (V), threonine (T), proline (P), tyrosine (Y), aspartate (D), and glutamate (E); in particular from alanine (A), glycine (G), threonine (T) and glutamate (E); X₅ is selected from serine (S), threonine (T) and tyrosine (Y); in particular is serine (S).
21. The antibody variable domain of any one of the preceding items, wherein said light chain complementarity-determining region LCDR2, i. e. amino acid positions 58, 67, 68, 69, 70, 71, 72 (AHo numbering), has a sequence selected from RGGILAS (SEQ ID NO: 667), KGGTLAS (SEQ ID NO: 668), RGGTLAS (SEQ ID NO: 669), SGGTLAS (SEQ ID NO: 670), LGGTLAS (SEQ ID NO: 671), TGGTLAS (SEQ ID NO: 672), GGGTLAS (SEQ ID NO: 673), RGGNLAS (SEQ ID NO: 674), DGGDLAS (SEQ ID NO: 675), DGGKLAS (SEQ ID NO: 676), QGGKLAS (SEQ ID NO: 677), RGGKLAS (SEQ ID NO: 678), LGGKLAS (SEQ ID NO: 679), SGGKLAS (SEQ ID NO: 680), GGGKLAS (SEQ ID NO: 681), DGGRLAS (SEQ ID NO: 682), DGGNRAT (SEQ ID NO: 683), RGGTLES (SEQ ID NO: 684), KGGTLES (SEQ ID NO: 685), DGGDLTS (SEQ ID NO: 686), SGGFLYS (SEQ ID NO: 687), SGGYRYT (SEQ ID NO: 688), SGGKLAA (SEQ ID NO: 689), SGGTLVS (SEQ ID NO: 690), RGGTLAY (SEQ ID NO: 691), AGGTLAS (SEQ ID NO: 692), AGGYLAS (SEQ ID NO: 693), RGGYLES (SEQ ID NO: 694), EGGKLAS (SEQ ID NO: 695), RGGNLES (SEQ ID NO: 696), RGGILES (SEQ ID NO: 697), RGGNRES (SEQ ID NO: 698), RGGTLDS (SEQ ID NO: 699), LGGKLES (SEQ ID NO: 700), LGGKMES (SEQ ID NO: 701), TGGSLAS (SEQ ID NO: 702) and GGGTLES (SEQ ID NO: 703).
22. The antibody variable domain of any one of the preceding items, wherein the format of said antibody variable domain is selected from a Fab, an scFab, an Fv, a dsFv, an scFv and a dsscFv, particularly from a Fab, Fv and scFv.
23. The antibody variable domain of any one of the preceding items, wherein the variable light chain of said antibody variable domain comprises no 15mer peptide stretch having a median percentile rank, as determined from the predicted individual binding strengths (scoring ranks) of said 15mer peptide to each one of the 27 MHC class II alleles that are most common in the general population using the algorithm of NetMHCIIpan-3.1, of less than 13, particularly less than 14, particularly less than 15, particularly less than 16, particularly less than 17, particularly less than 18, particularly less than 19, particularly less than 20.
24. The antibody variable domain of any one of the preceding items, wherein the variable heavy chain of said antibody variable domain comprises no 15mer peptide stretch having a median percentile rank, as determined from the predicted individual binding strengths (scoring ranks) of said 15mer peptide to each one of the 27 MHC class II alleles that are most common in the general population using the algorithm NetMHCIIpan-3.1, of less than 13, particularly less than 14, particularly less than 15, particularly less than 16, particularly less than 17, particularly less than 18, particularly less than 19, particularly less than 20.
25. The antibody variable domain of any one of the preceding items, wherein said antibody variable domain, when being in scFv format, is further characterized by one or more of the following features:
   a. has a melting temperature (Tm), when compared with the Tm of a version of said antibody variable domain that does not comprise the LCDR2 amino acids defined in item 1, that is not lower than 5° C., particularly not lower than 4.5° C., particularly not lower than 4° C., particularly not lower than 3.5° C., particularly not lower than 3.0° C., as determined by differential scanning fluorimetry (DSF) and when formulated in 20 mM Histidine, pH 6.0;
   b. binds its target antigen with a dissociation constant (KD) that is not greater than 2-folds, particularly not greater than 1.7-folds, particularly not greater than 1.5-folds, particularly not greater than 1.4-folds, particularly not greater than 1.3-folds, particularly not greater than 1.2-folds, when compared with the KD of a version of said antibody variable domain that does not comprise the LCDR2 amino acids defined in item 1, as measured by surface plasmon resonance (SPR).
26. The antibody variable domain of item 1, wherein said antibody variable domain comprises:
   (i) a VH sequence selected from SEQ ID NOs: 1 and 2 and from variants of SEQ ID NOs: 1 and 2 having 1, 2 or 3 mutations within the framework regions, and (ii) a VL sequence selected from SEQ ID NO: 4 and from variants of SEQ ID NO: 4 having 1, 2 or 3 mutations within the framework regions;
   or
   (i) a VH sequence selected from SEQ ID NOs: 8 and 9 and from variants of SEQ ID NOs: 8 and 9 having 1, 2 or 3 mutations within the framework regions, and
   (ii) a VL sequence selected from SEQ ID NO: 11 and from variants of SEQ ID NO: 11 having 1, 2 or 3 mutations within the framework regions;
   or
   (i) a VH sequence selected from SEQ ID NOs: 15-18 and from variants of SEQ ID NOs: 15-18 having 1, 2 or 3 mutations within the framework regions, and (ii) a VL sequence selected from SEQ ID NO: 20 and 22 and from variants of SEQ ID NO: 20 and 22 having 1, 2 or 3 mutations within the framework regions; or
   (i) a VH sequence selected from SEQ ID NOs: 31 and from variants of SEQ ID NOs: 31 having 1, 2 or 3 mutations within the framework regions, and
   (ii) a VL sequence selected from SEQ ID NO: 33 and from variants of SEQ ID NO: 33 having 1, 2 or 3 mutations within the framework regions;
   or
   (i) a VH sequence selected from SEQ ID NOs: 36 and 37 and from variants of SEQ ID NOs: 36 and 37 having 1, 2 or 3 mutations within the framework regions, and
   (ii) a VL sequence selected from SEQ ID NO: 39 and from variants of SEQ ID NO: 39 having 1, 2 or 3 mutations within the framework regions; or
   (i) a VH sequence selected from SEQ ID NOs: 43 and 44 and from variants of SEQ ID NOs: 43 and 44 having 1, 2 or 3 mutations within the framework regions, and
   (ii) a VL sequence selected from SEQ ID NO: 46 and from variants of SEQ ID NO: 46 having 1, 2 or 3 mutations within the framework regions;
   or
   (i) a VH sequence selected from SEQ ID NOs: 50 and 51 and from variants of SEQ ID NOs: 50 and 51 having 1, 2 or 3 mutations within the framework regions, and
   (ii) a VL sequence selected from SEQ ID NO: 53 and from variants of SEQ ID NO: 53 having 1, 2 or 3 mutations within the framework regions;
   or
   (i) a VH sequence selected from SEQ ID NOs: 57 and 58 and from variants of SEQ ID NOs: 57 and 58 having 1, 2 or 3 mutations within the framework regions, and
   (ii) a VL sequence selected from SEQ ID NO: 60 and from variants of SEQ ID NO: 60 having 1, 2 or 3 mutations within the framework regions;
   or
   (i) a VH sequence selected from SEQ ID NOs: 64-66 and from variants of SEQ ID NOs: 64-66 having 1, 2 or 3 mutations within the framework regions, and
   (ii) a VL sequence selected from SEQ ID NO: 68 and 69 and from variants of SEQ ID NO: 68 and 69 having 1, 2 or 3 mutations within the framework regions; or
   (i) a VH sequence selected from SEQ ID NOs: 74-77 and from variants of SEQ ID NOs: 74-77 having 1, 2 or 3 mutations within the framework regions, and
   (ii) a VL sequence selected from SEQ ID NO: 80 and 81 and from variants of SEQ ID NO: 80 and 81 having 1, 2 or 3 mutations within the framework regions; or
   (i) a VH sequence selected from SEQ ID NOs: 94 and 95 and from variants of SEQ ID NOs: 94 and 95 having 1, 2 or 3 mutations within the framework regions, and
   (ii) a VL sequence selected from SEQ ID NO: 97 and from variants of SEQ ID NO: 97 having 1, 2 or 3 mutations within the framework regions;
   or
   (i) a VH sequence selected from SEQ ID NOs: 101 and 102 and from variants of SEQ ID NOs: 101 and 102 having 1, 2 or 3 mutations within the framework regions, and
   (ii) a VL sequence selected from SEQ ID NO: 105 and 106 and from variants of SEQ ID NO: 105 and 106 having 1, 2 or 3 mutations within the framework regions;
   or
   (i) a VH sequence selected from SEQ ID NOs: 110 and 111 and from variants of SEQ ID NOs: 110 and 111 having 1, 2 or 3 mutations within the framework regions, and
   (ii) a VL sequence selected from SEQ ID NO: 113 and from variants of SEQ ID NO: 113 having 1, 2 or 3 mutations within the framework regions;
   or
   (i) a VH sequence selected from SEQ ID NOs: 117 and 118 and from variants of SEQ ID NOs: 117 and 118 having 1, 2 or 3 mutations within the framework regions, and
   (ii) a VL sequence selected from SEQ ID NO: 120 and from variants of SEQ ID NO: 120 having 1, 2 or 3 mutations within the framework regions;
   or
   (i) a VH sequence selected from SEQ ID NOs: 124 and 125 and from variants of SEQ ID NOs: 124 and 125 having 1, 2 or 3 mutations within the framework regions, and
   (ii) a VL sequence selected from SEQ ID NO: 127 and from variants of SEQ ID NO: 127 having 1, 2 or 3 mutations within the framework regions;
   or
   (i) a VH sequence selected from SEQ ID NOs: 131 and from variants of SEQ ID NOs: 131 having 1, 2 or 3 mutations within the framework regions, and
   (ii) a VL sequence selected from SEQ ID NO: 133 and from variants of SEQ ID NO: 133 having 1, 2 or 3 mutations within the framework regions;
   or
   (i) a VH sequence selected from SEQ ID NOs: 136 and 137 and from variants of SEQ ID NOs: 136 and 137 having 1, 2 or 3 mutations within the framework regions, and
   (ii) a VL sequence selected from SEQ ID NO: 140 and 141 and from variants of SEQ ID NO: 140 and 141 having 1, 2 or 3 mutations within the framework regions; or
   (i) a VH sequence selected from SEQ ID NOs: 145 and 146 and from variants of SEQ ID NOs: 145 and 146 having 1, 2 or 3 mutations within the framework regions, and
   (ii) a VL sequence selected from SEQ ID NO: 148 and from variants of SEQ ID NO: 148 having 1, 2 or 3 mutations within the framework regions;
   or
   (i) a VH sequence selected from SEQ ID NOs: 152 and 153 and from variants of SEQ ID NOs: 152 and 153 having 1, 2 or 3 mutations within the framework regions, and
   (ii) a VL sequence selected from SEQ ID NO: 155 and from variants of SEQ ID NO: 155 having 1, 2 or 3 mutations within the framework regions;
   or
   (i) a VH sequence selected from SEQ ID NOs: 159-161 and from variants of SEQ ID NOs: 159-161 having 1, 2 or 3 mutations within the framework regions, and
   (ii) a VL sequence selected from SEQ ID NO: 163 and 164 and from variants of SEQ ID NO: 163 and 164 having 1, 2 or 3 mutations within the framework regions;
   or
   (i) a VH sequence selected from SEQ ID NOs: 170-173 and from variants of SEQ ID NOs: 170-173 having 1, 2 or 3 mutations within the framework regions, and
   (ii) a VL sequence selected from SEQ ID NO: 175 and 176 and from variants of SEQ ID NO: 175 and 176 having 1, 2 or 3 mutations within the framework regions;
   or
   (i) a VH sequence selected from SEQ ID NOs: 182 and 183 and from variants of SEQ ID NOs: 182 and 183 having 1, 2 or 3 mutations within the framework regions, and
   (ii) a VL sequence selected from SEQ ID NO: 185 and from variants of SEQ ID NO: 185 having 1, 2 or 3 mutations within the framework regions;
   or
   (i) a VH sequence selected from SEQ ID NOs: 190 and 193 and from variants of SEQ ID NOs: 190 and 193 having 1, 2 or 3 mutations within the framework regions, and
   (ii) a VL sequence selected from SEQ ID NO: 195 and 196 and from variants of SEQ ID NO: 195 and 196 having 1, 2 or 3 mutations within the framework regions;
   or
   (i) a VH sequence selected from SEQ ID NOs: 202-205 and from variants of SEQ ID NOs: 202-205 having 1, 2 or 3 mutations within the framework regions, and
   (ii) a VL sequence selected from SEQ ID NO: 207 and 208 and from variants of SEQ ID NO: 207 and 208 having 1, 2 or 3 mutations within the framework regions;
   or
   (i) a VH sequence selected from SEQ ID NOs: 214-217 and from variants of SEQ ID NOs: 214-217 having 1, 2 or 3 mutations within the framework regions, and
   (ii) a VL sequence selected from SEQ ID NO: 219 and 220 and from variants of SEQ ID NO: 219 and 220 having 1, 2 or 3 mutations within the framework regions;
   or
   (i) a VH sequence selected from SEQ ID NOs: 226 and 227 and from variants of SEQ ID NOs: 226 and 227 having 1, 2 or 3 mutations within the framework regions, and
   (ii) a VL sequence selected from SEQ ID NO: 229 and from variants of SEQ ID NO: 229 having 1, 2 or 3 mutations within the framework regions;
   or
   (i) a VH sequence selected from SEQ ID NOs: 233-236 and from variants of SEQ ID NOs: 233-236 having 1, 2 or 3 mutations within the framework regions,
   and
   (ii) a VL sequence selected from SEQ ID NO: 239 and 240 and from variants of SEQ ID NO: 239 and 240 having 1, 2 or 3 mutations within the framework regions; or
   (i) a VH sequence selected from SEQ ID NOs: 247-250 and from variants of SEQ ID NOs: 247-250 having 1, 2 or 3 mutations within the framework regions, and
   (ii) a VL sequence selected from SEQ ID NO: 252 and from variants of SEQ ID NO: 252 having 1, 2 or 3 mutations within the framework regions;
   or
   (i) a VH sequence selected from SEQ ID NOs: 257 and 258 and from variants of SEQ ID NOs: 257 and 258 having 1, 2 or 3 mutations within the framework regions, and
   (ii) a VL sequence selected from SEQ ID NO: 260 and from variants of SEQ ID NO: 260 having 1, 2 or 3 mutations within the framework regions;
   or
   (i) a VH sequence selected from SEQ ID NOs: 264-267 and from variants of SEQ ID NOs: 264-267 having 1, 2 or 3 mutations within the framework regions, and
   (ii) a VL sequence selected from SEQ ID NO: 269 and 270 and from variants of SEQ ID NO: 269 and 270 having 1, 2 or 3 mutations within the framework regions;
   or
   (i) a VH sequence selected from SEQ ID NOs: 275-278 and from variants of SEQ ID NOs: 275-278 having 1, 2 or 3 mutations within the framework regions, and
   (ii) a VL sequence selected from SEQ ID NO: 280 and 281 and from variants of SEQ ID NO: 280 and 281 having 1, 2 or 3 mutations within the framework regions;
   or
   (i) a VH sequence selected from SEQ ID NOs: 286-291 and from variants of SEQ ID NOs: 286-291 having 1, 2 or 3 mutations within the framework regions, and
   (ii) a VL sequence selected from SEQ ID NO: 293 and 294 and from variants of SEQ ID NO: 293 and 294 having 1, 2 or 3 mutations within the framework regions;
   or
   (i) a VH sequence selected from SEQ ID NOs: 302 and from variants of SEQ ID NOs: 302 having 1, 2 or 3 mutations within the framework regions, and
   (ii) a VL sequence selected from SEQ ID NO: 304 and from variants of SEQ ID NO: 304 having 1, 2 or 3 mutations within the framework regions;
   or
   (i) a VH sequence selected from SEQ ID NOs: 307 and from variants of SEQ ID NOs: 307 having 1, 2 or 3 mutations within the framework regions, and
   (ii) a VL sequence selected from SEQ ID NO: 309 and from variants of SEQ ID NO: 309 having 1, 2 or 3 mutations within the framework regions;
   or
   (i) a VH sequence selected from SEQ ID NOs: 312 and 313 and from variants of SEQ ID NOs: 312 and 313 having 1, 2 or 3 mutations within the framework regions, and
   (ii) a VL sequence selected from SEQ ID NO: 317-319 and from variants of SEQ ID NO: 317-319 having 1, 2 or 3 mutations within the framework regions;
   or
   (i) a VH sequence selected from SEQ ID NOs: 327-330 and from variants of SEQ ID NOs: 327-330 having 1, 2 or 3 mutations within the framework regions, and
   (ii) a VL sequence selected from SEQ ID NO: 332 and from variants of SEQ ID NO: 332 having 1, 2 or 3 mutations within the framework regions;
   or
   (i) a VH sequence selected from SEQ ID NOs: 338-341 and from variants of SEQ ID NOs: 338-341 having 1, 2 or 3 mutations within the framework regions, and
   (ii) a VL sequence selected from SEQ ID NO: 343 and from variants of SEQ ID NO: 343 having 1, 2 or 3 mutations within the framework regions;
   or
   (i) a VH sequence selected from SEQ ID NOs: 349 and 350 and from variants of SEQ ID NOs: 349 and 350 having 1, 2 or 3 mutations within the framework regions, and
   (ii) a VL sequence selected from SEQ ID NO: 352 and from variants of SEQ ID NO: 352 having 1, 2 or 3 mutations within the framework regions;
   or
   (i) a VH sequence selected from SEQ ID NOs: 356 and 357 and from variants of SEQ ID NOs: 356 and 357 having 1, 2 or 3 mutations within the framework regions, and
   (ii) a VL sequence selected from SEQ ID NO: 359 and from variants of SEQ ID NO: 359 having 1, 2 or 3 mutations within the framework regions;
   or
   (i) a VH sequence selected from SEQ ID NOs: 363 and 364 and from variants of SEQ ID NOs: 363 and 364 having 1, 2 or 3 mutations within the framework regions, and
   (ii) a VL sequence selected from SEQ ID NO: 366 and from variants of SEQ ID NO: 366 having 1, 2 or 3 mutations within the framework regions;
   or
   (i) a VH sequence selected from SEQ ID NOs: 370 and 371 and from variants of SEQ ID NOs: 370 and 371 having 1, 2 or 3 mutations within the framework regions, and
   (ii) a VL sequence selected from SEQ ID NO: 373 and from variants of SEQ ID NO: 373 having 1, 2 or 3 mutations within the framework regions;
   or
   (i) a VH sequence selected from SEQ ID NOs: 377 and 378 and from variants of SEQ ID NOs: 377 and 378 having 1, 2 or 3 mutations within the framework regions, and
   (ii) a VL sequence selected from SEQ ID NO: 380 and from variants of SEQ ID NO: 380 having 1, 2 or 3 mutations within the framework regions;
   or
   (i) a VH sequence selected from SEQ ID NOs: 384 and 385 and from variants of SEQ ID NOs: 384 and 385 having 1, 2 or 3 mutations within the framework regions, and
   (ii) a VL sequence selected from SEQ ID NO: 387 and from variants of SEQ ID NO: 387 having 1, 2 or 3 mutations within the framework regions;
   or
   (i) a VH sequence selected from SEQ ID NOs: 391 and 392 and from variants of SEQ ID NOs: 391 and 392 having 1, 2 or 3 mutations within the framework regions, and
   (ii) a VL sequence selected from SEQ ID NO: 394 and from variants of SEQ ID NO: 394 having 1, 2 or 3 mutations within the framework regions;
   or
   (i) a VH sequence selected from SEQ ID NOs: 398 and 399 and from variants of SEQ ID NOs: 398 and 399 having 1, 2 or 3 mutations within the framework regions, and
   (ii) a VL sequence selected from SEQ ID NO: 401 and from variants of SEQ ID NO: 401 having 1, 2 or 3 mutations within the framework regions;
   or
   (i) a VH sequence selected from SEQ ID NOs: 405 and 406 and from variants of SEQ ID NOs: 405 and 406 having 1, 2 or 3 mutations within the framework regions, and
   (ii) a VL sequence selected from SEQ ID NO: 408 and from variants of SEQ ID NO: 408 having 1, 2 or 3 mutations within the framework regions;
   or
   (i) a VH sequence selected from SEQ ID NOs: 412 and 413 and from variants of SEQ ID NOs: 412 and 413 having 1, 2 or 3 mutations within the framework regions, and
   (ii) a VL sequence selected from SEQ ID NO: 415 and from variants of SEQ ID NO: 415 having 1, 2 or 3 mutations within the framework regions;
   or
   (i) a VH sequence selected from SEQ ID NOs: 419 and 420 and from variants of SEQ ID NOs: 419 and 420 having 1, 2 or 3 mutations within the framework regions, and
   (ii) a VL sequence selected from SEQ ID NO: 422 and from variants of SEQ ID NO: 422 having 1, 2 or 3 mutations within the framework regions;
   or
   (i) a VH sequence selected from SEQ ID NOs: 426 and 427 and from variants of SEQ ID NOs: 426 and 427 having 1, 2 or 3 mutations within the framework regions, and
   (ii) a VL sequence selected from SEQ ID NO: 429 and from variants of SEQ ID NO: 429 having 1, 2 or 3 mutations within the framework regions;
   or
   (i) a VH sequence selected from SEQ ID NOs: 433 and 434 and from variants of SEQ ID NOs: 433 and 434 having 1, 2 or 3 mutations within the framework regions, and
   (ii) a VL sequence selected from SEQ ID NO: 436 and from variants of SEQ ID NO: 436 having 1, 2 or 3 mutations within the framework regions;
   or
   (i) a VH sequence selected from SEQ ID NOs: 440 and 441 and from variants of SEQ ID NOs: 440 and 441 having 1, 2 or 3 mutations within the framework regions, and
   (ii) a VL sequence selected from SEQ ID NO: 443 and from variants of SEQ ID NO: 443 having 1, 2 or 3 mutations within the framework regions;
   or
   (i) a VH sequence selected from SEQ ID NOs: 447 and 448 and from variants of SEQ ID NOs: 447 and 448 having 1, 2 or 3 mutations within the framework regions, and
   (ii) a VL sequence selected from SEQ ID NO: 450 and from variants of SEQ ID NO: 450 having 1, 2 or 3 mutations within the framework regions;
   or
   (i) a VH sequence selected from SEQ ID NOs: 454 and 455 and from variants of SEQ ID NOs: 454 and 455 having 1, 2 or 3 mutations within the framework regions, and
   (ii) a VL sequence selected from SEQ ID NO: 457 and from variants of SEQ ID NO: 457 having 1, 2 or 3 mutations within the framework regions;
   or
   (i) a VH sequence selected from SEQ ID NOs: 461 and 462 and from variants of SEQ ID NOs: 461 and 462 having 1, 2 or 3 mutations within the framework regions, and
   (ii) a VL sequence selected from SEQ ID NO: 464 and from variants of SEQ ID NO: 464 having 1, 2 or 3 mutations within the framework regions;
   or
   (i) a VH sequence selected from SEQ ID NOs: 468 and 469 and from variants of SEQ ID NOs: 468 and 469 having 1, 2 or 3 mutations within the framework regions, and
   (ii) a VL sequence selected from SEQ ID NO: 471 and from variants of SEQ ID NO: 471 having 1, 2 or 3 mutations within the framework regions;
   or
   (i) a VH sequence selected from SEQ ID NOs: 475 and 476 and from variants of SEQ ID NOs: 475 and 476 having 1, 2 or 3 mutations within the framework regions, and
   (ii) a VL sequence selected from SEQ ID NO: 478 and from variants of SEQ ID NO: 478 having 1, 2 or 3 mutations within the framework regions;
   or
   (i) a VH sequence selected from SEQ ID NOs: 482 and 483 and from variants of SEQ ID NOs: 482 and 483 having 1, 2 or 3 mutations within the framework regions, and
   (ii) a VL sequence selected from SEQ ID NO: 485 and from variants of SEQ ID NO: 485 having 1, 2 or 3 mutations within the framework regions;
   or
   (i) a VH sequence selected from SEQ ID NOs: 489 and 490 and from variants of SEQ ID NOs: 489 and 490 having 1, 2 or 3 mutations within the framework regions, and
   (ii) a VL sequence selected from SEQ ID NO: 492 and from variants of SEQ ID NO: 492 having 1, 2 or 3 mutations within the framework regions;
   or
   wherein said antibody variable domain is an scFv, which has a sequence selected from SEQ ID NOs: 6, 7, 13, 14, 24, 25, 27, 28, 29, 30, 35, 41, 42, 48, 49, 55, 56, 62, 63, 71-73, 83, 84, 86, 87, 89, 90, 92, 93, 99, 100, 108, 109, 115, 116, 122, 123, 129, 130, 135, 143, 144, 150, 151, 157, 158, 166, 168, 169, 178-181, 189, 198-201, 210-213, 222-225, 231, 232, 242, 243, 245, 246, 254-256, 262, 263, 272-274, 283-285, 296-298, 300, 301, 306, 311, 321-326, 334-337, 345-348, 354, 355, 361, 362, 368, 369, 375, 376, 382, 383, 389, 390, 396, 397, 403, 404, 410, 411, 417, 418, 424, 425, 431, 432, 438, 439, 445, 446, 452, 453, 459, 460, 466, 467, 473, 474, 480, 481, 487, 488, 494, 495 and from variants of SEQ ID NOs: 6, 7, 13, 14, 24, 25, 27, 28, 29, 30, 35, 41, 42, 48, 49, 55, 56, 62, 63, 71-73, 83, 84, 86, 87, 89, 90, 92, 93, 99, 100, 108, 109, 115, 116, 122, 123, 129, 130, 135, 143, 144, 150, 151, 157, 158, 166, 168, 169, 178-181, 189, 198-201, 210-213, 222-225, 231, 232, 242, 243, 245, 246, 254-256, 262, 263, 272-274, 283-285, 296-298, 300, 301, 306, 311, 321-326, 334-337, 345-348, 354, 355, 361, 362, 368, 369, 375, 376, 382, 383, 389, 390, 396, 397, 403, 404, 410, 411, 417, 418, 424, 425, 431, 432, 438, 439, 445, 446, 452, 453, 459, 460, 466, 467, 473, 474, 480, 481, 487, 488, 494, 495 having 1, 2, 3, 4 or 5 mutations within the framework regions.
27. The antibody variable domain as defined in any one of items 1 to 26, wherein the variable heavy chain (VH) comprises a serine (S) at amino acid position 101 and a lysine (K) at amino acid position 146 (AHo numbering).
28. An antibody comprising one or more antibody variable domains as defined in any one of items 1 to 27.
29. The antibody of item 28, wherein the antibody is
   (i) monospecific and monovalent, bivalent or trivalent for its target antigen;
   (ii) bispecific and, independently of each other, monovalent or bivalent for each target antigen;
   (iii) trispecific and monovalent for each target antigen;
   (iv) trispecific and bivalent for one of the target antigens and monovalent for the other target antigens;
   (v) trispecific and bivalent for two of the target antigens and monovalent for the third target antigen;
   (vi) tetraspecific and monovalent for each target antigen;
   (vii) tetraspecific and bivalent for one of the target antigens and monovalent for the other target antigens; or
   (viii) tetraspecific and bivalent for two of the target antigens and monovalent for the other target antigens.
30. The antibody of any one of items 28 and 29, wherein said antibody does comprise an immunoglobulin Fc region.
31. The antibody of item 30, wherein the format of said antibody is selected from an Fc-bearing KiH-based format, DVD-Ig, CODV-IgG, a Morrison format (IgG CH₃-scFv fusion (Morrison-H) or IgG CL-scFv fusion (Morrison-L)), (scFv)₂-Fc-(scFv)₂ fusion (ADAPTIR), an Fc-bearing DART™ and an Fc-bearing TRIDENT™, particularly DVD-Ig, a Morrison format (IgG CH₃-scFv fusion (Morrison-H) or IgG CL-scFv fusion (Morrison-L)), BsAb (scFv linked to C-terminus of light chain), Bs1Ab (scFv linked to N-terminus of light chain), Bs2Ab (scFv linked to N-terminus of heavy chain), Bs3Ab (scFv linked to C-terminus of heavy chain), Ts1Ab (scFv linked to N-terminus of both heavy chain and light chain), Ts2Ab (dsscFv linked to C-terminus of heavy chain), an IgG CrossMAb format and an IgG scDB format (IgG CH₃-scDB fusion or IgG CL-scDB fusion).
32. The antibody of item 31, wherein case the format comprises an IgG region, the IgG region is selected from the IgG subclasses IgG1 and IgG4.
33. The antibody of any one of items 28 and 29, wherein said antibody does not comprise an immunoglobulin Fc region.
34. The antibody of item 33, wherein said antibody is in a format selected from the group consisting of: a tandem scDb (Tandab), a linear dimeric scDb (LD-scDb), a circular dimeric scDb (CD-scDb), a tandem tri-scFv, a tribody (Fab-(scFv)₂), a Fab-Fv₂, a triabody, an scDb-scFv, a tetrabody, a didiabody, a tandem-di-scFv and a MATCH.
35. The antibody of item 33, wherein said antibody does not comprise CH1 and/or CL regions.
36. The antibody of item 35, wherein said antibody is in a scDb-scFv, a triabody, a tetrabody or a MATCH format, in particular wherein said antibody is in a MATCH or scDb-scFv format, more particularly wherein said antibody is in a MATCH format, more particularly a MATCH3 or a MATCH4 format.
37. The antibody of item 27, wherein said antibody is a single chain antibody having a sequence selected from SEQ ID NOs: 545-550, 552, 553, 743, 555, 556, 558, 559, 561, 562, 564, 565, 567, 568, 570, 571, 573, 574, 576, 577, 579, 580, 582, 583, 585, 586, 588, 589, 591, 592, 594, 595, 603, 604, 606, 607, 645, 646, 648 and 649 and from variants of SEQ ID NOs: 545-550, 552, 553, 743, 555, 556, 558, 559, 561, 562, 564, 565, 567, 568, 570, 571, 573, 574, 576, 577, 579, 580, 582, 583, 585, 586, 588, 589, 591, 592, 594, 595, 603, 604, 606, 607, 645, 646, 648 and 649 having 1, 2, 3, 4 or 5 mutations within the framework regions;
   or wherein said antibody is a heterodimer consisting of two chains having a pair of sequences selected from the following SEQ ID NOs: 597 and 600; 598 and 601; 609 and 612; 610 and 613; 615 and 618; 616 and 619; 621 and 624; 622 and 625; 627 and 630; 627 and 631; 633 and 636; 634 and 637; 639 and 642; 640 and 643; 645 and 648; 646 and 649; 651 and 654; 652 and 655; 657 and 660; 658 and 661; and from variants of SEQ ID NOs: 597 and 600; 598 and 601; 609 and 612; 610 and 613; 615 and 618; 616 and 619; 621 and 624; 622 and 625; 627 and 630; 627 and 631; 633 and 636; 634 and 637; 639 and 642; 640 and 643; 645 and 648; 646 and 649; 651 and 654; 652 and 655; 657 and 660; 658 and 661; wherein each of the sequences in said sequence pairs has 1, 2, 3, 4 or 5 mutations within the framework regions.
38. A nucleic acid or two nucleic acids encoding the antibody variable domain of any one of items 1 to 26, or the antibody of any one of items 28 to 37.
39. A vector or two vectors comprising the nucleic acid or the two nucleic acids of item 38.
40. A host cell or host cells comprising the vector or the two vectors of item 39.
41. A method for producing the antibody variable domain of any one of items 1 to 27, or the antibody of any one of items 28 to 37, comprising (i) providing the nucleic acid or the two nucleic acids of item 38, or the vector or the two vectors of item 39, expressing said nucleic acid sequence or nucleic acids, or said vector or vectors, and collecting said antibody variable domain or said antibody from the expression system, or (ii) providing a host cell or host cells of item 40, culturing said host cell or said host cells; and collecting said antibody variable domain or said antibody from the cell culture.
42. A pharmaceutical composition comprising the antibody of any one of items 28 to 37 and a pharmaceutically acceptable carrier.
43. The antibody of any one of items 28 to 37, or the pharmaceutical composition of item 42, for use as a medicament.
44. A method for generating a modified antibody variable domain from an unmodified antibody variable domain, wherein said unmodified antibody variable domain binds to a target antigen and comprises:
   (i) a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region,
   and
   (ii) a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region, wherein the light chain complementarity-determining region LCDR2 does not comprise a glycine (G) at both amino acid position 67 and 68 (AHo numbering),
   the method comprises the step of introducing a glycine (G) at amino acid position 67 and/or a glycine (G) at amino acid position 68 (AHo numbering) in the light chain complementarity-determining region LCDR2, such that there is a glycine (G) at both amino acid positions 67 and 68,
   wherein said LCDR2 is defined by light chain amino acid positions 58 to 72 according to the AHo numbering scheme, resulting in a maximum length of 15 amino acids.
45. A method for generating a modified antibody variable domain, wherein the method comprises the steps of
   1) providing an unmodified antibody variable domain, which binds to a target antigen, comprising:
      (i) a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region;
      and
      (ii) a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region, wherein the light chain complementarity-determining region LCDR2 does not comprise a glycine (G) at both amino acid position 67 and 68 (AHo numbering);
   2) introducing into the light chain complementarity-determining region LCDR2 of the unmodified antibody variable domain provided in step 1) a glycine (G) at amino acid position 67 and/or a glycine (G) at amino acid position 68 (AHo numbering), such that there is a glycine (G) at both amino acid positions 67 and 68, wherein said LCDR2 is defined by light chain amino acid positions 58 to 72 according to the AHo numbering scheme, resulting in a maximum length of 15 amino acids.
46. The method of item 44 or 45, wherein said modified antibody variable domain comprises a decreased number of 15mer peptide stretches that exhibit a median percentile rank of less than 20, as determined from the predicted individual binding strengths (scoring ranks) of said 15mer peptide to each one of the 27 MHC class II alleles that are most common in the general population using the algorithm NetMHCIIpan-3.1, when compared to the respective unmodified antibody variable domain.
47. The method of any one of items 44 to 46, wherein the LCDR2 of the unmodified antibody variable domain comprises amino acid residues at positions 58, 67, 68, 69, 70, 71 and 72 (AHo numbering).
48. The method of any one of items 44 to 47, wherein the light chain complementarity-determining region LCDR2 of the unmodified antibody variable domain has a sequence selected from X₁GGX₂X₃X₄X₅(SEQ ID NO: 536), wherein X₁, X₂, X₃, X₄ and X₅ correspond to amino acid positions 58, 69, 70, 71 and 72 (AHo numbering), and wherein X₁, X₂, X₃, X₄ and X₅ of LCDR2 are selected from
   (i) the corresponding positions of an LCDR2 region of a mammalian antibody, in particular of an LCDR2 region of a lagomorph, rodent or human antibody, in particular of an LCDR2 region of a rabbit, mouse or human antibody, in particular of an LCDR2 region of a rabbit antibody; or
   (ii) the corresponding positions of an LCDR2 region of an antibody that has been tested in clinical trials; or
   (iii) the corresponding positions of an LCDR2 region of an antibody according to (i) or (ii), wherein one, two or three of said amino acid positions, in particular one or two of said amino acid positions, have been altered.
49. The method of any one of items 44 to 48, wherein the LCDR2 of the unmodified antibody variable domain comprises:
   (i) an alanine (A), threonine (T), serine (S) or glycine (G) at position 67 (AHo numbering);
   (ii) a serine (S), tyrosine (Y) or phenylalanine (F) at position 68 (AHo numbering); and optionally
   (iii) a lysine (K), threonine (T), serine (S), isoleucine (1), aspartate (D), asparagine (N), phenylalanine (F) or tyrosine (Y) at position 69 (AHo numbering);
   (iv) an alanine (A), glycine (G), valine (V), threonine (T), proline (P), tyrosine (Y), aspartate (D) or glutamate (E) at position 71 (AHo numbering).
50. The method of any one of items 44 to 48, wherein the LCDR2 of the unmodified antibody variable domain comprises:
   (i) an alanine (A) or threonine (T) at position 67 (AHo numbering);
   (ii) a serine (S) or tyrosine (Y) at position 68 (AHo numbering);
   and optionally
   (iii) a lysine (K), threonine (T), serine (S), aspartate (D), asparagine (N) or tyrosine (Y) at position 69 (AHo numbering);
   (iv) an alanine (A), glycine (G), threonine (T) or glutamate (E) at position 71 (AHo numbering).
51. The method of any one of items 44 to 48, wherein the LCDR2 of the unmodified antibody variable domain comprises:
   (i) lysine (K), arginine (R), serine (S), threonine (T), alanine (A), leucine (L), glycine (G), glutamine (Q), glutamate (E), tyrosine (Y) or aspartate (D) at position 58 (AHo numbering);
   (ii) an alanine (A), threonine (T), serine (S) or glycine (G) at position 67 (AHo numbering);
   (iii) a serine (S), tyrosine (Y) or phenylalanine (F) at position 68 (AHo numbering); (iv) a lysine (K), threonine (T), serine (S), isoleucine (1), aspartate (D), asparagine (N), phenylalanine (F) or tyrosine (Y) at position 69 (AHo numbering);
   (v) a lysine (K), leucine (L), methionine (M) or arginine (R) at position 70 (AHo numbering);
   (vi) an alanine (A), glycine (G), valine (V), threonine (T), proline (P), tyrosine (Y), aspartate (D) or glutamate (E) at position 71 (AHo numbering);
   (vii) a serine (S), threonine (T) or tyrosine (Y) at position 72 (AHo numbering).
52. The method of any one of items 44 to 48, wherein the LCDR2 of the unmodified antibody variable domain comprises:
   (i) a lysine (K), arginine (R), serine (S), tyrosine (Y), glycine (G) or aspartate (D) at position 58 (AHo numbering);
   (ii) an alanine (A) or threonine (T) at position 67 (AHo numbering);
   (iii) a serine (S) or tyrosine (Y) at position 68 (AHo numbering);
   (iv) a lysine (K), threonine (T), serine (S), aspartate (D), asparagine (N) or tyrosine (Y);
   (v) a leucine (L) at position 70 (AHo numbering);
   (vi) an alanine (A), glycine (G), threonine (T) or glutamate (E) at position 71 (AHo numbering);
   (vii) a serine (S) at position 72 (AHo numbering).
53. The method of any one of the items 44 to 46, wherein the light chain complementarity-determining region LCDR2 of the modified antibody variable domain has a sequence selected from X₁GGX₂X₃X₄X₅(SEQ ID NO: 536), wherein X₁, X₂, X₃, X₄ and X₅ correspond to amino acid positions 58, 69, 70, 71 and 72 (AHo numbering), and wherein said X₁, X₂, X₃, X₄ and X₅ of LCDR2 are selected from:
   (i) the corresponding positions of an LCDR2 region of a mammalian antibody, in particular of an LCDR2 region of a lagomorph, rodent or human antibody, in particular of an LCDR2 region of a rabbit, mouse or human antibody, in particular of an LCDR2 region of a rabbit antibody; or
   (ii) the corresponding positions of an LCDR2 region of an antibody that has been tested in clinical trials; or
   (iii) the corresponding positions of an LCDR2 region of an antibody according to (i) or (ii), wherein one, two or three of said amino acid positions, in particular one or two of said amino acid positions, have been altered.
54. The method of item 53, wherein the other light chain complementarity-determining regions LCDR1 and LCDR3 and the heavy chain complementarity-determining regions HCDR1, HCDR2 and HCDR3 are selected from
   (i) complementarity-determining regions of a mammalian antibody, in particular from complementarity-determining regions of a lagomorph, rodent or human antibody, in particular from complementarity-determining regions of a rabbit, mouse or human antibody, in particular from complementarity-determining regions of a rabbit antibody; or
   (ii) complementarity-determining regions of an antibody that has been tested in clinical trials; or
   (iii) complementarity-determining regions of an antibody according to (i) or (ii), wherein at least in one of said complementarity-determining regions, independently from each other, one, two or three amino acid positions, in particular one or two amino acid positions, have been altered.
55. The method of any one of items 44 to 54, wherein the heavy chain framework regions HFW1, HFW2, HFW3 and HFW4 and/or the light chain framework regions LFW1, LFW2, LFW3 and LFW4 are as defined in any one of the items 3 and 10 to 16.
56. The method of any one of items 44 to 55, wherein said modified and unmodified antibody variable domain is comprised in an antibody fragment selected from a Fab, an scFab, an Fv, a dsFv, an scFv and a dsscFv particularly from a Fab, Fv and scFv.
57. The method of any one of items 46 to 54, wherein in said modified antibody variable domain, the number of 15mer peptide stretches that exhibit a median percentile rank of less than 20, as determined from the predicted individual binding strengths (scoring ranks) of said 15mer peptide to each one of the 27 MHC class II alleles that are most common in the general population using the algorithm NetMHCIIpan-3.1, is decreased by at least 50%, particularly by at least 60%, particularly by at least 70%, particularly by at least 80%, particularly by at least 90%, when compared to the respective unmodified antibody variable domain.
58. The method of any one of items 44 to 57, wherein the method comprises that additional step of
   3) introducing into the variable heavy chain (VH) of the unmodified antibody variable domain provided in step 1) a serine (S) at amino acid position 101 and a lysine (K) at amino acid position 146 (AHo numbering).
59. A method for generating a modified antibody from an unmodified antibody, the method comprises the steps of
   1) selecting one or more antibody variable domains comprised in said unmodified antibody, which do not comprise a glycine (G) at both amino acid position 67 and 68 (AHo numbering) in the LCDR2, for modification;
   2) introducing into each of said one or more antibody variable domains selected in step 1) a glycine (G) at amino acid position 67 and/or a glycine (G) at amino acid position 68 (AHo numbering) in the LCDR2, such that there is a glycine (G) at both amino acid positions 67 and 68 (AHo numbering) in the LCDR2,
   wherein said LCDR2 is defined by light chain amino acid positions 58 to 72 according to the AHo numbering scheme, resulting in a maximum length of 15 amino acids.
60. The method of item 59, wherein the modified antibody comprises a decreased number of 15mer peptide stretches that exhibit a median percentile rank of less than 20, as determined from the predicted individual binding strengths (scoring ranks) of said 15mer peptide to each one of the 27 MHC class II alleles that are most common in the general population using the algorithm NetMHCIIpan-3.1, when compared to the respective unmodified antibody.
61. The method of item 59 or 60, wherein the method comprises that additional step of
   3) introducing into the variable heavy chain (VH) of each of said one or more antibody variable domains selected in step 1) a serine (S) at amino acid position 101 and a lysine (K) at amino acid position 146 (AHo numbering).
62. The method of any one of items 43 to 61, wherein said modified antibody variable domain or said modified antibody additionally has one or more of the features as defined in any one of items 1 to 26.

### BRIEF DESCRIPTION OF THE DRAWINGS

FIG. 1 shows a median percentile rank plot of the VL region of the anti-PDL1 scFv PRO2230, calculated with NetMHCIIpan-3.1. The dashed line indicates the median percentile rank 20. A median percentile rank of less than 20 is deemed to indicate significant binding to MHC class II proteins.

FIG. 2 shows median percentile rank plots of the VL regions of the multispecific anti-PDL1×CD137×hSA antibody PRO1480 (A) and of a GG variant of PRO1480 (B), calculated with NetMHCIIpan-3.1. The plots for VL regions of the PDL1, CD137 (4-1BB) and hSA binding domains are superimposed. The dashed line indicates the median percentile rank 20. A median percentile rank of less than 20 is deemed to indicate significant binding to MHC class II proteins.

FIG. 3 shows median percentile rank plots of the VL regions of 206 therapeutic antibodies (A) and of their corresponding GG variants (B), calculated with NetMHCIIpan-3.1. All VL region plots are superimposed. The dashed line indicates the median percentile rank 20. A median percentile rank of less than 20 is deemed to indicate significant binding to MHC class II proteins.

FIG. 4 shows the absorption levels of pre-existing and treatment-emerging ADAs against PRO1480 in five patients at various time points, determined with the ELISA-based pre-existing ADA-binding assay described in Example 3. Absorption levels were measured at 450 nm and are given in A.U.

FIG. 5 shows the inhibition of the absorption signal after spiking each serum with the respective molecule (MATCH-3 molecules PRO1480, PRO4180, PRO4181: 150 nM spiking; scFv PRO2230: 450 nM spiking), determined with the ELISA-based ADA-binding assay described in Example 3. The reduction in the signal for the spiked sera vs. non-spiked sera has to be greater than 30% (>30% inhibition) in order to be evaluated as specific.

### DETAILED DESCRIPTION OF THE INVENTION

The inventors have surprisingly found that antibody variable domains, which comprise a light chain complementarity-determining region 2 (LCDR2) that has a glycine (G) at amino acid position 67 and a glycine (G) at amino acid position 68 (AHo numbering), hereinafter also referred to as “GG variants” or “GG motif”, exhibit a significantly lower predicted binding to (human) MHC class II proteins, when compared to their original versions that do not comprise said GG motif. This advantageous property could be observed for all tested antibody variable domains comprising said GG motif, where their non-GG variants have been predicted to bind to (human) MHC class II proteins, irrespective of their antigen target specificity. It was further found that the stability of said GG variants as well as their functional properties, in particular their inhibition potency and/or their binding affinity to their respective antigen targets, are not or not significantly reduced, when compared to their unmodified versions. The inventors could further verify these findings by testing a subset of fragment based multispecific antibodies comprising at least one modified binding domain of the present invention against patient derived serum samples. These fragment-based multispecific antibodies exhibited a clear reduction in the binding to pre-existing ADAs and/or to treatment emerged ADAs, when compared to their unmodified versions.

Despite that fact that methods and strategies have been developed to reduce the immunogenicity of a given antibody variable domain, these methods are highly case dependent, i. e. their proposed solutions for reducing the immunogenicity of a given antibody variable domain are not generally applicable to other antibody variable domains. Thus, there is still a large unmet need for antibody variable domains, which exhibit low immunogenicity. More specifically, it would be desirable to have antibody variable domains at hand, which exhibiting low immunogenicity, in particular with regard to reduced binding to MHC class II proteins, and which can generally be applied in the construction of antibody fragments. It is furthermore desirable that these antibody variable domains provide a high stability, when integrated in the final antibody format, which would allow their application in the construction of stable antibody fragments and fragment-based multispecific antibodies suitable for therapeutic development.

The antibody variable domain of the present invention could be successfully used for the construction of several highly stable and functional antibody binding domains, such as Fab fragments and scFvs, that can readily be used in the construction of multispecific antibodies.

Besides, the universal applicability of this modification was tested with a set of 206 sequences of antibodies that have been clinically tested, i. e. clinically approved and clinical phase 1-3 antibody therapeutics. These sequences have been published in Marks et al. (Marks et al., Bioinformatics, Volume 37, Issue 22, November 2021, pp. 4041-4047), together with the corresponding percentage of anti-drug antibody (ADA) responses in patients for each of said therapeutics, obtained from clinical papers (referred in Marks et al. as immunogenicity levels). Most of these antibody sequences had no glycine at both light chain amino acid positions 67 and 68 (AHo numbering) and just a few of these antibody sequences had one glycine either at position 67 or 68 (AHo numbering). None of them had glycine residues at both positions. 86% of the corresponding GG variants of these 206 antibody sequences no longer exhibited predicted binding to MHC class II proteins and the other GG variants exhibited a significant reduction in the predicted binding to MHC class II proteins, except for one.

Unless defined otherwise, all technical and scientific terms used herein have the same meaning as commonly understood by those of ordinary skill in the art to which this invention pertains.

The terms “comprising” and “including” are used herein in their open-ended and non-limiting sense unless otherwise noted. With respect to such latter embodiments, the term “comprising” thus includes the narrower term “consisting of”.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, and the like, this is taken to mean also a single compound, salt, or the like.

In one aspect, the present invention relates to an antibody variable domain, which binds to a target antigen, comprising:
(i) a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region;
and
(ii) a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region;
wherein said LCDR2 is defined by light chain amino acid positions 58 to 72 according to the AHo numbering scheme, resulting in a maximum length of 15 amino acids, and wherein said LCDR2 has a glycine (G) at amino acid position 67 and a glycine (G) at amino acid position 68 (AHo numbering);
and wherein said light chain complementarity-determining region LCDR2, i. e. amino acid positions 58, 67, 68, 69, 70, 71, 72 (AHo numbering), does not have a sequence selected from LGGNRAA (SEQ ID NO: 665) and RGGERVS (SEQ ID NO: 666).

The term “antibody” and the like, as used herein, includes whole antibodies or single chains thereof; and any antigen-binding variable domain (i. e. “antigen-binding portion”) or single chains thereof; and molecules comprising antibody CDRs, VH regions or VL regions (including without limitation multispecific antibodies). A naturally occurring “whole antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), flanked by regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e. g., effector cells) and the first component (Clq) of the classical complement system.

The term “antibody variable domain”, as used herein, refers to one or more parts of an intact antibody that have the ability to specifically bind to a given antigen (e. g., PD-L1, CD137, ROR1, MSLN, CD3, hSA, IL-4R, IL-31, IL-23R or Her2). This can be any antigen-binding fragment (i. e. “antigen-binding portion”) of an intact antibody or single chains thereof; and molecules comprising antibody CDRs, VH regions or VL regions. Specifically, in case of the multispecific antibodies of the present invention, the term “antibody variable domain”, as used herein, refers in particular to a Fab, a single chain Fab (scFab), an Fv fragment consisting of the VL and VH domains of a single arm of an antibody (Fv); a disulfide stabilized Fv fragment (dsFv); a single chain Fv fragment (scFv); and a single chain Fv fragment having an additional light chain constant domain (C_{L}) fused to it (scAB). Preferably, the antibody variable domain of the present invention is selected from a Fab, an Fv fragment, a disulfide stabilized Fv fragment (dsFv) and an scFv fragment. In particular embodiments, the antibody variable domain of the present invention is a Fab. In other particular embodiments, the antibody variable domain of the present invention is a single-chain Fv fragment (scFv). In other particular embodiments, the VL and VH domains of the scFv fragment are stabilized by an interdomain disulfide bond, in particular said VH domain comprises a single cysteine residue in position 51 (AHo numbering) and said VL domain comprises a single cysteine residue in position 141 (AHo numbering).

The term “Complementarity Determining Regions” (“CDRs”) refers to amino acid sequences with boundaries determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); ImMunoGeneTics (IMGT) numbering (Lefranc, M.-P., The Immunologist, 7, 132-136 (1999); Lefranc, M.-P. et al., Dev. Comp. Immunol., 27, 55-77 (2003)) (“IMGT” numbering scheme); and the numbering scheme described in Honegger & Plückthun, J. Mol. Biol. 309 (2001) 657-670 (“AHo” numbering). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 24-34 (LCDR1), 50-56 (LCDR2), and 89-97 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to “Kabat”). Under IMGT, the CDRs of an antibody can be determined using the program IMGT/DomainGap Align.

In the context of the present invention, the numbering system suggested by Honegger & Plückthun (“AHo”) is used (Honegger & Plückthun, J. Mol. Biol. 309 (2001) 657-670), unless specifically mentioned otherwise. In particular, the following residues are defined as CDRs according to AHo numbering scheme: LCDR1 (also referred to as CDR-L1): L24-L42; LCDR2 (also referred to as CDR-L2): L58-L72; LCDR3 (also referred to as CDR-L3): L107-L138; HCDR1 (also referred to as CDR-H1): H27-H42; HCDR2 (also referred to as CDR-H2): H57-H76; HCDR3 (also referred to as CDR-H3): H108-H138. Correspondingly, in the context of the present invention, the following residues are defined as light chain frameworks 1 to 4 (LFW1-LFW4) and heavy chain frameworks 1 to 4 (HFW1-HFW4), respectively, according to AHo numbering scheme: LFW1: L1-L23; LFW2: L43-L57; LFW3: L73-L106; LFW4: L139-L149; HFW1: H1-H26; HFW2: H43-H56; HFW3: H77-H107; HFW4: H139-H149. For the sake of clarity, the numbering system according to Honegger & Plückthun takes the length diversity into account that is found in naturally occurring antibodies, both in the different VH and VL subfamilies and, in particular, in the CDRs, and provides for gaps in the sequences. Thus, in a given antibody variable domain usually not all positions 1 to 149 will be occupied by an amino acid residue.

In order to avoid any unclarity, it is stressed here that as a result from the above definition, said framework (FW) regions and said CDR regions comprised in the antibody fragments of the present invention have maximum length corresponding to the above residue numbering under the Aho numbering system. For example, in the case of the LCDR2, the maximum sequence length is limited to 15 amino acids (resulting from the Aho numbering L58-L72). Consequently, artificial antibody variable domains that have a longer amino acid stretches in one or more of said FW regions and/or said CDR regions, e.g. having an LCDR2 that is larger than 15 amino acids, are not considered as antibody variable domains according to the present invention.

In particular embodiments, the antibody variable domains of the present invention have an LCDR2, which has a length of at maximum 14, particularly of at maximum 12, particularly of at maximum 10, particularly of at maximum 9, particularly of at maximum 8, particularly of at maximum 7 amino acids.

In particular embodiments, the antibody variable domains of the present invention have an LCDR2 that consists of seven amino acids.

The term “binds to” as used herein refers to the ability of an individual antibody to react with an antigenic determinant. However, this does not exclude that said individual antibody can for example also react with homologues of said antigenic determinant (e. g. with antigen determinants from other species) or with other antigen determinants belonging to the same protein family.

The term “binding specificity” as used herein refers to the ability of an individual antibody to react with one antigenic determinant and not with a different antigenic determinant. As used herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. However, also these terms do not exclude that said individual antibody can bind with comparable affinity to the same antigen determinants from different species. In its most general form (and when no defined reference is mentioned), “specific binding” is referring to the ability of the antibody to discriminate between the target of interest and an unrelated molecule, as determined, for example, in accordance with specificity assay methods known in the art. Such methods comprise, but are not limited to Western blots, ELISA, RIA, ECL, IRMA, SPR (Surface plasmon resonance) tests and peptide scans. For example, a standard ELISA assay can be carried out. The scoring may be carried out by standard color development (e. g. secondary antibody with horseradish peroxide and tetramethyl benzidine with hydrogen peroxide). The reaction in certain wells is scored by the optical density, for example, at 450 nm. Typical background (=negative reaction) may be about 0.1 OD; typical positive reaction may be about 1 OD. This means the ratio between a positive and a negative score can be 10-fold or higher. In a further example, an SPR assay can be carried out, wherein at least 10-fold, particularly at least 100-fold difference between a background and signal indicates on specific binding. Typically, determination of binding specificity is performed by using not a single reference molecule, but a set of about three to five unrelated molecules, such as milk powder, transferrin or the like.

The antibody variable domains of the present invention bind to a target antigen, which can be any target antigen. Examples of target antigens include, but are not limited to: a transmembrane molecule; a receptor; a ligand; a growth factor; a growth hormone; a clotting factor; an anti-clotting factor; a plasminogen activator; a serum albumin; a receptor for a hormone or a growth factor; a neurotrophic factor; a nerve growth factor; a fibroblast growth factor; a CD protein; an interferon; a colony stimulating factor (CSF); an interleukin (IL); a T-cell receptor; a T-cell co-stimulatory receptor, such as CD137; a surface membrane protein; a viral protein; a tumor associated antigen; an integrin or an interleukin; VEGF; a renin; a human growth hormone; a bovine growth hormone; a growth hormone releasing factor; parathyroid hormone; thyroid stimulating hormone; a lipoprotein; alpha-1-antitrypsin; insulin A-chain; insulin B-chain; proinsulin; follicle stimulating hormone; calcitonin; luteinizing hormone; glucagon; clotting factor VIIIC; clotting factor IX; tissue factor (TP); von Willebrand's factor; Protein C; atrial natriuretic factor; a lung surfactant; urokinase; human urine; tissue-type plasminogen activator (t-PA); bombesin; thrombin; hemopoietic growth factor; tumor necrosis factor-alpha or -beta; enkephalinase; RANTES (Regulated on Activation Normally T-cell Expressed and Secreted); human macrophage inflammatory protein (MIP-I)-alpha; Muellerian-inhibiting substance; relaxin A-chain; relaxin B-chain; prorelaxin; mouse gonadotropin-associated peptide; a microbial protein, beta-lactamase; DNase; IgE; a cytotoxic T-lymphocyte associated antigen (CTLA); CTLA-4; inhibin; activin; vascular endothelial growth factor (VEGF); protein A or D; a rheumatoid factor; bone-derived neurotrophic factor (EDNF); neurotrophin-3, -4, -5, or -6 (NT-3, NT-4, NT-5, or NT-6); NGF-beta; platelet-derived growth factor (PDGF); aFGF; bFGF; epidermal growth factor (EGF); TGF-alpha; TGF-beta, including TGF-beta1, TGF-beta2, TGF-beta3, TGF-beta14, or TGF-beta5; insulin-like growth factor-I or -II (IGF-I or IGF-II); des(I-3)-IGF-I (brain IGF-1), an insulin-like growth factor binding protein, erythropoietin; an osteoinductive factor; an immunotoxin; a bone morphogenetic protein (EMP); interferon-alpha, -beta, or -gamma; M-CSF, GM-CSF or G-CSF; IL-1 to IL-10; superoxide dismutase; decay accelerating factor; an AIDS envelope protein; a transport protein; a homing receptor; an addressin; a regulatory protein; CD3, CD4, CD8, CD11a, CD11b, CD11c, CD18, CD19, CD20, CD34, CD40, or CD46, an ICAM, VLA-4 or VCAM; or HER2, HER3 or HER4 receptor; a member of the ErbB receptor family; an EGF receptor; HER2, HER3 or HER4 receptor; a cell adhesion molecule; LFA-1, Mac1, p150.95, VLA-4, ICAM-1, VCAM, alpha4/beta7 integrin or alphav/beta3 integrin; an alpha or beta subunit of a cell adhesion molecule; antibodies); a growth factor, VEGF; tissue factor (TF); TGF-beta; alpha interferon (alpha-IFN); IL-8; IgE; blood group antigens Apo2; death receptor, such as PD-1; death receptor ligands, such as PD-L1; flk2/flt3 receptor; obesity (CE) receptor; mpl receptor; CTLA4 or protein C.

The term “tumor-associated antigen (TAA)” refers to an antigen that is expressed on the surface of a tumor cell. In particular embodiments, a TAA is an antigen that is preferentially expressed on a tumor cell when compared to non-tumor cells, particularly wherein expression of the TAA on a tumor cell is at least more than 5-fold, at least more than 10-fold, at least more than 20-fold, at least more than 50-fold, or at least more than 100-fold higher than on non-tumor cells from the same organism or patient.

Examples of tumor associated antigen targets include, but are not limited to: ADRB3, AFP, ALK, BCMA, beta human chorionic gonadotropin, CA-125 (MUC16), CAIX, CD123, CD133, CD135, CD135 (FLT3), CD138, CD171, CD19, CD20, CD22, CD24, CD276, CD33, CD33, CD38, CD44v6, CD79b, CD97, CDH3 (cadherin 3), CEA, CEACAM6, CLDN6, CLEC12A (CLL1), CSPG4, CYP1B1, EGFR, EGFRvIII, EpCAM, EPHA2, Ephrin B2, ERBBs (e. g. ERBB2), FAP, FGFR1, folate receptor alpha, folate receptor beta, Fos-related antigen, GA733, GD2, GD3, GFRalpha4, globoH, GPC3, GPR20, GPRC5D, HAVCR1, Her2/neu (HER2), HLA-A2, HMWMAA, HPV E6 or E7, human telomerase reverse transcriptase, IL-11Ra, IL-13Ra2, intestinal carboxyl esterase, KIT, Legumain, LewisY, LMP2, Ly6k, MAD-CT-1, MAD-CT-2, ML-IAP, MN-CA IX, MSLN, MUC1, mut hsp 70-2, NA-17, NCAM, neutrophil elastase, NY-BR-1, NY-ESO-1, o-acetyl-GD2, OR51E2, PANX3, PDGFR-beta, PLAC1, Polysialic acid, PSCA, PSMA, RAGE1, ROR1, sLe, sperm protein 17, SSEA-4, SSTR2, sTn antigen, sTn-OGlycopeptides, TAG72, TARP, TEM1/CD248, TEM7R, thyroglobulin, Tn antigen, Tn-O-Glycopeptides, TPBG (5T4), TRP-2, TSHR, UPK2 and VEGFR2. Preferred examples are: CD138, CD79b, TPBG (5T4), HER2, MSLN, MUC1, CA-125 (MUC16), PSMA, BCMA, CD19, EpCAM, CLEC12A (CLL1), CD20, CD22, CEA, CD33, EGFR, GPC3, CD123, CD38, CD33, CD276, CDH3 (cadherin 3), FGFR1, SSTR2, CD133, EPHA2, HLA-A2, IL13RA2, ROR1, CEACAM6, CD135, GD-2, GA733, CD135 (FLT3), CSPG4 and TAG-72. Particular examples are: CD138, CD79b, CD123, MSLN, PSMA, BCMA, CD19, CD20, CEA, CD38, CD33, CLEC12a, and ROR1.

Suitably, the framework regions HFW1, HFW2, HFW3 and HFW4 comprised in the antibody variable domain of the invention are selected from a human VH framework. In particular, the framework regions HFW1, HFW2, HFW3 and HFW4 comprised in the antibody variable domain of the invention are selected from the human VH framework subtypes VH1a, VH1b, VH3, VH4, VH5 or VH6, particularly from the human VH framework subtypes VH1a, VH1b, VH3 or VH4. In particular embodiments, the framework regions HFW1, HFW2, HFW3 and HFW4 are selected from the human VH framework subtype VH3. HFW4 may be selected from a human germline sequence or from the HFW4 sequence of a rearranged human antibody sequence.

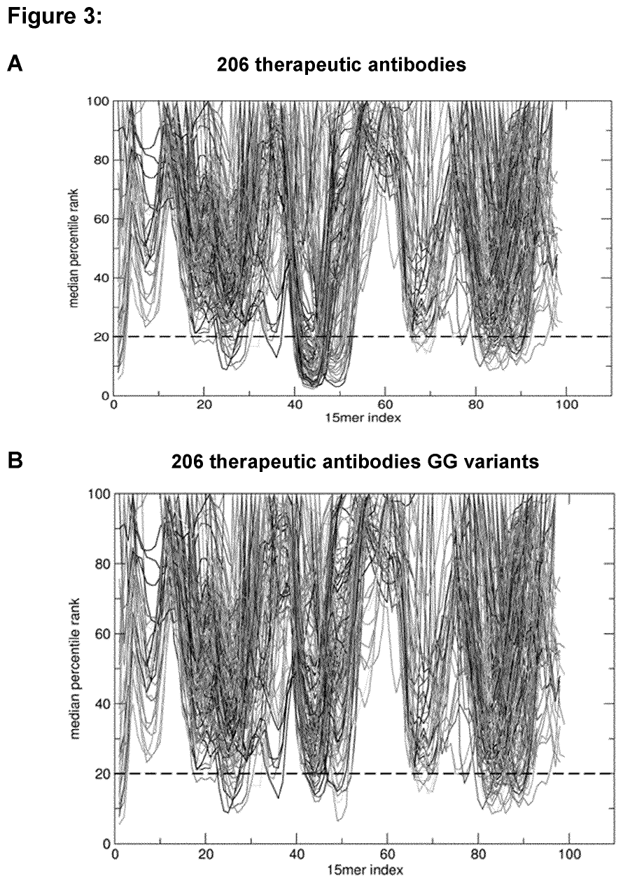

In the context of the present invention, the terms “belonging to a human VHx framework subtype (or a human antibody Vκ/Vλ framework)”, “selected from a human VHx framework subtype (or a human antibody Vκ/Vλ framework)” or “are of the human VHx framework subtype” mean that the framework sequences HFW1 to HFW3 (or LF1 to LFW3) show the highest degree of homology to the consensus sequence of said human antibody VH or VL framework subtype, as determined in Knappik et al., J. Mol. Biol. 296 (2000) 57-86 or in WO 2019/057787. In the context of the present invention, the sequences of human VH domains are grouped into seven distinct framework subtypes, i. e. the framework subtypes VH1a, VH1b, VH2, VH3, VH4, VH5 and VH6, herein also referred to as human subfamilies VH1a, VH1b, VH2, VH3, VH4, VH5 and VH6, based on sequence homology to the sequences as shown in FIG. 3 of Knappik et al., J. Mol. Biol. 296 (2000) 57-86 or in WO 2019/057787. Specific example of VH domains belonging to the VH3 framework subtype are represented by SEQ ID NOs: 496 to 509. Specific examples of a VH domain belonging to the VH1a, VH1b, VH4, VH5 and VH6 framework subtypes are represented by SEQ ID NOs: 510-514. Alternative examples of VH1a, VH1b, VH3 and VH4 sequences, and examples of other VHx sequences, may be found in Knappik et al., J. Mol. Biol. 296 (2000) 57-86 or in WO 2019/057787.

It is common practice in the generation of antibody variable domains from non-human sources that in the humanization process, also certain specific framework amino acids of the non-human source antibody are introduced into the human framework regions used for humanization, in order to maintain the binding properties of the resulting humanized antibody variable domains. The introduction of said source framework residues into the human framework regions is often referred to as “back mutations”. The positions of said specific framework amino-acids or “back mutations” are well known to those skilled in the art.

In cases where the antibody variable domains are obtained from human sources, such back mutations are generally not needed. Besides, the framework regions can comprise further substitutions as defined in the below description of the terms “humanized” antibody or “humanized” antibody variable domain. Apart from that, it is also common practice for the generation of antibody variable domains to use variable domains sequences of known antibodies that have been tested in clinical trials. This also includes antibodies that are currently in clinical trials.

Thus, in particular embodiments, the variable heavy chain framework regions HFW1, HFW2, HFW3 and HFW4 of the antibody variable domain of the present invention are selected from
(i) human antibody framework regions; and
(ii) human antibody framework regions comprising a total of 1 to 20, particularly 1 to 15, particularly 1 to 10, amino acid positions where the amino acids are taken from a non-human antibody, in particular from a lagomorph or rodent antibody, in particular from a rabbit or mouse antibody, in particular from a rabbit antibody (back mutations), said non-human antibody representing the source of the CDR sequences comprised in the antibody variable domain.

In other particular embodiments, the variable heavy chain framework regions HFW1, HFW2, HFW3 and HFW4 of the antibody variable domain of the present invention are selected from framework regions of antibodies that have been tested in clinical trials. As indicated above, the expression “antibodies that have been tested in clinical trials” refers to antibodies that have already been tested in clinical trials as well as to antibodies that are currently in clinical trials. This applies to antibodies that have successfully passed clinical testing as well as to antibodies that failed in clinical testing.

In particular embodiments, the variable heavy chain framework regions HFW1, HFW2, HFW3 and HFW4 of the antibody variable domain of the present invention are selected from the combination of framework regions (i. e. the residues corresponding to the non-italicized residues of SEQ ID NO: 1 or of the VH sequences shown in Table 2, i. e. all residues that are not marked as CDR residues) of any one of the SEQ ID NOs: 302, 306, 496, 497, 498, 499, 500, 501, 502, 503, 504, 505, 506, 507, 508, 509, 510, 511, 512, 513 and 514; and from the combination of framework regions (i. e. the residues corresponding to the non-italicized residues of SEQ ID NO: 1 or of the VH sequences shown in Table 2) of any one of the SEQ ID NOs: 302, 306, 496, 497, 498, 499, 500, 501, 502, 503, 504, 505, 506, 507, 508, 509, 510, 511, 512, 513 and 514, wherein no more than 5 amino acids, particularly no more than 4 amino acids, particularly no more than 3 amino acids, particularly no more than 2 amino acids, particularly no more than 1 amino acid within the framework regions have been mutated. In this connection, the term “mutation” means, as various non-limiting examples, an addition, substitution or deletion. The VH regions further include VH domains comprising at least positions 5 to 140 (AHo numbering), particularly at least positions 3 to 145, more particularly at least positions 2 to 147 of one of the sequences shown in the SEQ ID NOs: 302, 306, 496, 497, 498, 499, 500, 501, 502, 503, 504, 505, 506, 507, 508, 509, 510, 511, 512, 513 and 514, provided that such VH domains exhibit the functional features defined above in item 25.

In particular embodiments, the variable light chain frameworks LFW1, LFW2, LFW3 and LFW4 of the antibody variable domain of the present invention are selected from a human antibody Vκ framework (e. g. a Vκ1, Vκ2, Vκ3 or Vκ4 framework subtype) or a human antibody Vλ framework (e. g. a Vλ1, Vλ2 or Vλ3 framework subtype), in particular a human antibody Vκ framework. In the context of the present invention, the sequences of human VL domains are grouped into four distinct human Vκ framework subtypes, i. e. the framework subtypes Vκ1, Vκ2, Vκ3 and Vκ4, and three distinct human Vλ framework subtypes, i. e. the framework subtypes Vλ1, Vλ2 and Vλ3, herein also referred to as human Vκ subfamilies Vκ1, Vκ2, Vκ3, and Vκ4, and human Vλ subfamilies Vλ1, Vλ2 and Vλ3, based on sequence homology to the sequences as shown in FIG. 3 of Knappik et al., J. Mol. Biol. 296 (2000) 57-86 or in WO 2019/057787. In particular embodiments, the variable light chain frameworks LFW1, LFW2, LFW3 and LFW4 of the antibody variable domain of the present invention are of the Vκ1 framework subtype. Specific examples of Vκ1 framework subtypes are represented by SEQ ID NOs: 515, 516, 517 and 518. Alternative examples of Vκ1 sequences, and examples of Vκ2, Vκ3 or Vκ4 sequences, may be found in Knappik et al., J. Mol. Biol. 296 (2000) 57-86.

In other particular embodiments, the variable light chain frameworks LFW1, LFW2 and LFW3 are selected from a human antibody Vκ framework, preferably a Vκ1 framework subtype, and the variable light chain framework LFW4 is selected from a Vλ framework. In particular embodiments, the variable light chain framework LFW4 of the antibody variable domain of the present invention is selected from the group consisting of the Vλ framework 4 sequences of SEQ ID NOs: 527, 528, 529, 530, 531, 532, 533, 534 and 535. Vλ framework 4 sequence of SEQ ID NO: 534 comprises a single cysteine residue at the variable light (VL) chain position 144 (AHo numbering) and is in particular applied in cases where a second single cysteine is present in the corresponding variable heavy (VH) chain, particularly in position 51 (AHo numbering) of VH, for the formation of an inter-domain disulfide bond.

In other particular embodiments, the LFW1, LFW2 and LFW3, and optionally also LFW4, if LFW4 is selected from a human antibody Vκ framework subtype, are selected from the combination of framework regions LFW1, LFW2, LFW3 and optionally LFW4 (i. e. the residues corresponding to the non-italicized residues of SEQ ID NO: 3 or of the VL sequences shown in Table 2) of any one of the SEQ ID NOs: 515-526; and the combination of framework regions LFW1, LFW2, LFW3 and optionally LFW4 (i. e. the residues corresponding to the non-italicized residues of SEQ ID NO: 3 or of the VL sequences shown in Table 2) of any one of the SEQ ID NOs: 515-526, wherein no more than 5 amino acids, particularly no more than 4 amino acids, particularly no more than 3 amino acids, particularly no more than 2 amino acids, particularly no more than 1 amino acid within the framework regions have been mutated.

In particular embodiments, the variable light chain framework regions LFW1, LFW2, LFW3 and LFW4 of the antibody variable domain of the present invention independently from each other, are selected from
(i) human antibody framework regions; and
(ii) human antibody framework regions comprising a total of 1 to 20, particularly 1 to 15, particularly 1 to 10, amino acid positions where the amino acids are taken from a non-human antibody, in particular from a lagomorph or rodent antibody, in particular from a rabbit or mouse antibody, in particular from a rabbit antibody (back mutations), said non-human antibody representing the source of the CDR sequences comprised in the antibody variable domain.

In other particular embodiments, the variable light chain framework regions LFW1, LFW2, LFW3 and LFW4 of the antibody variable domain of the present invention are selected from framework regions of antibodies that have been tested in clinical trials.

In yet other particular embodiments, the variable light chain frameworks LFW1, LFW2, LFW3 and LFW4 of the antibody variable domain of the present invention are selected from the combination of framework regions LFW1, LFW2, LFW3 and LFW4 (i. e. the residues corresponding to the non-italicized residues of SEQ ID NO: 3 or of the VL sequences shown in Table 2) of any one of the SEQ ID NOs: 304, 309, 515, 516, 517, 518, 519, 520, 521, 522, 523, 524, 525 and 526; and the combination of framework regions LFW1, LFW2, LFW3 and LFW4 (i. e. the residues corresponding to the non-italicized residues of SEQ ID NO: 3 or of the VL sequences shown in Table 2) of any one of the SEQ ID NOs: 304, 309, 515, 516, 517, 518, 519, 520, 521, 522, 523, 524, 525 and 526, wherein no more than 5 amino acids, particularly no more than 4 amino acids, particularly no more than 3 amino acids, particularly no more than 2 amino acids, particularly no more than 1 amino acid within the framework regions have been mutated. In this connection, the term “mutation” means, as various non-limiting examples, an addition, substitution or deletion. The VL regions further include VL domains comprising at least positions 5 to 140 (AHo numbering), particularly at least positions 3 to 145, more particularly at least positions 2 to 147 of one of the sequences shown in the SEQ ID NOs: 304, 309, 515, 516, 517, 518, 519, 520, 521, 522, 523, 524, 525 and 526, provided that such VL domains exhibit the functional features defined above in item 25.

In preferred embodiments, the antibody variable domain of the present invention is in a format selected from a Fab, an Fv fragment consisting of the VL and VH domains of a single arm of an antibody; a disulfide stabilized Fv fragment (dsFv); and a single chain Fv fragment (scFv). In particular embodiments, the antibody variable domain of the present invention is selected from a Fab, an Fv fragment and a single-chain Fv fragment (scFv). In other particular embodiments, the VL and VH domains of the scFv fragment are stabilized by an interdomain disulfide bond, in particular said VH domain comprises a single cysteine residue in position 51 (AHo numbering) and said VL domain comprises a single cysteine residue in position 141 (AHo numbering).

In particular embodiments, the light chain complementarity-determining region LCDR2 has a sequence selected from X₁GGX₂X₃X₄X₅(SEQ ID NO: 536), wherein X₁, X₂, X₃, X₄ and X₅ correspond to amino acid positions 58, 69, 70, 71 and 72 (AHo numbering), and wherein said X₁, X₂, X₃, X₄ and X₅ of LCDR2 are selected from:
(i) the corresponding positions of an LCDR2 region of a mammalian antibody, in particular of an LCDR2 region of a lagomorph, rodent or human antibody, in particular of an LCDR2 region of a rabbit, mouse or human antibody, in particular of an LCDR2 region of a rabbit antibody; or
(ii) the corresponding positions of an LCDR2 region of an antibody that has been tested in clinical trials; or
(iii) the corresponding positions of an LCDR2 region of an antibody according to (i) or (ii), wherein one, two or three of said amino acid positions, in particular one or two of said amino acid positions, have been altered.

In further particular embodiments also the other light chain complementarity-determining regions LCDR1 and LCDR3 and, optionally, also the heavy chain complementarity-determining regions HCDR1, HCDR2 and HCDR3 are selected from the antibodies defined above for the LCDR2. In further particular embodiments, the LCDR2 has a glycine (G) at amino acid position 67, a glycine (G) at amino acid position 68, and a leucine (L) or an arginine (R) at amino acid position 70 (AHo numbering).

In further particular embodiments, the LCDR2 has a sequence selected from X₁GGX₂X₃X₄X₅(SEQ ID NO: 536), wherein
X₁ is selected from lysine (K), arginine (R), serine (S), threonine (T), alanine (A), leucine (L), glycine (G), glutamine (Q), glutamate (E), tyrosine (Y) and aspartate (D); in particular from lysine (K), arginine (R), serine (S), tyrosine (Y), glycine (G), and aspartate (D);
X₂ is selected from lysine (K), threonine (T), serine (S), isoleucine (I), aspartate (D), asparagine (N), phenylalanine (F) and tyrosine (Y); in particular from lysine (K), threonine (T), serine (S), aspartate (D), asparagine (N) and tyrosine (Y);
X₃ is selected from lysine (K), leucine (L), methionine (M) and arginine (R); in particular from lysine (K), methionine (M) and leucine (L); in particular is leucine (L);
X₄ is selected from alanine (A), glycine (G), valine (V), threonine (T), proline (P), tyrosine (Y), aspartate (D), and glutamate (E); in particular from alanine (A), glycine (G), threonine (T) and glutamate (E);
X₅ is selected from serine (S), threonine (T) and tyrosine (Y); in particular is serine (S).

Non limiting examples of LCDR2 sequences are RGGILAS (SEQ ID NO: 667), KGGTLAS (SEQ ID NO: 668), RGGTLAS (SEQ ID NO: 669), SGGTLAS (SEQ ID NO: 670), LGGTLAS (SEQ ID NO: 671), TGGTLAS (SEQ ID NO: 672), GGGTLAS (SEQ ID NO: 673), RGGNLAS (SEQ ID NO: 674), DGGDLAS (SEQ ID NO: 675), DGGKLAS (SEQ ID NO: 676), QGGKLAS (SEQ ID NO: 677), RGGKLAS (SEQ ID NO: 678), LGGKLAS (SEQ ID NO: 679), SGGKLAS (SEQ ID NO: 680), GGGKLAS (SEQ ID NO: 681), DGGRLAS (SEQ ID NO: 682), DGGNRAT (SEQ ID NO: 683), RGGTLES (SEQ ID NO: 684), KGGTLES (SEQ ID NO: 685), DGGDLTS (SEQ ID NO: 686), SGGFLYS (SEQ ID NO: 687), SGGYRYT (SEQ ID NO: 688), SGGKLAA (SEQ ID NO: 689), SGGTLVS (SEQ ID NO: 690), RGGTLAY (SEQ ID NO: 691), AGGTLAS (SEQ ID NO: 692), AGGYLAS (SEQ ID NO: 693), RGGYLES (SEQ ID NO: 694), EGGKLAS (SEQ ID NO: 695), RGGNLES (SEQ ID NO: 696), RGGILES (SEQ ID NO: 697), RGGNRES (SEQ ID NO: 698), RGGTLDS (SEQ ID NO: 699), LGGKLES (SEQ ID NO: 700), LGGKMES (SEQ ID NO: 701), TGGSLAS (SEQ ID NO: 702) and GGGTLES (SEQ ID NO: 703).

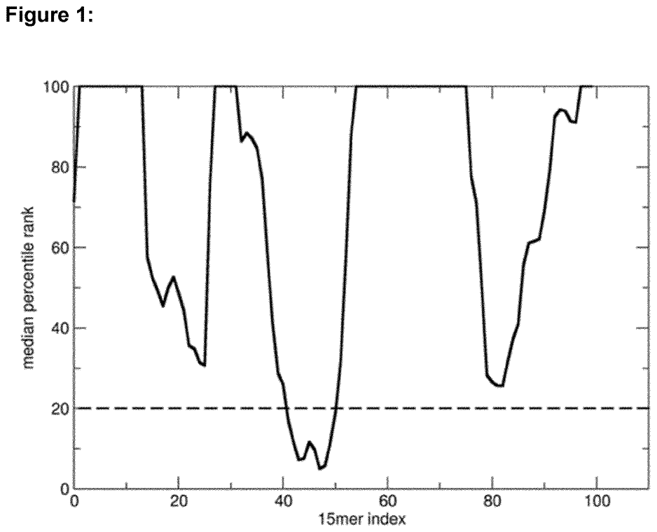

The antibody variable domains of the present invention exhibit a reduced predicted binding to MHC class II proteins, more specifically to human MHC class II molecules, when compared to their original versions that do not comprise said GG motif. For the prediction of the binding properties of the antibody variable domains of the present invention to MHC class II molecules, the NetMHCIIpan algorithm was used, more specifically version 3.1 of the NetMHCIIpan software and NetMHCIIpan algorithm, respectively, hereinafter referred to as “NetMHCIIpan-3.1” or “NetMHCIIpan-3.1 algorithm”, as described in detail in Example 2. Briefly, in this methodology, the immunogenicity risk of a given (therapeutic) protein is determined from the predicted binding strength of all 15mer peptide segments within the (therapeutic) protein to MHC class II molecules. These binding strengths are expressed by percentile ranks, which can be plotted over the analyzed sequence as for example shown in FIG. 1. The lower the percentile rank, the stronger is the predicted binding strength to MHC class II molecules.

Accordingly, the variable light chain and/or the variable heavy chain, in particular the variable light chain, of the antibody variable domain of the present invention comprises no 15mer peptide stretch having a median percentile rank, as determined from the predicted individual binding strengths (scoring ranks) of said 15mer peptide to each one of the 27 MHC class II alleles that are most common in the general population using the algorithm of NetMHCIIpan, of less than 13, particularly less than 14, particularly less than 15, particularly less than 16, particularly less than 17, particularly less than 18, particularly less than 19, particularly less than 20. The NetMHCIIpan-3.1 algorithm allows a reliable in silico prediction of immunogenicity risk of an antibody or antibody fragments.

Thus, for the purpose of the present invention, the term “immunogenicity” or “immunogenicity risk”, as used herein, refers to the capacity of a therapeutic protein, e. g. an antibody, an antibody fragment or an antibody variable domain, to bind to MHC class II proteins. Without being bound to theory, it is believed that the induction of the formation of ADAs during therapeutic treatment is linked with the occurrence of B cell and/or T cell epitopes on a therapeutic protein.

Apart from the use of in silico immunogenicity prediction tools, the extent of such immunogenicity can also be estimated by an ELISA assay and can be expressed by the percentage or the number of human serum samples, which contain measurable amounts of ADAs formed during therapeutic treatment, that recognize, i. e. bind to, the therapeutic protein in question, relative to the total number of tested human sera (percentage or number of positive serum samples). A reduction of immunogenicity between a therapeutic protein and a corresponding therapeutic protein being modified with the goal to reduce its immunogenicity, e. g. by implementing the GG motif described herein, can be measured by comparing the percentage of positive serum samples against the modified therapeutic protein, with the percentage of positive serum samples against the original therapeutic protein. A lower number or percentage of positive serum samples for the modified therapeutic protein indicates a reduction of immunogenicity relative to the original therapeutic protein.

The antibody variable domains of the present invention, when being in scFv format, further have advantageous biophysical properties, in particular an excellent stability. In particular, it was found by the inventors that the antibody variable domains of the present invention maintain their stability, as indicated by their melting temperature (Tm), when compared to their original versions that do not comprise the GG motif. In this regard, the expression “maintain their stability” means that their Tm is either equal or lower than the Tm of their respective unmodified versions (i. e. versions of said antibody variable domains that do not comprise the LCDR2 amino acids defined in item 1), or does not drop below 60° C. or, in particular is not lower than 5° C., when compared to the Tm of their original versions (i. e. versions of said antibody variable domains that do not comprise the LCDR2 amino acids defined in item 1). The determination of the individual Tm is described in detail in Example 5.

It was further found by the inventors that the antibody variable domains of the present invention maintain their binding affinity to their respective target antigens, when compared to their original versions that do not comprise the GG motif. In this regard, the expression “maintain their binding affinity” means that the monovalent dissociation constant (KD) for binding of the antibody variable domains of the invention to their target antigens, as measured by surface plasmon resonance (SPR), is either equal or lower than the KD of their respective unmodified versions (i. e. versions of said antibody variable domains that do not comprise the LCDR2 amino acids defined in item 1), or is not more than 2 times greater than the KD of their original versions (i. e. versions of said antibody variable domains that do not comprise the LCDR2 amino acids defined in item 1). The determination of the individual KD values by SPR is described in detail in Example 4.

Suitably, the antibody variable domain of the present invention, when being in scFv format, is further characterized by one or more of the following features:
(i) has a melting temperature (Tm), when compared with the Tm of a version of said antibody variable domain that does not comprise the LCDR2 amino acids defined in item 1, that is not lower than 5° C., particularly not lower than 4.5° C., particularly not lower than 4° C., particularly not lower than 3.5° C., particularly not lower than 3.0° C., as determined by differential scanning fluorimetry (DSF) and when formulated in 20 mM Histidine, pH 6.0;
(ii) binds its target antigen with a dissociation constant (KD) that is not greater than 2-folds, particularly not greater than 1.7-folds, particularly not greater than 1.5-folds, particularly not greater than 1.4-folds, particularly not greater than 1.3-folds, particularly not greater than 1.2-folds, when compared with the KD of a version of said antibody variable domain that does not comprise the LCDR2 amino acids defined in item 1, as measured by surface plasmon resonance (SPR).

DSF is described earlier (Egan, et al., MAbs, 9(1) (2017), 68-84; Niesen, et al., Nature Protocols, 2(9) (2007) 2212-2221). The midpoint of transition for the thermal unfolding of the scFv constructs is determined by nano Differential Scanning Fluorimetry as described in detail in Example 4. In brief, 1±0.1 mg/ml and 10±1 mg/ml solution in 20 mM Histidine buffer, pH 6, are prepared and subjected to a temperature ramp of from 20° C. to 95° C. with a 1° C./min increase. The unfolding event is monitored by using the intrinsic fluorescence of proteins, i. e. by the change of the fluorescence emission spectra of tryptophan (Trp). The midpoint of unfolding (Tm) is defined at the inflection point (thermal midpoint) of the unfolding curve observed as a local maximum or minimum of the first derivative.

As used herein, the term “affinity” refers to the strength of interaction between the antibody or the antibody variable domain and the antigen at single antigenic sites. Within each antigenic site, the variable region of the antibody variable domain or the antibody “arm” interacts through weak non-covalent forces with antigen at numerous sites; the more interactions, the stronger the affinity.

“Binding affinity” generally refers to the strength of the total sum of non-covalent interactions between a single binding site of a molecule (e. g., of an antibody or an antibody variable domain) and its binding partner (e. g., an antigen or, more specifically, an epitope on an antigen). Unless indicated otherwise, as used herein, “binding affinity”, “bind to”, “binds to” or “binding to” refers to intrinsic binding affinity that reflects a 1:1 interaction between members of a binding pair (e. g., an antibody variable domain and an antigen). The affinity of a molecule X for its partner Y can generally be represented by the dissociation constant (K_{D}). Affinity can be measured by common methods known in the art, including those described herein. Low-affinity antibodies and antibody variable domains generally bind antigens slowly and tend to dissociate readily, whereas high-affinity antibodies generally bind antigens faster and tend to remain bound longer. A variety of methods of measuring binding affinity are known in the art, any of which can be used for purposes of the present invention. Specific illustrative and exemplary embodiments for measuring binding affinity, i. e. binding strength are described in the following.

The term “K_{assoc}”, “Kₐ” or “Kon”, as used herein, are intended to refer to the association rate of a particular antibody-antigen interaction, whereas the term “K_{dis}”, “K_{d}” or “K_{off}”, as used herein, is intended to refer to the dissociation rate of a particular antibody-antigen interaction. In one embodiment, the term “Ko”, as used herein, is intended to refer to the dissociation constant, which is obtained from the ratio of K_{d} to Kₐ (i. e. K_{d}/Kₐ) and is expressed as a molar concentration (M). The “Ko” or “Ko value” or “KD” or “KD value” according to this invention is in one embodiment measured by using surface plasmon resonance assays.

The antibody variable domains of the present invention may have further modifications that can reduce immunogenicity. Thus, in some embodiments, the antibody variable domains of the present invention additionally comprise substitutions in the framework regions to reduce their potential binding to pre-existing ADAs, when being in an antibody fragment-based format, such as in an Fv or scFv format. Examples of such modifications are for example disclosed in the patent applications WO 2022/136693 and WO 2023/214047.

In particular embodiments, the antibody variable domains of the present invention comprise a variable heavy chain (VH) having a serine (S) at amino acid position 101 and a lysine (K) at amino acid position 146 (AHo numbering. These additional modifications in the framework regions of the variable heavy chain, herein also termed “SK modification” or “SK variant”, are disclosed in detail in the patent application WO 2023/214047.

In a second aspect, the present invention relates to an antibody comprising one or more antibody variable domains of the present invention.

In certain embodiments, the antibody of the present invention further comprises antibody variable domains that differ from the antibody variable domains of the present invention. More specifically, the antibody of the present invention further comprises antibody variable domains that do not have the GG motif as defined herein.

In preferred embodiments, the antibody of the present invention exclusively comprises antibody variable domains of the present invention. More specifically, in these preferred embodiments, the antibody of the present invention exclusively comprises antibody variable domains that have the substitutions in the framework regions as defined herein.

The term “monovalent antibody” or “antibody that is monovalent for its target antigen”, as used herein, refers to an antibody that binds to a single target molecule, and more specifically to a single epitope on a target molecule. Also, the term “binding domain” or “monovalent binding domain”, as used herein, refers to a binding domain that binds to a single epitope on a target molecule.

Furthermore, the term “binding domain” of an antibody, as used herein, or the terms “antigen-binding fragment thereof” or “antigen-binding portion” of an antibody, and the like, refer to one or more parts of an intact antibody that have the ability to bind to a given antigen, in particular to specifically bind to a given antigen. Antigen-binding functions of an antibody can be performed by fragments of an intact antibody. Specifically, in case of the antibodies of the present invention, the terms “binding domain”, as used herein, or the terms “antigen-binding fragment thereof” or “antigen-binding portion”, and the like, refer to a Fab fragment, i. e. a monovalent fragment consisting of the VL, VH, CL and CH1 domains; an Fv fragment consisting of the VL and VH domains of a single arm of an antibody; a disulfide stabilized Fv fragment (dsFv); and a single chain Fv fragment (scFv). Preferably, the binding domains of the antibodies of the present invention are independently of each other selected from a Fab fragment, an Fv fragment, a disulfide stabilized Fv fragment (dsFv) and a single-chain Fv fragment (scFv). In particular embodiments, the binding domains of the antibodies of the present invention are independently of each other selected from a Fab fragment an Fv fragment and a single-chain Fv fragment (scFv). In other particular embodiments, the VL and VH domains of the scFv fragment are stabilized by an interdomain disulfide bond, in particular said VH domain comprises a single cysteine residue in position 51 (AHo numbering) and said VL domain comprises a single cysteine residue in position 141 (AHo numbering).

The term “bivalent antibody” or “antibody that is bivalent for its target antigen”, as used herein, refers to a single antibody with two valencies, where “valency” is described as the number of antigen-binding moieties that binds to epitopes on a specific target molecule. As such, the single antibody can bind to two binding sites on a target molecule and/or to two target molecules due to the presence of two copies of the corresponding antigen-binding moieties.

Likewise, the term “trivalent antibody” or “antibody that is trivalent for its target antigen”, as used herein, refers to a single antibody with three valencies. As such, the single antibody can bind to three binding sites on a target molecule and/or can bind up to three target molecules due to the presence of three copies of the corresponding antigen-binding moieties.

In case the antibodies of the invention comprise two or three binding domains, said two or three binding domains either bind the same epitope or different epitopes on the target molecules. Preferably, the two or three binding domains bind the same epitope on the target molecule.

The term “same epitope”, as used herein, refers to an individual protein determinant on the protein capable of specific binding to more than one antibody, where that individual protein determinant is identical, i. e. consist of identical chemically active surface groupings of molecules such as amino acids or sugar side chains having identical three-dimensional structural characteristics, as well as identical charge characteristics for each of said antibodies.

The term “different epitope”, as used herein in connection with a specific protein target, refers to individual protein determinants on the protein, each capable of specific binding to a different antibody, where these individual protein determinants are not identical for the different antibodies, i. e. consist of non-identical chemically active surface groupings of molecules such as amino acids or sugar side chains having different three-dimensional structural characteristics, as well as different charge characteristics. These different epitopes can be overlapping or non-overlapping.

In one group of embodiments, the format of the antibody is selected from KiH-based IgGs, such as DuoBodies (bispecific IgGs prepared by the Duobody technology) (MAbs. 2017 February/March; 9(2):182-212. doi: 10.1080/19420862.2016.1268307); DVD-Ig; IgG-scFv fusions, such as CODV-IgG, Morrison (IgG CH₃-scFv fusion (Morrison-H) or IgG CL-scFv fusion (Morrison-L)), bsAb (scFv linked to C-terminus of light chain), Bs1Ab (scFv linked to N-terminus of light chain), Bs2Ab (scFv linked to N-terminus of heavy chain), Bs3Ab (scFv linked to C-terminus of heavy chain), Ts1Ab (scFv linked to N-terminus of both heavy chain and light chain) and Ts2Ab (dsscFv linked to C-terminus of heavy chain). More particularly, the format of said antibody is selected from KiH-based IgGs, such as DuoBodies; DVD-Ig; CODV-IgG and Morrison (IgG CH₃-scFv fusion (Morrison-H) or IgG CL-scFv fusion (Morrison-L)), even more particularly from DVD-Ig and Morrison (IgG CH₃-scFv fusion (Morrison-H) or IgG CL-scFv fusion (Morrison-L)).

In this group of embodiments, the IgG is preferably selected from the IgG subclasses IgG1 and IgG4, in particular IgG4.

In particular embodiments, the format of said antibody is selected from a Morrison format, i. e. a Morrison-L and a Morrison-H format. The Morrison-L and Morrison-H format used in the present invention are tetravalent and bispecific molecular formats bearing an IgG Fc region, in particular an IgG4 Fc region. Two highly stable scFv binding domains, wherein the light chain comprises Vκ FR1 to FR3 in combination with a Vλ FR4, and which are based on the antibody variable domains of the present invention, are fused via a linker L1 to the heavy chain (Morrison-H) or light chain (Morrison-L)C-termini.

The linker L1 is a peptide of 2-30 amino acids, more particularly 5-25 amino acids, and most particularly 10-20 amino acids. In particular embodiments, said linker L1 comprises one or more units of four (4) glycine amino acid residues and one (1) serine amino acid residue (GGGGS)ₙ, wherein n=1, 2, 3, 4 or 5 (SEQ ID NO: 537), particularly n=2 (SEQ ID NO: 538).

The VH regions and the VL regions of the two scFv domains are connected by a linker L2. The linker L2 is a peptide of 10-40 amino acids, more particularly 15-30 amino acids, and most particularly 20-25 amino acids. Particularly, said linker L2 comprises one or more units of four (4) glycine amino acid residues and one (1) serine amino acid residue (GGGGS)ₙ, wherein n=1, 2, 3, 4, 5, 6, 7 or 8 (SEQ ID NO: 539), particularly n=4 (SEQ ID NO: 540).

In another group of embodiments, the antibody of the invention does not comprise an immunoglobulin Fc region.

The term “immunoglobulin Fc region” or “Fc region”, as used herein, is used to define a C-terminal region of an immunoglobulin heavy chain, i. e. the CH2 and CH3 domains of the heavy chain constant regions. The term “Fc region” includes native-sequence Fc regions and variant Fc regions, i. e. Fc regions that are engineered to exhibit certain desired properties, such as for example altered Fc receptor binding function and/or reduced or suppressed Fab arm exchange. An example of such an engineered Fc region is the knob-into-hole (KiH) technology (see for example Ridgway et al., Protein Eng. 9:617-21 (1996) and Spiess et al., J Biol Chem. 288(37):26583-93 (2013)). Native-sequence Fc regions include human IgG1, IgG2 (IgG2A, IgG2B), IgG3 and IgG4. “Fc receptor” or “FcR” describes a receptor that binds to the Fc region of an antibody. Particularly, the FcR is a native sequence human FcR, which binds an IgG antibody (a gamma receptor) and includes receptors of the FcγRI, FcγRII, and FcγRIII subclasses, including allelic variants and alternatively spliced forms of these receptors, FcγRII receptors including FcγRIIA (an “activating receptor”) and FcγRI IB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain, (see M. Daeron, Annu. Rev. Immunol. 5:203-234 (1997). FcRs are reviewed in Ravetch and Kinet, Annu. Rev. Immunol. 9: 457-92 (1991); Capet et al, Immunomethods 4: 25-34 (1994); and de Haas et al, J. Lab. Clin. Med. 126: 330-41 (1995). Other FcRs, including those to be identified in the future, are encompassed by the term “FcR” herein. The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e. g., Ghetie and Ward, Immunol. Today 18: (12): 592-8 (1997); Ghetie et al., Nature Biotechnology 15 (7): 637-40 (1997); Hinton et al., J. Biol. Chem. TJI (8): 6213-6 (2004); WO 2004/92219 (Hinton et al). Binding to FcRn in vivo and serum half-life of human FcRn high-affinity binding polypeptides can be assayed, e. g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e. g., Shields et al., J. Biol. Chem. 9(2): 6591-6604 (2001).

In this group of embodiments, the antibody is preferably in a format selected from the group consisting of: a tandem scDb (Tandab), a linear dimeric scDb (LD-scDb), a circular dimeric scDb (CD-scDb), a tandem tri-scFv, a tribody (Fab-(scFv)₂), a Fab-Fv₂, a triabody, an scDb-scFv, a tetrabody, a di-diabody, a tandem-di-scFv and a MATCH (described in WO 2016/0202457; Egan T., et al., MABS 9 (2017) 68-84).

In particular embodiments, the antibody of the invention does further not comprise CH1 and/or CL regions. In these particular embodiments, the antibody is in a scDb-scFv, a triabody, a tetrabody or a MATCH format, particularly in a MATCH or scDb-scFv format. More particularly, the antibody of the invention is in a MATCH3 or a MATCH4 format.

In specific embodiments, the antibody of the invention is trispecific and tetravalent.

In further specific embodiments, the antibody of the invention is trispecific and trivalent.

The antibody variable domains comprised in the bispecific, trispecific tetraspecific or pentaspecific, antibodies of the invention are capable of binding to their respective antigens or receptors simultaneously. The term “simultaneously”, as used in this connection refers to the simultaneous binding of one of the antibody variable domains, which for example specifically binds to PD-L1, and of one or two further antibody variable domains, which for example have specificity for CD137 and hSA.

Suitably, the antibody variable domains comprised in the bispecific, trispecific tetraspecific or pentaspecific, antibodies of the invention are operably linked.

The term “operably linked”, as used herein, indicates that two molecules (e. g., polypeptides, domains, binding domains) are attached in a way that each molecule retains functional activity. Two molecules can be “operably linked” whether they are attached directly or indirectly (e. g., via a linker, via a moiety, via a linker to a moiety). The term “linker” refers to a peptide or other moiety that is optionally located between binding domains or antibody variable domains used in the invention. A number of strategies may be used to covalently link molecules together. These include, but are not limited to, polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. Choosing a suitable linker for a specific case where two polypeptide chains are to be connected depends on various parameters, including but not limited to the nature of the two polypeptide chains (e. g., whether they naturally oligomerize), the distance between the N- and the C-termini to be connected if known, and/or the stability of the linker towards proteolysis and oxidation. Furthermore, the linker may contain amino acid residues that provide flexibility.

In the context of the present invention, the term “polypeptide linker” refers to a linker consisting of a chain of amino acid residues linked by peptide bonds that is connecting two domains, each being attached to one end of the linker. The polypeptide linker should have a length that is adequate to link two molecules in such a way that they assume the correct conformation relative to one another so that they retain the desired activity. In particular embodiments, the polypeptide linker has a continuous chain of between 2 and 30 amino acid residues (e. g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues). In addition, the amino acid residues selected for inclusion in the polypeptide linker should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. In particular embodiments, the polypeptide linker is non-structured polypeptide. Useful linkers include glycine-serine, or GS linkers. By “Gly-Ser” or “GS” linkers is meant a polymer of glycines and serines in series (including, for example, (Gly-Ser)ₙ, (GSGGS)ₙ(SEQ ID NO: 541), (GGGGS)ₙ (SEQ ID NO: 542) and (GGGS)ₙ (SEQ ID NO: 543), where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since oligopeptides comprising these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single-chain antibodies.

Suitably, the antibody variable domain of the invention is an isolated variable domain. Likewise, the antibodies of the invention are isolated antibodies. The term “isolated variable domain” or “isolated antibody”, as used herein, refers to a variable domain or an antibody that is substantially free of other variable domains or other antibodies having different antigenic specificities (e. g., an isolated antibody variable domain that specifically binds mesothelin is substantially free of antibody variable domains that specifically bind antigens other than mesothelin). Moreover, an isolated antibody variable domain or isolated antibody may be substantially free of other cellular material and/or chemicals.

Suitably, the antibody variable domains and antibodies of the invention are monoclonal antibody variable domains and antibodies. The term “monoclonal antibody variable domains” or “monoclonal antibody” as used herein refers to variable domains or antibodies that have substantially identical amino acid sequences or are derived from the same genetic source. A monoclonal variable domain or antibody displays a binding specificity and affinity for a particular epitope, or binding specificities and affinities for specific epitopes.

The antibody variable domains and antibodies of the invention include, but are not limited to, chimeric, human and humanized antibody variable domains and antibodies.

The term “chimeric antibody” or “chimeric antibody variable domain”, as used herein, refers to an antibody molecule or antibody variable domain in which (a) the constant region, or a portion thereof, is altered, replaced or exchanged so that the antigen-binding site (variable region) is linked to a constant region of a different or altered class, effector function and/or species; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “human antibody” or “human antibody variable domain”, as used herein, is intended to include antibodies or antibody variable domains having variable regions in which both the framework and CDR regions are derived from sequences of human origin. Furthermore, if the antibody or antibody variable domain contains a constant region, the constant region also is derived from such human sequences, e. g., human germline sequences, or mutated versions of human germline sequences. The human antibodies and antibody variable domains of the invention may include amino acid residues not encoded by human sequences (e. g., mutations introduced by random or site-specific mutagenesis in vitro or by somatic mutation in vivo). This definition of a human antibody or antibody variable domain specifically excludes a humanized antibody or antibody variable domain comprising non-human antigen-binding residues. Human antibodies and antibody variable domains can be produced using various techniques known in the art, including phage-display libraries (Hoogenboom and Winter, J. Mol. Biol, 227:381 (1992); Marks et al, J. Mol. Biol, 222:581 (1991)). Also available for the preparation of human monoclonal antibodies and human monoclonal antibody variable domains are methods described in Cole et al, Monoclonal Antibodies and Cancer Therapy, Alan R. Liss, p. 77 (1985); Boemer et al, J. Immunol, 147(1):86-95 (1991). See also van Dijk and van de Winkel, Curr. Opin. Pharmacol, 5: 368-74 (2001). Human antibodies and human antibody variable domains can be prepared by administering the antigen to a transgenic animal that has been modified to produce such antibodies and antibody variable domains in response to antigenic challenge, but whose endogenous loci have been disabled, e. g., immunized xenomice (see, e. g., U.S. Pat. Nos. 6,075,181 and 6,150,584 regarding XENOMOUSE™ technology). See also, for example, Li et al, Proc. Natl. Acad. Sci. USA, 103:3557-3562 (2006) regarding human antibodies generated via a human B-cell hybridoma technology.

The term “humanized” antibody or “humanized” antibody variable domain, as used herein, refers to an antibody or antibody variable domain that retains the reactivity of a non-human antibody or antibody variable domain while being less immunogenic in humans. This can be achieved, for instance, by retaining the non-human CDR regions and replacing the remaining parts of the antibody or antibody variable domain with their human counterparts (i. e. the constant region as well as the framework portions of the variable region). Additional framework region modifications may be made within the human framework sequences as well as within the CDR sequences derived from the germline of another mammalian species. The humanized antibodies and antibody variable domains of the invention may include amino acid residues not encoded by human sequences (e. g., mutations introduced by random or site-specific mutagenesis in vitro or by somatic mutation in vivo, or a conservative substitution to promote stability or manufacturing). See, e. g., Morrison et al., Proc. Natl. Acad. Sci. USA, 81:6851-6855, 1984; Morrison and Oi, Adv. Immunol., 44:65-92, 1988; Verhoeyen et al., Science, 239: 1534-1536, 1988; Padlan, Molec. Immun., 28:489-498, 1991; and Padlan, Molec. Immun., 31: 169-217, 1994. Other examples of human engineering technology include but are not limited to the Xoma technology disclosed in U.S. Pat. No. 5,766,886.

The term “recombinant humanized antibody” or “recombinant humanized antibody variable domain” as used herein, includes all human antibodies and human antibody variable domains that are prepared, expressed, created or isolated by recombinant means, such as antibodies and antibody variable domains isolated from a host cell transformed to express the humanized antibody or humanized antibody variable domain, e. g., from a transfectoma, and antibodies and antibody variable domains prepared, expressed, created or isolated by any other means that involve splicing of all or a portion of a human immunoglobulin gene, sequences to other DNA sequences.

Preferably, the antibody variable domains and antibodies of the invention are humanized. More preferably, the antibody variable domains and antibodies of the invention are humanized and comprise rabbit-derived CDRs.

The term “bispecific antibody”, “trispecific antibody”, “tetraspecific antibody”, “pentaspecific antibody” or the more general term “multispecific antibody” as used herein, refers to an antibody that binds to two or more different epitopes on at least two or more different targets, for example 2 different targets (bispecific), 3 different targets (trispecific), 4 different targets (tetraspecific), or 5 different targets (pentaspecific). Preferably, the antibodies of the invention are bispecific, trispecific or tetraspecific, particularly bispecific or trispecific, more particularly trispecific. As indicated above, the term trispecific antibody refers to an antibody that binds to at least three different epitopes on three different targets (e. g., PD-L1, CD137 and hSA or mesothelin, CD3 and hSA).

The term “epitope” means a protein determinant capable of specific binding to an antibody. Epitopes usually consist of chemically active surface groupings of molecules such as amino acids or sugar side chains and usually have specific three-dimensional structural characteristics, as well as specific charge characteristics. “Conformational” and “linear” epitopes are distinguished in that the binding to the former but not the latter is lost in the presence of denaturing solvents.

The term “conformational epitope” as used herein refers to amino acid residues of an antigen that come together on the surface when the polypeptide chain folds to form the native protein.

The term “linear epitope” refers to an epitope, wherein all points of interaction between the protein and the interacting molecule (such as an antibody) occurring linearly along the primary amino acid sequence of the protein (continuous).

The term “recognize” as used herein refers to an antibody or antibody variable domain that finds and interacts (e. g., binds) with its conformational epitope.

The inventors found that the antibody variable domain of the invention can be successfully applied in the construction of diverse antibody fragments, e. g. scFv fragments, and multispecific antibodies, e. g. bispecific and trispecific antibodies, which exhibits significantly reduced immunogenicity, when compared to their unmodified counterparts, and which have an excellent stability.

The antibody variable domains and antibodies of the invention can be produced using any convenient antibody-manufacturing method known in the art (see, e. g., Fischer, N. & Leger, O., Pathobiology 74 (2007) 3-14 with regard to the production of bispecific constructs; Hornig, N. & Farber-Schwarz, A., Methods Mol. Biol. 907 (2012)713-727, and WO 99/57150 with regard to bispecific diabodies and tandem scFvs). Specific examples of suitable methods for the preparation of the multispecific constructs further include, inter alia, the Genmab (see Labrijn et al., Proc. Natl. Acad. Sci. USA 110 (2013) 5145-5150) and Merus (see de Kruif et al., Biotechnol. Bioeng. 106 (2010) 741-750) technologies.

These methods typically involve the generation of monoclonal antibodies or monoclonal antibody variable domains, for example by means of fusing myeloma cells with the spleen cells from a mouse that has been immunized with the desired antigen using the hybridoma technology (see, e. g., Yokoyama et al., Curr. Protoc. Immunol. Chapter 2, Unit 2.5, 2006) or by means of recombinant antibody engineering (repertoire cloning or phage display/yeast display) (see, e. g., Chames & Baty, FEMS Microbiol. Letters 189 (2000) 1-8), and the combination of the antigen-binding domains or fragments or parts thereof of two or more different monoclonal antibodies to give a bispecific or multispecific construct using known molecular cloning techniques.

The antibodies of the invention that are multispecific, e. g. bispecific, trispecific, tetraspecific or pentaspecific, and/or multivalent, can be prepared by conjugating the constituent binding specificities, using methods known in the art. For example, each binding specificity of these antibodies can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis (2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)-cyclohexane-1-carboxylate (sulfo-SMCC) (see e. g., Karpovsky et al., 1984 J. Exp. Med. 160: 1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83, and Glennie et al., 1987 J. Immunol. 139: 2367-2375. Conjugating agents are SATA and sulfo-SMCC, both available from Pierce Chemical Co. (Rockford, Ill).

Alternatively, two or more binding specificities can be encoded in the same vector and expressed and assembled in the same host cell. This method is particularly useful where the bispecific molecule is a mAb×Fab, a mAb×scFv, a mAb×dsFv or a mAb×Fv fusion protein. Methods for preparing multispecific and/or multivalent antibodies and molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the antibody variable domains and multispecific antibodies to their specific targets can be confirmed by, for example, enzyme-linked immunosorbent assay (ELISA), radioimmunoassay (REA), FACS analysis, bioassay (e. g., growth inhibition), or Western Blot assay. Each of these assays generally detects the presence of protein-antibody complexes of particular interest by employing a labeled reagent (e. g., an antibody) specific for the complex of interest.

In a further aspect, the invention provides a nucleic acid or two nucleic acids encoding the antibody variable domain or the antibody of the invention. Such nucleic acids can be optimized for expression in mammalian cells.

The term “nucleic acid” is used herein interchangeably with the term “polynucleotide(s)” and refers to one or more deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphorates, 2-O-methyl ribonucleotides, peptide-nucleic acids (PNAs). Unless otherwise indicated, a particular nucleic acid sequence also implicitly encompasses conservatively modified variants thereof (e. g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The invention provides substantially purified nucleic acid molecules which encode polypeptides comprising segments or domains of the antibody variable domains or the antibodies described above. When expressed from appropriate expression vectors, polypeptides encoded by these nucleic acid molecules are capable of exhibiting antigen-binding capacities of the antibody variable domains or the antibodies of the present invention.

The polynucleotide sequences can be produced by de novo solid-phase DNA synthesis or by PCR mutagenesis of an existing sequence (e. g., sequences as described in the Examples below) encoding the antibody variable domain or the antibody of the invention. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68: 109, 1979; the diethylphosphoramidite method of Beaucage et al., Tetrahedron Lett., 22: 1859, 1981; and the solid support method of U.S. Pat. No. 4,458,066. Introducing mutations to a polynucleotide sequence by PCR can be performed as described in, e. g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., Nucleic Acids Res. 19:967, 1991; and Eckert et al., PCR Methods and Applications 1:17, 1991.

Also provided in the invention are expression vectors and host cells for producing the antibody variable domain or the antibody of the invention.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e. g., bacterial vectors having a bacterial origin of replication and episomal mammalian vectors). Other vectors (e. g., non-episomal mammalian vectors) can be integrated into the genome of a host cell upon introduction into the host cell, and thereby are replicated along with the host genome.

Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e. g., replication defective retroviruses, adenoviruses and adeno-associated viruses), which serve equivalent functions. In this particular context, the term “operably linked” refers to a functional relationship between two or more polynucleotide (e. g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i. e. they are cis-acting. However, some transcriptional regulatory sequences, such as enhancers, need not be physically contiguous or located in close proximity to the coding sequences whose transcription they enhance.

Various expression vectors can be employed to express the polynucleotides encoding the antibody variable domain or the antibody chain(s). Both viral-based and non-viral expression vectors can be used to produce the antibodies or antibody variable domains in a mammalian host cell. Non-viral vectors and systems include plasmids, episomal vectors, typically with an expression cassette for expressing a protein or RNA, and human artificial chromosomes (see, e. g., Harrington et al., Nat Genet. 15:345, 1997). For example, non-viral vectors useful for expression of the PD-L1-, CD137-, CD3-, hSA- or MSLN-binding polypeptides, or of polynucleotides encoding such polypeptides, in mammalian (e. g., human) cells include pThioHis A, B and C, pcDNA3.1/His, pEBVHis A, B and C, (Invitrogen, San Diego, Calif.), MPS V vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, Vaccinia virus vectors and Semliki Forest virus (SFV). See, Brent et al., supra; Smith, Annu. Rev. Microbiol. 49:807, 1995; and Rosenfeld et al., Cell 68: 143, 1992.

The choice of expression vector depends on the intended host cells in which the vector is to be expressed. Typically, the expression vectors contain a promoter and other regulatory sequences (e. g., enhancers) that are operably linked to the polynucleotides encoding a multispecific antibody chain or a variable domain. In one embodiment, an inducible promoter is employed to prevent expression of inserted sequences except under inducing conditions. Inducible promoters include, e. g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of a multispecific antibody chain or a variable domain. These elements typically include an ATG initiation codon and adjacent ribosome binding site or other sequences. In addition, the efficiency of expression may be enhanced by the inclusion of enhancers appropriate to the cell system in use (see, e. g., Scharf et al., Results Probl. Cell Differ. 20: 125, 1994; and Bittner et al., Meth. Enzymol., 153:516, 1987). For example, the SV40 enhancer or CMV enhancer may be used to increase expression in mammalian host cells.

Vectors to be used typically encode the antibody variable domain or the antibody light and heavy chain including constant regions or parts thereof, if present. Such vectors allow expression of the variable regions as fusion proteins with the constant regions thereby leading to production of intact antibodies and antibody variable domains thereof. Typically, such constant regions are human.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The host cells for harboring and expressing the antibody variable domain or the antibody of the invention can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e. g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express the antibody variable domain or multispecific antibodies of the invention. Insect cells in combination with baculovirus vectors can also be used.

In one embodiment, mammalian host cells are used to express and produce the antibody variable domain or the antibody of the invention. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various COS cell lines, HeLa cells, myeloma cell lines, transformed B-cells and hybridomas. The use of mammalian tissue cell culture to express polypeptides is discussed generally in, e. g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e. g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPS V promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Green, M. R., and Sambrook, J., Molecular Cloning: A Laboratory Manual (Fourth Edition), Cold Spring Harbor Laboratory Press (2012)). Other methods include, e. g., electroporation, calcium phosphate treatment, liposome-mediated transformation, injection and microinjection, ballistic methods, virosomes, immunoliposomes, polycation-nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody variable domain or the antibody of the invention can be prepared using expression vectors of the invention which contain viral origins of replication or endogenous expression elements and a selectable marker gene. Following the introduction of the vector, cells may be allowed to grow for 1 to 2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. The present invention thus provides a method of producing the antibody variable domain or the antibody of the invention, wherein said method comprises the step of culturing a host cell comprising a nucleic acid or a vector encoding the antibody variable domain or the antibody of the invention, whereby said antibody variable domain or said antibody of the disclosure is expressed.

In one aspect, the present invention relates to a method of producing the antibody variable domain or the antibody of the invention, the method comprising the step of culturing a host cell expressing a nucleic acid or two nucleic acids encoding the antibody variable domain or the antibody of the invention. In particular, the present invention relates to a method of producing the antibody variable domain or the antibody of the invention, the method comprising (i) providing a nucleic acid or two nucleic acids encoding the antibody variable domain or the antibody of the invention or one or two vectors encoding the antibody variable domain or the antibody of the invention, expressing said nucleic acid or nucleic acids, or said vector or vectors, and collecting said antibody variable domain or said antibody from the expression system, or (ii) providing a host cell or host cells expressing a nucleic acid or two nucleic acids encoding the antibody variable domain or the antibody of the invention, culturing said host cell or said host cells; and collecting said antibody variable domain or said multispecific antibody from the cell culture.

In a further aspect, the present invention relates to a pharmaceutical composition comprising the antibody of the invention, and a pharmaceutically acceptable carrier. “Pharmaceutically acceptable carrier” means a medium or diluent that does not interfere with the structure of the antibodies. Pharmaceutically acceptable carriers enhance or stabilize the composition, or facilitate preparation of the composition. Pharmaceutically acceptable carriers include solvents, dispersion media, coatings, antibacterial and antifungal agents, isotonic and absorption delaying agents, and the like that are physiologically compatible.

Certain of such carriers enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection, infusion or topical administration. For example, a pharmaceutically acceptable carrier can be a sterile aqueous solution.

Pharmaceutical compositions in accordance with the present disclosure may further routinely contain pharmaceutically acceptable concentrations of salt, buffering agents, preservatives, supplementary immune potentiating agents such as adjuvants and cytokines and optionally other therapeutic agents. The composition may also include antioxidants and/or preservatives. As antioxidants may be mentioned thiol derivatives (e. g. thioglycerol, cysteine, acetylcysteine, cystine, dithioerythreitol, dithiothreitol, glutathione), tocopherols, butylated hydroxyanisole, butylated hydroxytoluene, sulfurous acid salts (e. g. sodium sulfate, sodium bisulfite, acetone sodium bisulfite, sodium metabisulfite, sodium sulfite, sodium formaldehyde sulfoxylate, sodium thiosulfate) and nordihydroguaiaretic acid. Suitable preservatives may for instance be phenol, chlorobutanol, benzylalcohol, methyl paraben, propyl paraben, benzalkonium chloride and cetylpyridinium chloride.

The pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e. g., by injection or infusion). Depending on the route of administration, the active compound, i. e. the antibody of the invention, may be coated in a material to protect the compound from the action of acids and other natural conditions that may inactivate the compound.

The pharmaceutical compositions of the invention can be prepared in accordance with methods well known and routinely practiced in the art. See, e. g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the antibody of the invention is employed in the pharmaceutical compositions of the invention. The antibodies of the invention are formulated into pharmaceutically acceptable dosage forms by conventional methods known to those of skill in the art. Dosage regimens are adjusted to provide the optimum desired response (e. g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

In one aspect, the present invention relates to the antibody of the invention or the pharmaceutical composition of the invention for use as a medicament. In a suitable embodiment, the present invention provides the multispecific antibody or the pharmaceutical composition for use in the treatment of a proliferative disease, such as cancer, or a disease selected from allergic, inflammatory and autoimmune diseases.

In another aspect, the present invention provides the pharmaceutical composition of the invention for use in the manufacture of a medicament for the treatment of a proliferative disease, such as cancer, or a disease selected from allergic, inflammatory and autoimmune diseases.

In another aspect, the present invention relates to the use of the antibody or the pharmaceutical composition of the present invention for treating a proliferative disease, such as cancer, or a disease selected from allergic, inflammatory and autoimmune diseases, in a subject in need thereof.

In another aspect, the present invention relates to a method of treating a subject comprising administering to the subject a therapeutically effective amount of the antibody of the present invention. In a suitable embodiment, the present invention relates to a method for the treatment of a proliferative disease, such as cancer, or a disease selected from allergic, inflammatory and autoimmune diseases, in a subject comprising administering to the subject a therapeutically effective amount of the antibody of the present invention.

The term “subject” includes human and non-human animals.

The term “animals” include all vertebrates, e. g., non-human mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, and the like, as used herein, refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e. g., in a human, and includes: (a) inhibiting the disease, i. e. arresting its development; and (b) relieving the disease, i. e. causing regression of the disease.

The term “therapeutically effective amount” or “efficacious amount” refers to the amount of an agent that, when administered to a mammal or other subject for treating a disease, is sufficient to affect such treatment for the disease. The “therapeutically effective amount” will vary depending on the agent, the disease and its severity and the age, weight, etc., of the subject to be treated.

In another aspect, the present invention relates to a method for generating a modified antibody variable domain, wherein the method comprises the steps of
1) providing an unmodified antibody variable domain, which binds to a target antigen, comprising:
   (i) a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region;
   and
   (ii) a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region, wherein the light chain complementarity-determining region LCDR2 does not comprise a glycine (G) at both amino acid position 67 and 68 (AHo numbering);
2) introducing into the light chain complementarity-determining region LCDR2 of the unmodified antibody variable domain provided in step 1) a glycine (G) at amino acid position 67 and/or a glycine (G) at amino acid position 68 (AHo numbering), such that there is a glycine (G) at both amino acid positions 67 and 68,
wherein said LCDR2 is defined by light chain amino acid positions 58 to 72 according to the AHo numbering scheme, resulting in a maximum length of 15 amino acids.

In particular embodiments of the method according to the present invention, the LCDR2 of the unmodified antibody variable domain that is provided in step 1) has one or more of the features as defined above in items 47 to 52.

In further particular embodiments of the method according to the present invention, the LCDR2 and optionally the other light chain complementarity-determining regions LCDR1 and LCDR3 and the heavy chain complementarity-determining regions HCDR1, HCDR2 and HCDR3 of the modified antibody variable domain obtained in step 2) has one or more of the features as defined above in items 53 and 54.

Likewise, in yet other particular embodiments of the method of the present invention, the heavy chain framework regions HFW1, HFW2, HFW3 and HFW4 and/or the light chain framework regions LFW1, LFW2, LFW3 and LFW4 of the unmodified antibody variable domain that is provided in step 1) are as defined in any one of the items 3 and 10 to 16.

In further particular embodiments, the method comprises that additional step of
3) introducing into the variable heavy chain (VH) of the unmodified antibody variable domain provided in step 1) a serine (S) at amino acid position 101 and a lysine (K) at amino acid position 146 (AHo numbering).

Partial sequence listing (residues designated according to AHo numbering scheme; the CDRs defined according to Numab CDR definition, unless specified otherwise)

TABLE 1
Examples of VH/VL sequences of antibody variable domains according to the present invention
(modifications relative to original sequences, i. e. reference sequences, are shown in bold and
underlined; CDR residues are shown in bold and italic letters).

| | | |
|---|---|---|
| SEQ | | |
| ID | | |
| NO: | Description: | Sequence: |

Anti-PD-L1 antibody variable domains
37-20-B03-sc01-based sequences

| | | |
|---|---|---|
| 1 | VH | QVQLQESGPGLVKPSETLSLTCKVSGFSFNSDYWIYWIRQPPGKGLEWIGSIYGGSSGNTQYASW |
| | 37-20-B03-sc01 | AQGRVTISVDSSKNQFSLKLSSVTAADTAVYYCARGYVDYGGATDLWGQGTLVTVSS |
| 2 | VH: 37-20-B03 | QVQLQESGPGLVKPSETLSLTCKVSGFSFNSDYWIYWIRQPPGKGLEWIGSIYGGSSGNTQYASW |
| | sc01_(T101S, T146K) | AQGRVTISVDSSKNQFSLKLSSVTAADSAVYYCARGYVDYGGATDLWGQGTLVKVSS |
| 3 | VL: 37-20-B03 sc01 | DIQMTQSPSSLSASVGDRVTITCQASQSIGTYLAWYQQKPGKAPKLLIYRAFILASGVPSRFSGSG |
| | (Reference) | SGTDFTLTISSLQPEDFATYYCQSNFYSDSTTIGPNAFGTGTKVTVLG |
| 4 | VL: 37-20-B03 | DIQMTQSPSSLSASVGDRVTITCQASQSIGTYLAWYQQKPGKAPKLLIYRGGILASGVPSRFSGSG |
| | sc01_(A67G_F68G) | SGTDFTLTISSLQPEDFATYYCQSNFYSDSTTIGPNAFGTGTKVTVLG |
| 5 | scFv | DIQMTQSPSSLSASVGDRVTITCQASQSIGTYLAWYQQKPGKAPKLLIYRAFILASGVPSRFSGSG |
| | 37-20-B03 sc01 | SGTDFTLTISSLQPEDFATYYCQSNFYSDSTTIGPNAFGTGTKVTVLGGGGGSGGGGSGGGGSG |
| | (Reference) | GGGSQVQLQESGPGLVKPSETLSLTCKVSGFSFNSDYWIYWIRQPPGKGLEWIGSIYGGSSGNTQ |
| | (PRO997) | YASWAQGRVTISVDSSKNQFSLKLSSVTAADTAVYYCARGYVDYGGATDLWGQGTLVTVSS |
| 6 | scFv | DIQMTQSPSSLSASVGDRVTITCQASQSIGTYLAWYQQKPGKAPKLLIYRGGILASGVPSRFSGSG |
| | 37-20-B03 sc01 | SGTDFTLTISSLQPEDFATYYCQSNFYSDSTTIGPNAFGTGTKVTVLGGGGGSGGGGSGGGGSG |
| | (A67G_F68G) | GGGSQVQLQESGPGLVKPSETLSLTCKVSGFSFNSDYWIYWIRQPPGKGLEWIGSIYGGSSGNTQ |
| | | YASWAQGRVTISVDSSKNQFSLKLSSVTAADTAVYYCARGYVDYGGATDLWGQGTLVTVSS |
| 7 | scFv | DIQMTQSPSSLSASVGDRVTITCQASQSIGTYLAWYQQKPGKAPKLLIYRGGILASGVPSRFSGSG |
| | 37-20-B03 sc01 | SGTDFTLTISSLQPEDFATYYCQSNFYSDSTTIGPNAFGTGTKVTVLGGGGGSGGGGSGGGGSG |
| | (T101S_T146K)_ | GGGSQVQLQESGPGLVKPSETLSLTCKVSGFSFNSDYWIYWIRQPPGKGLEWIGSIYGGSSGNTQ |
| | (A67G_F68G) | YASWAQGRVTISVDSSKNQFSLKLSSVTAADSAVYYCARGYVDYGGATDLWGQGTLVKVSS |

37-20-B03-sc02-based sequences

| | |
|---|---|
| 8 | VH: 37-20-B03-sc02 |
| 9 | VH: 37-20-B03-sc02_(T101S, T146K) |
| 10 | VL: 37-20-B03-sc02 (Reference) |
| 11 | VL: 37-20-B03-sc02_(A67G_F68G) |
| 12 | scFv: 37-20-B03 sc02 (Reference) (PRO1013) |
| 13 | scFv: 37-20-B03 sc02 (A67G_F68G) |
| 14 | scFv: 37-20-B03 sc02 (T101S_T146K)_(A67G_F68G) |

37-20-B03-sc09.1-based sequences

| | |
|---|---|
| 15 | VH: 37-20-B03-sc09.1 (PRO2230) |
| 16 | VH: 37-20-B03-sc09.1_(Y42S) (PRO3476) |
| 17 | VH: 37-20-B03-sc09.1_(T101S_T146K) |
| 18 | VH: 37-20-B03-sc09.1_(Y42S_T101S_T146K) |
| 19 | VL: 37-20-B03-sc09.1 (PRO2230) (Reference) |
| 20 | VL: 37-20-B03-sc09.1_(A67G_F68G) |
| 21 | VL: 37-20-B03-sc09.1_(A9S) (PRO3757) (Reference) |
| 22 | VL: 37-20-B03-sc09.1_(A9S_A67G_F68G) |
| 23 | scFv: 37-20-B03 sc09.1 (Reference) (PRO2230) |
| 24 | scFv: 37-20-B03 sc09.1 (A67G_F68G) |
| 25 | scFv: 37-20-B03 sc09.1 (T101S_T146K) (A67G_F68G) |
| 662 | scFv: 37-20-B03-sc09.1_(A9S) (Reference) (PRO3757) |
| 26 | scFv: 37-20-B03-sc09.1_(Y42S) (Reference) (PRO3476) |
| 27 | scFv: 37-20-B03-sc09.1_(Y42S) (A67G_F68G) |
| 28 | scFv: 37-20-B03-sc09.1_(Y42S) (T101S_T146K) (A67G_F68G) |
| 29 | scFv: 37-20-B03 sc09.1_(A9S) (A67G_F68G) |
| 30 | scFv: 37-20-B03 sc09.1_(A9S) (T101S_T146K) (A67G_F68G) |
| 663 | scFv: 37-20-B03-sc09.1_CDR2_human_cons (Reference) (PRO3477) |
| 664 | scFv: 37-20-B03-sc09.1_(Y42S)_CDR2_human_cons (Reference) (PRO3478) |

37-20-B03-sc16-based sequences

| | |
|---|---|
| 31 | VH: 37-20-B03-sc16 |
| 32 | VL: 37-20-B03-sc16 (Reference) |
| 33 | VL: 37-20-B03-sc16_(A67G_F68G) |
| 34 | scFv: 37-20-B03 sc16 (Reference) (PRO3757) |
| 35 | scFv: 37-20-B03 sc16 (A67G_F68G) (PRO4124) |

37-20-B03-sc17-based sequences

| | |
|---|---|
| 36 | VH: 37-20-B03-sc17 |
| 37 | VH: 37-20-B03-sc17_(T101S_T146K) |
| 38 | VL: 37-20-B03-sc17 (Reference) |
| 39 | VL: 37-20-B03-sc17_(A67G_F68G) |
| 40 | scFv: 37-20-B03 sc17 (Reference) (PRO3757) |
| 41 | scFv: 37-20-B03 sc17 (A67G_F68G) (PRO4125) |
| 42 | scFv: 37-20-B03 sc17 (T101S_T146K)_(A67G_F68G) |

37-20-B03-sc09-IF-based sequences

| | |
|---|---|
| 43 | VH: 37-20-B03-sc09-IF (PRO1347) |
| 44 | VH: 37-20-B03-sc09-IF_(T101S_T146K) |
| 45 | VL: 37-20-B03-sc09-IF (PRO1347) (Reference) |
| 46 | VL: 37-20-B03-sc09-IF_(A67G_F68G) |
| 47 | scFv: 37-20-B03 sc09-IF (Reference) (PRO1347) |
| 48 | scFv: 37-20-B03 sc09-IF (A67G_F68G) (PRO4125) |
| 49 | scFv: 37-20-B03 sc09-IF (T101S_T146K)_(A67G_F68G) |

33-03-G02-sc01-based sequences

| | |
|---|---|
| 50 | VH: 33-03-G02-sc01 |
| 51 | VH: 33-03-G02-sc01_(T101S, T146K) |
| 52 | VL: 33-03-G02-sc01 (Reference) |
| 53 | VL: 33-03-G02-sc01_(A67G_S68G) |
| 54 | scFv: 33-03-G02-sc01 (Reference) (PRO830) |
| 55 | scFv: 33-03-G02-sc01 (A67G_S68G) |
| 56 | scFv: 33-03-G02-sc01 (T101S_T146K)_(A67G_S68G) |

33-03-G02-sc03-based sequences

| | |
|---|---|
| 57 | VH: 33-03-G02-sc03 |
| 58 | VH: 33-03-G02-sc03_(T101S, T146K) |
| 59 | VL: 33-03-G02-sc03 (Reference) |
| 60 | VL: 33-03-G02-sc03_(A67G_S68G) |
| 61 | scFv: 33-03-G02-sc03 (Reference) (PRO997) |
| 62 | scFv: 33-03-G02-sc03 (A67G_S68G) |
| 63 | scFv: 33-03-G02-sc03 (T101S_T146K)_(A67G_S68G) |

33-03-G02-sc18-based sequences

| | |
|---|---|
| 64 | VH: 33-03-G02-sc18 (PRO1392) |
| 65 | VH: 33-03-G02-sc18_(S72D_W73S_A74V) |
| 66 | VH: 33-03-G02-sc18_(T101S_T146K) |
| 67 | VL: 33-03-G02-sc18 (PRO1392) (Reference) |
| 68 | VL: 33-03-G02-sc18_(A67G_S68G) |
| 69 | VL: 33-03-G02-sc18_(A67G_S68G_S81R) |
| 70 | scFv: 33-03-G02-sc18 (Reference) (PRO1392) |
| 71 | scFv: 33-03-G02-sc18 (A67G_S68G) (PRO3724) |
| 72 | scFv: 33-03-G02-sc18 (T101S_T146K)_(A67G_S68G) (PRO3891) |
| 73 | scFv: 33-03-G02-sc18 (S72D_W73S_A74V)_(A67G_S68G) (PRO3747) |

Anti-CD137 antibody variable domains
38-27-A11-sc02-based VH/VL sequences

| | |
|---|---|
| 74 | VH: 38-27-A11-sc02 (PRO1359) |
| 75 | VH: 38-27-A11-sc02_(G51C) |
| 76 | VH: 38-27-A11-sc02_(N72Q) |
| 77 | VH: 38-27-A11-sc02_(G51C_N72Q) |
| 78 | VL: 38-27-A11-sc02 (PRO1359) (Reference) |
| 79 | VL: 38-27-A11-sc02_(T141C) (Reference) |
| 80 | VL: 38-27-A11-sc02_(A67G_S68G) |
| 81 | VL: 38-27-A11-sc02_(A67G_S68G_T141C) |
| 82 | scFv: 38-27-A11-sc02 (Reference) (PRO1359) |
| 83 | scFv: 38-27-A11-sc02 (A67G_S68G) (PRO3726) |
| 84 | scFv: 38-27-A11-sc02 (T101S_T146K)_(A67G_S68G) |
| 85 | scFv: 38-27-A11-sc02_(N72Q) (Reference) |
| 86 | scFv: 38-27-A11-sc02_(N72Q) (A67G_S68G) |
| 87 | scFv: 38-27-A11-sc02_(N72Q) (T101S_T146K)_(A67G_S68G) |
| 88 | scFv: 38-27-A11-sc02_(G51C)_(T141C) (Reference) |
| 89 | scFv: 38-27-A11-sc02_(G51C)_(T141C) (A67G_S68G) |
| 90 | scFv: 38-27-A11-sc02_(G51C)_(T141C) (T101S_T146K)_(A67G_S68G) |
| 91 | scFv: 38-27-A11-sc02_(G51C_N72Q)_(T141C) (Reference) (PRO2194) |
| 92 | scFv: 38-27-A11-sc02_(G51C_N72Q)_(T141C) (A67G_S68G) (PRO3742) |
| 93 | scFv: 38-27-A11-sc02_(G51C_N72Q)_(T141C) (T101S_T146K)_(A67G_S68G) |

38-27-A11-sc03-based sequences

| | |
|---|---|
| 94 | VH: 38-27-A11-sc03 |
| 95 | VH: 38-27-A11-sc03_(T101S_T146K) |
| 96 | VL: 38-27-A11-sc03 (Reference) |
| 97 | VL: 38-27-A11-sc03_(A67G_S68G) |
| 98 | scFv: 38-27-A11-sc03 (Reference) (PRO1360) |
| 99 | scFv: 38-27-A11-sc03 (A67G_S68G) |
| 100 | scFv: 38-27-A11-sc03 (T101S_T146K) (A67G_S68G) |

38-27-A11-sc10-based sequences

| | |
|---|---|
| 101 | VH: 38-27-A11-sc10 |
| 102 | VH: 38-27-A11-sc10_(G51C) |
| 103 | VL: 38-27-A11-sc10 (Reference) |
| 104 | VL: 38-27-A11-sc10_(T141C) (Reference) |
| 105 | VL: 38-27-A11-sc10_(A67G_F68G) |
| 106 | VL: 38-27-A11-sc10_(A67G_F68G_T141C) |
| 107 | scFv: 38-27-A11-sc10 (Reference) |
| 108 | scFv: 38-27-A11-sc10 (A67G_S68G) |
| 109 | scFv: 38-27-A11-sc10_(G51C)_(T141C) (A67G_S68G) |

38-02-A04-sc01-based sequences

| | |
|---|---|
| 110 | VH: 38-02-A04-sc01 |
| 111 | VH: 38-02-A04-sc01_(T101S_T146K) |
| 112 | VL: 38-02-A04-sc01 (Reference) |
| 113 | VL: 38-02-A04-sc01_(A67G_S68G) |
| 114 | scFv: 38-02-A04-sc01 (Reference) (PRO) |
| 115 | scFv: 38-02-A04-sc01 (A67G_S68G) |
| 116 | scFv: 38-02-A04-sc01 (T101S_T146K)_(A67G_S68G) |

38-02-A04-sc05-based sequences

| | |
|---|---|
| 117 | VH: 38-02-A04-sc05 |
| 118 | VH: 38-02-A04-sc05_(T101S_T146K) |
| 119 | VL: 38-02-A04-sc05 (Reference) |
| 120 | VL: 38-02-A04-sc05_(A67G_S68G) |
| 121 | scFv: 38-02-A04-sc05 (Reference) (PRO) |
| 122 | scFv: 38-02-A04-sc05 (A67G_S68G) |
| 123 | scFv: 38-02-A04-sc05 (T101S_T146K)_(A67G_S68G) |

38-02-A04-sc06-based sequences

| | |
|---|---|
| 124 | VH: 38-02-A04-sc06 |
| 125 | VH: 38-02-A04-sc06_(T101S_T146K) |
| 126 | VL: 38-02-A04-sc06 (Reference) |
| 127 | VL: 38-02-A04-sc06_(A67G_S68G) |
| 128 | scFv: 38-02-A04-sc06 (Reference) (PRO) |
| 129 | scFv: 38-02-A04-sc06 (A67G_S68G) |
| 130 | scFv: 38-02-A04-sc06 (T101S_T146K)_(A67G_S68G) |

38-02-A04-sc13-based sequences

| | |
|---|---|
| 131 | VH: 38-02-A04-sc13 (PRO1352) |
| 132 | VL: 38-02-A04-sc13 (PRO1352) (Reference) |
| 133 | VL: 38-02-A04-sc13_(A67G_F68G) |
| 134 | scFv: 38-02-A04-sc13 (Reference) (PRO1352) |
| 135 | scFv: 38-02-A04-sc13 (A67G_S68G) (PRO3727) |

38-02-A04-sc16-based sequences

| | |
|---|---|
| 136 | VH: 38-02-A04-sc16 |
| 137 | VH: 38-02-A04-sc16_(G51C) |
| 138 | VL: 38-02-A04-sc16 (Reference) |
| 139 | VL: 38-02-A04-sc16_(T141C) (Reference) |
| 140 | VL: 38-02-A04-sc16_(A67G_F68G) |
| 141 | VL: 38-02-A04-sc16_(A67G_F68G_T141C) |
| 142 | scFv: 38-02-A04-sc16 (Reference) (PRO4148) |
| 143 | scFv: 38-02-A04-sc16 (A67G_S68G) (PRO4152) |
| 144 | scFv: 38-02-A04-sc16_(G51C) (T141C) (A67G_S68G) (PRO4156) |

38-27-C05-sc01-based sequences

| | |
|---|---|
| 145 | VH: 38-27-C05-sc01 |
| 146 | VH: 38-27-C05-sc01_(T101S_T146K) |
| 147 | VL: 38-27-C05-sc01 (Reference) |
| 148 | VL: 38-27-C05-sc01_(A67G_S68G) |
| 149 | scFv: 38-27-C05-sc01 (Reference) |
| 150 | scFv: 38-27-C05-sc01 (A67G_S68G) |
| 151 | scFv: 38-27-C05-sc01 (T101S_T146K) (A67G_S68G) |

Anti-CD3 antibody variable domains
28-21-D09-sc04-based sequences

| | |
|---|---|
| 152 | VH: 28-21-D09-sc04 (PRO2781) |
| 153 | VH: 28-21-D09-sc04_(T101S_T146K) |
| 154 | VL: 28-21-D09-sc04 (PRO2781) (Reference) |
| 155 | VL: 28-21-D09-sc04_(A67G_F68G) |
| 156 | scFv: 28-21-D09-sc04 (Reference) (PRO2781) |
| 157 | scFv: 28-21-D09-sc04 (A67G_S68G) (PRO3718) |
| 158 | scFv: 28-21-D09-sc04 (T101S_T146K) (A67G_S68G) |

28-21-D09-sc43-based sequences

| | |
|---|---|
| 159 | VH: 28-21-D09-sc43 |
| 160 | VH: 28-21-D09-sc43_(T101S_T146K) |
| 161 | VH: 28-21-D09-sc43_(G51C_T101S_T146K) |
| 162 | VL: 28-21-D09-sc43 (Reference) |
| 163 | VL: 28-21-D09-sc43_(A67G_F68G) |
| 164 | VL: 28-21-D09-sc43_(A67G_F68G_T141C) |
| 165 | scFv: 28-21-D09-sc43 (Reference) (PRO4187) |
| 166 | scFv: 28-21-D09-sc43 (A67G_S68G) (PRO4188) |
| 167 | scFv: 28-21-D09-sc43_(T101S_T146K) (Reference) (PRO4189) |
| 168 | scFv: 28-21-D09-sc43_(T101S_T146K) (A67G_S68G) (PRO4190) |
| 169 | scFv: 28-21-D09-sc43_(T101S_T146K) (G51C_T141C)_(A67G_S68G) (PRO4191) |

Anti-hSA antibody variable domains
19-04-A10-sc02-based sequences

| | |
|---|---|
| 170 | VH: 19-04-A10-sc02 (PRO2155) |
| 171 | VH: 19-04-A10-sc02_(G51C) |
| 172 | VH: 19-04-A10-sc02_(T101S_T146K) |
| 173 | VH: 19-04-A10-sc02_(G51C_T101S_T146K) |
| 174 | VL: 19-04-A10-sc02 (PRO2155) (Reference) |
| 175 | VL: 19-04-A10-sc02_(A67G_F68G) |
| 176 | VL: 19-04-A10-sc02_(A67G_F68G_T141C) |
| 177 | scFv: 19-04-A10-sc02 (Reference) (PRO2155) |
| 178 | scFv: 19-04-A10-sc02 (A67G_S68G) (PRO3721) |
| 179 | scFv: 19-04-A10-sc02 (T101S_T146K) (A67G_S68G) |
| 180 | scFv: 19-04-A10-sc02_(G51C_T141C) (A67G_S68G) (PRO3721) |
| 181 | scFv: 19-04-A10-sc02_(G51C_T141C) (T101S_T146K) (A67G_S68G) |

19-04-A10-sc11/12-based sequences

| | |
|---|---|
| 182 | VH: 19-04-A10-sc11 (PRO4112) |
| 183 | VH: 19-04-A10-sc12 (PRO4113) |
| 184 | VL: 19-04-A10-sc11/12 (PRO4112) (Reference) |
| 185 | VL: 19-04-A10-sc11/12_(A67G_F68G) |
| 186 | scFv: 19-04-A10-sc11 (Reference) (PRO4112) |
| 187 | scFv: 19-04-A10-sc11 (A67G_S68G) (PRO4116) |
| 188 | scFv: 19-04-A10-sc12 (Reference) (PRO4113) |
| 189 | scFv: 19-04-A10-sc12 (A67G_S68G) (PRO4117) |

19-01-H04-sc03-based sequences

| | |
|---|---|
| 190 | VH: 19-01-H04-sc03 |
| 191 | VH: 19-01-H04-sc03_(G51C) |
| 192 | VH: 19-01-H04-sc03_(T101S_T146K) |
| 193 | VH: 19-01-H04-sc03_(G51C_T101S_T146K) |
| 194 | VL: 19-01-H04-sc03 (Reference) |
| 195 | VL: 19-01-H04-sc03_(A67G_F68G) |
| 196 | VL: 19-01-H04-sc03_(A67G_F68G_T141C) |
| 197 | scFv: 19-01-H04-sc03 (Reference) |
| 198 | scFv: 19-01-H04-sc03 (A67G_S68G) |
| 199 | scFv: 19-01-H04-sc03 (T101S_T146K)_(A67G_S68G) |
| 200 | scFv: 19-01-H04-sc03_(G51C_T141C) (A67G_S68G) |
| 201 | scFv: 19-01-H04-sc03_(G51C_T141C) (T101S_T146K) (A67G_S68G) |

23-13-A01-sc03-based sequences

| | |
|---|---|
| 202 | VH: 23-13-A01-sc03 |
| 203 | VH: 23-13-A01-sc03_(G51C) |
| 204 | VH: 23-13-A01-sc03_(T101S_T146K) |
| 205 | VH: 23-13-A01-sc03_(G51C_T101S_T146K) |
| 206 | VL: 23-13-A01-sc03 (Reference) |
| 207 | VL: 23-13-A01-sc03_(A67G_F68G) |
| 208 | VL: 23-13-A01-sc03_(A67G_F68G_T141C) |
| 209 | scFv: 23-13-A01-sc03 (Reference) |
| 210 | scFv: 23-13-A01-sc03 (A67G_S68G) |
| 211 | scFv: 23-13-A01-sc03 (T101S_T146K) (A67G_S68G) |
| 212 | scFv: 23-13-A01-sc03_(G51C_T141C) (A67G_S68G) |
| 213 | scFv: 23-13-A01-sc03_(G51C_T141C) (T101S_T146K) (A67G_S68G) |

Anti-MSLN antibody variable domains
54-21-H03-sc01-based sequences

| | |
|---|---|
| 214 | VH: 54-21-H03-sc01 (PRO1922) |
| 215 | VH: 54-21-H03-sc01_(G51C) |
| 216 | VH: 54-21-H03-sc01_(T101S_T146K) |
| 217 | VH: 54-21-H03-sc01_(G51C_T101S_T146K) |
| 218 | VL: 54-21-H03-sc01 (PRO1922) (Reference) |
| 219 | VL: 54-21-H03-sc01_(A67G_F68G) |
| 220 | VL: 54-21-H03-sc01_(A67G_F68G_G141C) |
| 221 | scFv: 54-21-H03-sc01 (Reference) (PRO1922) |
| 222 | scFv: 54-21-H03-sc01 (A67G_S68G) (PRO3732) |
| 223 | scFv: 54-21-H03-sc01 (T101S_T146K) (A67G_S68G) |
| 224 | scFv: 54-21-H03-sc01_(G51C_T141C) (A67G_S68G) |
| 225 | scFv: 54-21-H03-sc01_(G51C_T141C) (T101S_T146K) (A67G_S68G) |

54-32-A07-sc02-based sequences

| | |
|---|---|
| 226 | VH: 54-32-A07-sc02 (PRO1925) |
| 227 | VH: 54-32-A07-sc02_(T101S_T146K) |
| 228 | VL: 54-32-A07-sc02 (PRO1925) (Reference) |
| 229 | VL: 54-32-A07-sc02_(A67G_S68G) |
| 230 | scFv: 54-32-A07-sc02 (Reference) (PRO1925) |
| 231 | scFv: 54-32-A07-sc02 (A67G_S68G) |
| 232 | scFv: 54-32-A07-sc02 (T101S_T146K)_(A67G_S68G) |

54-32-A07-sc06-based sequences

| | |
|---|---|
| 233 | VH: 54-32-A07-sc06 (PRO2306) |
| 234 | VH: 54-32-A07-sc06_(G51C) (PRO2309) |
| 235 | VH: 54-32-A07-sc06_(T101S_T146K) |
| 236 | VH: 54-32-A07-sc06_(G51C_T101S_T146K) |
| 237 | VL: 54-32-A07-sc06 (PRO2306) (Reference) |
| 238 | VL: 54-32-A07-sc06_(G141C) (PRO2309) (Reference) |
| 239 | VL: 54-32-A07-sc06_(A67G_S68G) |
| 240 | VL: 54-32-A07-sc06_(A67G_S68G_G141C) |
| 241 | scFv: 54-32-A07-sc06 (Reference) (PRO2306) |
| 242 | scFv: 54-32-A07-sc06 (A67G_S68G) |
| 243 | scFv: 54-32-A07-sc06 (T101S_T146K)_(A67G_S68G) |
| 244 | scFv: 54-32-A07-sc06_(G51C_T141C) (Reference) (PRO2309) |
| 245 | scFv: 54-32-A07-sc06_(G51C_T141C) (A67G_S68G) (PRO3733) |
| 246 | scFv: 54-32-A07-sc06_(G51C_T141C) (T101S_T146K) (A67G_S68G) |

54-01-G02-sc01-based sequences

| | |
|---|---|
| 247 | VH: 54-01-G02-sc01 (PRO1783) |
| 248 | VH: 54-01-G02-sc01_(N66A) (PRO2197) |
| 249 | VH: 54-01-G02-sc01_(T101S_T146K) |
| 250 | VH: 54-01-G02-sc01_(N66A_T101S_T146K) |
| 251 | VL: 54-01-G02-sc01 (Reference) |
| 252 | VL: 54-01-G02-sc01_(A67G_S68G) |
| 253 | scFv: 54-01-G02-sc01 (Reference) (PRO1783) |
| 254 | scFv: 54-01-G02-sc01 (A67G_S68G) |
| 255 | scFv: 54-01-G02-sc01_(N66A) (A67G_S68G) |
| 256 | scFv: 54-01-G02-sc01_(N66A_T101S_T146K) (A67G_S68G) |

54-22-H03-sc01-based sequences

| | |
|---|---|
| 257 | VH: 54-22-H03-sc01 (PRO1795) |
| 258 | VH: 54-22-H03-sc01_(T101S_T146K) |
| 259 | VL: 54-22-H03-sc01 (PRO1795) (Reference) |
| 260 | VL: 54-22-H03-sc01_(A67G_S68G) |
| 261 | scFv: 54-22-H03-sc01 (Reference) (PRO1795) |
| 262 | scFv: 54-22-H03-sc01 (A67G_S68G) |
| 263 | scFv: 54-22-H03-sc01 (T101S_T146K) (A67G_S68G) |

Anti-ROR1 antibody variable domains
55-39-G02-sc02-based sequences

| | |
|---|---|
| 264 | VH: 55-39-G02-sc02 (PRO2062) |
| 265 | VH: 55-39-G02-sc02_(G51C) |
| 266 | VH: 55-39-G02-sc02_(T101S_T146K) |
| 267 | VH: 55-39-G02-sc02_(G51C_T101S_T146K) |
| 268 | VL: 55-39-G02-sc02 (PRO2062) (Reference) |
| 269 | VL: 55-39-G02-sc02_(A67G_F68G) |
| 270 | VL: 55-39-G02-sc02_(A67G_F68G_T141C) |
| 271 | scFv: 55-39-G02-sc02 (Reference) (PRO2062) |
| 272 | scFv: 55-39-G02-sc02 (A67G_S68G) |
| 273 | scFv: 55-39-G02-sc02_(T101S_T146K) (A67G_S68G) |
| 274 | scFv: 55-39-G02-sc02_(G51C_T101S_T146K) (A67G_S68G_T141C) |

55-39-G02-sc03-based sequences

| | |
|---|---|
| 275 | VH: 55-39-G02-sc03 (PRO2271) |
| 276 | VH: 55-39-G02-sc03_(G51C) |
| 277 | VH: 55-39-G02-sc03_(T101S_T146K) |
| 278 | VH: 55-39-G02-sc03_(G51C_T101S_T146K) |
| 279 | VL: 55-39-G02-sc03 (PRO2271) (Reference) |
| 280 | VL: 55-39-G02-sc03_(A67G_F68G) |
| 281 | VL: 55-39-G02-sc03_(A67G_F68G_T141C) |
| 282 | scFv: 55-39-G02-sc03 (Reference) (PRO2271) |
| 283 | scFv: 55-39-G02-sc03 (A67G_S68G) |
| 284 | scFv: 55-39-G02-sc03_(T101S_T146K) (A67G_S68G) |
| 285 | scFv: 55-39-G02-sc03_(G51C_T101S_T146K) (A67G_S68G_T141C) |

55-38-D07-based VH/VL sequences

| | |
|---|---|
| 286 | VH: 55-38-D07-sc02 (PRO2060) |
| 287 | VH: 55-38-D07-sc02_(T101S_T146K) |
| 288 | VH: 55-38-D07-sc02_(G51C) |
| 289 | VH: 55-38-D07-sc02_(G51C_T101S_T146K) |
| 290 | VH: 55-38-D07-sc09 |
| 291 | VH: 55-38-D07-sc09_(G51C) |
| 292 | VL: 55-38-D07-sc02 (PRO2060) (Reference) |
| 293 | VL: 55-38-D07-sc02_(A67G_F68G) |
| 294 | VL: 55-38-D07-sc02_(A67G_F68G_T141C) |
| 295 | scFv: 55-38-D07-sc02 (Reference) (PRO2060) |
| 296 | scFv: 55-38-D07-sc02 (A67G_S68G) (PRO3735) |
| 297 | scFv: 55-38-D07-sc02_(T101S_T146K) (A67G_S68G) |
| 298 | scFv: 55-38-D07-sc02_(G51C_T101S_T146K) (A67G_S68G_T141C) |
| 299 | scFv: 55-38-D07-sc09 (Reference) (PRO4134) |
| 300 | scFv: 55-38-D07-sc09 (A67G_S68G) (PRO4139) |
| 301 | scFv: 55-38-D07-sc09_(G51C) (A67G_S68G_T141C) (PRO4143) |

Anti-HER2 antibody variable domains
Trastuzumab-based sequences

| | |
|---|---|
| 302 | VH: Trastuzumab_lambda-cap (PRO0286) |
| 303 | VL: Trastuzumab_lambda-cap (PRO0286) (Reference) |
| 304 | VL: Trastuzumab_lambda-cap_(A67G_S68G) |
| 305 | scFv: Trastuzumab_lambda-cap (Reference) (PRO0286) |
| 306 | scFv: Trastuzumab_lambda-cap (A67G_S68G) (PRO3716) |

Pertuzumab-based sequences

| | |
|---|---|
| 307 | VH: Pertuzumab_lambda-cap (PRO1812) |
| 308 | VL: Pertuzumab_lambda-cap (PRO1812) (Reference) |
| 309 | VL: Pertuzumab_lambda-cap (A67G_S68G) |
| 310 | scFv: Pertuzumab_lambda-cap (Reference) (PRO1812) |
| 311 | scFv: Pertuzumab_lambda-cap (A67G_S68G) (PRO3717) |

Anti-IL23R antibody variable domains
14-11-D07-sc04-based sequences

| | |
|---|---|
| 312 | VH: 14-11-D07-sc04 (PRO0517) |
| 313 | VH: 14-11-D07-sc04_(T101S_T146K) |
| 314 | VL: 14-11-D07-sc01 (Reference) |
| 315 | VL: 14-11-D07-sc02 (Reference) |
| 316 | VL: 14-11-D07-sc04 (PRO0517) (Reference) |
| 317 | VL: 14-11-D07-sc01_(A67G_S68G) |
| 318 | VL: 14-11-D07-sc02_(A67G_S68G) |
| 319 | VL: 14-11-D07-sc04_(A67G_S68G) |
| 320 | scFv: 14-11-D07-sc01 (Reference) (PRO0517) |
| 321 | scFv: 14-11-D07-sc01 (A67G_S68G) |
| 322 | scFv: 14-11-D07-sc02 (A67G_S68G) |
| 323 | scFv: 14-11-D07-sc04 (A67G_S68G) (PRO3719) |
| 324 | scFv: 14-11-D07-sc01_(T101S_T146K) (A67G_S68G) |
| 325 | scFv: 14-11-D07-sc02_(T101S_T146K) (A67G_S68G) |
| 326 | scFv: 14-11-D07-sc04_(T101S_T146K) (A67G_S68G) (PRO3719) |

Anti-IL17 antibody variable domains
27-07-G02-based sequences

| | |
|---|---|
| 327 | VH: 27-07-G02-sc01 (PRO0561) |
| 328 | VH: 27-07-G02-sc01_(T101S_T146K) |
| 329 | VH: 27-07-G02-sc02 |
| 330 | VH: 27-07-G02-sc02_(T101S_T146K) |
| 331 | VL: 27-07-G02-sc01 (PRO0561) (Reference) |
| 332 | VL: 27-07-G02-sc01_(A67G_F68G) |
| 333 | scFv: 27-07-G02-sc01 (Reference) (PRO0561) |
| 334 | scFv: 27-07-G02-sc01 (A67G_F68G) (PRO3722) |
| 335 | scFv: 27-07-G02-sc01_(T101S_T146K) (A67G_F68G) |
| 336 | scFv: 27-07-G02-sc02 (A67G_F68G) |
| 337 | scFv: 27-07-G02-sc02 (T101S_T146K) (A67G_F68G) |

27-31-C04-based sequences

| | |
|---|---|
| 338 | VH: 27-31-C04-sc01 |
| 339 | VH: 27-31-C04-sc01 (T101S_T146K) |
| 340 | VH: 27-31-C04-sc02 |
| 341 | VH: 27-31-C04-sc02_(T101S_T146K) |
| 342 | VL: 27-31-C04-sc01 (Reference) |
| 343 | VL: 27-31-C04-sc01_(A67G_F68G) |
| 344 | scFv: 27-31-C04-sc01 (Reference) |
| 345 | scFv: 27-31-C04-sc01 (A67G_F68G) |
| 346 | scFv: 27-31-C04-sc01_(T101S_T146K) (A67G_F68G) |
| 347 | scFv: 27-31-C04-sc02 (A67G_F68G) |
| 348 | scFv: 27-31-C04-sc02_(T101S_T146K) (A67G_F68G) |

Anti-PD-L1 low affinity antibody variable domains
33-03-G02_Q108A_G109A-based sequences

| | |
|---|---|
| 349 | VH: 33-03-G02 (PRO1434) |
| 350 | VH: 33-03-G02 (PRO1434) (T101S_T146K) |
| 351 | VL: 33-03-G02_Q108A_G109A (PRO1434) (Reference) |
| 352 | VL: 33-03-G02_Q108A_G109A (A67G_F68G) |
| 353 | scFv: 33-03-G02_Q108A_G109A (Reference) (PRO1434) |
| 354 | scFv: 33-03-G02_Q108A_G109A (A67G_F68G) (PRO3725) |
| 355 | scFv: 33-03-G02_Q108A_G109A (T101S_T146K) (A67G_F68G) |

Anti-TNFα antibody variable domains
16-19-B11-sc01-based sequences

| | |
|---|---|
| 356 | VH: 16-19-B11-sc01 |
| 357 | VH: 16-19-B11-sc01 (T101S, T146K) |
| 358 | VL: 16-19-B11-sc01 (Reference) |
| 359 | VL: 16-19-B11-sc01 (A67G_F68G) |
| 360 | scFv: 16-19-B11-sc01 (Reference) |
| 361 | scFv: 16-19-B11-sc01 (A67G_F68G) |
| 362 | scFv: 16-19-B11-sc01 (T101S_T146K)_(A67G_F68G) |

16-19-B11-sc02-based sequences

| | |
|---|---|
| 363 | VH: 16-19-B11-sc02 |
| 364 | VH: 16-19-B11-sc02 (T101S, T146K) |
| 365 | VL: 16-19-B11-sc02 (Reference) |
| 366 | VL: 16-19-B11-sc02 (A67G_F68G) |
| 367 | scFv: 16-19-B11-sc02 (Reference) |
| 368 | scFv: 16-19-B11-sc02 (A67G_F68G) |
| 369 | scFv: 16-19-B11-sc02 (T101S_T146K)_(A67G_F68G) |

16-19-B11-sc06-based sequences

| | |
|---|---|
| 370 | VH: 16-19-B11-sc06 |
| 371 | VH: 16-19-B11-sc06 (T101S, T146K) |
| 372 | VL: 16-19-B11-sc06 (Reference) |
| 373 | VL: 16-19-B11-sc06 (A67G_F68G) |
| 374 | scFv: 16-19-B11-sc06 (Reference) |
| 375 | scFv: 16-19-B11-sc06 (A67G_F68G) |
| 376 | scFv: 16-19-B11-sc06 (T101S_T146K)_(A67G_F68G) |

16-19-B11-sc10-based sequences

| | |
|---|---|
| 377 | VH: 16-19-B11-sc10 |
| 378 | VH: 16-19-B11-sc10 (T101S, T146K) |
| 379 | VL: 16-19-B11-sc10 (Reference) |
| 380 | VL: 16-19-B11-sc10 (A67G_F68G) |
| 381 | scFv: 16-19-B11-sc10 (Reference) |
| 382 | scFv: 16-19-B11-sc10 (A67G_F68G) |
| 383 | scFv: 16-19-B11-sc10 (T101S_T146K)_(A67G_F68G) |

16-19-B11-sc11-based sequences

| | |
|---|---|
| 384 | VH: 16-19-B11-sc11 |
| 385 | VH: 16-19-B11-sc11 (T101S, T146K) |
| 386 | VL: 16-19-B11-sc11 (Reference) |
| 387 | VL: 16-19-B11-sc11 (A67G_F68G) |
| 388 | scFv: 16-19-B11-sc11 (Reference) |
| 389 | scFv: 16-19-B11-sc11 (A67G_F68G) |
| 390 | scFv: 16-19-B11-sc11 (T101S_T146K)_(A67G_F68G) |

16-18-E11-sc01-based sequences

| | |
|---|---|
| 391 | VH: 16-18-E11-sc01 |
| 392 | VH: 16-18-E11-sc01 (T101S, T146K) |
| 393 | VL: 16-18-E11-sc01 (Reference) |
| 394 | VL: 16-18-E11-sc01 (A67G_F68G) |
| 395 | scFv: 16-18-E11-sc01 (Reference) |
| 396 | scFv: 16-18-E11-sc01 (A67G_F68G) |
| 397 | scFv: 16-18-E11-sc01 (T101S_T146K)_(A67G_F68G) |

16-18-E11-sc02-based sequences

| | |
|---|---|
| 398 | VH: 16-18-E11-sc02 |
| 399 | VH: 16-18-E11-sc02 (T101S, T146K) |
| 400 | VL: 16-18-E11-sc02 (Reference) |
| 401 | VL: 16-18-E11-sc02 (A67G_F68G) |
| 402 | scFv: 16-18-E11-sc02 (Reference) |
| 403 | scFv: 16-18-E11-sc02 (A67G_F68G) |
| 404 | scFv: 16-18-E11-sc02 (T101S_T146K)_(A67G_F68G) |

16-18-E11-sc06-based sequences

| | |
|---|---|
| 405 | VH: 16-18-E11-sc06 |
| 406 | VH: 16-18-E11-sc06 (T101S, T146K) |
| 407 | VL: 16-18-E11-sc06 (Reference) |
| 408 | VL: 16-18-E11-sc06 (A67G_F68G) |
| 409 | scFv: 16-18-E11-sc06 (Reference) |
| 410 | scFv: 16-18-E11-sc06 (A67G_F68G) |
| 411 | scFv: 16-18-E11-sc06 (T101S_T146K)_(A67G_F68G) |

17-21-A01-sc01-based sequences

| | |
|---|---|
| 412 | VH: 17-21-A01-sc01 |
| 413 | VH: 17-21-A01-sc01 (T101S, T146K) |
| 414 | VL: 17-21-A01-sc01 (Reference) |
| 415 | VL: 17-21-A01-sc01 (A67G_F68G) |
| 416 | scFv: 17-21-A01-sc01 (Reference) |
| 417 | scFv: 17-21-A01-sc01 (A67G_F68G) |
| 418 | scFv: 17-21-A01-sc01 (T101S_T146K)_(A67G_F68G) |

17-21-A01-sc02-based sequences

| | |
|---|---|
| 419 | VH: 17-21-A01-sc02 |
| 420 | VH: 17-21-A01-sc02 (T101S, T146K) |
| 421 | VL: 17-21-A01-sc02 (Reference) |
| 422 | VL: 17-21-A01-sc02 (A67G_F68G) |
| 423 | scFv: 17-21-A01-sc02 (Reference) |
| 424 | scFv: 17-21-A01-sc02 (A67G_F68G) |
| 425 | scFv: 17-21-A01-sc02 (T101S_T146K)_(A67G_F68G) |

17-21-A01-sc03-based sequences

| | |
|---|---|
| 426 | VH: 17-21-A01-sc03 |
| 427 | VH: 17-21-A01-sc03 (T101S, T146K) |
| 428 | VL: 17-21-A01-sc03 (Reference) |
| 429 | VL: 17-21-A01-sc03 (A67G_F68G) |
| 430 | scFv: 17-21-A01-sc03 (Reference) |
| 431 | scFv: 17-21-A01-sc03 (A67G_F68G) |
| 432 | scFv: 17-21-A01-sc03 (T101S_T146K)_(A67G_F68G) |

16-14-G10-sc01-based sequences

| | |
|---|---|
| 433 | VH: 16-14-G10-sc01 |
| 434 | VH: 16-14-G10-sc01 (T101S, T146K) |
| 435 | VL: 16-14-G10-sc01 (Reference) |
| 436 | VL: 16-14-G10-sc01 (A67G_F68G) |
| 437 | scFv: 16-14-G10-sc01 (Reference) |
| 438 | scFv: 16-14-G10-sc01 (A67G_F68G) |
| 439 | scFv: 16-14-G10-sc01 (T101S_T146K)_(A67G_F68G) |

16-14-G10-sc02-based sequences

| | |
|---|---|
| 440 | VH: 16-14-G10-sc02 |
| 441 | VH: 16-14-G10-sc02 (T101S, T146K) |
| 442 | VL: 16-14-G10-sc02 (Reference) |
| 443 | VL: 16-14-G10-sc02 (A67G_F68G) |
| 444 | scFv: 16-14-G10-sc02 (Reference) |
| 445 | scFv: 16-14-G10-sc02 (A67G_F68G) |
| 446 | scFv: 16-14-G10-sc02 (T101S_T146K)_(A67G_F68G) |

16-14-G10-sc03-based sequences

| | |
|---|---|
| 447 | VH: 16-14-G10-sc03 |
| 448 | VH: 16-14-G10-sc03 (T101S, T146K) |
| 449 | VL: 16-14-G10-sc03 (Reference) |
| 450 | VL: 16-14-G10-sc03 (A67G_F68G) |
| 451 | scFv: 16-14-G10-sc03 (Reference) |
| 452 | scFv: 16-14-G10-sc03 (A67G_F68G) |
| 453 | scFv: 16-14-G10-sc03 (T101S_T146K)_(A67G_F68G) |

16-02-B03-sc02-based sequences

| | |
|---|---|
| 454 | VH: 16-02-B03-sc02 |
| 455 | VH: 16-02-B03-sc02 (T101S, T146K) |
| 456 | VL: 16-02-B03-sc02 (Reference) |
| 457 | VL: 16-02-B03-sc02 (A67G_F68G) |
| 458 | scFv: 16-02-B03-sc02 (Reference) |
| 459 | scFv: 16-02-B03-sc02 (A67G_F68G) |
| 460 | scFv: 16-02-B03-sc02 (T101S_T146K)_(A67G_F68G) |

16-02-B03-sc03-based sequences

| | |
|---|---|
| 461 | VH: 16-02-B03-sc03 |
| 462 | VH: 16-02-B03-sc03 (T101S, T146K) |
| 463 | VL: 16-02-B03-sc03 (Reference) |
| 464 | VL: 16-02-B03-sc03 (A67G_F68G) |
| 465 | scFv: 16-02-B03-sc03 (Reference) |
| 466 | scFv: 16-02-B03-sc03 (A67G_F68G) |
| 467 | scFv: 16-02-B03-sc03 (T101S_T146K)_(A67G_F68G) |

16-02-B03-sc06-based sequences

| | |
|---|---|
| 468 | VH: 16-02-B03-sc06 |
| 469 | VH: 16-02-B03-sc06 (T101S, T146K) |
| 470 | VL: 16-02-B03-sc06 (Reference) |
| 471 | VL: 16-02-B03-sc06 (A67G_F68G) |
| 472 | scFv: 16-02-B03-sc06 (Reference) |
| 473 | scFv: 16-02-B03-sc06 (A67G_F68G) |
| 474 | scFv: 16-02-B03-sc06 (T101S_T146K)_(A67G_F68G) |

16-23-C06-sc02-based sequences

| | |
|---|---|
| 475 | VH: 16-23-C06-sc02 |
| 476 | VH: 16-23-C06-sc02 (T101S, T146K) |
| 477 | VL: 16-23-C06-sc02 (Reference) |
| 478 | VL: 16-23-C06-sc02 (A67G_F68G) |
| 479 | scFv: 16-23-C06-sc02 (Reference) |
| 480 | scFv: 16-23-C06-sc02 (A67G_F68G) |
| 481 | scFv: 16-23-C06-sc02 (T101S_T146K)_(A67G_F68G) |

16-23-C06-sc03-based sequences

| | |
|---|---|
| 482 | VH: 16-23-C06-sc03 |
| 483 | VH: 16-23-C06-sc03 (T101S, T146K) |
| 484 | VL: 16-23-C06-sc03 (Reference) |
| 485 | VL: 16-23-C06-sc03 (A67G_F68G) |
| 486 | scFv: 16-23-C06-sc03 (Reference) |
| 487 | scFv: 16-23-C06-sc03 (A67G_F68G) |
| 488 | scFv: 16-23-C06-sc03 (T101S_T146K)_(A67G_F68G) |

16-23-C06-sc06-based sequences

| | |
|---|---|
| 489 | VH: 16-23-C06-sc06 |
| 490 | VH: 16-23-C06-sc06 (T101S, T146K) |
| 491 | VL: 16-23-C06-sc06 (Reference) |
| 492 | VL: 16-23-C06-sc06 (A67G_F68G) |
| 493 | scFv: 16-23-C06-sc06 (Reference) |
| 494 | scFv: 16-23-C06-sc06 (A67G_F68G) |
| 495 | scFv: 16-23-C06-sc06 (T101S_T146K)_(A67G_F68G) |

TABLE 2
Other sequences related to the present invention.

| SEQ ID | | |
|---|---|---|
| NO: | Description | Sequence |
| 496 | VH3 | EVQLVESGGGLVQPGGSLRLSCAASGFSFSANYYPCWVRQAPGKGLEWIGCIYGGSSDITYD |
| | | ANWTKGRFTISRDNSKNTVYLQMNSLRAEDTAVYYCARSAWYSGWGGDLWGQGTLVTVSS |
| 497 | VH3_(51C) | EVQLVESGGGLVQPGGSLRLSCAASGFSFSANYYPCWVRQAPGKCLEWIGCIYGGSSDITYD |
| | | ANWTKGRFTISRDNSKNTVYLQMNSLRAEDTAVYYCARSAWYSGWGGDLWGQGTLVTVSS |
| 498 | VH3_(56A) | EVQLVESGGGLVQPGGSLRLSCAASGFSFSANYYPCWVRQAPGKGLEWIACIYGGSSDITYD |
| | | ANWTKGRFTISRDNSKNTVYLQMNSLRAEDTAVYYCARSAWYSGWGGDLWGQGTLVTVSS |
| 499 | VH3_(51C, 56A) | EVQLVESGGGLVQPGGSLRLSCAASGFSFSANYYPCWVRQAPGKCLEWIACIYGGSSDITYD |
| | | ANWTKGRFTISRDNSKNTVYLQMNSLRAEDTAVYYCARSAWYSGWGGDLWGQGTLVTVSS |
| 500 | VH3_(105F) | EVQLVESGGGLVQPGGSLRLSCAASGFSFSANYYPCWVRQAPGKGLEWIGCIYGGSSDITYD |
| | | ANWTKGRFTISRDNSKNTVYLQMNSLRAEDTAVYFCARSAWYSGWGGDLWGQGTLVTVSS |
| 501 | VH3_(51C, 105F) | EVQLVESGGGLVQPGGSLRLSCAASGFSFSANYYPCWVRQAPGKCLEWIGCIYGGSSDITYD |
| | | ANWTKGRFTISRDNSKNTVYLQMNSLRAEDTAVYFCARSAWYSGWGGDLWGQGTLVTVSS |
| 502 | VH3_(89L) | EVQLVESGGGLVQPGGSLRLSCAASGFSFSANYYPCWVRQAPGKGLEWIGCIYGGSSDITYD |
| | | ANWTKGRFTISRDNSKNTLYLQMNSLRAEDTAVYYCARSAWYSGWGGDLWGQGTLVTVSS |
| 503 | VH3_(51C, 89L) | EVQLVESGGGLVQPGGSLRLSCAASGFSFSANYYPCWVRQAPGKCLEWIGCIYGGSSDITYD |
| | | ANWTKGRFTISRDNSKNTLYLQMNSLRAEDTAVYYCARSAWYSGWGGDLWGQGTLVTVSS |
| 504 | VH3_(56A, 89L) | EVQLVESGGGLVQPGGSLRLSCAASGFSFSANYYPCWVRQAPGKGLEWIACIYGGSSDITYD |
| | | ANWTKGRFTISRDNSKNTLYLQMNSLRAEDTAVYYCARSAWYSGWGGDLWGQGTLVTVSS |
| 505 | VH3_(51C, 56A, 89L) | EVQLVESGGGLVQPGGSLRLSCAASGFSFSANYYPCWVRQAPGKCLEWIACIYGGSSDITYD |
| | | ANWTKGRFTISRDNSKNTLYLQMNSLRAEDTAVYYCARSAWYSGWGGDLWGQGTLVTVSS |
| 506 | VH3_(56A, 105F) | EVQLVESGGGLVQPGGSLRLSCAASGFSFSANYYPCWVRQAPGKGLEWIACIYGGSSDITYD |
| | | ANWTKGRFTISRDNSKNTVYLQMNSLRAEDTAVYFCARSAWYSGWGGDLWGQGTLVTVSS |
| 507 | VH3_(51C, 56A, 105F) | EVQLVESGGGLVQPGGSLRLSCAASGFSFSANYYPCWVRQAPGKCLEWIACIYGGSSDITYD |
| | | ANWTKGRFTISRDNSKNTVYLQMNSLRAEDTAVYFCARSAWYSGWGGDLWGQGTLVTVSS |
| 508 | VH3_(56A, 89L, 105F) | EVQLVESGGGLVQPGGSLRLSCAASGFSFSANYYPCWVRQAPGKGLEWIACIYGGSSDITYD |
| | | ANWTKGRFTISRDNSKNTLYLQMNSLRAEDTAVYFCARSAWYSGWGGDLWGQGTLVTVSS |
| 509 | VH3_(51C, 56A, 89L, | EVQLVESGGGLVQPGGSLRLSCAASGFSFSANYYPCWVRQAPGKCLEWIACIYGGSSDITYD |
| | 105F) | ANWTKGRFTISRDNSKNTLYLQMNSLRAEDTAVYFCARSAWYSGWGGDLWGQGTLVTVSS |
| 510 | VH1a | QVQLVQSGAEVKKPGSSVKVSCKASGIDFNSNYYMCWVRQAPGQGLEWMGCIYVGSHVNT |
| | | YYANWAKGRVTITADESTSTAYMELSSLRSEDTAVYYCATSGSSVLYFKFWGQGTLVTVSS |
| 511 | VH1b | QVQLVQSGAEVKKPGASVKVSCKASGIDFNSNYYMCWVRQAPGQGLEWMGCIYVGSHVNT |
| | | YYANWAKGRVTMTRDTSISTAYMELSSLRSEDTAVYYCATSGSSVLYFKFWGQGTLVTVSS |
| 512 | VH4 | QVQLQESGPGLVKPSETLSLTCTVSGIDFNSNYYMCWIRQPPGKGLEWIGCIYVGSHVNTYYA |
| | | NWAKGRVTISVDTSKNQFSLKLSSVTAADTAVYYCATSGSSVLYFKFWGQGTLVTVSS |
| 513 | VH5 | EVQLVQSGAEVKKPGESLKISCKGSGIDFNSNYYMCWVRQMPGKGLEWMGCIYVGSHVNTY |
| | | YANWAKGQVTISADKSISTAYLQWSSLKASDTAMYYCATSGSSVLYFKFWGQGTLVTVSS |
| 514 | VH6 | QVQLQQSGPGLVKPSQTLSLTCAISGIDFNSNYYMCWIRQSPGRGLEWLGCIYVGSHVNTYY |
| | | ANWAKGRITINPDTSKNQFSLQLNSVTPEDTAVYYCATSGSSVLYFKFWGQGTLVTVSS |
| 515 | Vkappa1_V1 | DIQMTQSPSSLSASVGDRVTITCQASQSINNVLAWYQQKPGKAPKLLIYRASTLASGVPSRFS |
| | | GSGSGTDFTLTISSLQPEDFATYYCQSSYGNYGDFGQGTKVEIKR |
| 516 | Vkappa1_V1_(Q24R) | DIQMTQSPSSLSASVGDRVTITCRASQSINNVLAWYQQKPGKAPKLLIYRASTLASGVPSRFS |
| | | GSGSGTDFTLTISSLQPEDFATYYCQSSYGNYGDFGQGTKVEIKR |
| 517 | Vkappa1_V2 | DIQMTQSPASLSASVGDRVTITCQASQSIGTYLAWYQQKPGKPPKLLIYRAFILASGVPSRFSG |
| | | SGSGTDFTLTISSLQPEDFATYYCQSNFYSDSTTIGPNAFGQGTKVEIKR |
| 518 | Vkappa1_V2_(Q24R) | DIQMTQSPASLSASVGDRVTITCRASQSIGTYLAWYQQKPGKPPKLLIYRAFILASGVPSRFSG |
| | | SGSGTDFTLTISSLQPEDFATYYCQSNFYSDSTTIGPNAFGQGTKVEIKR |
| 519 | Vkappa1_V1_λ-LFW4 | DIQMTQSPSSLSASVGDRVTITCQASQSINNVLAWYQQKPGKAPKLLIYRASTLASGVPSRFS |
| | | GSGSGTDFTLTISSLQPEDFATYYCQSSYGNYGDFGTGTKVTVLG |
| 520 | Vkappa1_V1_λ- | DIQMTQSPSSLSASVGDRVTITCQASQSINNVLAWYQQKPGKAPKLLIYRASTLASGVPSRFS |
| | LFW4 (141C) | GSGSGTDFTLTISSLQPEDFATYYCQSSYGNYGDFGCGTKVTVLG |
| 521 | Vkappa1_V1_λ- | DIQMTQSPSSLSASVGDRVTITCRASQSINNVLAWYQQKPGKAPKLLIYRASTLASGVPSRFS |
| | LFW4_(Q24R) | GSGSGTDFTLTISSLQPEDFATYYCQSSYGNYGDFGTGTKVTVLG |
| 522 | Vkappa1_V1_λ- | DIQMTQSPSSLSASVGDRVTITCRASQSINNVLAWYQQKPGKAPKLLIYRASTLASGVPSRFS |
| | LFW4_(Q24R, 141C) | GSGSGTDFTLTISSLQPEDFATYYCQSSYGNYGDFGCGTKVTVLG |
| 523 | Vkappa1_V2_λ-LFW4 | DIQMTQSPASLSASVGDRVTITCQASQSIGTYLAWYQQKPGKPPKLLIYRAFILASGVPSRFSG |
| | | SGSGTDFTLTISSLQPEDFATYYCQSNFYSDSTTIGPNAFGTGTKVTVLG |
| 524 | Vkappa1_V2_λ- | DIQMTQSPASLSASVGDRVTITCQASQSIGTYLAWYQQKPGKPPKLLIYRAFILASGVPSRFSG |
| | LFW4_(141C) | SGSGTDFTLTISSLQPEDFATYYCQSNFYSDSTTIGPNAFGCGTKVTVLG |
| 525 | Vkappa1_V2_λ- | DIQMTQSPASLSASVGDRVTITCRASQSIGTYLAWYQQKPGKPPKLLIYRAFILASGVPSRFSG |
| | LFW4 (Q24R) | SGSGTDFTLTISSLQPEDFATYYCQSNFYSDSTTIGPNAFGTGTKVTVLG |
| 526 | Vkappa1_V2_λ- | DIQMTQSPASLSASVGDRVTITCRASQSIGTYLAWYQQKPGKPPKLLIYRAFILASGVPSRFSG |
| | LFW4 (Q24R, 141C) | SGSGTDFTLTISSLQPEDFATYYCQSNFYSDSTTIGPNAFGCGTKVTVLG |
| 527 | Vλ germline-based | FGTGTKVTVLG |
| | FR4 (Sk17) | |
| 528 | Vλ germline-based | FGGGTKLTVLG |
| | FR4 (Sk12) | |
| 529 | Vλ germline-based | FGGGTQLIILG |
| | FR4 | |
| 530 | Vλ germline-based | FGEGTELTVLG |
| | FR4 | |
| 531 | Vλ germline-based | FGSGTKVTVLG |
| | FR4 | |
| 532 | Vλ germline-based | FGGGTQLTVLG |
| | FR4 | |
| 533 | Vλ germline-based | FGGGTQLTALG |
| | FR4 | |
| 534 | Vλ germline-based | FGCGTKVTVLG |
| | FR4_G141C | |
| 535 | Vλ germline-based | FGTGTKLTVLG |
| | FR4_G141T | |
| 536 | LCDR2 | X1GGX2X3X4X5 |
| 537 | Linker L1 | (GGGGS)n, wherein n = 1, 2, 3, 4 or 5 |
| 538 | Linker L1 | GGGGSGGGGS |
| 539 | Linker L2 | (GGGGS)n, wherein n = 1, 2, 3, 4, 5, 6, 7 or 8 |
| 540 | Linker L2 | GGGGSGGGGSGGGGGGGGS |

TABLE 3
Examples of anti-PDL1 × CD137 × hSA multispecific
antibodies, according to the present invention.

| | |
|---|---|
| SEQ ID NO: | Description |

PRO1480 variants

| | |
|---|---|
| 544 | PRO1480_unmodified (Reference) |
| 545 | PRO1480: “GG”-PDL1 BD |
| 743 | PRO1480: “GG”-PDL1 BD, “GG”-CD137 BD |
| 546 | PRO1480: “GG”-PDL1 BD, “GG”-CD137 BD, “GG”-hSA BD |
| 547 | PRO1480_“SK”_(T301S, T346K, T701S, T746K, T1101S, T1146K): |
| | “GG”-PDL1 BD |
| 548 | PRO1480_“SK”_(T301S, T346K, T701S, T746K, T1101S, T1146K): |
| | “GG”-PDL1 BD, “GG”-CD137 BD, “GG”-hSA BD |
| 549 | PRO1480_“SK”_(T301S, T346K, T701S, T746K, T1101S, |
| | T1146K)_N272Q: “GG”-PDL1 BD |
| 550 | PRO1480_“SK”_(T301S, T346K, T701S, T746K, T1101S, |
| | T1146K)_N272Q: “GG”-PDL1 BD; “GG”-CD137 BD; “GG”-hSA BD |
| 551 | PRO1480_“SK”_(T301S, T346K, T701S, T746K, T1101S, |
| | T1146K)_N272Q: “DiS” hSA-BD, (Reference) |
| 552 | PRO1480_“SK”_(T301S, T346K, T701S, T746K, T1101S, |
| | T1146K)_N272Q: “GG”-PDL1 BD: “DiS” hSA-BD |
| 553 | PRO1480_“SK”_(T301S, T346K, T701S, T746K, T1101S, |
| | T1146K)_N272Q: “GG” PDL1-BD: “GG” CD137-BD: “GG” hSA-BD; |
| | “DiS” hSA-BD |

TABLE 4
Description of SEQ ID NOs of specific examples of multispecific antibody sequences
according to the present invention, as present in the Sequence Listing. *⁾

| | |
|---|---|
| SEQ ID NO: | Description: |

PDL1 × CD137 × HSA

| | |
|---|---|
| 554 | scDb-scFv of PRO963 (=PRO1051)_(38-02-A04 sc01 scDb-i/33-03- |
| | G02 sc01 scDb-o/19-01-H04-sc03 scFv) (Reference) |
| 555 | scDb-scFv of PRO963 (=PRO1051)_(38-02-A04 sc01 scDb-i/33-03- |
| | G02 sc01 scDb-o/19-01-H04-sc03 scFv) (A67G_F68G) |
| 556 | scDb-scFv of PRO963 (=PRO1051)_(38-02-A04 sc01 scDb-i/33-03- |
| | G02 sc01 scDb-o/19-01-H04-sc03 scFv) (T101S, |
| | T146K)_(A67G_F68G) |
| 557 | scDb-scFv of PRO966 (=PRO1052) (38-27-C05 sc01 scDb-i/33-03- |
| | G02 sc01 scDb-o/19-01-H04-sc03 scFv) (Reference) |
| 558 | scDb-scFv of PRO966 (=PRO1052) (38-27-C05 sc01 scDb-i/33-03- |
| | G02 sc01 scDb-o/19-01-H04-sc03 scFv) (A67G_F68G) |
| 559 | scDb-scFv of PRO966 (=PRO1052) (38-27-C05 sc01 scDb-i/33-03- |
| | G02 sc01 scDb-o/19-01-H04-sc03 scFv) (T101S, |
| | T146K)_(A67G_F68G) |
| 560 | scDb-scFv of PRO1057 (38-02-A04 sc01 scDb-i/33-03-G02 sc01 |
| | scDb-o/23-12-A01-sc03, sk17sh4) (Reference) |
| 561 | scDb-scFv of PRO1057 (38-02-A04 sc01 scDb-i/33-03-G02 sc01 |
| | scDb-o/23-12-A01-sc03, sk17sh4) (A67G_F68G)_{—} |
| 562 | scDb-scFv of PRO1057 (38-02-A04 sc01 scDb-i/33-03-G02 sc01 |
| | scDb-o/23-12-A01-sc03, sk17sh4) (T101S, T146K) (A67G_F68G) |
| 563 | scDb-scFv of PRO1058 (38-27-C05 sc01 scDb-i/33-03-G02 sc01 |
| | scDb-o/23-13-A01-sc03, sk17sh4) (Reference) |
| 564 | scDb-scFv of PRO1058 (38-27-C05 sc01 scDb-i/33-03-G02 sc01 |
| | scDb-o/23-13-A01-sc03, sk17sh4) (A67G_F68G) |
| 565 | scDb-scFv of PRO1058 (38-27-C05 sc01 scDb-i/33-03-G02 sc01 |
| | scDb-o/23-13-A01-sc03, sk17sh4) (T101S, T146K)_ (A67G_F68G) |
| 566 | scDb-scFv of PRO1175 (37-20-B03-sc01-0/38-02-A04 sc01-i/19-01- |
| | H04sc03 scFv) (Reference) |
| 567 | scDb-scFv of PRO1175 (37-20-B03-sc01-0/38-02-A04 sc01-i/19-01- |
| | H04sc03 scFv) (A67G_F68G) |
| 568 | scDb-scFv of PRO1175 (37-20-B03-sc01-0/38-02-A04 sc01-i/19-01- |
| | H04sc03 scFv) (T101S, T146K)_ (A67G_F68G) |
| 569 | scDb-scFv of PRO1186 (38-02-A04 sc01 scDb-i/37-20-B03sc01 |
| | scDb-o/23-13-A01-sc03 scFv) (Reference) |
| 570 | scDb-scFv of PRO1186 (38-02-A04 sc01 scDb-i/37-20-B03sc01 |
| | scDb-o/23-13-A01-sc03 scFv) (A67G_F68G) |
| 571 | scDb-scFv of PRO1186 (38-02-A04 sc01 scDb-i/37-20-B03sc01 |
| | scDb-o/23-13-A01-sc03 scFv) (T101S, T146K)_ (A67G_F68G) |
| 572 | scDb-scFv of PRO1430 (38-02-A04 sc13 scDb-i/37-20-B03 sc01 |
| | scDb-o/19-01-H04 sc03 scFv) (Reference) |
| 573 | scDb-scFv of PRO1430 (38-02-A04 sc13 scDb-i/37-20-B03 sc01 |
| | scDb-o/19-01-H04 sc03 scFv) (A67G_F68G) |
| 574 | scDb-scFv of PRO1430 (38-02-A04 sc13 scDb-i/37-20-B03 sc01 |
| | scDb-o/19-01-H04 sc03 scFv) (T101S, T146K)_ (A67G_F68G) |
| 575 | scDb-scFv of PRO1479 (38-02-A04 sc13 scDb-i/37-20-B03 sc09.1 |
| | scDb-o/19-01-H04 sc03 scFv) (Reference) |
| 576 | scDb-scFv of PRO1479 (38-02-A04 sc13 scDb-i/37-20-B03 sc09.1 |
| | scDb-o/19-01-H04 sc03 scFv) (A67G_F68G) |
| 577 | scDb-scFv of PRO1479 (38-02-A04 sc13 scDb-i/37-20-B03 sc09.1 |
| | scDb-o/19-01-H04 sc03 scFv) (T101S, T146K)_ (A67G_F68G) |
| 578 | scDb-scFv of PRO1482 (37-20-B03 sc09.1 scDb-i/38-02-A04 sc13 |
| | scDb-o//19-01-H04 sc03 scFv) (Reference) |
| 579 | scDb-scFv of PRO1482 (37-20-B03 sc09.1 scDb-i/38-02-A04 sc13 |
| | scDb-o//19-01-H04 sc03 scFv) (A67G_F68G) |
| 580 | scDb-scFv of PRO1482 (37-20-B03 sc09.1 scDb-i/38-02-A04 sc13 |
| | scDb-o//19-01-H04 sc03 scFv) (T101S, T146K)_ (A67G_F68G) |
| 581 | scDb-scFv of PRO1431 (38-02-A04 sc13 scDb-i/33-03-G02 sc18 |
| | scDb-o/19-01-H04 sc03 scFv) (Reference) |
| 582 | scDb-scFv of PRO1431 (38-02-A04 sc13 scDb-i/33-03-G02 sc18 |
| | scDb-o/19-01-H04 sc03 scFv) (A67G_F68G) |
| 583 | scDb-scFv of PRO1431 (38-02-A04 sc13 scDb-i/33-03-G02 sc18 |
| | scDb-o/19-01-H04 sc03 scFv) (T101S, T146K)_ (A67G_F68G) |
| 584 | scDb-scFv of PRO1473 (38-02-A04 sc13 scDb-i/33-03-G02 sc03 |
| | scDb-o/19-01-H04 sc03 scFv) (Reference) |
| 585 | scDb-scFv of PRO1473 (38-02-A04 sc13 scDb-i/33-03-G02 sc03 |
| | scDb-o/19-01-H04 sc03 scFv) (A67G_F68G) |
| 586 | scDb-scFv of PRO1473 (38-02-A04 sc13 scDb-i/33-03-G02 sc03 |
| | scDb-o/19-01-H04 sc03 scFv) (T101S, T146K)_ (A67G_F68G) |
| 587 | scDb-scFv of PRO1476 (33-03-G02 sc03 scDb-i/38-02-A04 sc13 |
| | scDb-o/19-01-H04 sc03 scFv) (Reference) |
| 588 | scDb-scFv of PRO1476 (33-03-G02 sc03 scDb-i/38-02-A04 sc13 |
| | scDb-o/19-01-H04 sc03 scFv) (A67G_F68G) |
| 589 | scDb-scFv of PRO1476 (33-03-G02 sc03 scDb-i/38-02-A04 sc13 |
| | scDb-o/19-01-H04 sc03 scFv) (T101S, T146K)_ (A67G_F68G) |
| 590 | scDb-scFv of PRO1432 (33-03-G02 sc18 scDb-i/38-02-A04 sc13 |
| | scDb-o/19-01-H04 sc03 scFv) (Reference) |
| 591 | scDb-scFv of PRO1432 (33-03-G02 sc18 scDb-i/38-02-A04 sc13 |
| | scDb-o/19-01-H04 sc03 scFv) (A67G_F68G)_{—} |
| 592 | scDb-scFv of PRO1432 (33-03-G02 sc18 scDb-i/38-02-A04 sc13 |
| | scDb-o/19-01-H04 sc03 scFv) (T101S, T146K)_ (A67G_F68G) |
| 593 | scDb-scFv of PRO1481 (38-27-A11 sc03 scDb-i/37-20-B03 sc09.1 |
| | scDb-o/19-01-H04 sc03 scFv) (Reference) |
| 594 | scDb-scFv of PRO1481 (38-27-A11 sc03 scDb-i/37-20-B03 sc09.1 |
| | scDb-o/19-01-H04 sc03 scFv) (A67G_F68G) |
| 595 | scDb-scFv of PRO1481 (38-27-A11 sc03 scDb-i/37-20-B03 sc09.1 |
| | scDb-o/19-01-H04 sc03 scFv) (T101S, T146K)_ (A67G_F68G) |

IL17A × TNFα × HSA

| | |
|---|---|
| 596 | LC of A5 (Reference) |
| 597 | LC of A5 (A67G_F68G) |
| 598 | LC of A5 (T101S, T146K)_ (A67G_F68G) |
| 599 | HC of A5 (Reference) |
| 600 | HC of A5 (A67G_F68G) |
| 601 | HC of A5 (T101S, T146K)_ (A67G_F68G) |
| 602 | Sequence of A7_(Reference) |
| 603 | Sequence of A7_(A67G_F68G) |
| 604 | Sequence of A7_(T101S, T146K)_ (A67G_F68G) |
| 605 | Sequence of A8_(Reference) |
| 606 | Sequence of A8_(A67G_F68G) |
| 607 | Sequence of A8_(T101S, T146K)_ (A67G_F68G) |

anti-CD3 × HSA × PDL1 × HER2

| | |
|---|---|
| 608 | Chain 1 of PRO 1543 (Reference) |
| 609 | Chain 1 of PRO 1543 (A67G_F68G) |
| 610 | Chain 1 of PRO 1543 (T101S, T146K)_(A67G_F68G) |
| 611 | Chain 2 of PRO 1543 (Reference) |
| 612 | Chain 2 of PRO 1543 (A67G_F68G) |
| 613 | Chain 2 of PRO 1543 (T101S, T146K)_(A67G_F68G) |
| 614 | Chain 1 of PRO 1895 (Reference) |
| 615 | Chain 1 of PRO 1895 (A67G_F68G) |
| 616 | Chain 1 of PRO 1895 (T101S, T146K)_(A67G_F68G) |
| 617 | Chain 2 of PRO 1895 (Reference) |
| 618 | Chain 2 of PRO 1895 (A67G_F68G) |
| 619 | Chain 2 of PRO 1895 (T101S, T146K)_(A67G_F68G) |

MSLN × CD3 × hSA

| | |
|---|---|
| 620 | Chain 1 of PRO2562 (Reference) |
| 621 | Chain 1 of PRO2562 (A67G_F68G) |
| 622 | Chain 1 of PRO2562 (T101S, T146K)_(A67G_F68G) |
| 623 | Chain 2 of PRO2562 (Reference) |
| 624 | Chain 2 of PRO2562 (A67G_F68G) |
| 625 | Chain 2 of PRO2562 (T101S, T146K)_(A67G_F68G) |
| 626 | Chain 1 of PRO2566 (Reference) |
| 627 | Chain 1 of PRO2566 (A67G_F68G) |
| 628 | Chain 1 of PRO2566 (T101S, T146K)_(A67G_F68G) |
| 629 | Chain 2 of PRO2566 (Reference) |
| 630 | Chain 2 of PRO2566 (A67G_F68G) |
| 631 | Chain 2 of PRO2566 (T101S, T146K)_(A67G_F68G) |
| 632 | Chain 1 of PRO2567 (Reference) |
| 633 | Chain 1 of PRO2567 (A67G_F68G) |
| 634 | Chain 1 of PRO2567 (T101S, T146K)_(A67G_F68G) |
| 635 | Chain 2 of PRO2567 (Reference) |
| 636 | Chain 2 of PRO2567 (A67G_F68G) |
| 637 | Chain 2 of PRO2567 (T101S, T146K)_(A67G_F68G) |
| 638 | Chain 1 of PRO2660 (Reference) |
| 639 | Chain 1 of PRO2660 (A67G_F68G) |
| 640 | Chain 1 of PRO2660 (T101S, T146K)_(A67G_F68G) |
| 641 | Chain 2 of PRO2660 (Reference) |
| 642 | Chain 2 of PRO2660 (A67G_F68G) |
| 643 | Chain 2 of PRO2660 (T101S, T146K)_(A67G_F68G) |

ROR1 × CD3 × hSA

| | |
|---|---|
| 644 | scMATCH3 of PRO2667 (Reference) |
| 645 | scMATCH3 of PRO2667 (A67G_F68G) |
| 646 | scMATCH3 of PRO2667 (T101S, T146K)_(A67G_F68G) |
| 647 | scMATCH3 of PRO2668 (Reference) |
| 648 | scMATCH3 of PRO2668 (A67G_F68G) |
| 649 | scMATCH3 of PRO2668 (T101S, T146K)_(A67G_F68G) |
| 650 | Chain 1 of PRO2669_MATCH4 (Reference) |
| 651 | Chain 1 of PRO2669_MATCH4 (A67G_F68G) |
| 652 | Chain 1 of PRO2669_MATCH4 (T101S, T146K)_(A67G_F68G) |
| 653 | Chain 2 of PRO2669_MATCH4 (Reference) |
| 654 | Chain 2 of PRO2669_MATCH4 (A67G_F68G) |
| 655 | Chain 2 of PRO2669_MATCH4 (T101S, T146K)_(A67G_F68G) |
| 656 | Chain 1 of PRO2670_MATCH4 (Reference) |
| 657 | Chain 1 of PRO2670_MATCH4 (A67G_F68G) |
| 658 | Chain 1 of PRO2670_MATCH4 (T101S, T146K)_(A67G_F68G) |
| 659 | Chain 2 of PRO2670_MATCH4 (Reference) |
| 660 | Chain 2 of PRO2670_MATCH4 (A67G_F68G) |
| 661 | Chain 2 of PRO2670_MATCH4 (T101S, T146K)_(A67G_F68G) |

*⁾ Original sequences (sequences without modifications) are disclosed in WO2014180577, WO2017158101, WO2017158079, WO2017158084, WO2018224443, WO2019072868, WO2018224439, WO2019072870, WO2019072869, WO2018224441, WO2020157305, WO2020074584, WO2021089609, WO2021239987, WO2022136669, WO2022084440, WO2022136675, WO2022136672, WO2022167460.

Throughout the text of this application, should there be a discrepancy between the text of the specification (e. g., Tables 1 to 4) and the sequence listing, the text of the specification shall prevail.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

The following Examples illustrates the invention described above, but is not, however, intended to limit the scope of the invention in any way. Other test models known as such to the person skilled in the pertinent art can also determine the beneficial effects of the claimed invention.

### EXAMPLES

### Example 1: Manufacturing of GG Variants of scFvs and Multispecific Antibodies According to the Present Invention and Unmodified Versions Thereof (References)

1.1. Manufacturing of GG Variants of scFvs According to the Present Invention and Unmodified Variants Thereof (References):

The scFv GG variants according to the present invention as well as their respective unmodified versions (non-GG variants) have been produced following the methods described in detail in the patent applications WO 2019/072868 and WO 2021/239987. The produced scFv GG variants and corresponding non-GG versions are listed in Table 5.

Briefly, the expression of the scFv GG variants, as defined herein, and their respective unmodified versions (References) was performed in CHO cells using the ExpiCHO Expression System (ThermoFisher). Expression was conducted according to manufacturer's instructions. Proteins were purified from clarified harvest by affinity chromatography (Protein L and/or Protein A). If necessary, variant scFvs were polished by SE-chromatography to a final monomeric content >95%. For quality control of the manufactured material, standard analytical methods such as ESI-MS, SE-HPLC, UV₂₈₀ and SDS-PAGE were applied.

The mass of the scFvs has for example been verified by the following ESI-MS standard method. Manufactured scFvs were 5-fold diluted with 1% TFA. Two μl of sample were injected into an ACQUITY UPLC@BioResolve-RP-mAb 2.7 μm 2.1×150 mm, 450 Å column (Waters, USA) and desalted using a gradient from 15% to 85% buffer B (0.1% formic acid, 25% propan-2-ol in acetonitrile) at a flow rate of 200 μl/min at 50° C. The MS analysis was performed on a Synapt G2 mass spectrometer directly coupled to the UPLC station. Mass spectra were acquired in the positive-ion mode by scanning the m/z range from 400 to 5,000 da with a scan duration of 1 s and an interscan delay of 0.1s. The data were recorded with the MassLynx 4.2 Software (both Waters, UK). Where possible, the recorded m/z data of single peaks were deconvoluted into mass spectra by applying the maximum entropy algorithm MaxEnt1 (MaxLynx).

The monomeric content of the manufactured scFvs has been determined for example by using the following standard SE-HPLC method. Five μg of the respective scFv, typically present at a concentration of from 0.1 to 10 mg/ml, were injected onto a Shodex KW402.5-4F column using a Hitachi Chromaster HPLC system at 25° C. and a flow rate of 0.35 ml/min. The mobile phase was 50 mM sodium acetate, 250 mM sodium chloride in water at pH 6.0. Elution profile was monitored at 280 nm.

The manufacturing details of the GG variant scFvs and their unmodified versions (non-GG variants) that were produced are summarized in Table 6.

### 1.2. Manufacturing of PRO1480 (Anti-PDL1×CD137×hSA Multispecific Antibody) and GG Variants of PRO1480:

The characterization and manufacturing of PRO1480 and its binding domains is disclosed in detail in the patent application WO 2019/072868. The PRO1480 GG variants of the present invention and its non-modified versions (References) have been produced according to the methods described therein. The produced PRO1480 GG variants and corresponding non-GG versions are listed in Table 5.

Briefly, the expression of PRO1480 GG variants and non-modified versions thereof (scMATCH3 constructs) has been performed at 0.5 I scale using CHOgro expression kit (Mirus) and mammalian CHO-S cells. After 7 days of expression, proteins were purified from clarified culture supernatants by Protein A (MabSelect PrismA, Cytiva) affinity chromatography either followed by size exclusion chromatography (SEC) in 50 mM phosphate-citrate buffer with 300 mM sucrose at pH 6.5 or, where applicable, capture fractions with >95% purity were directly pooled and buffer exchanged to 50 mM phosphate-citrate buffer with 300 mM sucrose at pH 6.5 buffer. Monomeric content of SEC fractions was assessed by SE-HPLC analysis and fractions with a monomeric content >95% were pooled. For quality control of the manufactured material, standard analytical methods such as SE-HPLC, UV₂₈₀ and SDS-PAGE were applied.

The manufacturing details of PRO1480 and the PRO1480 GG variants that were produced are summarized in Table 7.

TABLE 5
Produced scFv GG variants and PRO1480 GG variants according to the present invention and
non-GG versions thereof (References).

| SEQ | | | | |
|---|---|---|---|---|
| ID | | | | |
| NO: | Target | PRO ID | Clone ID | LCDR2 |
| 663 | PD-L1 | PRO3477 | 37-20-B03_human consensus | AASSLQS (SEQ ID |
| | | | | NO: 704) |
| 664 | PD-L1 | PRO3478 | 37-20-B03_human consensus_Y42S | AASSLQS (SEQ ID |
| | | | | NO: 704) |
| 662 | PD-L1 | PRO3757 | 37-20-B03 sc09.1_(A9S) (Reference) | RAFILAS (SEQ ID NO: |
| | | | | 705) |
| 23 | PD-L1 | PRO2230 | 37-20-B03 sc09.1 (Reference) | RAFILAS (SEQ ID NO: |
| | | | | 705) |
| 26 | PD-L1 | PRO3476 | 37-20-B03-sc09.1_(Y42S) (Reference) | RAFILAS (SEQ ID NO: |
| | | | | 705) |
| 35 | PD-L1 | PRO4124 | 37-20-B03 sc16 (A67G_F68G) | RGGILAS (SEQ ID NO: |
| | | | | 667) |
| 41 | PD-L1 | PRO4125 | 37-20-B03 sc17 (A67G_F68G) | RGGILAS (SEQ ID NO: |
| | | | | 667) |
| 47 | PD-L1 | PRO1347 | 37-20-B03 sc09-IF | RAFILAS (SEQ ID NO: |
| | | | | 705) |
| 48 | PD-L1 | PRO3723 | 37-20-B03 sc09-IF (A67G_F68G) | RGGILAS (SEQ ID NO: |
| | | | | 667) |
| 70 | PD-L1 | PRO1392 | 33-03-G02 sc18 (Reference) | KASTLAS (SEQ ID NO: |
| | | | | 706) |
| 71 | PD-L1 | PRO3724 | 33-03-G02-sc18 (A67G_S68G) | KGGTLAS (SEQ ID |
| | | | | NO: 668) |
| 72 | PD-L1 | PRO3891 | 33-03-G02-sc18 (T101S_T146K)_(A67G_S68G) | KGGTLAS (SEQ ID |
| | | | | NO: 668) |
| 73 | PD-L1 | PRO3747 | 33-03-G02-sc18 (S72D_W73S_A74V) (A67G_S68G) | KGGTLAS (SEQ ID |
| | | | | NO: 668) |
| 82 | 4-1BB | PRO1359 | 38-27-A11-sc02 (Reference) | SASTLAS (SEQ ID NO: |
| | | | | 707) |
| 83 | 4-1BB | PRO3726 | 38-27-A11-sc02 (A67G_S68G) | SGGTLAS (SEQ ID |
| | | | | NO: 670) |
| 91 | 4-1BB | PRO2194 | 38-27-A11-sc02 (G51C_N72Q) (T141C) (Reference) | SASTLAS (SEQ ID NO: |
| | | | | 707) |
| 92 | 4-1BB | PRO3742 | 38-27-A11-sc02 (G51C_N72Q) (T141C) (A67G_S68G) | SGGTLAS (SEQ ID |
| | | | | NO: 670) |
| 107 | 4-1BB | PRO4147 | 38-27-A11-sc10 (Reference) | SASTLAS (SEQ ID NO: |
| | | | | 707) |
| 108 | 4-1BB | PRO4151 | 38-27-A11-sc10 (A67G_S68G) | SGGTLAS (SEQ ID |
| | | | | NO: 670) |
| 109 | 4-1BB | PRO4155 | 38-27-A11-sc10_(G51C) (T141C) (A67G_S68G) | SGGTLAS (SEQ ID |
| | | | | NO: 670) |
| 134 | 4-1BB | PRO1352 | 38-02-A04-sc13 (Reference) | RASTLAS (SEQ ID NO: |
| | | | | 708) |
| 135 | 4-1BB | PRO3727 | 38-02-A04-sc13 (A67G_S68G) | RGGTLAS (SEQ ID |
| | | | | NO: 669) |
| 142 | 4-1BB | PRO4148 | 38-02-A04-sc16 (Reference) | RASTLAS (SEQ ID NO: |
| | | | | 708) |
| 143 | 4-1BB | PRO4152 | 38-02-A04-sc16 (A67G_S68G) | RGGTLAS (SEQ ID |
| | | | | NO: 669) |
| 144 | 4-1BB | PRO4156 | 38-02-A04-sc16_(G51C) (T141C) (A67G_S68G) | RGGTLAS (SEQ ID |
| | | | | NO: 669) |
| 156 | CD3e | PRO2781 | 28-21-D09-sc04 (Reference) | SASTLAS (SEQ ID NO: |
| | | | | 707) |
| 157 | CD3e | PRO3718 | 28-21-D09-sc04 (A67G_S68G) | SGGTLAS (SEQ ID |
| | | | | NO: 670) |
| 165 | CD3e | PRO4187 | 28-21-D09-sc43 (Reference) | SASTLAS (SEQ ID NO: |
| | | | | 707) |
| 166 | CD3e | PRO4188 | 28-21-D09-sc43 (A67G_S68G) | SGGTLAS (SEQ ID |
| | | | | NO: 670) |
| 167 | CD3e | PRO4189 | 28-21-D09-sc43_(T101S_T146K) (Reference) | SASTLAS (SEQ ID NO: |
| | | | | 707) |
| 168 | CD3e | PRO4190 | 28-21-D09-sc43_(T101S_T146K) (A67G_S68G) | SGGTLAS (SEQ ID |
| | | | | NO: 670) |
| 169 | CD3e | PRO4191 | 28-21-D09-sc43_(T101S_T146K) | SGGTLAS (SEQ ID |
| | | | (G51C_T141C) (A67G_S68G) | NO: 670) |
| 177 | HSA | PRO2155 | 19-04-A10-sc02 (Reference) | DASDLTS (SEQ ID |
| | | | | NO: 709) |
| 178 | HSA | PRO3721 | 19-04-A10-sc02 (A67G_S68G) | DGGDLTS (SEQ ID |
| | | | | NO: 686) |
| 186 | HSA | PRO4112 | 19-04-A10-sc11 (Reference) | DASDLTS (SEQ ID |
| | | | | NO: 709) |
| 187 | HSA | PRO4116 | 19-04-A10-sc11 (A67G_S68G) | DGGDLTS (SEQ ID |
| | | | | NO: 686) |
| 188 | HSA | PRO4113 | 19-04-A10-sc12 (Reference) | DASDLTS (SEQ ID |
| | | | | NO: 709) |
| 189 | HSA | PRO4117 | 19-04-A10-sc12 (A67G_S68G) | DGGDLTS (SEQ ID |
| | | | | NO: 686) |
| 221 | MSLN | PRO1922 | 54-21-H03-sc01 (Reference) | LASTLAS (SEQ ID NO: |
| | | | | 710) |
| 222 | MSLN | PRO3732 | 54-21-H03-sc01 (A67G_S68G) | LGGTLAS (SEQ ID |
| | | | | NO: 671) |
| 244 | MSLN | PRO2309 | 54-32-A07-sc06_(G51C_T141C) (Reference) | DASDLAS (SEQ ID |
| | | | | NO: 711) |
| 245 | MSLN | PRO3733 | 54-32-A07-sc06_(G51C_T141C) (A67G_S68G) | DGGDLAS (SEQ ID |
| | | | | NO: 675) |
| 271 | ROR1 | PRO2062 | 55-39-G02-sc02 (Reference) | TASTLAS (SEQ ID NO: |
| | | | | 712) |
| 272 | ROR1 | PRO3734 | 55-39-G02-sc02 (A67G_S68G) | TGGTLAS (SEQ ID |
| | | | | NO: 672) |
| 295 | ROR1 | PRO2060 | 55-38-D07-sc02 (Reference) | RASTLAS (SEQ ID NO: |
| | | | | 708) |
| 296 | ROR1 | PRO3735 | 55-38-D07-sc02 (A67G_S68G) | RGGTLAS (SEQ ID |
| | | | | NO: 669) |
| 299 | ROR1 | PRO4135 | 55-38-D07-sc09 (Reference) | RASTLAS (SEQ ID NO: |
| | | | | 708) |
| 300 | ROR1 | PRO4139 | 55-38-D07-sc09 (A67G_S68G) | RGGTLAS (SEQ ID |
| | | | | NO: 669) |
| 301 | ROR1 | PRO4143 | 55-38-D07-sc09_(G51C) (A67G_S68G_T141C) | RGGTLAS (SEQ ID |
| | | | | NO: 669) |
| 305 | Her2 | PRO0286 | Trastuzumab_lambda-cap (Reference) | SASFLYS (SEQ ID NO: |
| | | | | 713) |
| 306 | Her2 | PRO3716 | Trastuzumab_lambda-cap (A67G_S68G) | SGGFLYS (SEQ ID |
| | | | | NO: 687) |
| 310 | Her2 | PRO1812 | Pertuzumab_lambda-cap (Reference) | SASYRYT (SEQ ID |
| | | | | NO: 714) |
| 311 | Her2 | PRO3717 | Pertuzumab_lambda-cap (A67G_S68G) | SGGYRYT (SEQ ID |
| | | | | NO: 688) |
| 320 | IL23R | PRO0517 | 14-11-D07-sc01 (Reference) | SASKLAA (SEQ ID NO: |
| | | | | 715) |
| 323 | IL23R | PRO3719 | 14-11-D07-sc04 (A67G_S68G) | SGGKLAA (SEQ ID |
| | | | | NO: 689) |
| 333 | IL17 | PRO0561 | 27-07-G02-sc01 (Reference) | RASTLAS (SEQ ID NO: |
| | | | | 708) |
| 334 | IL17 | PRO3722 | 27-07-G02-sc01 (A67G_F68G) | RGGTLAS (SEQ ID |
| | | | | NO: 669) |
| 353 | PD-L1 | PRO1434 | 33-03-G02_Q108A_G109A (Reference) | KASTLAS (SEQ ID NO: |
| | | | | 706) |
| 354 | PD-L1 | PRO3725 | 33-03-G02_Q108A_G109A (A67G_F68G) | KGGTLAS (SEQ ID |
| | | | | NO: 668) |
| 544 | PD-L1 x | PRO1480 | PRO1480_unmodified (Reference) | RAFILAS (SEQ ID NO: |
| | CD137 x | | | 705)/SASTLAS (SEQ |
| | hSA | | | ID NO: 707)/DASDLAS |
| | | | | (SEQ ID NO: 711) |
| 545 | PD-L1 x | PRO5033 | PRO1480_″GG″-PDL1 BD | RGGILAS (SEQ ID NO: |
| | CD137 x | | | 667)/SASTLAS (SEQ |
| | hSA | | | ID NO: 707)/DASDLAS |
| | | | | (SEQ ID NO: 711) |
| 743 | PD-L1 x | PRO5034 | PRO1480_″GG″-PDL1 BD ″GG″ CD137-BD | RGGILAS (SEQ ID NO: |
| | CD137 x | | | 667)/SGGTLAS (SEQ |
| | hSA | | | ID NO: 670)/DASDLAS |
| | | | | (SEQ ID NO: 711) |
| 551 | PD-L1 x | PRO3378 | PRO1480_″SK″_(T301S, T346K, T701S, T746K, T1101S, | RAFILAS (SEQ ID NO: |
| | CD137 x | | T1146K)_N272Q ″DiS“ hSA-BD (Reference) | 705)/ SASTLAS (SEQ |
| | hSA | | | ID NO: 707)/DASDLAS |
| | | | | (SEQ ID NO: 711) |
| 552 | PD-L1 x | PRO4180 | PRO1480_″SK″_(T301S, T346K, T701S, T746K, T1101S, | RGGILAS (SEQ ID NO: |
| | CD137 x | | T1146K)_N272Q ″GG″-PDL1 BD ″DiS″ hSA-BD | 667)/SASTLAS(SEQ |
| | hSA | | | ID NO: 707)/ |
| | | | | DASDLAS (SEQ ID |
| | | | | NO: 711) |
| 553 | PD-L1 x | PRO4181 | PRO1480_″SK″_(T301S, T346K, T701S, T746K, T1101S, | RGGILAS (SEQ ID NO: |
| | CD137 x | | T1146K)_N272Q ″GG″ PDL1-BD ″GG″ CD137-BD ″GG″ | 667)/SGGTLAS (SEQ |
| | hSA | | hSA-BD ″DiS″ hSA-BD | ID NO: 670)/ |
| | | | | DGGDLAS (SEQ ID |
| | | | | NO: 675) |

TABLE 6
Manufacturing details for produced scFv GG variants
and non-GG versions thereof (References).

| | | | Purified | Final | Final |
|---|---|---|---|---|---|
| SEQ | | | volume | titer | MC *⁾ |
| ID | Target | PRO ID | [ml] | [mg/l] | [%] |
| 663 | PD-L1 | PRO3477 | 12 | 122 | 100 |
| 664 | PD-L1 | PRO3478 | 12 | 82 | 100 |
| 662 | PD-L1 | PRO3757 | 15 | 85 | 100 |
| 23 | PD-L1 | PRO2230 | 219 | 242 | 100 |
| 26 | PD-L1 | PRO3476 | 12 | 83 | 100 |
| 35 | PD-L1 | PRO4124 | 50 | 470 | 100 |
| 41 | PD-L1 | PRO4125 | 50 | 444 | 100 |
| 47 | PD-L1 | PRO1347 | 200 | 12 | 98 |
| 48 | PD-L1 | PRO3723 | 13 | 50 | 99 |
| 70 | PD-L1 | PRO1392 | 200 | 37 | 97 |
| 71 | PD-L1 | PRO3724 | 13 | 26 | 89 |
| 72 | PD-L1 | PRO3891 | 50 | 216 | 99 |
| 73 | PD-L1 | PRO3747 | 100 | 329 | 97 |
| 82 | 4-1BB | PRO1359 | 200 | 17 | 92 |
| 83 | 4-1BB | PRO3726 | 13 | 233 | 99 |
| 91 | 4-1BB | PRO2194 | 250 | 8 | 96 |
| 92 | 4-1BB | PRO3742 | 13 | 74 | 99 |
| 107 | 4-1BB | PRO4147 | 42 | 201 | 99 |
| 108 | 4-1BB | PRO4151 | 45 | 104 | 100 |
| 109 | 4-1BB | PRO4155 | 46 | 102 | 100 |
| 134 | 4-1BB | PRO1352 | 800 | 3 | 97.2 |
| 135 | 4-1BB | PRO3727 | 13 | 131 | 99 |
| 142 | 4-1BB | PRO4148 | 44 | 215 | 95 |
| 143 | 4-1BB | PRO4152 | 40 | 325 | 97 |
| 144 | 4-1BB | PRO4156 | 44 | 175 | 98 |
| 156 | CD3e | PRO2781 | 200 | 190 | 100 |
| 157 | CD3e | PRO3718 | 13 | 256 | 98 |
| 165 | CD3e | PRO4187 | 46 | 330 | 95 |
| 166 | CD3e | PRO4188 | 46 | 171 | 96 |
| 167 | CD3e | PRO4189 | 46 | 354 | 96 |
| 168 | CD3e | PRO4190 | 44 | 187 | 97 |
| 169 | CD3e | PRO4191 | 43 | 10 | 99 |
| 177 | HSA | PRO2155 | 500 | 30 | 98.0 |
| 178 | HSA | PRO3721 | 13 | 172 | 99 |
| 186 | HSA | PRO4112 | 40 | 103 | 99 |
| 187 | HSA | PRO4116 | 42 | 104 | 99 |
| 188 | HSA | PRO4113 | 42 | 82 | 99 |
| 189 | HSA | PRO4117 | 42 | 101 | 99 |
| 221 | MSLN | PRO1922 | 580 | 49 | 100 |
| 222 | MSLN | PRO3732 | 13 | 50 | 100 |
| 244 | MSLN | PRO2309 | 100 | 144 | 99.6 |
| 245 | MSLN | PRO3733 | 13 | 18 | 98 |
| 271 | ROR1 | PRO2062 | 300 | 16 | 99.9 |
| 272 | ROR1 | PRO3734 | 13 | 23 | 99 |
| 295 | ROR1 | PRO2060 | 500 | 25 | 98 |
| 296 | ROR1 | PRO3735 | 13 | 121 | 99 |
| 299 | ROR1 | PRO4135 | 42 | 223 | 95 |
| 300 | ROR1 | PRO4139 | 44 | 68 | 100 |
| 301 | ROR1 | PRO4143 | 44 | 97 | 100 |
| 305 | Her2 | PRO0286 | nd | nd | nd |
| 306 | Her2 | PRO3716 | nd | nd | nd |
| 310 | Her2 | PRO1812 | 280 | 13 | 100 |
| 311 | Her2 | PRO3717 | | | |
| 320 | IL23R | PRO0517 | 1500 | 64 | 99.0 |
| 323 | IL23R | PRO3719 | | | |
| 333 | IL17 | PRO0561 | 55 | 95 | 95.1 |
| 334 | IL17 | PRO3722 | | | |
| 353 | PD-L1 | PRO1434 | 1000 | 8 | 100 |
| 354 | PD-L1 | PRO3725 | 13 | 216 | 99 |

*⁾ MC: monomeric content

TABLE 7
Manufacturing details for produced PRO1480 and PRO1480 GG variants thereof.

| | | | Purified volume | Post-capture titer | Final titer | Final MC * |
|---|---|---|---|---|---|---|
| SEQ ID | Target | PRO ID | [ml] | [mg/l] | [mg/l] | [%] |
| 544 | PD-L1 × CD137 × hSA | PRO1480 | 8800 | 43** | 25 | 98.7 |
| 545 | PD-L1 × CD137 × hSA | PRO5033 | 236 | 350 | 239 | 98.7 |
| 743 | PD-L1 × CD137 × hSA | PRO5034 | 247 | 330 | 230 | 98.8 |
| 551 | PD-L1 × CD137 × hSA | PRO3378 | 246 | 188 | 146 | 96.7 |
| 552 | PD-L1 × CD137 × hSA | PRO4180 | 114 | 242 | 48*** | 97.0 |
| 553 | PD-L1 × CD137 × hSA | PRO4181 | 102 | 259 | 162 | 99.9 |

* MC: monomeric content
**captured on protein L
***only purest post-capture fractions were buffer-exchanged

### Example 2: Determination of Binding Potencies to MHC Class II Molecules

### 2.1. General Procedure:

In silico prediction of immunogenicity risk, often in terms of predicted peptide-MHC binding strength, has become common practice for biotherapeutics development (Gokemeijer et al., AAPS J., 2017, 19, pp. 1587-1592) since peptide-MHC class II binding is a necessary and important condition for immunogenicity. NetMHCIIpan (Andreatta et al., Bioinformatics, 2018, 34, pp. 1522-1528; Andreatta et al., Immunogenetics, 2015, 67, pp. 641-650) is one of several different pieces of software, herein also referred to as NetMHCIIpan algorithm, that are useful for immunogenicity risk prediction. Low percentile ranks, as determined by NetMHCIIpan, correspond to high MHC binding strengths. Similar to other software for predicting MHC class II epitopes (Sturniolo et al., Nat Biotechnol, 1999, 17, pp. 555-561; De Groot et al., Vaccine, 2001, 19, pp. 4385-4395; EP2125883A2; Jensen et al., Immunology, 2018, 154, pp. 394-406; Nielsen et al., BMC Bioinformatics, 2007, 8, p. 238), NetMHCIIpan operates by splitting each amino acid sequence into consecutive overlapping fixed-length frames. NetMHCIIpan uses 15mer frames, and within each such frame, the lowest scoring 9mer is identified and reported as the score of the 15mer, for each chosen allele. For the prediction results reported herein, a set of 27 alleles was used, which covers a substantial majority of MHC class II molecules (Greenbaum et al., Immunogenetics, 2011, 63, pp. 325-335). The per-allele percentile rank of a 15mer is obtained from its score by comparing the score against the scores of 200,000 random natural 15mers (Karosiene et al., Immunogenetics, 2013, 65, pp. 711-724). For these prediction results, external to the NetMHCIIpan software, any reported 15mer percentile rank, for which the 9mer sequence that gives rise to the 15mer's percentile rank corresponds to a human germline segment, is reset to 100.0, since those are unlikely to be problematic. Thereafter, the median percentile rank for each 15mer frame is calculated from the 27 per-allele percentile ranks. We use a median percentile rank less than 20 as an indicator of immunogenicity risk, which is the commonly chosen threshold for this purpose (Oseroff et al., J. Immunol., 2010, 185, pp. 943-955; Paul et al., J. Immunol. Methods, 2015, 422, pp. 28-34).

The NetMHCIIpan software predicts binding of each individual peptide to MHC class II by applying an ensemble of artificial neural networks with 906-neuron input layers, that have been trained on quantitative peptide binding data that covers multiple MHC class II molecules. For the presentation to the 906-neuron input layer of each network, the amino acids of each 9mer core peptide are encoded as the corresponding 20-element vector from the BLOSUM50 matrix (Henikoff et al., Proc. Natl. Acad. Sci. USA, 1992, 89, pp. 10915-10919). The peptide flanking regions are encoded as one 20-element vector for each side of the binding core, formed as the average BLOSUM50 score vector for the residues in each flanking region. MHC class II molecules are represented by 34-residue pseudo-sequences, where each residue is again encoded as the corresponding 20-element vector from the BLOSUM50 matrix. Finally, peptide and peptide flanking region lengths are each encoded to two different input neurons. In total this leads to 9×20+2×20+34×20+3×2=906 inputs, for each pair of peptide and MHC class II molecule.

All binding potency predictions to MHC class II molecule have been performed with the NetMHCIIpan software version 3.1, hereinafter referred to as “NetMHCIIpan-3.1” or “NetMHCIIpan-3.1 algorithm”. NetMHCIIpan software version 3.1 is specifically disclosed in Andreatta et al., Immunogenetics, 2015, 67(0), pp. 641-650.

2.2. Illustrative Example of Predicting the MCH Class II Binding Potency of a Reference PD-L1 Binding Domain in scFv Format Using NetMHCIIpan-3.1:

A peptide sequence of interest, here as an example the VL part of the antibody variable domain of the anti-PD-L1 scFv PRO2230 (clone ID 37-20-B03-sc09.1), i. e. SEQ ID NO: 19, is first decomposed into consecutive 15mer subsequences with an overlap of 14 amino acids starting with position 1 (15mers with amino acid positions 1-15, 2-16, 3-17, 4-18, 5-19, etc.). For SEQ ID NO: 19, this results in 99 consecutive 15mer subsequences.

Each of these 15mer subsequences is then processed by NetMHCIIpan-3.1 for binding potency prediction towards each of the 27 MHC class II alleles that are most common in the general population. For each of said 15mer subsequences NetMHCIIpan-3.1 produces a percentile rank for each of said 27 MHC class II alleles. NetMHCIIpan-3.1 further indicates for each of said 27 MHC class II alleles the respective 9mer within the 15mer subsequence that exhibits the strongest predicted binding (9mere binding core). For each 15mer, this leads to a set of results as for example shown in Table 8 below. Table 8 contains the results obtained for the 15mer IYRAFILASGVPSRF (SEQ ID NO: 716) (15mer index: 47, start: 48, stop: 62).

TABLE 8
Output example of NetMHCIIpan-3.1 for 15 mer IYRAFILASGVPSRF (SEQ ID
NO: 716):

| allele | percentile rank | 9mer binding core |
|---|---|---|
| HLA-DRB1*01:01 | 0.45 | FILASGVPS (SEQ ID NO: |
| | | 717) |
| HLA-DRB1*03:01 | 5.01 | ILASGVPSR (SEQ ID NO: |
| | | 718) |
| HLA-DRB1*04:01 | 2.12 | FILASGVPS (SEQ ID NO: |
| | | 717) |
| HLA-DRB1*04:05 | 1.63 | FILASGVPS (SEQ ID NO: |
| | | 717) |
| HLA-DRB1*07:01 | 2.02 | FILASGVPS (SEQ ID NO: |
| | | 717) |
| HLA-DRB1*08:02 | 2.51 | FILASGVPS (SEQ ID NO: |
| | | 717) |
| HLA-DRB1*09:01 | 0.71 | FILASGVPS (SEQ ID NO: |
| | | 717) |
| HLA-DRB1*11:01 | 4.93 | FILASGVPS (SEQ ID NO: |
| | | 717) |
| HLA-DRB1*12:01 | 11.36 | FILASGVPS (SEQ ID NO: |
| | | 717) |
| HLA-DRB1*13:02 | 4.99 | ILASGVPSR (SEQ ID NO: |
| | | 718) |
| HLA-DRB1*15:01 | 2.79 | FILASGVPS (SEQ ID NO: |
| | | 717) |
| HLA-DRB3*01:01 | 15.67 | ILASGVPSR (SEQ ID NO: |
| | | 718) |
| HLA-DRB3*02:02 | 1.81 | FILASGVPS (SEQ ID NO: |
| | | 717) |
| HLA-DRB4*01:01 | 13.27 | ILASGVPSR (SEQ ID NO: |
| | | 718) |
| HLA-DRB5*01:01 | 5.41 | FILASGVPS (SEQ ID NO: |
| | | 717) |
| HLA-DQA1*05:01/DQB1*02:01 | 20.58 | AFILASGVP (SEQ ID NO: |
| | | 719) |
| HLA-DQA1*05:01/DQB1*03:01 | 3.42 | FILASGVPS (SEQ ID NO: |
| | | 717) |
| HLA-DQA1*03:01/DQB1*03:02 | 16.28 | AFILASGVP (SEQ ID NO: |
| | | 719) |
| HLA-DQA1*04:01/DQB1*04:02 | 15.26 | AFILASGVP (SEQ ID NO: |
| | | 719) |
| HLA-DQA1*01:01/DQB1*05:01 | 28.62 | YRAFILASG (SEQ ID NO: |
| | | 720) |
| HLA-DQA1*01:02/DQB1*06:02 | 4.26 | YRAFILASG (SEQ ID NO: |
| | | 720) |
| HLA-DPA1*02:01/DPB1*01:01 | 17.03 | FILASGVPS (SEQ ID NO: |
| | | 717) |
| HLA-DPA1*01:03/DPB1*02:01 | 17.3 | FILASGVPS (SEQ ID NO: |
| | | 717) |
| HLA-DPA1*01:03/DPB1*04:01 | 16.88 | FILASGVPS (SEQ ID NO: |
| | | 717) |
| HLA-DPA1*03:01/DPB1*04:02 | 17.12 | FILASGVPS (SEQ ID NO: |
| | | 717) |
| HLA-DPA1*02:01/DPB1*05:01 | 17.67 | FILASGVPS (SEQ ID NO: |
| | | 717) |
| HLA-DPA1*02:01/DPB1*14:01 | 4.09 | FILASGVPS (SEQ ID NO: |
| | | 717) |

For each 15mer, the median of these percentile ranks is extracted. These percentile ranks can be plotted for all 15mers resulting in a graph as shown in FIG. 1. The lower the median percentile rank, the stronger is the predicted binding of the 15mer to MHC class II molecules. Typically, a median percentile rank of less than 20 is deemed to indicate significant binding to MHJC class II proteins (median percentile rank 20 is represented in FIG. 1 as a dashed line).

2.3. Prediction of the MCH Class II Binding Potency of the Produced scFv GG Variants and Non-GG Versions Thereof (References) Using NetMHCIIpan-3.1:

The methodology described above in section 2.2 has been applied to the GG variants of the produced scFvs according to the present invention and their respective non-GG versions (References). For each scFv, the VL as well as the VH was analyses. Results of the predicted MHC class II biding potencies are summarized in Tables 9 and 10.

TABLE 9
Predicted T cell epitopes for VL sequences of the GG variants and the non-GG versions of the produced
scFvs.

| SEQ | | | | | | 15 mer Hotspot |
|---|---|---|---|---|---|---|
| ID | | Clusters | Cluster | Cluster | 15 mer Hotspot | median perc |
| NO: | Clone ID | VL | size VL | sum VL | sequence(s) VL | rank(s) VL |
| 663 | 37-20-B03_human cons | 0 | 0 | 0 | NONE | NONE |
| 664 | 37-20-B03_human | 0 | 0 | 0 | NONE | NONE |
| | cons_Y42S | | | | | |
| 662 | 37-20-B03 sc09.1_(A9S) | 1 | 10 | 10 | 48-62-IYRAFILASGVPSRF (SEQ | 5.01 |
| | (Reference) | | | | ID NO: 716) | |
| 23 | 37-20-B03 sc09.1 | 1 | 10 | 10 | 48-62-IYRAFILASGVPSRF (SEQ | 5.01 |
| | (Reference) | | | | ID NO: 716) | |
| 26 | 37-20-B03-sc09.1_(Y42S) | 1 | 10 | 10 | 48-62-IYRAFILASGVPSRF (SEQ | 5.01 |
| | (Reference) | | | | ID NO: 716) | |
| 35 | 37-20-B03 sc16 | 0 | 0 | 0 | NONE | NONE |
| | (A67G_F68G) | | | | | |
| 41 | 37-20-B03 sc17 | 0 | 0 | 0 | NONE | NONE |
| | (A67G_F68G) | | | | | |
| 47 | 37-20-B03 sc09-IF | 1 | 10 | 10 | 48-62-IYRAFILASGVPSRF (SEQ | 5.01 |
| | | | | | ID NO: 716) | |
| 48 | 37-20-B03 sc09-IF | 0 | 0 | 0 | NONE | NONE |
| | (A67G_F68G) | | | | | |
| 70 | 33-03-G02 sc18 | 1 | 6 | 6 | 44-58-PKLLIYKASTLASGV (SEQ | 7.45 |
| | | | | | ID NO: 721) | |
| 71 | 33-03-G02-sc18 | 0 | 0 | 0 | NONE | NONE |
| | (A67G_S68G) | | | | | |
| 72 | 33-03-G02-sc18 | 0 | 0 | 0 | NONE | NONE |
| | (T101S_T146K) | | | | | |
| | (A67G_S68G) | | | | | |
| 73 | 33-03-G02-sc18 | 0 | 0 | 0 | NONE | NONE |
| | (S72D_W73S_A74V) | | | | | |
| | (A67G_S68G) | | | | | |
| 82 | 38-27-A11-sc02 | 1 | 6 | 6 | 44-58-PKLLIYSASTLASGV (SEQ | 7.41 |
| | (Reference) | | | | ID NO: 722 | |
| 83 | 38-27-A11-sc02 | 0 | 0 | 0 | NONE | NONE |
| | (A67G_S68G) | | | | | |
| 91 | 38-27-A11-sc02 | 1 | 6 | 6 | 44-58-PKLLIYSASTLASGV (SEQ | 7.41 |
| | (G51C_N72Q)_(T141C) | | | | | |
| | (Reference) | | | | ID NO: 722) | |
| 92 | 38-27-A11-sc02 | 0 | 0 | 0 | NONE | NONE |
| | (G51C_N72Q)_(T141C) | | | | | |
| | (A67G_S68G) | | | | | |
| 107 | 38-27-A11-sc10 | 1 | 5 | 5 | 44-58-PKLLIYSASTLASGV (SEQ | 7.41 |
| | (Reference) | | | | ID NO: 722) | |
| 108 | 38-27-A11-sc10 | 0 | 0 | 0 | NONE | NONE |
| | (A67G_S68G) | | | | | |
| 109 | 38-27-A11-sc10 | 0 | 0 | 0 | NONE | NONE |
| | (G51C)_(T141C) | | | | | |
| | (A67G_S68G) | | | | | |
| 134 | 38-02-A04-sc13 (Reference) | 1 | 7 | 7 | 45-59-KLLIYRASTLASGVP (SEQ | 4.58 |
| | | | | | ID NO: 723) | |
| 135 | 38-02-A04-sc13 | 0 | 0 | 0 | NONE | NONE |
| | (A67G_S68G) | | | | | |
| 142 | 38-02-A04-sc16 (Reference) | 1 | 7 | 7 | 45-59-KLLIYRASTLASGVP (SEQ | 4.58 |
| | | | | | ID NO: 723) | |
| 143 | 38-02-A04-sc16 | 0 | 0 | 0 | NONE | NONE |
| | (A67G_S68G) | | | | | |
| 144 | 38-02-A04- | 0 | 0 | 0 | NONE | NONE |
| | sc16_(G51C)_(T141C) | | | | | |
| | (A67G_S68G) | | | | | |
| 156 | 28-21-D09-sc04 (Reference) | 1 | 5 | 5 | 46-60-PKRLIYSASTLASGV (SEQ | 6.85 |
| | | | | | ID NO: 724) | |
| 157 | 28-21-D09-sc04 | 0 | 0 | 0 | NONE | NONE |
| | (A67G_S68G) | | | | | |
| 165 | 28-21-D09-sc43 (Reference) | 1 | 5 | 5 | 46-60-PKRLIYSASTLASGV (SEQ | 6.85 |
| | | | | | ID NO: 724) | |
| 166 | 28-21-D09-sc43 | 0 | 0 | 0 | NONE | NONE |
| | (A67G_S68G) | | | | | |
| 167 | 28-21-D09- | 1 | 5 | 5 | 46-60-PKRLIYSASTLASGV (SEQ | 6.85 |
| | sc43_(T101S_T146K) | | | | ID NO: 724) | |
| | (Reference) | | | | | |
| 168 | 28-21-D09- | 0 | 0 | 0 | NONE | NONE |
| | sc43_(T101S_T146K) | | | | | |
| | (A67G_S68G) | | | | | |
| 169 | 28-21-D09- | 0 | 0 | 0 | NONE | NONE |
| | sc43_(T101S_T146K) | | | | | |
| | (G51C_T141C)_(A67G_S68G) | | | | | |
| 177 | 19-04-A10-sc02 (Reference) | 0 | 0 | 0 | NONE | NONE |
| 178 | 19-04-A10-sc02 | 0 | 0 | 0 | NONE | NONE |
| | (A67G_S68G) | | | | | |
| 186 | 19-04-A10-sc11 (Reference) | 0 | 0 | 0 | NONE | NONE |
| 187 | 19-04-A10-sc11 | 0 | 0 | 0 | NONE | NONE |
| | (A67G_S68G) | | | | | |
| 188 | 19-04-A10-sc12 (Reference) | 0 | 0 | 0 | NONE | NONE |
| 189 | 19-04-A10-sc12 | 0 | 0 | 0 | NONE | NONE |
| | (A67G_S68G) | | | | | |
| 22 | 54-21-H03-sc01 | 2 | 4\|7 | 11 | 25-39-ASESIYSSLAWYQQK (SEQ | 14.55\|5.99 |
| | (Reference) | | | | ID NO: 725)\|44-58- | |
| | | | | | PKLLIYLASTLASGV (SEQ ID NO: | |
| | | | | | 726) | |
| 222 | 54-21-H03-sc01 | 1 | 4 | 4 | 25-39-ASESIYSSLAWYQQK (SEQ | 14.55 |
| | (A67G_S68G) | | | | ID NO: 725) | |
| 244 | 54-32-A07- | 1 | 4 | 4 | 44-58-PKFLIYDASDLASGV (SEQ | 14.97 |
| | sc06_(G51C_T141C) | | | | ID NO: 727) | |
| | (Reference) | | | | | |
| 245 | 54-32-A07- | 0 | 0 | 0 | NONE | NONE |
| | sc06_(G51C_T141C) | | | | | |
| | (A67G_S68G) | | | | | |
| 271 | 55-39-G02-sc02 (Reference) | 1 | 5 | 5 | 46-60-PKLLIVTASTLASGV (SEQ | 10.74 |
| | | | | | ID NO: 728) | |
| 272 | 55-39-G02-sc02 | 0 | 0 | 0 | NONE | NONE |
| | (A67G_S68G) | | | | | |
| 295 | 55-38-D07-sc02 (Reference) | 1 | 7 | 7 | 45-59-KLLIYRASTLASGVS (SEQ | 4.12 |
| | | | | | ID NO: 729) | |
| 296 | 55-38-D07-sc02 | 0 | 0 | 0 | NONE | NONE |
| | (A67G_S68G) | | | | | |
| 299 | 55-38-D07-sc09 (Reference) | 1 | 7 | 7 | 45-59-KLLIYRASTLASGVS (SEQ | 4.12 |
| | | | | | ID NO: 729) | |
| 300 | 55-38-D07-sc09 | 0 | 0 | 0 | NONE | NONE |
| | (A67G_S68G) | | | | | |
| 301 | 55-38-D07-sc09_(G51C) | 0 | 0 | 0 | NONE | NONE |
| | (A67G_S68G_T141C) | | | | | |
| 305 | Trastuzumab_lambda-cap | 1 | 12 | 12 | 45-59-PKLLIYSASFLYSGV (SEQ | 10.33 |
| | (Reference) | | | | ID NO: 730) | |
| 306 | Trastuzumab_lambda-cap | 1 | 4 | 4 | 50-64-SGGFLYSGVPSRFSG | 14.8 |
| | (A67G_S68G) | | | | (SEQ ID NO: 731) | |
| 310 | Pertuzumab_lambda-cap | 1 | 7 | 7 | 44-58-PKLLIYSASYRYTGV (SEQ | 4.24 |
| | (Reference) | | | | ID NO: 732) | |
| 311 | Pertuzumab_lambda-cap | 0 | 0 | 0 | NONE | NONE |
| | (A67G_S68G) | | | | | |
| 320 | 14-11-D07-sc01 (Reference) | 2 | 5\|8 | 13 | 27-41-SENIYSFLAWYQQKP (SEQ | 11.9\|3.17 |
| | | | | | ID NO: 733)\|45-59- | |
| | | | | | PKLLIYSASKLAAGV (SEQ ID | |
| | | | | | NO: 734) | |
| 323 | 14-11-D07-sc04 | 1 | 5 | 5 | 26-40-SENIYSFLAWYQQKP (SEQ | 11.9 |
| | (A67G_S68G) | | | | ID NO: 733) | |
| 333 | 27-07-G02-sc01 (Reference) | 3 | 3\|7\|3 | 13 | 27-41-SQTINYWLAWYQQKP | 18.72\|4.58\| |
| | | | | | (SEQ ID NO: 735)\|46-60- | 17.19 |
| | | | | | KLLIYRASTLASGVP (SEQ ID | |
| | | | | | NO: 723)\|88-102- | |
| | | | | | YCQNYYYSTSRSYGF (SEQ ID | |
| | | | | | NO: 736) | |
| | | | | | 26-40-SQTINYWLAWYQQKP | |
| 334 | 27-07-G02-sc01 | 2 | 3\|3 | 6 | (SEQ ID NO: 735)\|87-101- | 18.72\|17.19 |
| | (A67G_F68G) | | | | YCQNYYYSTSRSYGF (SEQ ID | |
| | | | | | NO: 736) | |
| 353 | 33-03-G02_Q108A_G109A | 2 | 6\|5 | 11 | 44-58-PKLLIYKASTLASGV (SEQ | 7.45\|13.28 |
| | (Reference) | | | | ID NO: 721)\|83-97- | |
| | | | | | FATYYCQAAYIITDI (SEQ ID NO: | |
| | | | | | 737) | |
| 354 | 33-03-G02_Q108A_G109A | 1 | 5 | 5 | 83-97-FATYYCQAAYIITDI (SEQ | 13.28 |
| | (A67G_F68G) | | | | ID NO: 737) | |

TABLE 10
Predicted T cell epitopes for VL sequences of the GG variants and the non-GG versions of the
produced scFvs.

| SEQ | | | | | | 15 mer Hotspot |
|---|---|---|---|---|---|---|
| ID | | Clusters | Cluster | Cluster | 15 mer Hotspot | median perc |
| NO | Clone ID | VL | size VL | sum VL | sequence(s) VL | rank(s) VL |
| 663 | 37-20-B03_human cons | 0 | 0 | 0 | NONE | NONE |
| 664 | 37-20-B03_human cons_Y42S | 0 | 0 | 0 | NONE | NONE |
| 662 | 37-20-B03 sc09.1_(A9S) | 0 | 0 | 0 | NONE | NONE |
| | (Reference) | | | | | |
| 23 | 37-20-B03 sc09.1 | 0 | 0 | 0 | NONE | NONE |
| | (Reference) | | | | | |
| 26 | 37-20-B03-sc09.1_(Y42S) | 0 | 0 | 0 | NONE | NONE |
| | (Reference) | | | | | |
| 35 | 37-20-B03 sc16 | 0 | 0 | 0 | NONE | NONE |
| | (A67G_F68G) | | | | | |
| 41 | 37-20-B03 sc17 | 0 | 0 | 0 | NONE | NONE |
| | (A67G_F68G) | | | | | |
| 47 | 37-20-B03 sc09-IF | 0 | 0 | 0 | NONE | NONE |
| 48 | 37-20-B03 sc09-IF | 0 | 0 | 0 | NONE | NONE |
| | (A67G_F68G) | | | | | |
| 70 | 33-03-G02 sc18 | 1 | 4 | 4 | 57-71-VDITYYASWAKGRVT | 17.15 |
| | | | | | (SEQ ID NO: 738) | |
| 71 | 33-03-G02-sc18 | 1 | 4 | 4 | 57-71-VDITYYASWAKGRVT | 17.15 |
| | (A67G_S68G) | | | | (SEQ ID NO: 738) | |
| 72 | 33-03-G02-sc18 | 1 | 4 | 4 | 57-71-VDITYYASWAKGRVT | 17.15 |
| | (T101S_T146K) | | | | (SEQ ID NO: 738) | |
| | (A67G_S68G) | | | | | |
| 73 | 33-03-G02-sc18 | 0 | 0 | 0 | NONE | NONE |
| | (S72D_W73S_A74V) | | | | | |
| | (A67G_S68G) | | | | | |
| 82 | 38-27-A11-sc02 | 0 | 0 | 0 | NONE | NONE |
| | (Reference) | | | | | |
| 83 | 38-27-A11-sc02 | 0 | 0 | 0 | NONE | NONE |
| | (A67G_S68G) | | | | | |
| 91 | 38-27-A11-sc02 | 0 | | 0 | NONE | NONE |
| | (G51C_N72Q)_(T141C) | | | | | |
| | (Reference) | | 0 | | | |
| 92 | 38-27-A11-sc02 | 0 | 0 | 0 | NONE | NONE |
| | (G51C_N72Q)_(T141C) | | | | | |
| | (A67G_S68G) | | | | | |
| 107 | 38-27-A11-sc10 | 0 | 0 | 0 | NONE | NONE |
| | (Reference) | | | | | |
| 108 | 38-27-A11-sc10 | 0 | 0 | 0 | NONE | NONE |
| | (A67G_S68G) | | | | | |
| 109 | 38-27-A11-sc10 | 0 | 0 | 0 | NONE | NONE |
| | (G51C)_(T141C) | | | | | |
| | (A67G_S68G) | | | | | |
| 134 | 38-02-A04-sc13 (Reference) | 0 | 0 | 0 | NONE | NONE |
| 135 | 38-02-A04-sc13 | 0 | 0 | 0 | NONE | NONE |
| | (A67G_S68G) | | | | | |
| 142 | 38-02-A04-sc16 (Reference) | 0 | 0 | 0 | NONE | NONE |
| 143 | 38-02-A04-sc16 | 0 | 0 | 0 | NONE | NONE |
| | (A67G_S68G) | | | | | |
| 144 | 38-02-A04- | 0 | 0 | 0 | NONE | NONE |
| | sc16_(G51C)_(T141C) | | | | | |
| | (A67G_S68G) | | | | | |
| 156 | 28-21-D09-sc04 (Reference) | 0 | 0 | 0 | NONE | NONE |
| 157 | 28-21-D09-sc04 | 0 | 0 | 0 | NONE | NONE |
| | (A67G_S68G) | | | | | |
| 165 | 28-21-D09-sc43 (Reference) | 0 | 0 | 0 | NONE | NONE |
| 166 | 28-21-D09-sc43 | 0 | 0 | 0 | NONE | NONE |
| | (A67G_S68G) | | | | | |
| 167 | 28-21-D09- | 0 | 0 | 0 | NONE | NONE |
| | sc43_(T101S_T146K) | | | | | |
| | (Reference) | | | | | |
| 168 | 28-21-D09- | 0 | 0 | 0 | NONE | NONE |
| | sc43_(T101S_T146K) | | | | | |
| | (A67G_S68G) | | | | | |
| 169 | 28-21-D09- | 0 | 0 | 0 | NONE | NONE |
| | sc43_(T101S_T146K) | | | | | |
| | (G51C_T141C) (A67G_S68G) | | | | | |
| 177 | 19-04-A10-sc02 (Reference) | 1 | 3 | 3 | 24-38-ASGFSLSSYAMNWVR | 14.06 |
| | | | | | (SEQ ID NO: 739) | |
| 178 | 19-04-A10-sc02 | 1 | 3 | 3 | 24-38-ASGFSLSSYAMNWVR | 14.06 |
| | (A67G_S68G) | | | | (SEQ ID NO: 739) | |
| 186 | 19-04-A10-sc11 (Reference) | 1 | 3 | 3 | 23-37-ASGFSLSSYAMNWVR | 14.06 |
| | | | | | (SEQ ID NO: 739) | |
| 187 | 19-04-A10-sc11 | 1 | 3 | 3 | 23-37-ASGFSLSSYAMNWVR | 14.06 |
| | (A67G_S68G) | | | | (SEQ ID NO: 739) | |
| 188 | 19-04-A10-sc12 (Reference) | 1 | 3 | 3 | 23-37-ASGFSLSSYAMNWVR | 14.06 |
| | | | | | (SEQ ID NO: 739) | |
| 189 | 19-04-A10-sc12 | 1 | 3 | 3 | 23-37-ASGFSLSSYAMNWVR | 14.06 |
| | (A67G_S68G) | | | | (SEQ ID NO: 739) | |
| 221 | 54-21-H03-sc01 | 1 | 3 | 3 | 58-72-VRTYYATWAKGRFTI | 18.18 |
| | (Reference) | | | | (SEQ ID NO: 740) | |
| 222 | 54-21-H03-sc01 | 1 | 3 | 3 | 58-72-VRTYYATWAKGRFTI | 18.18 |
| | (A67G_S68G) | | | | (SEQ ID NO: 740) | |
| 244 | 54-32-A07- | 0 | 0 | 0 | NONE | NONE |
| | sc06_(G51C_T141C) | | | | | |
| | (Reference) | | | | | |
| 245 | 54-32-A07- | 0 | 0 | 0 | NONE | NONE |
| | sc06_(G51C_T141C) | | | | | |
| | (A67G_S68G) | | | | | |
| 271 | 55-39-G02-sc02 (Reference) | 2 | 5\|5 | 10 | 24-38-VSGLSLSRNAMSWVR | 13.78\|18.75 |
| | | | | | (SEQ ID NO: 741)\|46-60- | |
| | | | | | EWIGIILTSGSTYYA (SEQ ID | |
| | | | | | NO: 742) | |
| 272 | 55-39-G02-sc02 | 2 | 5\|5 | 10 | 24-38-VSGLSLSRNAMSWVR | 13.78\|18.75 |
| | (A67G_S68G) | | | | (SEQ ID NO: 741)\|46-60- | |
| | | | | | EWIGIILTSGSTYYA (SEQ ID | |
| | | | | | NO: 742) | |
| 295 | 55-38-D07-sc02 (Reference) | 0 | 0 | 0 | NONE | NONE |
| 296 | 55-38-D07-sc02 | 0 | 0 | 0 | NONE | NONE |
| | (A67G_S68G) | | | | | |
| 299 | 55-38-D07-sc09 (Reference) | 0 | 0 | 0 | NONE | NONE |
| 300 | 55-38-D07-sc09 | 0 | 0 | 0 | NONE | NONE |
| | (A67G_S68G) | | | | | |
| 301 | 55-38-D07-sc09_(G51C) | 0 | 0 | 0 | NONE | NONE |
| | (A67G_S68G_T141C) | | | | | |
| 305 | Trastuzumab_lambda-cap | 0 | 0 | 0 | NONE | NONE |
| | (Reference) | | | | | |
| 306 | Trastuzumab_lambda-cap | 0 | 0 | 0 | NONE | NONE |
| | (A67G_S68G) | | | | | |
| 310 | Pertuzumab_lambda-cap | 0 | 0 | 0 | NONE | NONE |
| | (Reference) | | | | | |
| 311 | Pertuzumab_lambda-cap | 0 | 0 | 0 | NONE | NONE |
| | (A67G_S68G) | | | | | |
| 320 | 14-11-D07-sc01 (Reference) | 0 | 0 | 0 | NONE | NONE |
| 323 | 14-11-D07-sc04 | 0 | 0 | 0 | NONE | NONE |
| | (A67G_S68G) | | | | | |
| 333 | 27-07-G02-sc01 (Reference) | 0 | 0 | 0 | NONE | NONE |
| 334 | 27-07-G02-sc01(A67G_F68G) | 0 | 0 | 0 | NONE | NONE |
| 353 | 33-03-G02_Q108A_G109A | 1 | 4 | 4 | 57-71-VDITYYASWAKGRVT | 17.15 |
| | (Reference) | | | | (SEQ ID NO: 738) | |
| 354 | 33-03-G02_Q108A_G109A | 1 | 4 | 4 | 57-71-VDITYYASWAKGRVT | 17.15 |
| | (A67G_F68G) | | | | (SEQ ID NO: 738) | |

### 2.4. Prediction of the MCH Class II Binding Potency of PRO1480 and of a GG Variant of PRO1480 Using NetMHCIIpan-3.1:

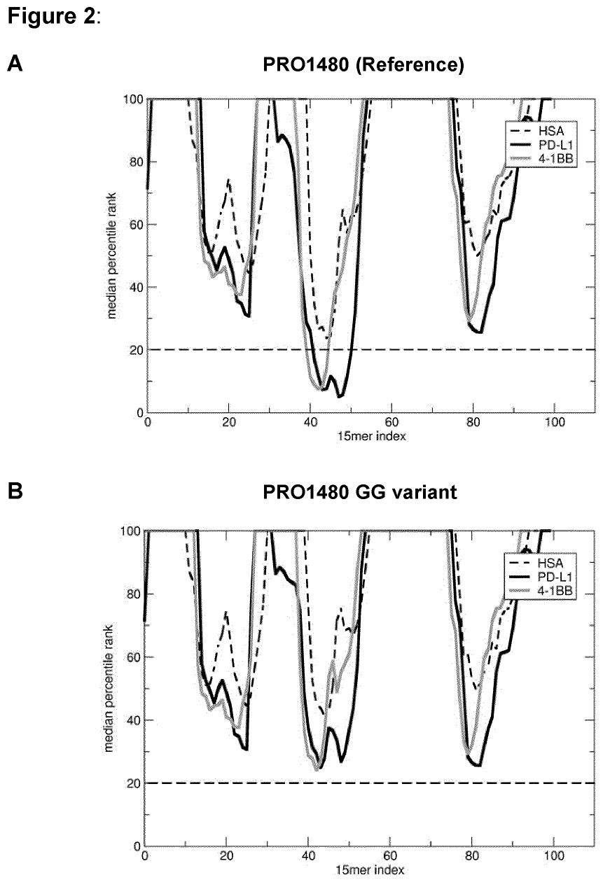

The methodology described above in section 2.2 has been applied to the multispecific anti-PDL1×CD137×hSA antibody PRO1480 as well as for a GG variant of PRO1480. The respective median percentile rank plots are shown in FIG. 2.

As apparent from FIG. 2, the introduction of the GG motif into the LCDR2 of the PDL1-, 4-1BB- (CD137-) and hSA-binding domains generally reduced the predicted binding to MHC class II molecules significantly, resulting in a final PRO1480 variant that no longer has any predicted T-cell immunogenicity risk.

2.5. Prediction of the MCH Class II Binding Potency of 206 Clinical Antibody Therapeutics and their GG Variants Using NetMHCIIpan-3.1:

The methodology described above in section 2.2 has further been applied to a set of 206 sequences of therapeutic antibodies that have been clinically tested, i. e. approved antibody therapeutics or clinical stage antibody therapeutics. These sequences have been published in Marks et al. (Marks et al., Bioinformatics, Volume 37, Issue 22, November 2021, pp. 4041-4047), together with the corresponding percentage of anti-drug antibody (ADA) responses in patients for each of said therapeutics, obtained from clinical papers (referred to as immunogenicity levels in Marks et al.). Most of these antibody sequences had no glycine at both light chain amino acid positions 67 and 68 (AHo numbering), while just some antibody sequences had one glycine, either at position 67 or 68 (AHo numbering). None of them had glycine residues at both positions. 86% of the corresponding GG variants of those 206 antibody sequences no longer exhibited predicted binding to MHC class II proteins and the other GG variants exhibited a significantly reduced predicted binding to MHC class II proteins, except for one.

The median percentile rank plot of the VL regions of these 206 therapeutic antibodies as well as a median percentile rank plot for their corresponding GG variants are shown in FIG. 3.

### Example 3: ADA-Binding Assay

### 3.1. General Assay Procedure:

A method was developed at Numab to detect anti-drug-antibodies in human serum, using a direct assay format.

Typically, 96 well half-area plates were coated with 100 ng/ml of the test molecule (e. g. scFv molecules) for 2 h at room temperature. The plates were blocked for 1 h with PBS containing 0.2% Tween and 1% BSA. Individual human sera were then added at a dilution of 1:20 (5% serum) or 1:100 (1% serum), either unspiked (screening assay) or spiked (confirmatory assay) with the same molecule as coated in the corresponding well. The spiking concentration ranged from 60 to 115 nM and spiked samples were pre-incubated for 1 h. Antibodies bound to the molecules coated on the plate where then detected with 100 ng/ml rabbit anti-human IgG-HRP for 1 h. TMB substrate was added as substrate and after a short incubation, the enzymatic reaction was stopped with 1 M HCl. The optical density of each well was read at 450 nm.

All steps were performed at room temperature. Between each step, plates were washed three times with 450 μl wash buffer. Except for the blocking and washing steps, all assay components were added in a volume of 25 μl/well and duplicates were used. For the incubation steps, the ELISA plates were placed on a rotating mixer (40 rpm).

Typically, two measurements were performed for each test molecule: A first measurement, where untreated original human sera, i. e. unspiked human sera (screening assay), were applied to the plates coated with the individual test molecules; and a second measurement, where human sera that had been pre-incubated with the same compounds as coated in the corresponding well, i. e. spiked human sera (confirmatory assay), were applied to the plates coated with the individual test molecules, to determine whether the initially detected binding of antibodies in the respective sera sample is specific to the test molecule. A decrease of the absorbance signal in the spiked wells indicates that the signal observed in the unspiked well of the initial screening assay is specific to the molecule coated on the plate. The resulting percent inhibition (% inhibition) was calculated as reduction of the initial signal obtained for unspiked serum (screening assay) as follows:

% inhibition=100*(1−(signal spiked serum/signal unspiked serum))

Typically, only spiked samples having % inhibition values above a certain threshold, referred to as confirmatory cut-point in percent (% CCP), are deemed to represent specific binding (screening positive). The % CCP required to confirm specificity was either set at 20 to 30% inhibition or the % CCP was calculated from the mean % inhibition of all serum samples measured as defined in section 2.2. Typically, the % CCP was set to 30% for the analysis of the pre-ADA binding signals. Samples, where percent inhibition was above the % CCP, were regarded as positive.

In some specific cases, when necessary, a screening cut-point (SCP) was calculated from the spiked sera measurements for each plate, to eliminate false positives resulting from binders that initially exhibit very low or no binding to ADAs in the test serum samples (low or no binding for unspiked and spiked serum samples). Unspiked samples with signal below the SCP were termed “screening negative” and were not taken into account. The SCP was calculated as indicated below in section 3.2.

### 3.2. Determination/Calculation of Screening Cut Point (SCP) and Confirmatory Cut-Point in % (% CCP)

### Screening Cut-Point (SCP):

The screening cut point (SCP) is the threshold at which a signal is considered positive (screening positive). The screening cut point (SCP) was calculated from the spiked sera measurements as follows:

SCP=mean N+3×SDN
Wherein:
a. “Mean N” corresponds to mean signal from all spiked individual sera measured for a specific test compound;
b. “SDN” corresponds to standard deviation calculated from all spiked individual sera measured for a specific test compound.

### Calculation of the Confirmatory Cut-Point in Percent (% CCP):

The confirmatory cut point in percent (% CCP) is the threshold at which a % inhibition is considered to be specific (specific inhibition).

The confirmatory cut point in percent (% CCP) is either set to a specific value, e. g. to a value in the range of 20.0 to 30.0%, or is calculated as follows:

% CCP=mean [% inhibition]+2.33×SDN
wherein:
c. “Mean [% inhibition]” corresponds to mean inhibition value in percent for all spiked individual sera measured for a specific test compound;
d. “SDN” corresponds to standard deviation calculated from [% inhibition] values of all spiked individual sera measured for a specific test compound.

### 3.3. ADA-Binding Assay Results for Unmodified PRO1480 and PRO1480 GG Variant According to the Present Invention Using Patient-Derived Individual Human Serum Samples

To analyze the ability of the antibody GG variants of the present invention to reduce the risk of pre-existing ADA-binding and, in particular, to investigate their binding properties towards ADAs that have been formed during therapeutic treatment with PRO1480, a set of patient-derived individual human serum samples were used. All sera were derived from clinical human dose escalation studies with PRO1480. PRO1480 is a fragment-based multispecific anti-PD-L1×CD137×hSA antibody developed by Numab Therapeutics AG. PRO1480 is disclosed in the patent application WO 2019/072868.

In a first study, sera from five individuals, i. e. patient 1 (Subject Number 1003002), 3 (Subject Number 10010002), 8 (Subject Number 10030006), 11 (Subject Number 10030007) and 12 (Subject Number 10010006), were used (see Table 11). The sera were selected based on previous ADA assays in which treatment-emerging ADAs were identified. Patient 1 (Subject Number 1003002), 8 (Subject Number 1003006) and 11 (Subject Number1003007) also showed evidence for the presence of pre-existing ADAs. The sera were taken at different time points of a first interpatient dose escalation study. Different patients received different dose levels.

TABLE 11
List of individual patient-derived serum
samples used for ADA binding assay.

| Patient | Subject | Study timepoint | Dose |
|---|---|---|---|
| Number | Number | (Days) | level |
| 1 | 10030002 | 0 | C1 |
| 1 | 10030002 | 8 | C1 |
| 1 | 10030002 | 29 | C2 |
| 1 | 10030002 | 43 | C2 |
| 2 | 10010001 | 0 | C1 |
| 3 | 10010002 | 0 | C1 |
| 3 | 10010002 | 15 | C1 |
| 3 | 10010002 | 29 | C2 |
| 3 | 10010002 | 43 | C2 |
| 6 | 10030004 | 0 | C1 |
| 6 | 10030004 | 57 | C3 |
| 8 | 10030006 | 0 | C1 |
| 8 | 10030006 | 15 | C1 |
| 8 | 10030006 | 57 | C3 |
| 8 | 10030006 | 169 | C7 |
| 8 | 10030006 | 225 | C9 |
| 11 | 10030007 | 0 | C1 |
| 11 | 10030007 | 15 | C1 |
| 12 | 10010006 | 0 | C1 |
| 12 | 10010006 | 8 | C1 |
| 12 | 10010006 | 15 | C1 |
| 12 | 10010006 | 29 | C2 |
| 12 | 10010006 | 43 | C2 |
| 13 | 10010009 | 0 | C1 |
| 13 | 10010009 | 57 | C3 |
| 14 | 10010010 | 0 | C1 |
| 15 | 10010011 | 57 | C3 |

In this first study, the following molecules were tested: PRO 2230 (positive control) corresponding to the PD-L1 BD of PRO1480, as well as PRO4180 and PRO4181, two GG variants of PRO1480.

In this first study, the ELISA-based binding assay for evaluating the binding properties of the scFv PRO2230 (positive control) and the PRO1480 variants was performed as follows. 96 well half-area plates were coated with PRO1480 (100 ng/ml diluted in PBS pH 7.4) for 2 h at RT. The plates were blocked for 1 h with PBS containing 0.2% Tween and 1% BSA. Individual patient-derived human sera were then added at a dilution of 1:20 (5% serum). The sera were spiked either with the MATCH-3 molecules (150 nM final concentration in serum) or with the scFv PRO2230 (450 nM final concentration in serum). Spiked samples were pre-incubated for 1 h. Identically as in all other ADA assays, antibodies bound to PRO1480 were then detected with 100 ng/ml rabbit anti-human IgG-HRP for 1 h. TMB substrate was added as substrate and after a short incubation, the enzymatic reaction was stopped with 1 M HCl. The optical density of each well was read at 450 nm.

All steps were performed at RT. Between each step, plates were washed three times with 450 μl wash buffer. Except for the blocking and washing steps, all assay components were added in a volume of 25 μl/well and duplicates were used. For the incubation steps, the ELISA plates were placed on a rotating mixer (40 rpm).

Data evaluation and calculations were done as described above, except that no SCP and no CCP were calculated and used in the evaluation.

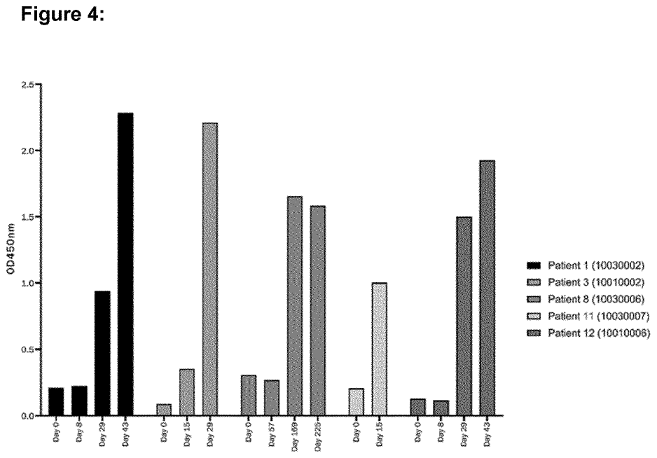

The absorption signals obtained for unmodified PRO1480 against the 17 tested native patient sera are shown in FIG. 4. The higher the absorption level, the more ADAs are present in the serum samples that bind PRO1480. As can be deduced from FIG. 4, ADAs against PRO1480 are present in serum samples collected after 3-week treatment. The slight increased absorption level observed for sera from patients 1, 8 and 11 taken at day 0, indicate the presence of some pre-existing ADAs that bind to PRO1480.

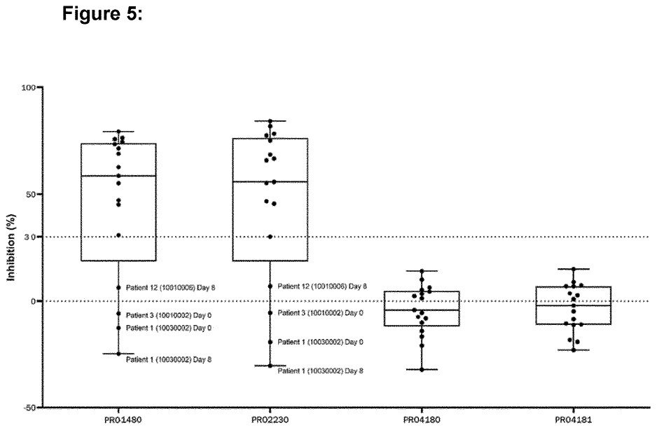

Results of the ADA binding assay for GG modified PRO1480 versions against the patient-derived individual human serum samples are summarized in Table 12 and FIG. 5. Apparently, for the GG variants PRO4180 and PRO4181, no significant ADA binding (% inhibition below 30%) could be detected, whereas for PRO1480, significant amounts of ADA binding (% inhibition above 30%) could be detected for 13 of the 17 serum samples. Table 13 summarizes the individual % inhibition values obtained for PRO1480, PRO2230, PRO4180 and PRO4181 against the 17 patient sera.

TABLE 12
Total number of positive serum samples for
PRO1480, PRO2230, PRO4180 and PRO4181.

| | | | No of serum |
|---|---|---|---|
| | | | samples >30% |
| | | | inhibition |
| | Parental | | (without |
| Molecule | molecule | Substitutions | SCP or CCP) |
| PRO2230 | — | wt | 13 |
| PRO1480 | — | wt | 13 |
| PRO4180 | PRO1480 | T301S, T346K, T701S, | 0 |
| | | T746K, T1101S, T1146K; | |
| | | N272Q; “GG”-PDL1 | |
| | | BD; “DiS” hSA-BD | |
| PRO4181 | PRO1480 | T301S, T346K, T701S, T746K, | 0 |
| | | T1101S, T1146K; N272Q; | |
| | | “GG”-PDL1 BD; | |
| | | “GG” CD137-BD; | |
| | | “GG” hSA-BD; | |
| | | “DiS” hSA-BD | |

TABLE 13
% inhibition values for PRO1480, PRO2230, PRO4180 and
PRO4181 against individual 17 patient serum samples.

| Patient | Timepoint | Native serum | Inhibition of signal after spiking (%) | | | |
|---|---|---|---|---|---|---|
| Number | (Days) | (OD450 nm) | PRO1480 | PRO2230 | PRO4180 | PRO4181 |
| 1 | 0 | 0.210 | −12.6 | −19.2 | −16.7 | −19.1 |
| | 8 | 0.223 | −24.8 | −30.4 | −32.2 | −8.5 |
| | 29 | 0.943 | 58.6 | 45.5 | −4.2 | −4.8 |
| | 43 | 2.287 | 75.7 | 66.6 | 14.0 | 14.9 |
| 3 | 0 | 0.089 | −5.8 | −5.5 | −7.4 | −10.6 |
| | 15 | 0.353 | 76.3 | 77.4 | −14.0 | −11.2 |
| | 29 | 2.212 | 79.3 | 84.2 | 4.4 | 0.9 |
| 8 | 0 | 0.307 | 68.9 | 68.5 | −20.9 | −23.0 |
| | 57 | 0.269 | 47.1 | 46.7 | 2.3 | 2.7 |
| | 169 | 1.656 | 71.4 | 78.2 | 10.1 | 6.9 |
| | 225 | 1.583 | 74.4 | 81.8 | 6.3 | 8.9 |
| 11 | 0 | 0.207 | 55.1 | 55.7 | −10.1 | −18.2 |
| | 15 | 1.004 | 73.4 | 75.1 | 1.3 | 7.3 |
| 12 | 0 | 0.128 | 30.8 | 30.1 | −8.1 | −2.1 |
| | 8 | 0.115 | 6.3 | 6.9 | −5.5 | −11.0 |
| | 29 | 1.501 | 62.6 | 65.8 | 3.4 | 3.6 |
| | 43 | 1.926 | 45.1 | 55.2 | 5.2 | 6.9 |

In a second study, sera from ten individuals, i. e. patient 1 (Subject Number 1003002), patient 2 (Subject Number 10010001), 3 (Subject Number 10010002), patient 6 (Subject Number 10030004), 8 (Subject Number 10030006), 11 (Subject Number 10030007), 12 (Subject Number 10010006), 13 (Subject Number 10010009), 14 (Subject Number 10010010) and 15 (Subject Number 10010011), were used (see Table 11). The sera were selected based on previous ADA assays in which treatment-emerging ADAs were identified. Patient 6 (Subject Number 10030004), 8 (Subject Number 1003006), 11 (Subject Number1003007), 12 (Subject Number 10010006) and 13 (Subject Number 10010009) also showed evidence for the presence of medium to high levels of pre-existing ADAs. The sera were taken at different time points of a first interpatient dose escalation study. Different patients received different dose levels.

In this second study, the following molecules were tested: PRO 2230 (positive control) corresponding to the PD-L1 BD of PRO1480, as well as PRO4180, PRO4181, PRO5033 and PRO5034, four GG variants of PRO1480.

The ELISA-based binding assay for evaluating the binding properties of the scFv PRO2230 (positive control) and the PRO1480 variants has been performed similar to the first study. 96 well half-area plates were coated with PRO1480 or PRO2230, PRO4180, PRO4181, PRO5033, PRO5034 respectively (100 ng/ml diluted in PBS pH 7.4) for 2 h at RT. The plates were blocked for 1 h with PBS containing 0.2% Tween and 1% BSA. Individual patient-derived human sera were then added at a dilution of 1:20 (5% serum). The sera were spiked either with the MATCH-3 molecules (150 nM final concentration in serum) or with the scFv PRO2230 (450 nM final concentration in serum). Spiked samples were pre-incubated for 1 h. Identically as in all other ADA assays, antibodies bound to PRO1480 and its variants were then detected with 100 ng/ml rabbit anti-human IgG-HRP for 1 h. TMB substrate was added as substrate and after a short incubation, the enzymatic reaction was stopped with 1 M HCl. The optical density of each well was read at 450 nm.

All steps were performed at RT. Between each step, plates were washed three times with 450 μl wash buffer. Except for the blocking and washing steps, all assay components were added in a volume of 25 μl/well and duplicates were used. For the incubation steps, the ELISA plates were placed on a rotating mixer (40 rpm).

Data evaluation and calculations were done as described above, except that no SCP and no CCP were calculated and used in the evaluation.

Results of the ADA binding assay for GG modified PRO1480 versions against the patient-derived individual human serum samples of this second study are summarized in Table 14. The values in Table 14 correspond to the individual % inhibition values obtained for PRO1480, PRO2230, PRO4180, PRO4181, PRO5033 and PRO5034 against 25 patient sera.

As can be deduced from the data of Table 14, PRO5033, which contains a “GG modification” in the PDL1-BD and PRO5034, which contains “GG modifications” in the PDL1 and CD137-BD, show clearly lower inhibition rates when compared to their unmodified version PRO1480. For patient sera that comprise high levels of pre-existing ADAs, i. e. sera samples from patients 8, 11 and 12, the absolute reduction of the inhibition rates is relatively small due to the fact that the “GG modification” has only a small effect on pre-existing ADA binding. PRO4180 and PRO4181 generally show stronger reduction in the inhibition rates, when compared to their unmodified version PRO1480, since these variants also comprise the “SK modification” in the heavy chain variable region of the individual binding domains, which typically eliminates the binding to pre-existing ADAs very efficiently.

### Example 4: KD Determination by SPR

For determination of the binding kinetics and affinity of the GG modified scFvs and the corresponding non modified references to their respective targets, the following general procedure was applied.

The respective target proteins of the tested scFvs were immobilized on a CMD200M SPR sensor prism chip (Xantec) in an SPR 24 system (Sierra Sensor-Bruker). The mutation variants were then injected at concentrations of 10 nM, 2 nM and 0.4 nM over the ligand-immobilized spots and reference spots. The wild type variants were used as control, while an empty spot was used as reference.

After each analyte cycle, the surface was regenerated with 3 M MgCl₂ solution, thereby allowing the next analyte cycle to have only free ligand available. Binding affinity K_{D} was globally calculated by the Bruker SPR software based on the binding association (kₒₙ) and dissociation (k_{off}) rate, by fitting a 1:1 Langmuir model to the curves obtained by the cycles with the three different concentrations of the analyte.

TABLE 14
% inhibition values for PRO1480, PRO2230, PRO4180, PRO4181, PRO5033
and PRO5034 against individual 25 patient serum samples.

| | Time | | | | | | |
|---|---|---|---|---|---|---|---|
| Patient | Point | Inhibition of signal after spiking (%) | | | | | |
| number | (Days) | PRO1480 | PRO2230 | PRO4180 | PRO4181 | PRO5033 | PRO5034 |
| 1 | 0 | 0.1 | −34.3 | −12.9 | −5.3 | −4.9 | −0.7 |
| | 8 | 8.5 | 22.5 | −7.6 | 2.1 | 6.9 | 1.8 |
| | 29 | 55.4 | 55.4 | 29.7 | 31.0 | 53.1 | 52.9 |
| 2 | 0 | 14.3 | 27.4 | −8.4 | −2.0 | −2.5 | 11.2 |
| 3 | 0 | −2.4 | 7.9 | −18.1 | −5.9 | −6.0 | −12.6 |
| | 15 | 80.1 | 63.8 | 9.9 | 15.2 | 12.8 | 16.5 |
| | 29 | 85.6 | 87.4 | 53.5 | 57.0 | 62.8 | 64.3 |
| | 43 | 87.4 | 87.9 | 82.9 | 84.0 | 85.2 | 85.7 |
| 6 | 0 | 15.5 | 70.9 | −5.6 | −9.7 | 10.8 | 11.7 |
| | 57 | 15.6 | 51.2 | −6.8 | −5.3 | 11.7 | −3.4 |
| 8 | 0 | 76.2 | 91.0 | −21.8 | −21.7 | 76.2 | 73.0 |
| | 15 | 82.7 | 88.8 | 75.3 | 76.3 | 79.8 | 80.6 |
| | 57 | 87.7 | 90.1 | 78.0 | 79.9 | 82.5 | 83.8 |
| | 225 | 61.8 | 88.2 | 3.1 | 4.6 | 53.2 | 43.5 |
| 11 | 0 | 77.2 | 93.1 | −4.2 | −12.6 | 74.9 | 70.5 |
| | 15 | 78.9 | 81.9 | 13.0 | 26.2 | 82.3 | 81.4 |
| 12 | 0 | 59.8 | 86.7 | −7.0 | −1.8 | 53.0 | 53.2 |
| | 8 | 28.0 | n.d. | −6.5 | −11.1 | 17.6 | 20.0 |
| | 15 | 86.6 | n.d. | 49.3 | 47.6 | 85.9 | 87.0 |
| | 29 | 60.3 | 67.8 | 49.2 | 44.8 | 72.0 | 74.6 |
| | 43 | 57.4 | 70.1 | 62.8 | 63.1 | 74.4 | 75.9 |
| 13 | 0 | 34.0 | n.d. | 5.3 | 2.4 | 24.8 | 33.5 |
| | 57 | 70.0 | 41.2 | 38.8 | 68.9 | 50.5 | 43.0 |
| 14 | 0 | 5.1 | 52.6 | −48.8 | −12.2 | −6.8 | −29.8 |
| 15 | 57 | 42.0 | 38.5 | 32.9 | 39.4 | 40.7 | 36.2 |

n.d.: not determined

As apparent from Table 15, the KD values of the GG modified scFvs variants for binding to their respective targets generally showed only little variation, when compared to their reference compounds. This means that no significant difference in binding affinity between the variants and the corresponding reference compounds could be identified. These results indicate that the GG modification (67G and 68G) in the CDRL2 of the tested scFvs does not significantly influence their binding affinities to their targets. This is in contrast to the KD measurements performed with the anti-PD-L1 scFvs PRO3477 and PRO3478, where their LCDR2s have been changed into a human consensus LCDR2 sequence, which showed no more binding to PD-L1.

### Example 5: Thermal Stability Measurement Using nanoDSF

The GG modified scFvs according to the present invention and their respective unmodified versions were analyzed for thermal stability using nanoDSF. The aim of these thermal stability measurement was to determine the influence of the GG modification on the stability of the respective scFvs.

During nanoDSF measurement, molecules in solution were subjected to increasing temperature which leads to the unfolding of the molecules. The unfolding event causes a shift of the fluorescence emission spectra of tryptophan (Trp) by a change of its environment, e. g., from the hydrophobic core to solvent-exposed region. In the curve of the shift of the fluorescence emission, the inflection point of the unfolding curve is defined as the thermal midpoint (Tm). Tm is an important factor to assess the stability of protein. Higher Tm suggests a more stable protein. A protein can have multiple Tm based on the number of domains that unfold separately. The thermal stability is further influenced by the protein concentration and buffer conditions.

The following general procedure was applied. Proteins in 20 mM histidine buffer (pH 6) at 1±0.1 mg/ml were prepared for thermal stability. Melting curves were generated with a temperature increase from 20° C. to 95° C. with a 1° C./min. The intrinsic fluorescence of the proteins was monitored and recorded. The spectral shift upon unfolding was recorded at two wavelengths, 330 nm and 350 nm, and the ratio of 350 nm/330 nm was taken to analyze the data. The protein unfolding was conducted using the Prometheus device (Nanotemper Technologies) and analyzed by the “PR.ThermControl” v2.3.1 and “PR.Stability Analysis” v1.1 software.

As apparent from Table 15, the Tm values obtained for the GG modified scFvs variants do not vary much from the Tm values obtained for the corresponding reference compounds. Almost all GG variants exhibit high Tm values indicating excellent stability also for the GG modified scFvs variants. These results indicates that the GG modification (67G and 68G) in the CDRL2 of the tested scFvs does not significantly influence their thermal stability.

TABLE 15
Overview of melting temperatures and binding affinities of GG-modified scFvs and their non-modified version (References).

| | | | | | | | | | KD ratio to |
|---|---|---|---|---|---|---|---|---|---|
| SEQ | | | | Tm1 | | Tm2 | | KD | parental (>1 |
| ID | Target | PRO-ID | Clone ID | [°C] | ΔTm1 | [°C] | ΔTm2 | [M] | is worse) |
| 23 | PD-L1 | PRO2230 | 37-20-B03 sc09.1 (Reference) | 72.8 | | 81.7 | | 1.34E−11 | 1 |
| 663 | PD-L1 | PRO3477 | 37-20-B03-sc09.1_human cons | 68.2 | −4.6 | 76.9 | −4.8 | NB *⁾ | — |
| 26 | PD-L1 | PRO3476 | 37-20-B03-sc09.1_(Y42S) (Reference) | 73.6 | | 81.9 | | 2.11E−11 | 1 |
| 664 | PD-L1 | PRO3478 | 37-20-B03-sc09.1_(Y42S) human cons | 66.5 | −7.1 | 77.2 | −4.7 | NB *⁾ | — |
| 662 | PD-L1 | PRO3757 | 37-20-B03-sc09.1_(A9S) (Reference) | 72.9 | | 81.9 | | 1.37E−11 | 1 |
| 35 | PD-L1 | PRO4124 | 37-20-B03 sc16 (A67G_F68G) | 70.2 | N/A | 81.1 | −0.8 | 9.94E−11 | N/A |
| 41 | PD-L1 | PRO4125 | 37-20-B03 sc17 (A67G_F68G) | 71.1 | N/A | 81.1 | −0.8 | 1.96E−10 | N/A |
| 47 | PD-L1 | PRO1347 | 37-20-B03 sc09-IF (Reference) | 77.2 | | | | 1.09E−11 | 1 |
| 48 | PD-L1 | PRO3723 | 37-20-B03 sc09-IF (A67G_F68G) | 67.4 | — | 76.5 | −0.7 | 2.58E−11 | 2.37 |
| 70 | PD-L1 | PRO1392 | 33-03-G02 sc18 (Reference) | 70.9 | | 79.2 | | 9.47E−12 | 1 |
| 71 | PD-L1 | PRO3724 | 33-03-G02-sc18 (A67G_S68G) | 66.2 | −4.8 | 74.9 | −4.3 | N/A | N/A |
| 72 | PD-L1 | PRO3891 | 33-03-G02-sc18_(T101S_T146K) | 64.9 | N/A | 74.1 | N/A | N/A | N/A |
| | | | (A67G_S68G) | | | | | | |
| 73 | PD-L1 | PRO3747 | 33-03-G02-sc18_(S72D_W73S_A74V) | 66.3 | N/A | N/A | N/A | N/A | N/A |
| | | | (A67G_S68G) | | | | | | |
| 82 | 4-1BB | PRO1359 | 38-27-A11-sc02 (Reference) | 66.0 | | | | 2.49E−09 | 1 |
| 83 | 4-1BB | PRO3726 | 38-27-A11-sc02 (A67G_S68G) | 62.9 | −3.1 | | | 3.69E−09 | 1.18 |
| 91 | 4-1BB | PRO2194 | 38-27-A11-sc02_(G51C_N72Q)_(T141C) | 68.9 | — | | | 4.63E−09 | 1 |
| | | | Reference) | | | | | | |
| 92 | 4-1BB | PRO3742 | 38-27-A11-sc02_(G51C_N72Q)_(T141C) | 66.4 | −2.5 | | | 6.59E−09 | 1.43 |
| | | | (A67G_S68G) | | | | | | |
| 107 | 4-1BB | PRO4147 | 38-27-A11-sc10 (Reference) | 63.2 | | | | N/A | N/A |
| 108 | 4-1BB | PRO4151 | 38-27-A11-sc10 (A67G_S68G) | 60.1 | −3.1 | | | N/A | N/A |
| 109 | 4-1BB | PRO4155 | 38-27-A11-sc10_(G51C)_(T141C) | N/A | N/A | N/A | N/A | N/A | N/A |
| | | | (A67G_S68G) | | | | | | |
| 134 | 4-1BB | PRO1352 | 38-02-A04-sc13 (Reference) | 65.5 | | | | 1.39E−09 | 1 |
| 135 | 4-1BB | PRO3727 | 38-02-A04-sc13 (A67G_S68G) | 61.2 | −4.3 | 70.8 | N/A | 1.60E−09 | 1.15 |
| 142 | 4-1BB | PRO4148 | 38-02-A04-sc16 (Reference) | 64.8 | | | | N/A | N/A |
| 143 | 4-1BB | PRO4152 | 38-02-A04-sc16 (A67G_S68G) | 59.8 | −5.1 | | | N/A | N/A |
| | | | 38-02-A04-sc16_(G51C)_(T141C) | | | | | | |
| 144 | 4-1BB | PRO4156 | (A67G_S68G) | 61.5 | −3.4 | | | N/A | N/A |
| 156 | CD3e | PRO2781 | 28-21-D09-sc04 (Reference) | 62.8 | | | | 5.88E−09 | 1 |
| 157 | CD3e | PRO3718 | 28-21-D09-sc04 (A67G_S68G) | 60.0 | −2.8 | | | 5.20E−09 | 0.88 |
| 165 | CD3e | PRO4187 | 28-21-D09-sc43 (Reference) | 59.2 | | 73.3 | | N/A | N/A |
| 166 | CD3e | PRO4188 | 28-21-D09-sc43 (A67G_S68G) | 55.9 | −3.3 | 73.1 | −0.1 | N/A | N/A |
| 167 | CD3e | PRO4189 | 28-21-D09-sc43_(T101S_T146K) | 57.9 | | 73.5 | | N/A | N/A |
| | | | (Reference) | | | | | | |
| 168 | CD3e | PRO4190 | 28-21-D09-sc43_(T101S_T146K) | 54.6 | −3.3 | 74.0 | 0.5 | N/A | N/A |
| | | | (A67G_S68G) | | | | | | |
| 169 | CD3e | PRO4191 | 28-21-D09-sc43_(T101S_T146K) | 56.9 | −1.0 | 71.4 | −2.1 | N/A | N/A |
| | | | (G51C_T141C)_(A67G_S68G) | | | | | | |
| 177 | HSA | PRO2155 | 19-04-A10-sc02 (Reference) | 75.3 | | | | 2.62E−09 | 1 |
| 178 | HSA | PRO3721 | 19-04-A10-sc02 (A67G_S68G) | 73.5 | −1.8 | | | 3.40E−09 | 1.30 |
| 186 | HSA | PRO4112 | 19-04-A10-sc11 (Reference) | 78.4 | | | | 1.74E−10 | 1 |
| 187 | HSA | PRO4116 | 19-04-A10-sc11 A67G_S68G) | 76.4 | −2.0 | | | 3.20E−10 | 1.84 |
| 188 | HSA | PRO4113 | 19-04-A10-sc12 (Reference) | 75.9 | | | | 9.01E−11 | 1 |
| 189 | HSA | PRO4117 | 19-04-A10-sc12 (A67G_S68G) | 73.8 | −2.1 | | | 1.38E−10 | 1.53 |
| 221 | MSLN | PRO1922 | 54-21-H03-sc01 (Reference) | 69.9 | | | | 1.51E−10 | 1 |
| 222 | MSLN | PRO3732 | 54-21-H03-sc01 (A67G_S68G) | 66.6 | −3.3 | | | 1.72E−10 | 1.14 |
| 244 | MSLN | PRO2309 | 54-32-A07-sc06_(G51C_T141C) | 62.3 | | | | 5.61E−10 | 1 |
| | | | (Reference) | | | | | | |
| 245 | MSLN | PRO3733 | 54-32-A07-sc06_(G51C_T141C) | 60.8 | −1.5 | | | 1.58E−09 | 2.83 |
| | | | (A67G_S68G) | | | | | | |
| 271 | ROR1 | PRO2062 | 55-39-G02-sc02 (Reference) | 61.5 | | | | 1.08E−10 | 1 |
| 272 | ROR1 | PRO3734 | 55-39-G02-sc02 (A67G_S68G) | 61.4 | −0.1 | | | 1.20E−10 | 1.11 |
| 295 | ROR1 | PRO2060 | 55-38-D07-sc02 (Reference) | 74.4 | | | | 1.19E−10 | 1 |
| 296 | ROR1 | PRO3735 | 55-38-D07-sc02 (A67G_S68G) | 72.5 | −2.0 | | | 3.36E−10 | 2.82 |
| 299 | ROR1 | PRO4135 | 55-38-D07-sc09 (Reference) | 68.3 | | 75.0 | | 1.22E−10 | 1 |
| 300 | ROR1 | PRO4139 | 55-38-D07-sc09 (A67G_S68G) | 67.5 | −0.7 | 72.8 | −2.2 | 2.12E−10 | 1.74 |
| 301 | ROR1 | PRO4143 | 55-38-D07-sc09_(G51C) | 69.3 | 1.0 | 75.8 | 0.8 | 6.31E−10 | 5.17 |
| | | | (A67G_S68G_T141C) | | | | | | |
| 305 | Her2 | PRO0286 | Trastuzumab_lambda-cap (Reference) | N/A | | N/A | | N/A | N/A |
| 306 | Her2 | PRO3716 | Trastuzumab_lambda-cap (A67G_S68G) | N/A | N/A | N/A | N/A | N/A | N/A |
| 310 | Her2 | PRO1812 | Pertuzumab_lambda-cap (Reference) | 68.4 | | | | N/A | N/A |
| 311 | Her2 | PRO3717 | Pertuzumab_lambda-cap (A67G_S68G) | N/A | N/A | | | N/A | N/A |
| 320 | IL23R | PRO0517 | 14-11-D07-sc01 (Reference) | N/A | | N/A | | N/A | N/A |
| 323 | IL23R | PRO3719 | 14-11-D07-sc04 (A67G_S68G) | N/A | N/A | N/A | N/A | N/A | N/A |
| 333 | IL17 | PRO0561 | 27-07-G02-sc01 (Reference) | N/A | | N/A | | N/A | N/A |
| 334 | IL17 | PRO3722 | 27-07-G02-sc01(A67G_F68G) | N/A | N/A | N/A | N/A | N/A | N/A |
| 353 | PD-L1 | PRO1434 | 33-03-G02_Q108A_G109A (Reference) | N/A | | N/A | | 4.91E−07 | 1 |
| 354 | PD-L1 | PRO3725 | 33-03-G02_Q108A_G109A (A67G_F68G) | 73.22 | N/A | N/A | N/A | 4.91E−07 | 1.0 |

1. An antibody variable domain, which binds to a target antigen, comprising:
a. a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region;
and
b. a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region;
wherein said LCDR2 is defined by light chain amino acid positions 58 to 72 according to the AHo numbering scheme, resulting in a maximum length of 15 amino acids, and wherein said LCDR2 has a glycine (G) at amino acid position 67 and a glycine (G) at amino acid position 68 (AHo numbering);
and wherein said light chain complementarity-determining region LCDR2, i. e. amino acid positions 58, 67, 68, 69, 70, 71, 72 (AHo numbering), does not have a sequence selected from LGGNRAA (SEQ ID NO: 665) and RGGERVS (SEQ ID NO: 666).

2. The antibody variable domain of claim 1, wherein
a. said variable light chain framework regions LFW1, LFW2, LFW3 and LFW4 together are selected from a human antibody Vκ framework or a human antibody Vλ framework, in particular a human antibody Vκ framework; or
b. said variable light chain framework regions LFW1, LFW2 and LFW3 together are selected from a human antibody Vκ framework, and said variable light chain framework region LFW4 is selected from a human antibody Vλ framework.

3. The antibody variable domain claim 2, wherein said variable light chain framework regions LFW1, LFW2, LFW3 and LFW4 are selected from
a. the combination of framework regions LFW1, LFW2, LFW3 and LFW4 (i. e. the residues corresponding to the non-italicized residues of SEQ ID NO: 3) of any one of the SEQ ID NOs: 304, 309, 515, 516, 517, 518, 519, 520, 521, 522, 523, 524, 525 and 526; and
b. the combination of framework regions LFW1, LFW2, LFW3 and LFW4 (i. e. the residues corresponding to the non-italicized residues of SEQ ID NO: 3) of any one of the SEQ ID NOs: 304, 309, 515, 516, 517, 518, 519, 520, 521, 522, 523, 524, 525 and 526 having 1, 2, 3, 4 or 5 mutations within the framework regions.

4. The antibody variable domain of claim 1, wherein said variable heavy chain framework regions HFW1, HFW2, HFW3 and HFW4 are of the human VH framework subtype VH3.

5. The antibody variable domain of claim 4, wherein said variable heavy chain framework regions HFW1, HFW2, HFW3 and HFW4 are selected from
a. the combination of framework regions HFW1, HFW2, HFW3 and HFW4 (i. e. the residues corresponding to the non-italicized residues of SEQ ID NO: 1) of any one of the SEQ ID NOs: 302, 306, 496, 497, 498, 499, 500, 501, 502, 503, 504, 505, 506, 507, 508, 509, 510, 511, 512, 513 and 514; and
b. the combination of framework regions HFW1, HFW2, HFW3 and HFW4 (i. e. the residues corresponding to the non-italicized residues of SEQ ID NO: 1) of any one of the SEQ ID NOs: 302, 306, 496, 497, 498, 499, 500, 501, 502, 503, 504, 505, 506, 507, 508, 509, 510, 511, 512, 513 and 514 having 1, 2, 3, 4 or 5 mutations within the framework regions.

6. The antibody variable domain of claim 1, wherein the light chain complementarity-determining region LCDR2 has a sequence selected from X₁GGX₂X₃X₄X₅(SEQ ID NO: 536), wherein X₁, X₂, X₃, X₄ and X₅ correspond to amino acid positions 58, 69, 70, 71 and 72 (AHo numbering), and wherein said X₁, X₂, X₃, X₄ and X₅ of LCDR2 are selected from:
(i) the corresponding positions of an LCDR2 region of a mammalian antibody, in particular of an LCDR2 region of a lagomorph, rodent or human antibody, in particular of an LCDR2 region of a rabbit, mouse or human antibody, in particular of an LCDR2 region of a rabbit antibody; or
(ii) the corresponding positions of an LCDR2 region of an antibody that has been tested in clinical trials; or
(iii) the corresponding positions of an LCDR2 region of an antibody according to (i) or (ii), wherein one, two or three of said amino acid positions, in particular one or two of said amino acid positions, have been altered.

7. The antibody variable domain of claim 6, wherein said light chain complementarity-determining region LCDR2 has one or more of the following features:
(a) has a lysine (K), leucine (L), methionine (M) or arginine (R), in particular a lysine (K), leucine (L) or methionine (M), in particular a leucine (L), at amino acid position 70 (AHo numbering);
(b) has a sequence selected from X₁GGX₂X₃X₄X₅(SEQ ID NO: 536), wherein
X₁ is selected from lysine (K), arginine (R), serine (S), threonine (T), alanine (A), leucine (L), glycine (G), glutamine (Q), glutamate (E), tyrosine (Y) and aspartate (D); in particular from lysine (K), arginine (R), serine (S), tyrosine (Y), glycine (G), and aspartate (D);
X₂ is selected from lysine (K), threonine (T), serine (S), isoleucine (I), aspartate (D), asparagine (N), phenylalanine (F) and tyrosine (Y); in particular from lysine (K), threonine (T), serine (S), aspartate (D), asparagine (N) and tyrosine (Y);
X₃ is selected from lysine (K), leucine (L), methionine (M) and arginine (R); in particular from lysine (K), methionine (M) and leucine (L); in particular is leucine (L);
X₄ is selected from alanine (A), glycine (G), valine (V), threonine (T), proline (P), tyrosine (Y), aspartate (D), and glutamate (E); in particular from alanine (A), glycine (G), threonine (T) and glutamate (E);
X₅ is selected from serine (S), threonine (T) and tyrosine (Y); in particular is serine (S).

8. The antibody variable domain of claim 6, wherein said light chain complementarity-determining region LCDR2, i. e. amino acid positions 58, 67, 68, 69, 70, 71, 72 (AHo numbering), has a sequence selected from RGGILAS (SEQ ID NO: 667), KGGTLAS (SEQ ID NO: 668), RGGTLAS (SEQ ID NO: 669), SGGTLAS (SEQ ID NO: 670), LGGTLAS (SEQ ID NO: 671), TGGTLAS (SEQ ID NO: 672), GGGTLAS (SEQ ID NO: 673), RGGNLAS (SEQ ID NO: 674), DGGDLAS (SEQ ID NO: 675), DGGKLAS (SEQ ID NO: 676), QGGKLAS (SEQ ID NO: 677), RGGKLAS (SEQ ID NO: 678), LGGKLAS (SEQ ID NO: 679), SGGKLAS (SEQ ID NO: 680), GGGKLAS (SEQ ID NO: 681), DGGRLAS (SEQ ID NO: 682), DGGNRAT (SEQ ID NO: 683), RGGTLES (SEQ ID NO: 684), KGGTLES (SEQ ID NO: 685), DGGDLTS (SEQ ID NO: 686), SGGFLYS (SEQ ID NO: 687), SGGYRYT (SEQ ID NO: 688), SGGKLAA (SEQ ID NO: 689), SGGTLVS (SEQ ID NO: 690), RGGTLAY (SEQ ID NO: 691), AGGTLAS (SEQ ID NO: 692), AGGYLAS (SEQ ID NO: 693), RGGYLES (SEQ ID NO: 694), EGGKLAS (SEQ ID NO: 695), RGGNLES (SEQ ID NO: 696), RGGILES (SEQ ID NO: 697), RGGNRES (SEQ ID NO: 698), RGGTLDS (SEQ ID NO: 699), LGGKLES (SEQ ID NO: 700), LGGKMES (SEQ ID NO: 701), TGGSLAS (SEQ ID NO: 702) and GGGTLES (SEQ ID NO: 703).

9. The antibody variable domain claim 1, wherein said antibody variable domain comprises:
(i) a VH sequence selected from SEQ ID NOs: 1 and 2 and from variants of SEQ ID NOs: 1 and 2 having 1, 2 or 3 mutations within the framework regions, and
(ii) a VL sequence selected from SEQ ID NO: 4 and from variants of SEQ ID NO: 4 having 1, 2 or 3 mutations within the framework regions;
or
(i) a VH sequence selected from SEQ ID NOs: 8 and 9 and from variants of SEQ ID NOs: 8 and 9 having 1, 2 or 3 mutations within the framework regions, and
(ii) a VL sequence selected from SEQ ID NO: 11 and from variants of SEQ ID NO: 11 having 1, 2 or 3 mutations within the framework regions;
or
(i) a VH sequence selected from SEQ ID NOs: 15-18 and from variants of SEQ ID NOs: 15-18 having 1, 2 or 3 mutations within the framework regions, and
(ii) a VL sequence selected from SEQ ID NO: 20 and 22 and from variants of SEQ ID NO: 20 and 22 having 1, 2 or 3 mutations within the framework regions;
or
(i) a VH sequence selected from SEQ ID NOs: 31 and from variants of SEQ ID NOs: 31 having 1, 2 or 3 mutations within the framework regions, and
(ii) a VL sequence selected from SEQ ID NO: 33 and from variants of SEQ ID NO: 33 having 1, 2 or 3 mutations within the framework regions;
or
(i) a VH sequence selected from SEQ ID NOs: 36 and 37 and from variants of SEQ ID NOs: 36 and 37 having 1, 2 or 3 mutations within the framework regions, and
(ii) a VL sequence selected from SEQ ID NO: 39 and from variants of SEQ ID NO: 39 having 1, 2 or 3 mutations within the framework regions;
or
(i) a VH sequence selected from SEQ ID NOs: 43 and 44 and from variants of SEQ ID NOs: 43 and 44 having 1, 2 or 3 mutations within the framework regions, and
(ii) a VL sequence selected from SEQ ID NO: 46 and from variants of SEQ ID NO: 46 having 1, 2 or 3 mutations within the framework regions;
or
(i) a VH sequence selected from SEQ ID NOs: 50 and 51 and from variants of SEQ ID NOs: 50 and 51 having 1, 2 or 3 mutations within the framework regions,
and
(ii) a VL sequence selected from SEQ ID NO: 53 and from variants of SEQ ID NO: 53 having 1, 2 or 3 mutations within the framework regions;
or
(i) a VH sequence selected from SEQ ID NOs: 57 and 58 and from variants of SEQ ID NOs: 57 and 58 having 1, 2 or 3 mutations within the framework regions, and
(ii) a VL sequence selected from SEQ ID NO: 60 and from variants of SEQ ID NO: 60 having 1, 2 or 3 mutations within the framework regions;
or
(i) a VH sequence selected from SEQ ID NOs: 64-66 and from variants of SEQ ID NOs: 64-66 having 1, 2 or 3 mutations within the framework regions, and
(ii) a VL sequence selected from SEQ ID NO: 68 and 69 and from variants of SEQ ID NO: 68 and 69 having 1, 2 or 3 mutations within the framework regions;
or
(i) a VH sequence selected from SEQ ID NOs: 74-77 and from variants of SEQ ID NOs: 74-77 having 1, 2 or 3 mutations within the framework regions, and
(ii) a VL sequence selected from SEQ ID NO: 80 and 81 and from variants of SEQ ID NO: 80 and 81 having 1, 2 or 3 mutations within the framework regions;
or
(i) a VH sequence selected from SEQ ID NOs: 94 and 95 and from variants of SEQ ID NOs: 94 and 95 having 1, 2 or 3 mutations within the framework regions, and
(ii) a VL sequence selected from SEQ ID NO: 97 and from variants of SEQ ID NO: 97 having 1, 2 or 3 mutations within the framework regions;
or
(i) a VH sequence selected from SEQ ID NOs: 101 and 102 and from variants of SEQ ID NOs: 101 and 102 having 1, 2 or 3 mutations within the framework regions, and
(ii) a VL sequence selected from SEQ ID NO: 105 and 106 and from variants of SEQ ID NO: 105 and 106 having 1, 2 or 3 mutations within the framework regions;
or
(i) a VH sequence selected from SEQ ID NOs: 110 and 111 and from variants of SEQ ID NOs: 110 and 111 having 1, 2 or 3 mutations within the framework regions, and
(ii) a VL sequence selected from SEQ ID NO: 113 and from variants of SEQ ID NO: 113 having 1, 2 or 3 mutations within the framework regions;
or
(i) a VH sequence selected from SEQ ID NOs: 117 and 118 and from variants of SEQ ID NOs: 117 and 118 having 1, 2 or 3 mutations within the framework regions, and
(ii) a VL sequence selected from SEQ ID NO: 120 and from variants of SEQ ID NO: 120 having 1, 2 or 3 mutations within the framework regions;
or
(i) a VH sequence selected from SEQ ID NOs: 124 and 125 and from variants of SEQ ID NOs: 124 and 125 having 1, 2 or 3 mutations within the framework regions, and
(ii) a VL sequence selected from SEQ ID NO: 127 and from variants of SEQ ID NO: 127 having 1, 2 or 3 mutations within the framework regions;
or
(i) a VH sequence selected from SEQ ID NOs: 131 and from variants of SEQ ID NOs: 131 having 1, 2 or 3 mutations within the framework regions, and
(ii) a VL sequence selected from SEQ ID NO: 133 and from variants of SEQ ID NO: 133 having 1, 2 or 3 mutations within the framework regions;
or
(i) a VH sequence selected from SEQ ID NOs: 136 and 137 and from variants of SEQ ID NOs: 136 and 137 having 1, 2 or 3 mutations within the framework regions, and
(ii) a VL sequence selected from SEQ ID NO: 140 and 141 and from variants of SEQ ID NO: 140 and 141 having 1, 2 or 3 mutations within the framework regions;
or
(i) a VH sequence selected from SEQ ID NOs: 145 and 146 and from variants of SEQ ID NOs: 145 and 146 having 1, 2 or 3 mutations within the framework regions, and
(ii) a VL sequence selected from SEQ ID NO: 148 and from variants of SEQ ID NO: 148 having 1, 2 or 3 mutations within the framework regions;
or
(i) a VH sequence selected from SEQ ID NOs: 152 and 153 and from variants of SEQ ID NOs: 152 and 153 having 1, 2 or 3 mutations within the framework regions, and
(ii) a VL sequence selected from SEQ ID NO: 155 and from variants of SEQ ID NO: 155 having 1, 2 or 3 mutations within the framework regions;
or
(i) a VH sequence selected from SEQ ID NOs: 159-161 and from variants of SEQ ID NOs: 159-161 having 1, 2 or 3 mutations within the framework regions, and
(ii) a VL sequence selected from SEQ ID NO: 163 and 164 and from variants of SEQ ID NO: 163 and 164 having 1, 2 or 3 mutations within the framework regions;
or
(i) a VH sequence selected from SEQ ID NOs: 170-173 and from variants of SEQ ID NOs: 170-173 having 1, 2 or 3 mutations within the framework regions, and
(ii) a VL sequence selected from SEQ ID NO: 175 and 176 and from variants of SEQ ID NO: 175 and 176 having 1, 2 or 3 mutations within the framework regions;
or
(i) a VH sequence selected from SEQ ID NOs: 182 and 183 and from variants of SEQ ID NOs: 182 and 183 having 1, 2 or 3 mutations within the framework regions, and
(ii) a VL sequence selected from SEQ ID NO: 185 and from variants of SEQ ID NO: 185 having 1, 2 or 3 mutations within the framework regions;
or
(i) a VH sequence selected from SEQ ID NOs: 190 and 193 and from variants of SEQ ID NOs: 190 and 193 having 1, 2 or 3 mutations within the framework regions, and
(ii) a VL sequence selected from SEQ ID NO: 195 and 196 and from variants of SEQ ID NO: 195 and 196 having 1, 2 or 3 mutations within the framework regions;
or
(i) a VH sequence selected from SEQ ID NOs: 202-205 and from variants of SEQ ID NOs: 202-205 having 1, 2 or 3 mutations within the framework regions, and
(ii) a VL sequence selected from SEQ ID NO: 207 and 208 and from variants of SEQ ID NO: 207 and 208 having 1, 2 or 3 mutations within the framework regions;
or
(i) a VH sequence selected from SEQ ID NOs: 214-217 and from variants of SEQ ID NOs: 214-217 having 1, 2 or 3 mutations within the framework regions, and
(ii) a VL sequence selected from SEQ ID NO: 219 and 220 and from variants of SEQ ID NO: 219 and 220 having 1, 2 or 3 mutations within the framework regions;
or
(i) a VH sequence selected from SEQ ID NOs: 226 and 227 and from variants of SEQ ID NOs: 226 and 227 having 1, 2 or 3 mutations within the framework regions, and
(ii) a VL sequence selected from SEQ ID NO: 229 and from variants of SEQ ID NO: 229 having 1, 2 or 3 mutations within the framework regions;
or
(i) a VH sequence selected from SEQ ID NOs: 233-236 and from variants of SEQ ID NOs: 233-236 having 1, 2 or 3 mutations within the framework regions, and
(ii) a VL sequence selected from SEQ ID NO: 239 and 240 and from variants of SEQ ID NO: 239 and 240 having 1, 2 or 3 mutations within the framework regions;
or
(i) a VH sequence selected from SEQ ID NOs: 247-250 and from variants of SEQ ID NOs: 247-250 having 1, 2 or 3 mutations within the framework regions, and
(ii) a VL sequence selected from SEQ ID NO: 252 and from variants of SEQ ID NO: 252 having 1, 2 or 3 mutations within the framework regions;
or
(i) a VH sequence selected from SEQ ID NOs: 257 and 258 and from variants of SEQ ID NOs: 257 and 258 having 1, 2 or 3 mutations within the framework regions, and
(ii) a VL sequence selected from SEQ ID NO: 260 and from variants of SEQ ID NO: 260 having 1, 2 or 3 mutations within the framework regions;
or
(i) a VH sequence selected from SEQ ID NOs: 264-267 and from variants of SEQ ID NOs: 264-267 having 1, 2 or 3 mutations within the framework regions, and
(ii) a VL sequence selected from SEQ ID NO: 269 and 270 and from variants of SEQ ID NO: 269 and 270 having 1, 2 or 3 mutations within the framework regions;
or
(i) a VH sequence selected from SEQ ID NOs: 275-278 and from variants of SEQ ID NOs: 275-278 having 1, 2 or 3 mutations within the framework regions, and
(ii) a VL sequence selected from SEQ ID NO: 280 and 281 and from variants of SEQ ID NO: 280 and 281 having 1, 2 or 3 mutations within the framework regions;
or
(i) a VH sequence selected from SEQ ID NOs: 286-291 and from variants of SEQ ID NOs: 286-291 having 1, 2 or 3 mutations within the framework regions, and
(ii) a VL sequence selected from SEQ ID NO: 293 and 294 and from variants of SEQ ID NO: 293 and 294 having 1, 2 or 3 mutations within the framework regions;
or
(i) a VH sequence selected from SEQ ID NOs: 302 and from variants of SEQ ID NOs: 302 having 1, 2 or 3 mutations within the framework regions, and
(ii) a VL sequence selected from SEQ ID NO: 304 and from variants of SEQ ID NO: 304 having 1, 2 or 3 mutations within the framework regions;
or
(i) a VH sequence selected from SEQ ID NOs: 307 and from variants of SEQ ID NOs: 307 having 1, 2 or 3 mutations within the framework regions, and
(ii) a VL sequence selected from SEQ ID NO: 309 and from variants of SEQ ID NO: 309 having 1, 2 or 3 mutations within the framework regions;
or
(i) a VH sequence selected from SEQ ID NOs: 312 and 313 and from variants of SEQ ID NOs: 312 and 313 having 1, 2 or 3 mutations within the framework regions, and
(ii) a VL sequence selected from SEQ ID NO: 317-319 and from variants of SEQ ID NO: 317-319 having 1, 2 or 3 mutations within the framework regions;
or
(i) a VH sequence selected from SEQ ID NOs: 327-330 and from variants of SEQ ID NOs: 327-330 having 1, 2 or 3 mutations within the framework regions, and
(ii) a VL sequence selected from SEQ ID NO: 332 and from variants of SEQ ID NO: 332 having 1, 2 or 3 mutations within the framework regions;
or
(i) a VH sequence selected from SEQ ID NOs: 338-341 and from variants of SEQ ID NOs: 338-341 having 1, 2 or 3 mutations within the framework regions, and
(ii) a VL sequence selected from SEQ ID NO: 343 and from variants of SEQ ID NO: 343 having 1, 2 or 3 mutations within the framework regions;
or
(i) a VH sequence selected from SEQ ID NOs: 349 and 350 and from variants of SEQ ID NOs: 349 and 350 having 1, 2 or 3 mutations within the framework regions, and
(ii) a VL sequence selected from SEQ ID NO: 352 and from variants of SEQ ID NO: 352 having 1, 2 or 3 mutations within the framework regions;
or
(i) a VH sequence selected from SEQ ID NOs: 356 and 357 and from variants of SEQ ID NOs: 356 and 357 having 1, 2 or 3 mutations within the framework regions, and
(ii) a VL sequence selected from SEQ ID NO: 359 and from variants of SEQ ID NO: 359 having 1, 2 or 3 mutations within the framework regions;
or
(i) a VH sequence selected from SEQ ID NOs: 363 and 364 and from variants of SEQ ID NOs: 363 and 364 having 1, 2 or 3 mutations within the framework regions, and
(ii) a VL sequence selected from SEQ ID NO: 366 and from variants of SEQ ID NO: 366 having 1, 2 or 3 mutations within the framework regions;
or
(i) a VH sequence selected from SEQ ID NOs: 370 and 371 and from variants of SEQ ID NOs: 370 and 371 having 1, 2 or 3 mutations within the framework regions, and
(ii) a VL sequence selected from SEQ ID NO: 373 and from variants of SEQ ID NO: 373 having 1, 2 or 3 mutations within the framework regions;
or
(i) a VH sequence selected from SEQ ID NOs: 377 and 378 and from variants of SEQ ID NOs: 377 and 378 having 1, 2 or 3 mutations within the framework regions, and
(ii) a VL sequence selected from SEQ ID NO: 380 and from variants of SEQ ID NO: 380 having 1, 2 or 3 mutations within the framework regions;
or
(i) a VH sequence selected from SEQ ID NOs: 384 and 385 and from variants of SEQ ID NOs: 384 and 385 having 1, 2 or 3 mutations within the framework regions, and
(ii) a VL sequence selected from SEQ ID NO: 387 and from variants of SEQ ID NO: 387 having 1, 2 or 3 mutations within the framework regions;
or
(i) a VH sequence selected from SEQ ID NOs: 391 and 392 and from variants of SEQ ID NOs: 391 and 392 having 1, 2 or 3 mutations within the framework regions, and
(ii) a VL sequence selected from SEQ ID NO: 394 and from variants of SEQ ID NO: 394 having 1, 2 or 3 mutations within the framework regions;
or
(i) a VH sequence selected from SEQ ID NOs: 398 and 399 and from variants of SEQ ID NOs: 398 and 399 having 1, 2 or 3 mutations within the framework regions, and
(ii) a VL sequence selected from SEQ ID NO: 401 and from variants of SEQ ID NO: 401 having 1, 2 or 3 mutations within the framework regions;
or
(i) a VH sequence selected from SEQ ID NOs: 405 and 406 and from variants of SEQ ID NOs: 405 and 406 having 1, 2 or 3 mutations within the framework regions, and
(ii) a VL sequence selected from SEQ ID NO: 408 and from variants of SEQ ID NO: 408 having 1, 2 or 3 mutations within the framework regions;
or
(i) a VH sequence selected from SEQ ID NOs: 412 and 413 and from variants of SEQ ID NOs: 412 and 413 having 1, 2 or 3 mutations within the framework regions, and
(ii) a VL sequence selected from SEQ ID NO: 415 and from variants of SEQ ID NO: 415 having 1, 2 or 3 mutations within the framework regions;
or
(i) a VH sequence selected from SEQ ID NOs: 419 and 420 and from variants of SEQ ID NOs: 419 and 420 having 1, 2 or 3 mutations within the framework regions, and
(ii) a VL sequence selected from SEQ ID NO: 422 and from variants of SEQ ID NO: 422 having 1, 2 or 3 mutations within the framework regions;
or
(i) a VH sequence selected from SEQ ID NOs: 426 and 427 and from variants of SEQ ID NOs: 426 and 427 having 1, 2 or 3 mutations within the framework regions, and
(ii) a VL sequence selected from SEQ ID NO: 429 and from variants of SEQ ID NO: 429 having 1, 2 or 3 mutations within the framework regions;
or
(i) a VH sequence selected from SEQ ID NOs: 433 and 434 and from variants of SEQ ID NOs: 433 and 434 having 1, 2 or 3 mutations within the framework regions, and
(ii) a VL sequence selected from SEQ ID NO: 436 and from variants of SEQ ID NO: 436 having 1, 2 or 3 mutations within the framework regions;
or
(i) a VH sequence selected from SEQ ID NOs: 440 and 441 and from variants of SEQ ID NOs: 440 and 441 having 1, 2 or 3 mutations within the framework regions, and
(ii) a VL sequence selected from SEQ ID NO: 443 and from variants of SEQ ID NO: 443 having 1, 2 or 3 mutations within the framework regions;
or
(i) a VH sequence selected from SEQ ID NOs: 447 and 448 and from variants of SEQ ID NOs: 447 and 448 having 1, 2 or 3 mutations within the framework regions, and
(ii) a VL sequence selected from SEQ ID NO: 450 and from variants of SEQ ID NO: 450 having 1, 2 or 3 mutations within the framework regions;
or
(i) a VH sequence selected from SEQ ID NOs: 454 and 455 and from variants of SEQ ID NOs: 454 and 455 having 1, 2 or 3 mutations within the framework regions, and
(ii) a VL sequence selected from SEQ ID NO: 457 and from variants of SEQ ID NO: 457 having 1, 2 or 3 mutations within the framework regions;
or
(i) a VH sequence selected from SEQ ID NOs: 461 and 462 and from variants of SEQ ID NOs: 461 and 462 having 1, 2 or 3 mutations within the framework regions, and
(ii) a VL sequence selected from SEQ ID NO: 464 and from variants of SEQ ID NO: 464 having 1, 2 or 3 mutations within the framework regions;
or
(i) a VH sequence selected from SEQ ID NOs: 468 and 469 and from variants of SEQ ID NOs: 468 and 469 having 1, 2 or 3 mutations within the framework regions, and
(ii) a VL sequence selected from SEQ ID NO: 471 and from variants of SEQ ID NO: 471 having 1, 2 or 3 mutations within the framework regions;
or
(i) a VH sequence selected from SEQ ID NOs: 475 and 476 and from variants of SEQ ID NOs: 475 and 476 having 1, 2 or 3 mutations within the framework regions, and
(ii) a VL sequence selected from SEQ ID NO: 478 and from variants of SEQ ID NO: 478 having 1, 2 or 3 mutations within the framework regions;
or
(i) a VH sequence selected from SEQ ID NOs: 482 and 483 and from variants of SEQ ID NOs: 482 and 483 having 1, 2 or 3 mutations within the framework regions, and
(ii) a VL sequence selected from SEQ ID NO: 485 and from variants of SEQ ID NO: 485 having 1, 2 or 3 mutations within the framework regions;
or
(i) a VH sequence selected from SEQ ID NOs: 489 and 490 and from variants of SEQ ID NOs: 489 and 490 having 1, 2 or 3 mutations within the framework regions, and
(ii) a VL sequence selected from SEQ ID NO: 492 and from variants of SEQ ID NO: 492 having 1, 2 or 3 mutations within the framework regions;
or
wherein said antibody variable domain is an scFv, which has a sequence selected from SEQ ID NOs: 6, 7, 13, 14, 24, 25, 27, 28, 29, 30, 35, 41, 42, 48, 49, 55, 56, 62, 63, 71-73, 83, 84, 86, 87, 89, 90, 92, 93, 99, 100, 108, 109, 115, 116, 122, 123, 129, 130, 135, 143, 144, 150, 151, 157, 158, 166, 168, 169, 178-181, 189, 198-201, 210-213, 222-225, 231, 232, 242, 243, 245, 246, 254-256, 262, 263, 272-274, 283-285, 296-298, 300, 301, 306, 311, 321-326, 334-337, 345-348, 354, 355, 361, 362, 368, 369, 375, 376, 382, 383, 389, 390, 396, 397, 403, 404, 410, 411, 417, 418, 424, 425, 431, 432, 438, 439, 445, 446, 452, 453, 459, 460, 466, 467, 473, 474, 480, 481, 487, 488, 494, 495 and from variants of SEQ ID NOs: 6, 7, 13, 14, 24, 25, 27, 28, 29, 30, 35, 41, 42, 48, 49, 55, 56, 62, 63, 71-73, 83, 84, 86, 87, 89, 90, 92, 93, 99, 100, 108, 109, 115, 116, 122, 123, 129, 130, 135, 143, 144, 150, 151, 157, 158, 166, 168, 169, 178-181, 189, 198-201, 210-213, 222-225, 231, 232, 242, 243, 245, 246, 254-256, 262, 263, 272-274, 283-285, 296-298, 300, 301, 306, 311, 321-326, 334-337, 345-348, 354, 355, 361, 362, 368, 369, 375, 376, 382, 383, 389, 390, 396, 397, 403, 404, 410, 411, 417, 418, 424, 425, 431, 432, 438, 439, 445, 446, 452, 453, 459, 460, 466, 467, 473, 474, 480, 481, 487, 488, 494, 495 having 1, 2, 3, 4 or 5 mutations within the framework regions.

10. The antibody variable domain of claim 1, wherein the variable heavy chain (VH) comprises a serine (S) at amino acid position 101 and a lysine (K) at amino acid position 146 (AHo numbering).

11. An antibody comprising one or more antibody variable domains as defined in claim 1.

12. The antibody of claim 11, wherein said antibody is a single chain antibody having a sequence selected from SEQ ID NOs: 545-550, 552, 553, 743, 555, 556, 558, 559, 561, 562, 564, 565, 567, 568, 570, 571, 573, 574, 576, 577, 579, 580, 582, 583, 585, 586, 588, 589, 591, 592, 594, 595, 603, 604, 606, 607, 645, 646, 648 and 649 and from variants of SEQ ID NOs: 545-550, 552, 553, 743, 555, 556, 558, 559, 561, 562, 564, 565, 567, 568, 570, 571, 573, 574, 576, 577, 579, 580, 582, 583, 585, 586, 588, 589, 591, 592, 594, 595, 603, 604, 606, 607, 645, 646, 648 and 649 having 1, 2, 3, 4 or 5 mutations within the framework regions;
or wherein said antibody is a heterodimer consisting of two chains having a pair of sequences selected from the following SEQ ID NOs: 597 and 600; 598 and 601; 609 and 612; 610 and 613; 615 and 618; 616 and 619; 621 and 624; 622 and 625; 627 and 630; 627 and 631; 633 and 636; 634 and 637; 639 and 642; 640 and 643; 645 and 648; 646 and 649; 651 and 654; 652 and 655; 657 and 660; 658 and 661; and from variants of SEQ ID NOs: 597 and 600; 598 and 601; 609 and 612; 610 and 613; 615 and 618; 616 and 619; 621 and 624; 622 and 625; 627 and 630; 627 and 631; 633 and 636; 634 and 637; 639 and 642; 640 and 643; 645 and 648; 646 and 649; 651 and 654; 652 and 655; 657 and 660; 658 and 661; wherein each of the sequences in said sequence pairs has 1, 2, 3, 4 or 5 mutations within the framework regions.

13. A pharmaceutical composition comprising the antibody of claim 1 and a pharmaceutically acceptable carrier.

14. A method for generating a modified antibody variable domain, wherein the method comprises the steps of
1) providing an unmodified antibody variable domain, which binds to a target antigen, comprising:
(i) a variable heavy chain (VH) comprising from N-terminus to C-terminus, the regions HFW1-HCDR1-HFW2-HCDR2-HFW3-HCDR3-HFW4, wherein each HFW designates a heavy chain framework region, and each HCDR designates a heavy chain complementarity-determining region; and
(ii) a variable light chain (VL), wherein the variable light chain comprises, from N-terminus to C-terminus, the regions LFW1-LCDR1-LFW2-LCDR2-LFW3-LCDR3-LFW4, wherein each LFW designates a light chain framework region, and each LCDR designates a light chain complementarity-determining region, wherein the light chain complementarity-determining region LCDR2 does not comprise a glycine (G) at both amino acid position 67 and 68 (AHo numbering);
2) introducing into the light chain complementarity-determining region LCDR2 of the unmodified antibody variable domain provided in step 1) a glycine (G) at amino acid position 67 and/or a glycine (G) at amino acid position 68 (AHo numbering), such that there is a glycine (G) at both amino acid positions 67 and 68;
wherein said LCDR2 is defined by light chain amino acid positions 58 to 72 according to the AHo numbering scheme, resulting in a maximum length of 15 amino acids.

15. The method of claim 14, wherein the LCDR2 of the unmodified antibody variable domain comprises:
(i) an alanine (A), threonine (T), serine (S) or glycine (G), in particular an alanine (A) or threonine (T) at position 67 (AHo numbering);
(ii) a serine (S), tyrosine (Y) or phenylalanine (F), in particular a serine (S) or tyrosine (Y) at position 68 (AHo numbering);
and optionally
(iii) a lysine (K), threonine (T), serine (S), isoleucine (1), aspartate (D), asparagine (N), phenylalanine (F) or tyrosine (Y), in particular a lysine (K), threonine (T), serine (S), aspartate (D), asparagine (N) or tyrosine (Y) at position 69 (AHo numbering);
(iv) an alanine (A), glycine (G), valine (V), threonine (T), proline (P), tyrosine (Y), aspartate (D) or glutamate (E), in particular an alanine (A), glycine (G), threonine (T) or glutamate (E) at position 71 (AHo numbering).

16. The method of claim 14, wherein the LCDR2 of the unmodified antibody variable domain comprises:
(i) lysine (K), arginine (R), serine (S), threonine (T), alanine (A), leucine (L), glycine (G), glutamine (Q), glutamate (E), tyrosine (Y) or aspartate (D), in particular a lysine (K), arginine (R), serine (S), tyrosine (Y), glycine (G) or aspartate (D) at position 58 (AHo numbering);
(ii) an alanine (A), threonine (T), serine (S) or glycine (G), in particular an alanine (A) or threonine (T) at position 67 (AHo numbering);
(iii) a serine (S), tyrosine (Y) or phenylalanine (F), in particular a serine (S) or tyrosine (Y) at position 68 (AHo numbering);
(iv) a lysine (K), threonine (T), serine (S), isoleucine (1), aspartate (D), asparagine (N), phenylalanine (F) or tyrosine (Y), in particular a lysine (K), threonine (T), serine (S), aspartate (D), asparagine (N) or tyrosine (Y) at position 69 (AHo numbering);
(v) a lysine (K), leucine (L), methionine (M) or arginine (R), in particular a leucine (L) at position 70 (AHo numbering);
(vi) an alanine (A), glycine (G), valine (V), threonine (T), proline (P), tyrosine (Y), aspartate (D) or glutamate (E), in particular an alanine (A), glycine (G), threonine (T) or glutamate (E) at position 71 (AHo numbering);
(vii) a serine (S), threonine (T) or tyrosine (Y), in particular a serine (S) at position 72 (AHo numbering).

17. The method of claim 14, wherein the method comprises that additional step of
3) Introducing into the variable heavy chain (VH) of the unmodified antibody variable domain provided in step 1) a serine (S) at amino acid position 101 and a lysine (K) at amino acid position 146 (AHo numbering).

18. A method for generating a modified antibody from an unmodified antibody, the method comprises the steps of
1) selecting one or more antibody variable domains comprised in said unmodified antibody, which do not comprise a glycine (G) at both amino acid position 67 and 68 (AHo numbering) in the LCDR2, for modification;
2) introducing into each of said one or more antibody variable domains selected in step 1) a glycine (G) at amino acid position 67 and/or a glycine (G) at amino acid position 68 (AHo numbering) in the LCDR2, such that there is a glycine (G) at both amino acid positions 67 and 68 (AHo numbering) in the LCDR2,
wherein said LCDR2 is defined by light chain amino acid positions 58 to 72 according to the AHo numbering scheme, resulting in a maximum length of 15 amino acids.

19. The method of claim 18, wherein the method comprises that additional step of
3) introducing into the variable heavy chain (VH) of each of said one or more antibody variable domains selected in step 1) a serine (S) at amino acid position 101 and a lysine (K) at amino acid position 146 (AHo numbering).

20. The method of claim 14, wherein said modified antibody variable domain or said modified antibody comprises a decreased number of 15mer peptide stretches that exhibit a median percentile rank of less than 20, as determined from the predicted individual binding strengths (scoring ranks) of said 15mer peptide to each one of the 27 MHC class II alleles that are most common in the general population using the algorithm NetMHCIIpan-3.1, when compared to the respective unmodified antibody variable domain.

21. (canceled)
